(19) United States
(12) Patent Application Publication
(10) Pub. No.: US 2026/0070878 A1
(43) Pub. Date: Mar. 12, 2026

(54) TNKS2 INHIBITORS

(71) Applicants: CORNELL UNIVERSITY, Ithaca, NY (US); SANDERS TRI-INSTITUTIONAL THERAPEUTICS DISCOVERY INSTITUTE, New York, NY (US)

(72) Inventors: Lukas DOW, Larchmont, NY (US); Jill ZIMMERMAN, New York, NY (US); David J. HUGGINS, New York, NY (US); Efrat FINKIN-GRONER, Pleasantville, NY (US); John GINN, New Milford, CT (US); Nigel LIVERTON, San Antonio, TX (US); Rui LIANG, East Brunswick, NJ (US); Shan SUN, Sherman, CT (US); Tanweer A. KHAN, Bridgewater, NJ (US); Yoshiyuki FUKASE, San Diego, CA (US)

(21) Appl. No.: 19/258,561

(22) Filed: Jul. 2, 2025

Related U.S. Application Data
(60) Provisional application No. 63/667,306, filed on Jul. 3, 2024.

(51) Int. Cl. C07D 239/91; A61K 31/517; A61K 31/5377; A61K 31/538; A61K 31/541; A61P 35/00; C07D 401/12; C07D 401/14; C07D 403/12; C07D 405/12; C07D 413/12; C07D 417/12; C07D 471/04

(52) U.S. Cl. CPC C07D 239/91; A61K 31/517; A61K 31/5377; A61K 31/538; A61K 31/541; A61P 35/00; C07D 401/12; C07D 401/14; C07D 403/12; C07D 405/12; C07D 413/12; C07D 417/12; C07D 471/04

(57) ABSTRACT

The present disclosure is directed to compounds of Formula (I):

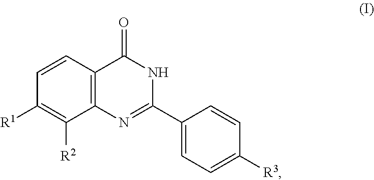

where R¹, R², and R³ are as defined herein. The present disclosure is also directed to a pharmaceutical composition comprising these compounds, as well as the use of these compounds for the treatment of cancer or the inhibition of Tankyrase 2 (TNKS2) activity.

This application claims the priority benefit of U.S. Provisional Patent Application Ser. No. 63/667,306, filed Jul. 3, 2024, which is hereby incorporated by reference in its entirety.

This invention was made with government support under 1R01CA273106 awarded by the National Institutes of Health. The government has certain rights in the invention.

The instant application contains a computer readable Sequence Listing which has been submitted electronically in XML format (“Sequence Listing XML”) and is hereby incorporated by reference in its entirety. The Sequence Listing XML, created on Nov. 11, 2025, is named 147402009432.xml and is 78,614 bytes in size.

### FIELD

The present application relates to TNKS2 inhibitors.

### BACKGROUND

Hyperactivation of the WNT pathway is a near-ubiquitous feature of colorectal cancer (CRC). Suppressing WNT hyperactivation through genetic or pharmacologic approaches can prevent tumor growth and/or drive sustained tumor regression (Dow et al., “Apc Restoration Promotes Cellular Differentiation and Reestablishes Crypt Homeostasis in Colorectal Cancer,” Cell 161:1539-1552 (2015); O'Rourke et al., “Transplantation of Engineered Organoids Enables Rapid Generation of Metastatic Mouse Models of Colorectal Cancer,” Nat. Biotechnol. 35:577-582 (2017); Schatoff et al., “Distinct Colorectal Cancer-Associated APC Mutations Dictate Response to Tankyrase Inhibition,” Cancer Discov. 9:1358-1371 (2019); Han et al., “Lineage Reversion Drives WNT Independence in Intestinal Cancer,” Cancer Discov. 10:1590-1609 (2020); Storm et al., “Targeting PTPRK-RSPO3 Colon Tumours Promotes Differentiation and Loss of Stem-Cell Function,” Nature 529, 97-100 (2016); Madan et al., “Wnt Addiction of Genetically Defined Cancers Reversed by PORCN Inhibition,” Oncogene 35:2197-2207 (2016); Scholer-Dahirel et al., “Maintenance of Adenomatous Polyposis Coli (APC)-Mutant Colorectal Cancer is Dependent on Wnt/Beta-Catenin Signaling,” Proc. Natl. Acad. Sci. USA 108: 17135-17140 (2011); Faux et al., “Restoration of Full-Length Adenomatous Polyposis Coli (APC) Protein in a Colon Cancer Cell Line Enhances Cell Adhesion,” J. Cell Sci. 117:427-439 (2004)). WNT activation is also implicated in the pathogenesis of numerous other tumor types, including breast, lung, prostate, gastric, ovarian, and hepatocellular cancers (P. Polakis, “The Many Ways of Wnt in Cancer,” Curr. Opin. Genet. Dev. 17:45-51 (2007)), drives therapy resistance (Arqués et al., “Tankyrase Inhibition Blocks Wnt/β-Catenin Pathway and Reverts Resistance to PI3K and AKT Inhibitors in the Treatment of Colorectal Cancer,” Clin. Cancer Res. 22:644-656 (2016); Fong et al., “BET Inhibitor Resistance Emerges from Leukaemia Stem Cells,” Nature 525:538-542 (2015); Rathert et al., “Transcriptional Plasticity Promotes Primary and Acquired Resistance to BET Inhibition,” Nature 525:543-547 (2015); Schoumacher et al., “Inhibiting Tankyrases Sensitizes KRAS-Mutant Cancer Cells to MEK Inhibitors Via FGFR2 Feedback Signaling,” Cancer Res. 74:3294-3305 (2014)), and can suppress activity of immune checkpoint inhibitors (Spranger et al., “Melanoma-Intrinsic β-Catenin Signalling Prevents Anti-Tumour Immunity,” Nature 523, 231-235 (2015); Ruiz de Galarreta et al., “β-Catenin Activation Promotes Immune Escape and Resistance to Anti-PD-1 Therapy in Hepatocellular Carcinoma,” Cancer Discov. 9:1124-1141 (2019)). Thus, while there is substantial clinical potential for targeting WNT signaling in cancer, early clinical studies, and in vivo pre-clinical work with WNT inhibitors have revealed significant on-target toxicities in normal tissues (Lau et al., “A Novel Tankyrase Small-Molecule Inhibitor Suppresses APC Mutation-Driven Colorectal Tumor Growth,” Cancer Res. 73:3132-3144 (2013); Zhong et al., “Tankyrase Inhibition Causes Reversible Intestinal Toxicity in Mice with a Therapeutic Index <1,” Toxicol. Pathol. 44:267-278 (2016)).

One strategy to suppress WNT signaling is through inhibition of Tankyrase activity. Tankyrase (TNKS; hereafter TNKS1 for clarity) and Tankyrase 2 (TNKS2) are functionally redundant PARP-family enzymes that promote WNT signaling through the PARylation and degradation of the negative regulator AXIN1 (Huang et al., “Tankyrase Inhibition Stabilizes Axin and Antagonizes Wnt Signalling,” Nature 461:614-620 (2009)) and have been shown to modulate YAP signaling by targeting AMOT proteins (Wang et al., “Tankyrase Inhibitors Target YAP by Stabilizing Angiomotin Family Proteins,” Cell Rep. 13:524-532 (2015)). Many small molecule compounds have been developed that target TNKS1/TNKS2 enzymes and these compounds can effectively stabilize AXIN1, downregulate WNT signaling, and suppress tumor cell proliferation in multiple cancer types (Lau et al., “A Novel Tankyrase Small-Molecule Inhibitor Suppresses APC Mutation-Driven Colorectal Tumor Growth,” Cancer Res. 73:3132-3144 (2013); Zhong et al., “Tankyrase Inhibition Causes Reversible Intestinal Toxicity in Mice with a Therapeutic Index <1,” Toxicol. Pathol. 44:267-278 (2016); Huang et al., “Tankyrase Inhibition Stabilizes Axin and Antagonizes Wnt Signalling,” Nature 461:614-620 (2009); Chen et al., “Small Molecule-Mediated Disruption of Wnt-Dependent Signaling in Tissue Regeneration and Cancer,” Nat. Chem. Biol. 5:100-107 (2009); Waaler et al., “A Novel Tankyrase Inhibitor Decreases Canonical Wnt Signaling in Colon Carcinoma Cells and Reduces Tumor Growth in Conditional Apc Mutant Mice,” Cancer Res. 72:2822-2832 (2012); Shultz et al., “Identification of NVP-TNKS656: the Use of Structure-Efficiency Relationships to Generate a Highly Potent, Selective, and Orally Active Tankyrase Inhibitor,” J. Med. Chem. 56:6495-6511 (2013); C. C. Mehta and H. G. Bhatt, “Tankyrase Inhibitors as Antitumor Agents: A Patent Update (2013-2020),” Expert Opin. Ther. Pat. 31:645-661 (2021)). However, like other broad WNT inhibitors, potent TNKS1/TNKS2 inhibitors cause dose-limiting tissue toxicity in vivo, limiting their therapeutic potential (Lau et al., “A Novel Tankyrase Small-Molecule Inhibitor Suppresses APC Mutation-Driven Colorectal Tumor Growth,” Cancer Res. 73:3132-3144 (2013); Zhong et al., “Tankyrase Inhibition Causes Reversible Intestinal Toxicity in Mice with a Therapeutic Index <1,” Toxicol. Pathol. 44:267-278 (2016)).

The present disclosure is directed to overcoming these and other deficiencies in the art.

### SUMMARY

A first aspect of the present disclosure relates to a compound of Formula (I):

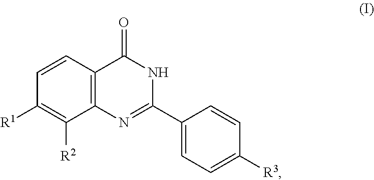

where
R¹ is selected from the group consisting of H, C₁₋₆ alkyl, C₁₋₆ alkoxy, —Oaryl, aryl, heterocyclyl, —NR⁴R⁵, —NHC(O)NHR⁵, —CONR⁶R⁷, and —NR⁸CO(CH₂)ₘR⁹, wherein the C₁₋₆ alkyl, C₁₋₆ alkoxy, —Oaryl, aryl, and heterocyclyl can be optionally substituted from 1 to 3 times with R′;

R² is selected from the group consisting of H, C₁₋₆ alkyl, C₁₋₆ alkoxy, —Oaryl, aryl, heterocyclyl, —NR⁴R⁵, —NHC(O)NHR⁵, —CONR⁶R⁷, and —NR⁸CO(CH₂)ₘR⁹, wherein the C₁₋₆ alkyl, C₁₋₆ alkoxy, —Oaryl, aryl, and heterocyclyl can be optionally substituted from 1 to 3 times with R′;

R′ is independently selected at each occurrence thereof from the group consisting of —OH, ═O, —NH2, halogen, —NHCO₂R¹⁰, —CONR¹¹R¹², —NHCOR¹³, —NHR¹⁴, C₁₋₆ alkoxy, C₁₋₆ alkyl, aryl, biaryl, heteroaryl, —Oaryl, —Oheteroaryl, and heterocyclyl, wherein heteroaryl and heterocyclyl can be optionally substituted from 1 to 3 times with ═O;
R³ is C₁₋₆ alkyl optionally substituted from 1 to 3 times with a substituent independently selected at each occurrence thereof from the group consisting of OH, CF₃, and —O—(CH₂)ₙOH;
R⁴ is selected from the group consisting of H and C₁₋₆ alkyl;
R⁵ is selected from the group consisting of H, C₁₋₆ alkyl, aryl, benzyl, and heteroaryl, wherein C₁₋₆ alkyl can be optionally substituted from 1 to 3 times with a substituent selected independently at each occurrence thereof from the group consisting of —OH and aryl;
R⁶ is H or C₁₋₆ alkyl;
R⁷ is H or C₁₋₆ alkyl;
R⁸ is H or C₁₋₆ alkyl;
R⁹ is selected from the group consisting of C₁₋₆ alkyl, C₁₋₆ alkoxy, —Oaryl, —Oheteroaryl, C₃₋₁₂ cycloalkyl, aryl, biaryl, heterocyclyl, arylalkyl, heteroaryl, and —NHC₁₋₆ alkyl, wherein C₁₋₆ alkyl, Oaryl, C₃₋₁₂ cycloalkyl, heterocyclyl, aryl, biaryl, arylalkyl, and heteroaryl can be optionally substituted from 1 to 3 times with R¹⁵;
R¹⁰ is selected from the group consisting of H, C₁₋₆ alkyl, and benzyl;
R¹¹ is H or C₁₋₆ alkyl;
R¹² is selected from the group consisting of H, C₁₋₆ alkyl, and aryl, wherein C₁₋₆ alkyl can be optionally substituted with C₁₋₆ alkoxy;
R¹³ is selected from the group consisting of H, C₁₋₆ alkyl, aryl, and —Obenzyl, wherein C₁₋₆ alkyl can be optionally substituted from 1 to 3 times with a substituent independently selected from —NHCOR¹⁶, aryl, benzyl, and —NR¹⁷R¹⁸;
R¹⁴ is selected from the group consisting of H, C₁₋₆ alkyl, heterocyclyl, and heteroaryl, wherein heterocyclyl and heteroaryl can be optionally substituted with C₁₋₆ alkyl or —COR¹⁹;
R¹⁵ is independently selected at each occurrence from the group consisting of ═O, —OH, halogen, —CN, —COC₁₋₆ alkyl, —COOH, —COOC₁₋₆ alkyl, aryl, C₁₋₆ alkyl, C₁₋₆ alkoxy, —CONHR¹⁶, —C(O)R²⁰, —C(O)aryl, benzyl, —Oaryl, —Obenzyl, —SO₂Me, heterocyclyl, heteroaryl, —OCHF₂, —OCF₂CHF₂, and —OCH₂CF₃, wherein C₁₋₆ alkyl, —Oaryl, aryl, heterocyclyl, and heteroaryl can be optionally substituted from 1 to 3 times with a substituent independently selected at each occurrence thereof from the group consisting of —OH, halogen, ═O, —C(O) CH₃, C₁₋₆ alkyl, CF₃, heterocyclyl, C₁₋₆ alkoxy, —NR⁴R⁵, and —CN;
R¹⁶ is C₁₋₆ alkyl optionally substituted with C₁₋₆ alkoxy;
R¹⁷ is H or C₁₋₆ alkyl;
R¹⁸ is H or C₁₋₆ alkyl;
R¹⁹ is selected from the group consisting of C₁₋₆ alkyl, aryl, and —NHC₁₋₆ alkyl;
R²⁰ is —NMe₂ or heterocyclyl, wherein heterocyclyl can be optionally substituted 1 to 3 times with OH or C₁₋₆ alkyl;
n is 1, 2, 3, 4, or 5; and
m is 0 or 1;
with the proviso that both R¹ and R² cannot be H, or an oxide thereof, a pharmaceutically acceptable salt thereof, a solvate thereof, or a prodrug thereof.

A second aspect of the present disclosure relates to a pharmaceutical composition comprising a therapeutically effective amount of the compound according to the present disclosure and a pharmaceutically acceptable carrier.

A third aspect of the present disclosure relates to a method of treating cancer in a subject. This method comprises administering to the subject in need thereof the compound according to the present disclosure.

A fourth aspect of the present disclosure relates to a method of inhibiting Tankyrase 2 (TNKS2) activity. This method comprises contacting a TNKS2 with the compound according to the present disclosure under conditions effective to inhibit TNKS2 activity.

A fifth aspect of the present disclosure relates to a method of treating a condition where it is desired to inhibit TNKS2 activity. This method comprises administering to the subject in need thereof the compound according to the present disclosure.

A sixth aspect of the present disclosure relates to a method of treating a proliferation-related disorder in a subject. This method comprises administering to the subject in need thereof the compound according to the present disclosure.

A seventh aspect of the present disclosure relates to a method of treating abnormal cell growth in a subject. This method comprises administering to the subject in need thereof the compound according to the present disclosure.

During malignant progression, many cancers acquire large-scale chromosomal amplifications and deletions. One of the most frequent genomic alterations in human epithelial cancers are deletions on the short arm of chromosome 8 (8p), which contains the TNKS1 locus (Cai et al., “Loss of Chromosome 8p Governs Tumor Progression and Drug Response by Altering Lipid Metabolism,” Cancer Cell 29:751-766 (2016); Gustafson et al., “Functional Evidence for a Colorectal Cancer Tumor Suppressor Gene at Chromosome 8p22-23 by Monochromosome Transfer,” Cancer Res. 56:5238-5245 (1996); Yaremko et al., “Westbrook, Loss of Heterozygosity from the Short Arm of Chromosome 8 is an Early Event in Breast Cancers,” Genes Chromosomes Cancer 13:186-191 (1995); Wistuba et al., “Allelic Losses at Chromosome 8p21-23 are Early and Frequent Events in the Pathogenesis of Lung Cancer,” Cancer Res. 59:1973-1979 (1999); Xue et al., “A Cluster of Cooperating Tumor-Suppressor Gene Candidates in Chromosomal Deletions,” Proc. Natl. Acad. Sci. USA 109:8212-8217 (2012), which are hereby incorporated by reference in their entirety). Herein is shown that tumors with 8p deletions, which express reduced or no TNKS1 protein, carry an acquired tumor-specific dependence on TNKS2 for cellular Tankyrase activity. In these cells, WNT pathway activity is regulated primarily by TNKS2 and can be diminished by selective suppression of TNKS2. Through rational drug design, a first-in-class TNKS2-selective small molecule inhibitor that suppresses the growth of TNKS1-deleted but not TNKS1-diploid cells was developed. This work represents the first description of a targetable collateral vulnerability in the WNT pathway and demonstrates the feasibility for the development of potent and selective TNKS2 inhibitors as a therapeutic modality.

Hyperactive WNT signaling is a potent cancer driver, but clinical translation of WNT inhibitors has been hampered on-target toxicities. WNT signaling can be constrained through inhibition of the PARP family enzymes Tankyrase 1 (TNKS1) and Tankyrase 2 (TNKS2), however, existing TNKS inhibitors suppress WNT signaling in both tumor and healthy tissues. As shown herein, the loss of chromosome 8p that occurs in approximately half of advanced epithelial malignancies, creates a collateral vulnerability that enables tumor-selective inhibition of Tankyrase activity. 8p loss depletes expression of TNKS1 and creates a tumor-specific dependency on the functionally redundant TNKS2 protein. Through structure-guided drug design, a first-in-class TNKS2-selective inhibitor that can drive selective WNT inhibition in TNKS1-deficient oncogenic cell and organoid models was identified. This work demonstrates a targetable vulnerability in multiple cancer types, providing a new approach to potent and selective WNT-targeted therapies.

### BRIEF DESCRIPTION OF THE DRAWINGS

FIG. 1 shows the ¹³C NMR spectrum for N-[2-[4-(1-hydroxy-1-methyl-ethyl)phenyl]-4-oxo-3H-quinazolin-7-yl]-4-methoxy-6-phenyl-pyridine-3-carboxamide.

FIG. 2 shows the LC-MS chromatram for N-[2-[4-(1-hydroxy-1-methyl-ethyl)phenyl]-4-oxo-3H-quinazolin-7-yl]-4-methoxy-6-phenyl-pyridine-3-carboxamide.

FIGS. 3A-3I show 8p deletion and TNKS1 loss sensitizes cells to TNKS2-selective suppression. FIG. 3A shows the fraction of common epithelial cancers carrying heterozygous or homozygous 8p deletions. Data is derived from the ICGC/TGCA PanCan dataset. FIG. 3B shows that TNKS1 genomic copy number is correlated with TNKS1 transcript expression in CRC. Data is derived from the ICGC/TCGA PanCan dataset. Center line, median; box limits, upper and lower quartiles; whiskers, min to max.

FIG. 3C shows a western blot showing reduced TNKS1 protein expression with 8p deletions in a panel of colorectal (DLD1, SW480, SW1417) and lung (PC9, HCC1171) cancer cell lines. FIG. 3D shows a western blot showing AXIN1 and TNKS1/2 levels following TNKS1, TNKS2, or TNKS1/2 suppression. shTNKS2 induced AXIN1 stabilization in 8p null cells (HCC1171) but not 8p diploid cells (DLD1). FIG. 3E shows a western blot showing TNKS1 protein expression in TNKS1^{WT}, TNKS1^{Het}, and TNKS1^{KO} DLD1s. FIG. 3F shows a western blot showing AXIN1 levels following TNKS1, TNKS2, or TNKS1/2 suppression. shTNKS2 induced AXIN1 stabilization in DLD1 TNKS1^{Het} and TNKS1^{KO} isogenic clones but not TNKS1^{WT} cells. FIG. 3G shows the fraction of viable organoids 4 days after crypt isolation from mice expressing shRNAs for two weeks. shTnks1/2.A and shTnks1/2.B represent two different shRNA combinations (n=7-15, mean with SEM, one-way ANOVA with Tukey correction).

FIG. 3H shows in situ hybridization of Lgr5 expression in the small intestine of Tnks1^{WT} or Tnks1^{KO} mice on dox (shTnks2) or off dox (no shRNA). FIG. 3I shows the fraction of viable organoids 4 days after crypt isolation from mice on dox (shTnks2) or off dox (no shRNA) for three weeks. (n=3-6, mean with SEM, one-way ANOVA with Tukey correction).

FIGS. 4A-4B show TNKS1 expression in 8p-deleted cancers. FIG. 4A shows normalized TNKS1 copy numbers across cancer cell lines. FIG. 4B shows a scatter plot showing TNKS1 copy number (log 2) vs transcript expression across the cancer cell lines. Primary data from DepMap.org.

FIGS. 5A-5C show generation and validation of a Tnks1 KO mouse. FIG. 5A shows a schematic showing dual sgRNAs targeting 5′ and 3′ ends of Tnks1 gene. Created with Biorender.com. FIG. 5B shows examples of modified alleles in founder animals, showing full gene deletion, isolated indels, or no editing. Most full deletion animals showed the same fusion sequence due to microhornology within guide sequences. Created with Biorender.com. FIG. 5C shows PCR genotyping of Tnks1 KO mice. The upper band is amplification of the 5′ terminal. The lower band is amplification of the genomic fusion product following KO.

FIGS. 6A-6B show selective loss of Lgr5 expression in small intestinal crypts. FIG. 6A shows a western blot showing loss of TNKS1 protein in small intestinal organoids derived from Tnks1^{wt}, Tnks1^{Het}, or Tnks1^{KO} mice. FIG. 6B shows in situ hybridization of Lgr5 mRNA expression in the small intestine of shTnks2/Tnks1^{WT}, shTnks2/Tnks1^{Het}, and shTnks2/Tnks1^{KO} mice fed doxycycline chow (200 mg/kg) for 3 weeks or maintained on regular chow (no dox; no shRNA induction). Each image shows a representative section from an independent mouse.

FIGS. 7A-7K show development of TNKS2-selective small molecule inhibitors. FIG. 7A shows surface displacement of TNKS2 catalytic domain with non-conserved residues highlighted. FIG. 7B shows XAV939 bound to the TNKS2 donor site. FIG. 7C shows dose response curves of in vitro PARylation assay measuring the enzymatic activity of TNKS1 and TNKS2 incubated with XAV939 or TDI-012804 (n=4, error bars=SEM). FIG. 7D shows a scatterplot showing the TNKS2 IC₅₀ and TNKS2 selectivity (TNKS1 IC₅₀/TNKS2 IC₅₀) of synthesized compounds (N=2-4). Box highlights potent (TNKS2 IC₅₀<50 nM) and selective (>25-fold) compounds. Dark grey point=XAV939. Light grey points=previously published TNKS2-selective compounds. Black points=novel compounds. FIG. 7E shows an AXIN1-GFP reporter construct to measure stabilization of AXIN1 protein. FIG. 7F shows a western blot of 3T3 AXIN1-GFP reporter cells showing stabilization of AXIN1 and GFP protein following incubation with 1 uM XAV939 or 1 uM G007-LK for 24 hours. FIG. 7G shows AXIN1-GFP stabilization following incubation with XAV939 or TDI-012804 for 24 hours (n=3, error bars=SEM). GFP stabilization is quantified as the increase in fluorescence over DMSO and normalized to the highest concentration. FIG. 7H shows EC₅₀ values in shRenilla and shTnks1 AXIN1-GFP reporter cells of the six hit compounds, XAV939, and G007-LK (N=3). Selectivity is shRenilla EC₅₀/shTnks1 EC₅₀. FIG. 7I shows a dose response curves of transcriptional activity measured using a TOPFlash reporter expressed in DLD1s (n=3 [XAV939] or 2 [TDI-012804], error bars=SEM). Luciferase is normalized to DMSO. FIG. 7J shows a western blot showing selective AXIN1 stabilization in DLD1s with TDI-012804 treatment (500 nM or 250 nM for 24 hours). FIG. 7K shows the structure of TDI-012804. The image in FIG. 7E was created with Biorender.com.

FIG. 8 shows dose response curves from in vitro PARylation assays measuring the enzymatic activity of TNKS and TNKS2 incubated with published TNKS2 inhibitors (n=2, error bars=SEM). Enzymatic activity is normalized to DMSO.

FIGS. 9A-9B show a docking model of TNKS2 compounds. FIG. 9A shows a FEP validation plot. FIG. 9B shows aligned grids used for TNKS2 ensemble docking.

FIG. 10 shows dose response curves from in vitro PARylation assays measuring the enzymatic activity of TNKS1 and TNKS2 incubated with indicated compounds (n=2, error bars=SEM). Enzymatic activity is normalized to DMSO.

FIGS. 11A-11C show AXIN1-GFP reporter cell line to quantify AXIN1 stabilization. FIG. 11A shows a flow cytometry gating strategy to determine average GFP intensity in AXIN1-GFP reporter cells. FIG. 11B shows a western blot showing a decrease in Tnks protein levels AXIN1-GFP cells expressing a Tnks shRNA. FIG. 11C shows AXIN1-GFP stabilization following incubation with compounds for 24 hours as indicated (n=3, error bars=SEM). GFP stabilization is quantified as the increase in fluorescence over DMSO and normalized to the highest value. The boxed region on FIG. 11C shows AXIN1-GFP stabilization of inactive compounds normalized to maximum GFP increase following treatment with XAV.

FIG. 12 shows AXIN1 stabilization and WNT inhibition with hit compounds. The dose response curves of transcriptional activity were measured suing a TOPFlash reporter expressed in isogenic DLD1 cell lines (n=3-4, error bars=SEM). Luciferase is normalized to DMSO.

FIG. 13 shows that TDI-012804 does not inhibit PARP1/2/3. The dose response curves from in vitro PARylation assays measured the enzymatic activity of PARP1, PARP2, and PARP3 incubated with TDI-012804, Olaparib, or AZD-5305 as indicated (n=2, error bars=SD). Enzymatic activity is normalized to DMSO.

FIGS. 14A-14H show the mechanism of TNKS2-selective small molecule inhibitors. FIG. 14A shows a volume overview, shown as an isosurface, depicting the TNKS2 SAM-PARP filament and associated TDI-012804 binding sites. FIG. 14B shows the TDI-012804 binding site in relation to conserved tankyrase active-site motifs. TDI-012804 is shown as sticks and overlaid with the cryo-EM map density. FIG. 14C shows active-site residues, involved in the recognition of TDI-012804, depicted as sticks with associated hydrogen bonds shown as dashed connections. FIG. 14D shows superimposition of the +XAV and +2804 models. FIG. 14E shows superimposition of the +XAV, as in FIG. 14D, however TDI-012804 and residue Leu1134 (“closed” B-loop conformation) are depicted as spheres. FIG. 14F shows the fold selectivity of IC₅₀ values of XAV939 and TDI-012804 comparing the effect on TNKS1^{WT} to TNKS2^{WT} and TNKS2^{L1136Y} enzymatic activity using an in vitro PARylation assay (n=3, mean with SD, two-way ANOVA with Sidak correction). FIG. 14G shows a western blot showing AXIN1 and TNKS1/2 levels in TNKS^{KO} DLD1s expressing no cDNA, TNKS2^{WT} cDNA, or TNKS2^{L1136Y} cDNA, compared to TNKS1^{WT} DLD1s. Cells were treated with DMSO, 250 nM XAV939, or 250 nM TDI-012804 for 24 hours. FIG. 14H shows quantification of AXIN1 western blot band intensity normalized to Actin (n=3, mean with SD, two-way ANOVA with Tukey correction).

FIGS. 15A-15E show cryo-EM processing for the +2804 dataset. FIG. 15A shows the Cryo-EM processing pipeline. FIG. 15B shows a 3D-FSC plot for map1. FIG. 15C shows gold-standard FSC plots for map1. FIG. 15D shows a 3D-FSC plot for map2. FIG. 15E shows gold-standard FSC plots for map2.

FIGS. 16A-16C show local resolution analysis of the final cryo-EM maps for +2804 (FIG. 16A), +XAV (FIG. 16B), and apo (FIG. 16C).

FIGS. 17A-17E show cryo-EM processing for the +XAV dataset. FIG. 17A shows the Cryo-EM processing pipeline. FIG. 17B shows a 3D-FSC plot for map3.

FIG. 17C shows gold-standard FSC plots for map3. FIG. 17D shows a 3D-FSC plot for map4. FIG. 17E shows gold-standard FSC plots for map4.

FIGS. 18A-18E show cryo-EM processing for the apo dataset. FIG. 18A shows the Cryo-EM processing pipeline. FIG. 18B shows a 3D-FSC plot for map5. FIG. 18C shows gold-standard FSC plots for map5. FIG. 18D shows a 3D-FSC plot for map6.

FIG. 18E shows gold-standard FSC plots for map6.

FIG. 19A shows analysis of TDI-2804 contacts and clashes with active-site residues. FIG. 19B shows that TDI-2804 clashes with active-site residues when modelled bound to the “closed” B-loop state. FIG. 19C shows a scatter plot depicting the VDW overlap between the TDI-2804 atoms and surrounding protein residues, plotted interactions from FIG. 19A. FIG. 19D shows a scatter plot depicting the VDW overlap between the TDI-2804 atoms and surrounding protein residues, plotted interactions from FIG. 19B.

FIGS. 20A-20B show L1136Y mutation reduces TNKS2 selectivity of TDI-012804. FIG. 20A shows dose response curves from in vitro PARylation assays measuring the enzymatic activity of TNKS1, TNKS2^{WT}, and TNKS2^{L1136Y} incubated with XAV939 or TDI-012804 as indicated (n=3, error bars=SEM). Enzymatic activity is normalized to DMSO. FIG. 20B shows additional replicates of the western blot shown in FIG. 14G and quantified in FIG. 14H.

FIGS. 21A-21E show TNKS2-selective inhibitors suppress WNT in TNKS-depleted cells. FIG. 21A shows the generation of small intestinal organoid lines carrying Apc^{Q1405X} mutations. FIG. 21B shows C>T mutations in Apc allele in organoid lines, and brightfield images of Apc mutant organoids. Scale bar=200 μm. FIG. 21C shows a heatmap showing average log 2 normalized expression of all differentially expressed genes (log 2 fold change>1, padj<0.05) between G007-LK and DMSO treatments (n=3 replicates/condition). FIG. 21D shows volcano plots highlighting average log 2 fold change of genes enriched in Lgr5-high cells (“Lgr5 signature”) or differentiated enterocytes (“Enterocyte signature”), comparing treatments as indicated to DMSO treated organoids (n=3/condition). FIG. 21E shows a summary of the top 20 positively and negatively enriched gene sets enriched (from the G007-LK vs DMSO comparison) ranked by normalized enrichment score (NES). The plot shows NES (color) and false discovery rate qval (size) for all treatment conditions compared to DMSO treated cells of the same genotype. The image in FIG. 21A was created with Biorender.com.

FIGS. 22A-22G show that TNKS2 inhibitors reduce proliferation and viability in TNKS1-depleted cells. FIG. 22A shows a western blot showing stabilization of AXIN1 protein levels in SW403 cells treated with G007-LK (250 nM) or TDI-012804 (250 nM) for 24 hours. FIG. 22B shows quantification of SW403 colony forming assays treated with 250 nM G007-LK or 250 nM TDI-012804 normalized to DMSO (n=3, mean with SD, unpaired t test with Welch's correction). Quantification of H23 and OVCAR4 colony forming assays treated with 250 nM G007-LK or 250 nM TDI-012804 normalized to DMSO (n=3, mean with SD, two-way ANOVA with Tukey correction). FIG. 22C shows brightfield images of APC-mutant small intestinal organoids treated with DMSO, G007-LK (250 nM), or TDI-012804 (250 nM) for six days. Scale bar=800 μm. FIG. 22D shows the dose response curve of viability of Apc^{Q1405X} small intestinal organoids treated with G007-LK or TDI-012804 for six days measured using AlamarBlue (n=3, error bars =SEM). Fluorescence is normalized to DMSO. FIG. 22E shows organoid viability of Apc^{Q1405X} small intestinal organoids treated with G007-LK (250 nM) or TDI-012804 (250 nM) for 20 days measured using AlamarBlue (n=3, mean with SEM, two-way ANOVA with Tukey correction). FIG. 22F shows Dose response curve of viability of Apc^{Q1405X}/Kras^{G13D}/p53^{Q97X} small intestinal organoids treated with G007-LK or TDI-012804 for six days measured using AlamarBlue (n=3-6, error bars=SEM). Fluorescence is normalized to DMSO. FIG. 22G shows the dose response curve of viability of Apc^{Q1405X}/Kras^{G12D}/p53^{Q97X} small intestinal organoids treated with G007-LK or TDI-012804 for six days measured using AlamarBlue (n=3-6, error bars=SEM). Fluorescence is normalized to DMSO.

FIG. 23 shows that TDI-012804 selectively reduces cell viability. The does response curve shows quantitation of relative organoid area from incucyte imaging of Apc^{Q1405X} small intestinal organoids treated with G007-LK or TDI-012804 for 6 days (n=3, error bars=SEM). Organoid area is normalized to DMSO.

FIGS. 24A-24B show TNKS/TNKS2 stabilization following treatment with TDI-012804. FIG. 24A shows a western blot showing TNKS1 and TNKS2 levels following treatment of DLD1 cells for 24 hours with indicated doses (nM). FIG. 24B shows quantitation of TNKS1 (upper) and TNKS2 (lower) protein from three independent experiments. Data show dose-dependent stabilization of TNKS2 for both TDI-012804 and XAV939, but only XAV939 induces marked stabilization of TNKS1.

FIGS. 25A-25B show that mutation of L1136 in TNKS2 reduces drug-induced protein stabilization. FIG. 25A shows western blots of TNKS1-knockout DLD1 cells expressing WT (upper) or L1136Y (lower) mutant TNKS2, treated with XAV939 or TDI-012804, as indicated. FIG. 25B shows quantitation of TNKS1 (upper) and TNKS2 (lower) protein from three independent experiments. Data show dose-dependent stabilization of TNKS2 is reduced in cells that carry the L1136Y mutation, which mimics the amino acid sequence of TNKS1 in the B-loop of the acceptor site.

FIG. 26 shows dose response curves showing quantitation of relative organoid area from incucyte imaging of Apc^{Q1405X} small intestinal organoids treated with G007-LK or TDI-012804 for 6 days (n=3, error bars=SEM). Organoid area is normalized to DMSO. TDI-012804 selectively reduces cell viability.

### DETAILED DESCRIPTION

One aspect of the present disclosure relates to a compound of Formula (I):

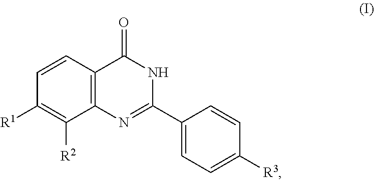

where
R¹ is selected from the group consisting of H, C₁₋₆ alkyl, C₁₋₆ alkoxy, —Oaryl, aryl, heterocyclyl, —NR⁴R⁵, —NHC(O)NHR⁵, —CONR⁶R⁷, and —NR⁸CO(CH₂)ₘR⁹, wherein the C₁₋₆ alkyl, C₁₋₆ alkoxy, —Oaryl, aryl, and heterocyclyl can be optionally substituted from 1 to 3 times with R′;
R² is selected from the group consisting of H, C₁₋₆ alkyl, C₁₋₆ alkoxy, —Oaryl, aryl, heterocyclyl, —NR⁴R⁵, —NHC(O)NHR⁵, —CONR⁶R⁷, and —NR⁸CO(CH₂)ₘR⁹, wherein the C₁₋₆ alkyl, C₁₋₆ alkoxy, —Oaryl, aryl, and heterocyclyl can be optionally substituted from 1 to 3 times with R′;
R′ is independently selected at each occurrence thereof from the group consisting of —OH, ═O, —NH₂, halogen, —NHCO₂R¹⁰, —CONR¹¹R¹², —NHCOR¹³, —NHR¹⁴, C₁₋₆ alkoxy, C₁₋₆ alkyl, aryl, biaryl, heteroaryl, —Oaryl, —Oheteroaryl, and heterocyclyl, wherein heteroaryl and heterocyclyl can be optionally substituted from 1 to 3 times with ═O;
R³ is C₁₋₆ alkyl optionally substituted from 1 to 3 times with a substituent independently selected at each occurrence thereof from the group consisting of OH, CF₃, and —O—(CH₂)ₙOH;
R⁴ is selected from the group consisting of H and C₁₋₆ alkyl;
R⁵ is selected from the group consisting of H, C₁₋₆ alkyl, aryl, benzyl, and heteroaryl, wherein C₁₋₆ alkyl can be optionally substituted from 1 to 3 times with a substituent selected independently at each occurrence thereof from the group consisting of —OH and aryl;
R⁶ is H or C₁₋₆ alkyl;
R⁷ is H or C₁₋₆ alkyl;
R⁸ is H or C₁₋₆ alkyl;
R⁹ is selected from the group consisting of C₁₋₆ alkyl, C₁₋₆ alkoxy, —Oaryl, —Oheteroaryl, C₃₋₁₂ cycloalkyl, aryl, biaryl, heterocyclyl, arylalkyl, heteroaryl, and —NHC₁₋₆ alkyl, wherein C₁₋₆ alkyl, —Oaryl, C₃₋₁₂ cycloalkyl, heterocyclyl, aryl, biaryl, arylalkyl, and heteroaryl can be optionally substituted from 1 to 3 times with R¹⁵;
R¹⁰ is selected from the group consisting of H, C₁₋₆ alkyl, and benzyl;
R¹¹ is H or C₁₋₆ alkyl;
R¹² is selected from the group consisting of H, C₁₋₆ alkyl, and aryl, wherein C₁₋₆ alkyl can be optionally substituted with C₁₋₆ alkoxy;
R¹³ is selected from the group consisting of H, C₁₋₆ alkyl, aryl, and —Obenzyl, wherein C₁₋₆ alkyl can be optionally substituted from 1 to 3 times with a substituent independently selected from —NHCOR¹⁶, aryl, benzyl, and —NR¹⁷R¹⁸,
R¹⁴ is selected from the group consisting of H, C₁₋₆ alkyl, heterocyclyl, and heteroaryl, wherein heterocyclyl and heteroaryl can be optionally substituted with C₁₋₆ alkyl or —COR¹⁹;
R¹⁵ is independently selected at each occurrence from the group consisting of ═O, —OH, halogen, —CN, —COC₁₋₆ alkyl, —COOH, —COOC₁₋₆ alkyl, aryl, C₁₋₆ alkyl, C₁₋₆ alkoxy, —CONHR¹⁶, —C(O)R²⁰, —C(O)aryl, benzyl, —Oaryl, —Obenzyl, —SO₂Me, heterocyclyl, heteroaryl, —OCHF₂, —OCF₂CHF₂, and —OCH₂CF₃, wherein C₁₋₆ alkyl, —Oaryl, aryl, heterocyclyl, and heteroaryl can be optionally substituted from 1 to 3 times with a substituent independently selected at each occurrence thereof from the group consisting of —OH, halogen, ═O, —C(O) CH₃, C₁₋₆ alkyl, —CF₃, heterocyclyl, C₁₋₆ alkoxy, —NR⁴R⁵, and —CN;
R¹⁶ is C₁₋₆ alkyl optionally substituted with C₁₋₆ alkoxy;
R¹⁷ is H or C₁₋₆ alkyl;
R¹⁸ is H or C₁₋₆ alkyl;
R¹⁹ is selected from the group consisting of C₁₋₆ alkyl, aryl, and —NHC₁₋₆ alkyl;
R²⁰ is —NMe₂ or heterocyclyl, wherein heterocyclyl can be optionally substituted 1 to 3 times with OH or C₁₋₆ alkyl;
n is 1, 2, 3, 4, or 5; and
m is 0 or 1;
with the proviso that both R¹ and R² cannot be H,
   or an oxide thereof, a pharmaceutically acceptable salt thereof, a solvate thereof, or a prodrug thereof.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

In this specification and the appended claims, the singular forms “a,” “an,” and “the” include plural references unless the context clearly dictates otherwise.

The terms “comprising,” “comprises,” and “comprised of” as used herein are synonymous with “including,” “includes,” or “containing,” “contains,” and are inclusive or open-ended and do not exclude additional, non-recited members, elements, or method steps.

The terms “comprising,” “comprises,” and “comprised of” also encompass the term “consisting of.” The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of.”

Terms of degree such as “substantially,” “about,” and “approximately” and the symbol “˜” as used herein mean a reasonable amount of deviation of the modified term such that the end result is not significantly changed. These terms of degree should be construed as including a deviation of at least ±0.1% (and up to ±1%, ±5%, or ±10%) of the modified term if this deviation would not negate the meaning of the word it modifies. Unless otherwise clear from context, all numerical values provided herein are modified by the term about. All numerical values provided herein that are modified by terms of degree set forth in this paragraph (e.g., “substantially,” “about,” “approximately,” and “˜”) are also explicitly disclosed without the term of degree. For example, “about 1%” is also explicitly disclosed as “1%”.

The term “and/or” as used herein means that the listed items are present, or used, individually or in combination. In effect, this term means that “at least one of” or “one or more” of the listed items is used or present.

The recitation of numerical ranges by endpoints includes all numbers and fractions subsumed within the respective ranges, as well as the recited endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

The term “alkyl” means an aliphatic hydrocarbon group which may be straight or branched having about 1 to about 12 carbon atoms in the chain. Particular alkyl groups have 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

The term “cycloalkyl” means a non-aromatic mono- or multicyclic ring system of about 3 to about 12 carbon atoms, preferably of about 3 to about 8 carbon atoms. Exemplary monocyclic cycloalkyls include cyclopentyl, cyclohexyl, cycloheptyl, bicyclo[1.1.1]pentyl, and the like.

The term “alkoxy” means groups of from 1 to 8 carbon atoms of a straight, branched, or cyclic configuration and combinations thereof attached to the parent structure through an oxygen. Examples include methoxy, ethoxy, propoxy, isopropoxy, cyclopropyloxy, cyclohexyloxy, and the like. Lower-alkoxy refers to groups containing one to four carbons. For the purposes of the present patent application, alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

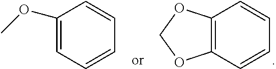

The term “aryl” means an aromatic monocyclic or multicyclic ring system of 6 to about 14 carbon atoms, preferably of 6 to about 10 carbon atoms. Representative aryl groups include phenyl and naphthyl.

The term “arylalkyl” or “alkylaryl” means an alkyl substituted with one or more aryl groups, wherein the alkyl and aryl groups are as herein described. One particular example is an arylmethyl or arylethyl group, in which a single or a double carbon spacer unit is attached to an aryl group, where the carbon spacer and the aryl group can be optionally substituted as described herein. Representative arylalkyl groups include

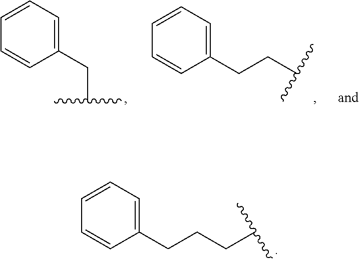 [SMILES: *Cc1ccccc1 ; *CCc1ccccc1 ; *CCCc1ccccc1]

The term “heteroaryl” means an aromatic monocyclic or multicyclic ring system of about 5 to about 14 ring atoms, preferably about 5 to about 10 ring atoms, in which one or more of the atoms in the ring system is/are element(s) other than carbon, for example, nitrogen, oxygen, or sulfur. In the case of multicyclic ring system, only one of the rings needs to be aromatic for the ring system to be defined as “Heteroaryl”. Preferred heteroaryls contain about 5 to 6 ring atoms. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2 (3H)-yl, and the like. The term “heteroarylene” refers to a group obtained by removal of a hydrogen atom from a heteroaryl group. Exemplary heteroarylene groups include, but are not limited to, groups derived from the heteroaryl groups described above.

As used herein, “heterocyclyl” refers to a stable 3- to 18-membered ring (radical) which consists of carbon atoms and from one to five heteroatoms selected from the group consisting of nitrogen, oxygen and sulfur. For purposes of this application, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. For purposes of this application, when the heterocycle is a polycyclic ring system, one of the rings can be an aromatic ring and another one is non-aromatic. Examples of heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

The term “phenyl” means a phenyl group as shown below

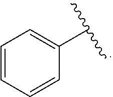 [SMILES: *c1ccccc1]

The term “benzyl” means a benzyl group as shown below

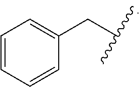 [SMILES: *Cc1ccccc1]

The term “halogen” means fluoro, chloro, bromo, or iodo.

The term “substituted” or “substitution” of an atom means that one or more hydrogen on the designated atom is replaced with a selection from the indicated group, provided that the designated atom's normal valency is not exceeded.

“Unsubstituted” atoms bear all of the hydrogen atoms dictated by their valency. When a substituent is keto (i.e., ═O), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The term “method of treating” means amelioration or relief from the symptoms and/or effects associated with the disorders described herein. As used herein, reference to “treatment” of a patient is intended to include prophylaxis.

The term “compounds of the invention”, and equivalent expressions, are meant to embrace compounds of general Formula (I), Formula (Ia), and Formula (Ib), as hereinbefore described, which expression includes the prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

The term “pharmaceutically acceptable salts” means the relatively non-toxic, inorganic, and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like (see, for example, Berge et al., “Pharmaceutical Salts,” J. Pharm. Sci., 66:1-9 (1977) and Remington's Pharmaceutical Sciences, 17th ed., Mack Publishing Company, Easton, Pa., 1985, p. 1418, which are hereby incorporated by reference in their entirety). Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include, for example, sodium hydride, sodium hydroxide, potassium hydroxide, calcium hydroxide, aluminum hydroxide, lithium hydroxide, magnesium hydroxide, and zinc hydroxide. Suitable amine base addition salts are prepared from amines which have sufficient basicity to form a stable salt, and preferably include those amines which are frequently used in medicinal chemistry because of their low toxicity and acceptability for medical use, such as ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylarnine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, dicyclohexylamine, and the like.

The term “pharmaceutically acceptable prodrugs” as used herein means those prodrugs of the compounds useful according to the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” means compounds that are rapidly transformed in vivo to yield the parent compound of the above formula, for example by hydrolysis in blood. Functional groups which may be rapidly transformed, by metabolic cleavage, in vivo form a class of groups reactive with the carboxyl group of the compounds of this invention. They include, but are not limited to, such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ed., Elsevier (1985); Methods in Enzymology, K. Widder et al, Ed., Academic Press, 42, p. 309-396 (1985); A Textbook of Drug Design and Development, Krogsgaard-Larsen and H. Bundgaard, ed., Chapter 5; “Design and Applications of Prodrugs” p. 113-191 (1991); Advanced Drug Delivery Reviews, H. Bundgard, 8, p. 1-38 (1992); J. Pharm. Sci., 77:285 (1988); Nakeya et al, Chem. Pharm. Bull., 32:692 (1984); Higuchi et al., “Pro-drugs as Novel Delivery Systems,” Vol. 14 of the A.C.S. Symposium Series, and Bioreversible Carriers in Drug Design, Edward B. Roche, ed., American Pharmaceutical Association and Pergamon Press (1987), which are incorporated herein by reference in their entirety. Examples of prodrugs include, but are not limited to, acetate, formate, and benzoate derivatives of alcohol and amine functional groups in the compounds of the invention.

The term “solvate” refers to a compound of Formula (I), Formula (Ia), and Formula (Ib) in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The term “therapeutically effective amounts” is meant to describe an amount of compound of the present invention effective to produce the desired therapeutic effect. Such amounts generally vary according to a number of factors well within the purview of ordinarily skilled artisans given the description provided herein to determine and account for. These include, without limitation: the particular subject, as well as its age, weight, height, general physical condition, and medical history; the particular compound used, as well as the carrier in which it is formulated and the route of administration selected for it; and the nature and severity of the condition being treated.

The term “pharmaceutical composition” means a composition comprising a compound of Formula (I), Formula (Ia), and Formula (Ib) and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

The term “pharmaceutically acceptable” means it is, within the scope of sound medical judgement, suitable for use in contact with the cells of humans and lower animals without undue toxicity, irritation, allergic response and the like, and are commensurate with a reasonable benefit/risk ratio.

The term “pharmaceutically acceptable dosage forms” means dosage forms of the compound of the invention, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. This technology is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

This technology also envisions the “quaternization” of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

In some embodiments, the compound has the Formula (Ia):

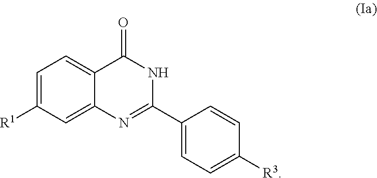

In some embodiments, the compound has the Formula (Ib):

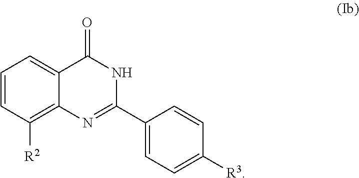

In some embodiments, R¹ is selected from the group consisting of H,

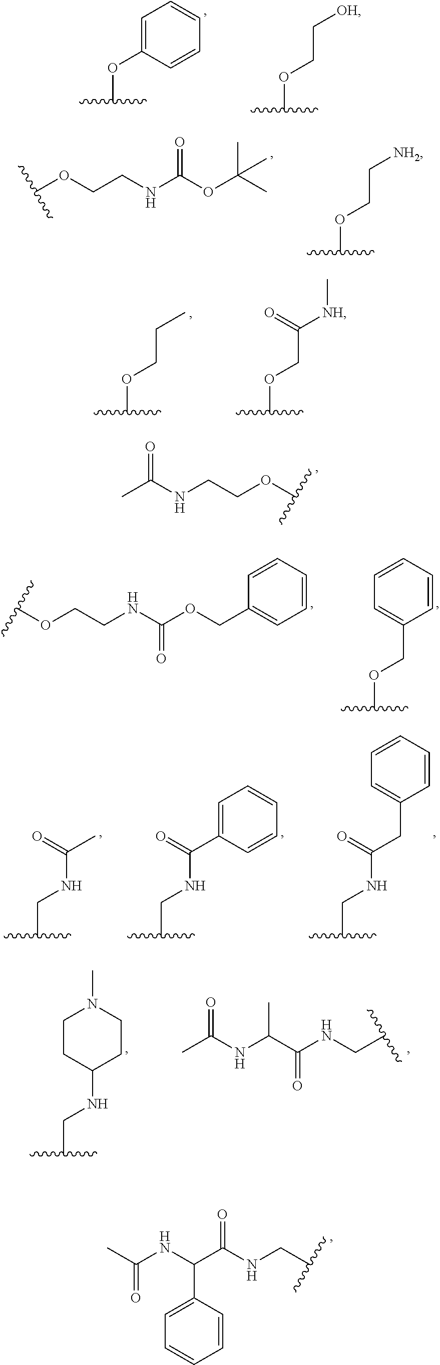

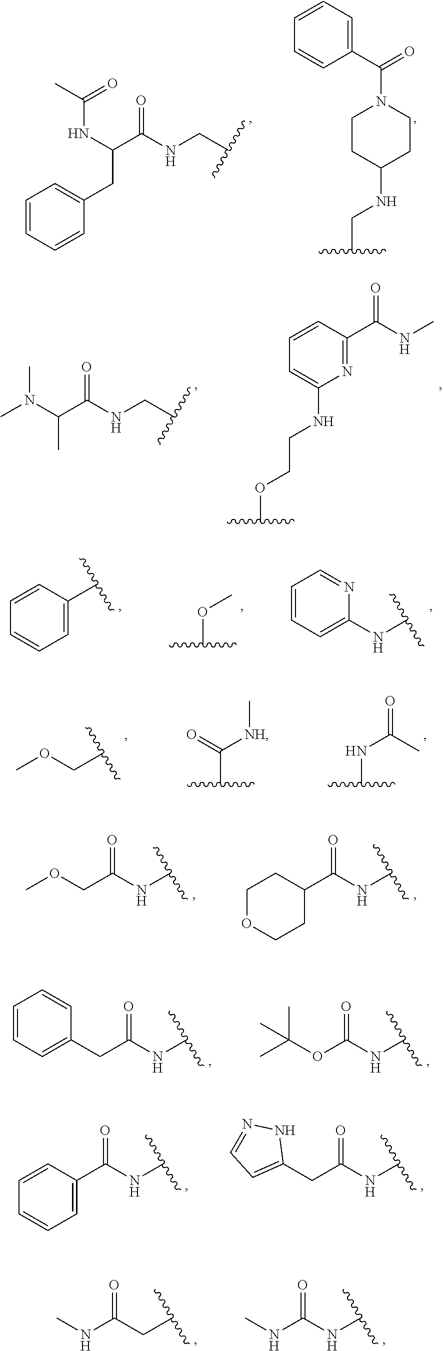

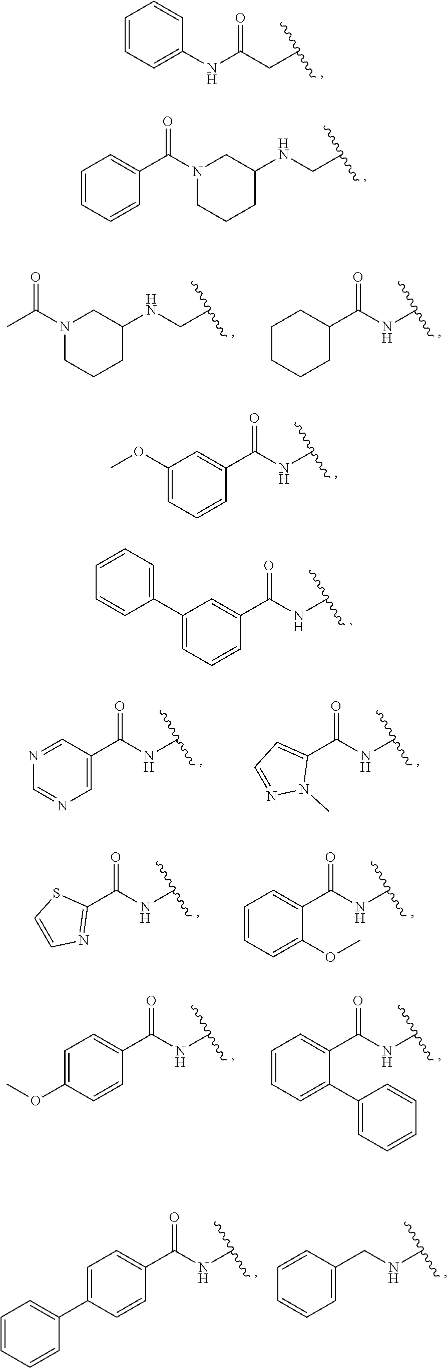

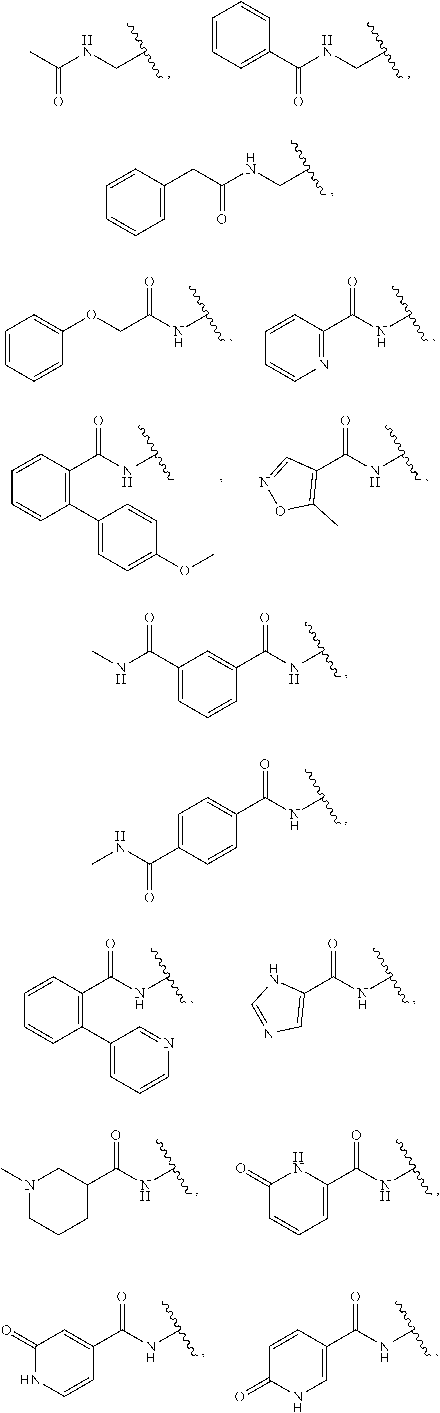

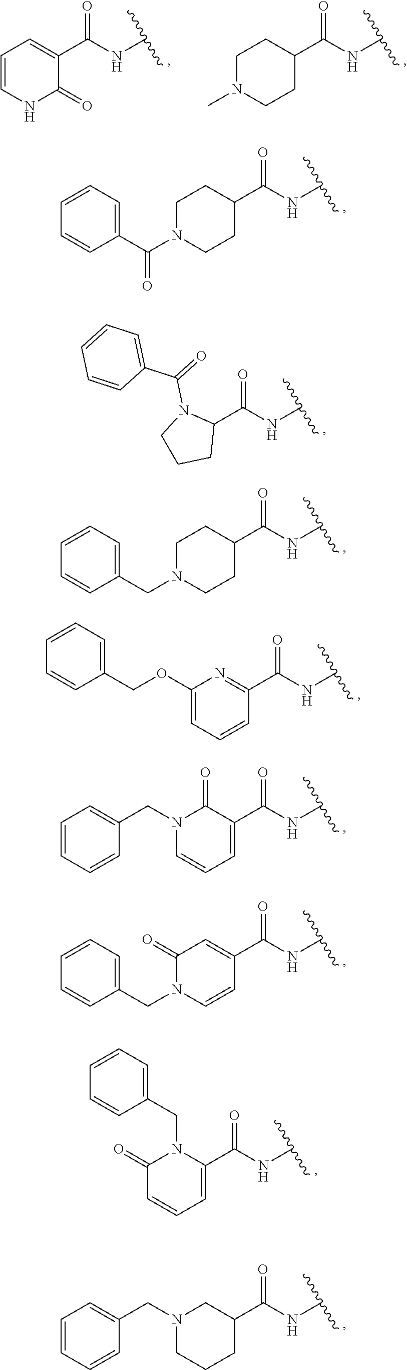

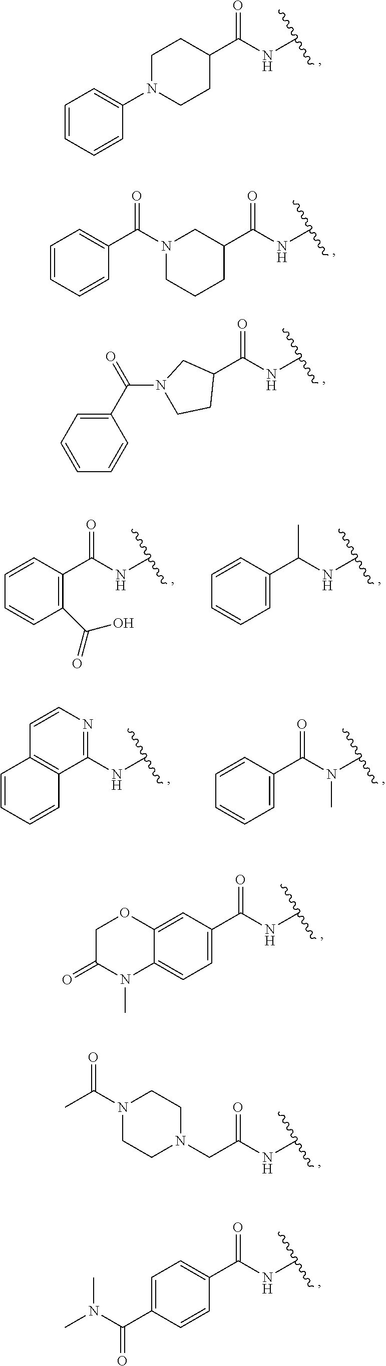

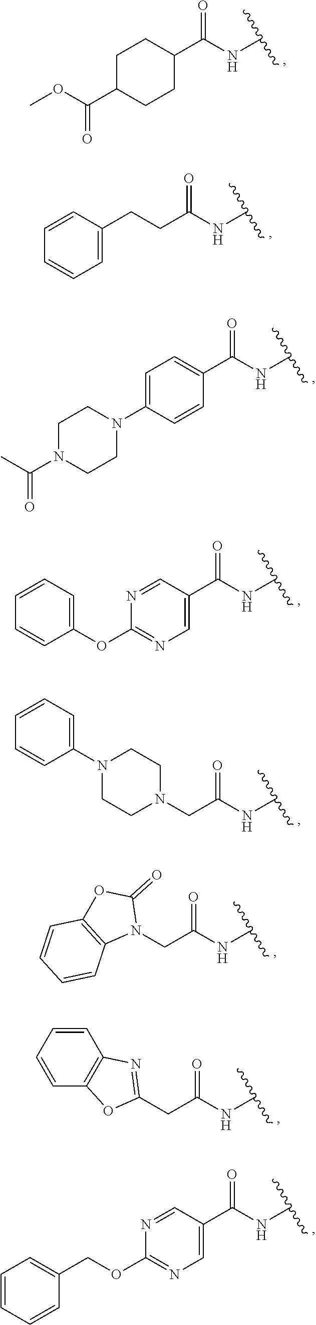

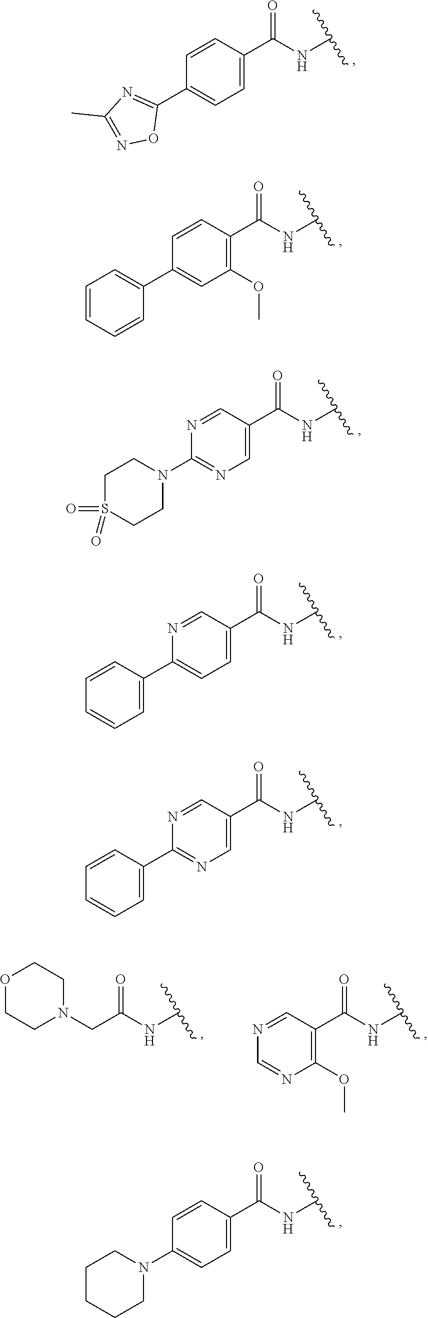

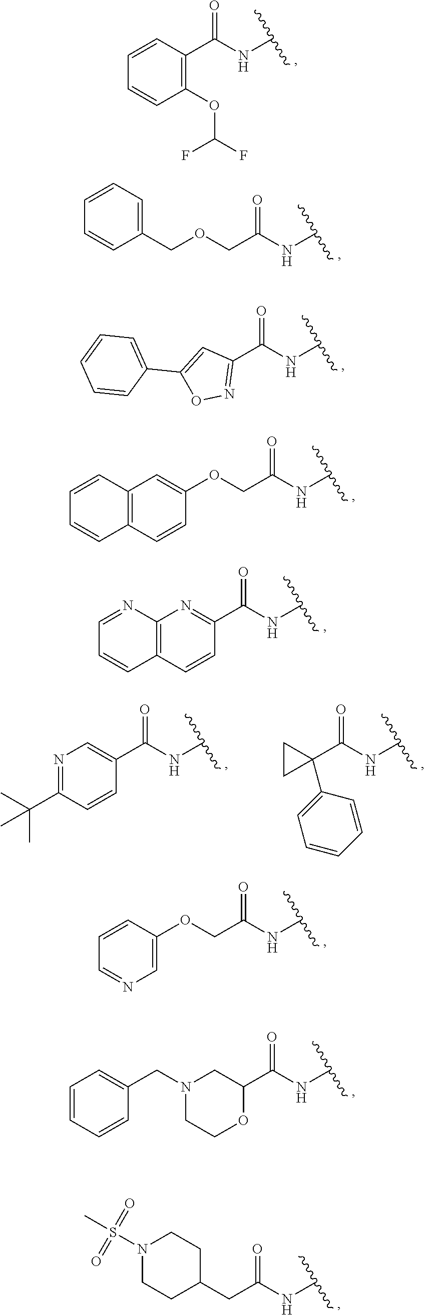

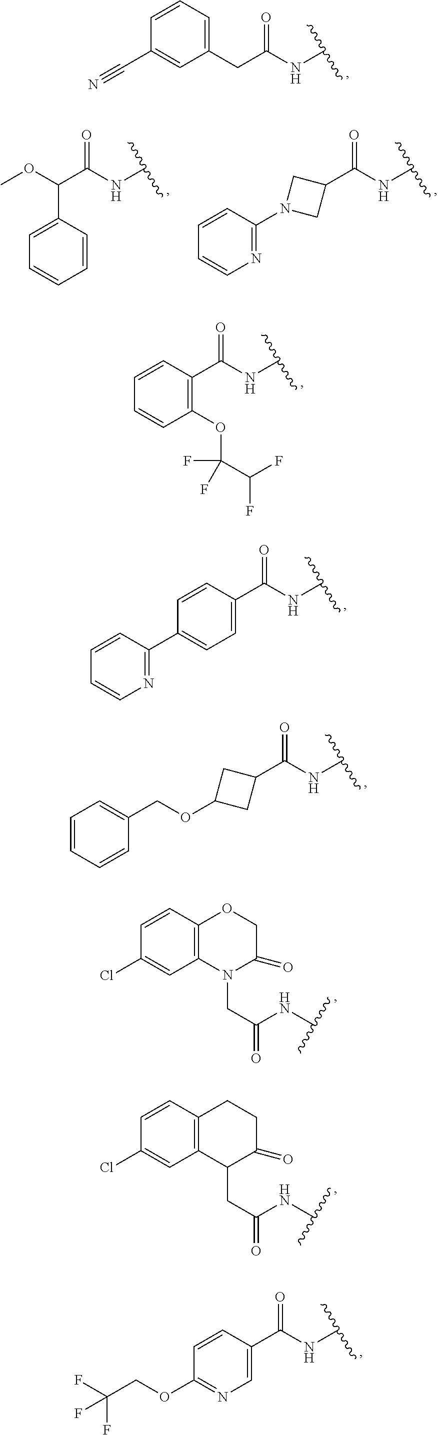

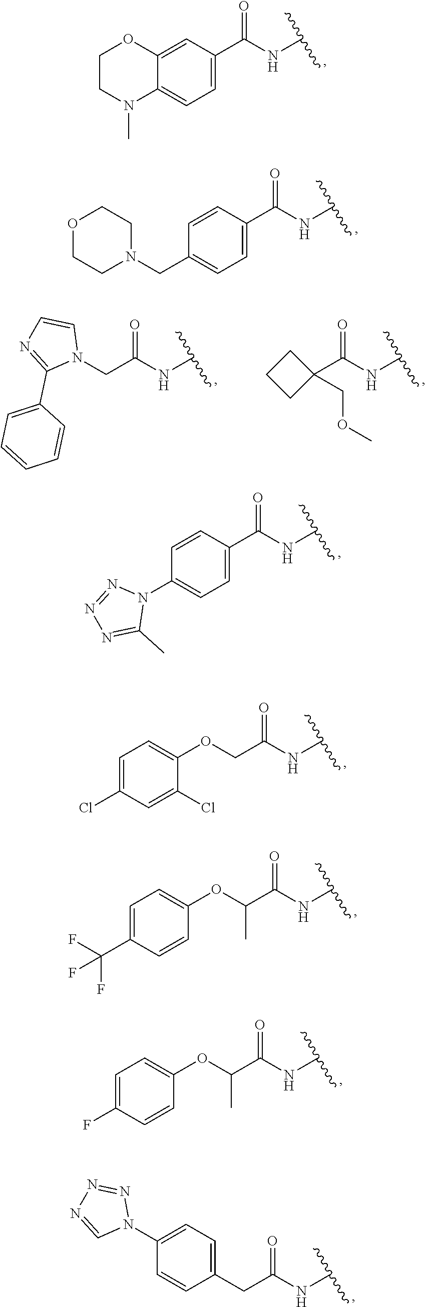

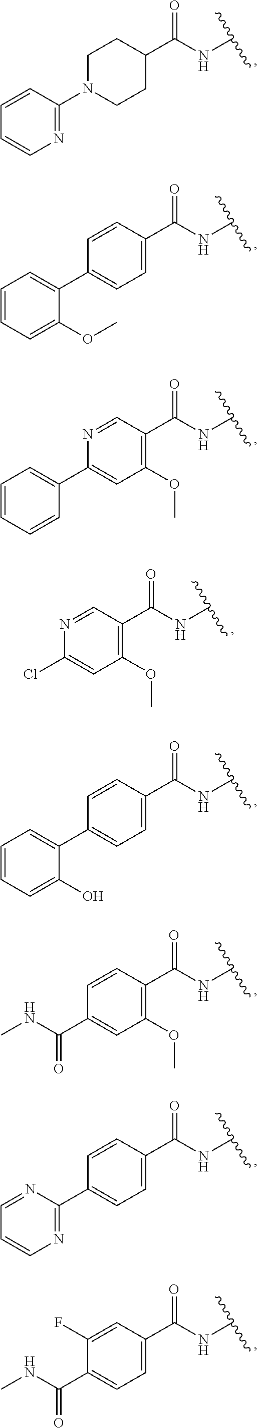

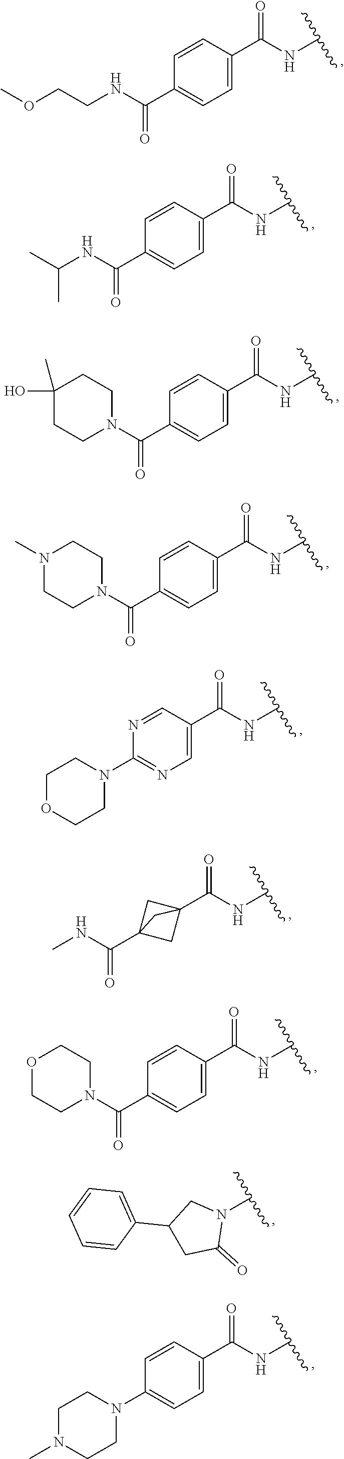

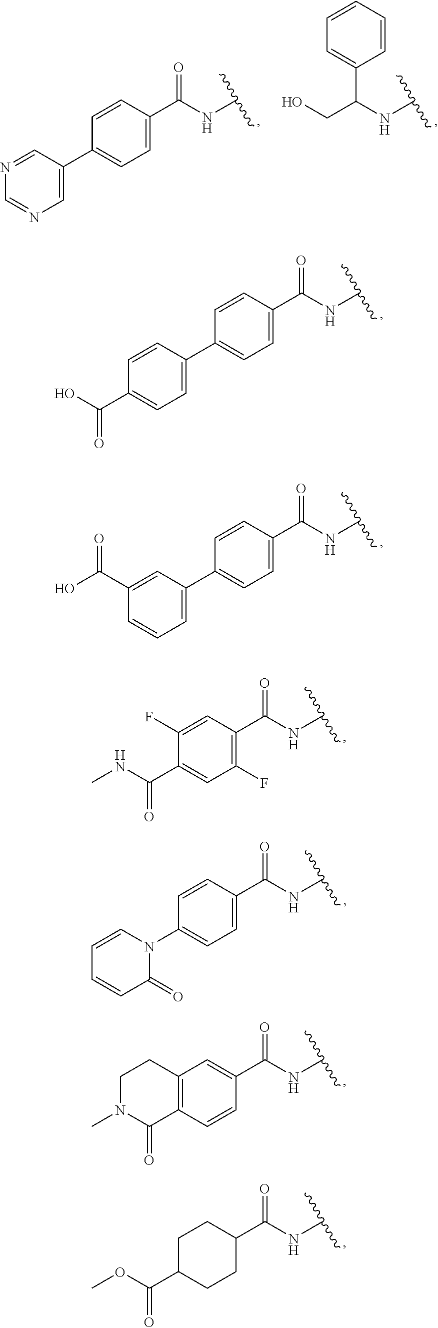

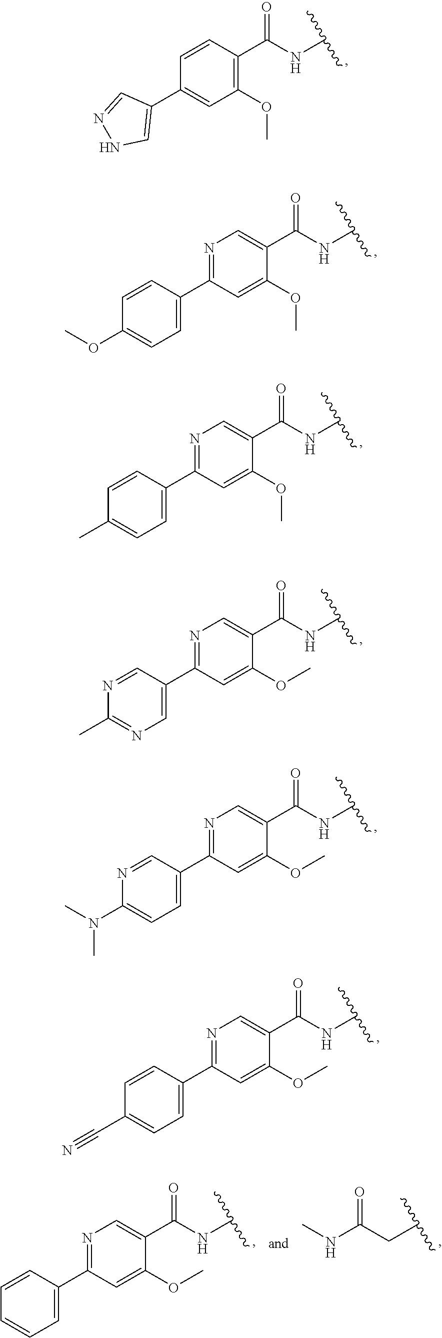

where

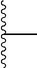

is the point of attachment to the corresponding carbon atom of the structure of Formula (I).

In some embodiments, R² is selected from the group consisting of H,

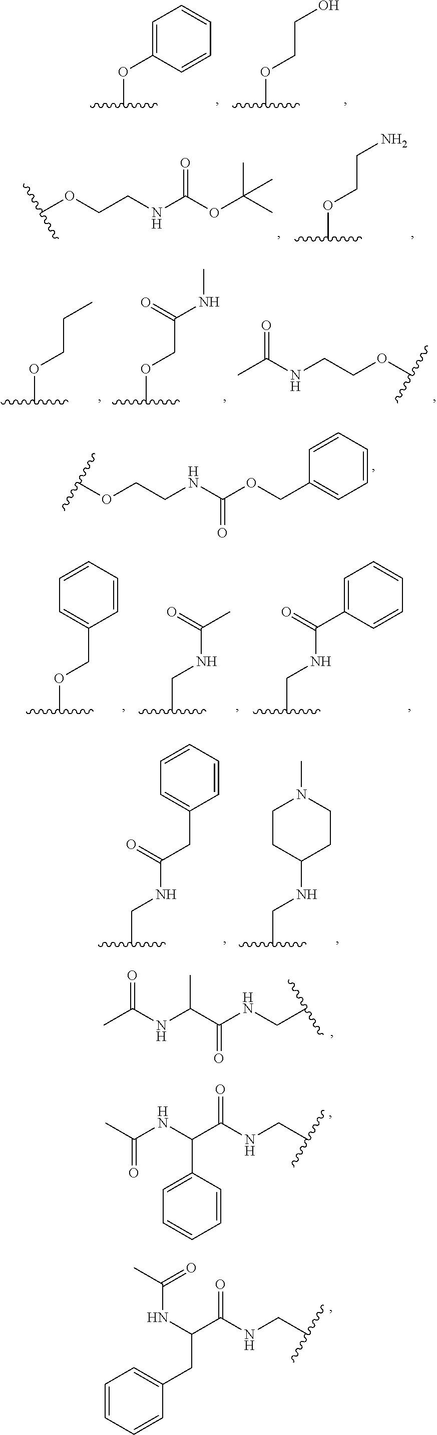

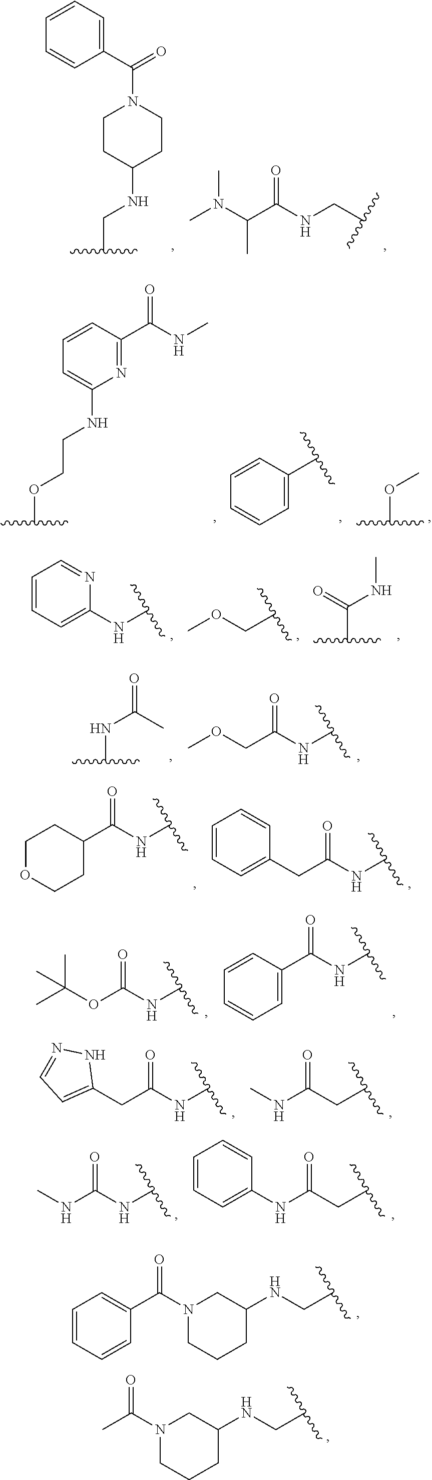

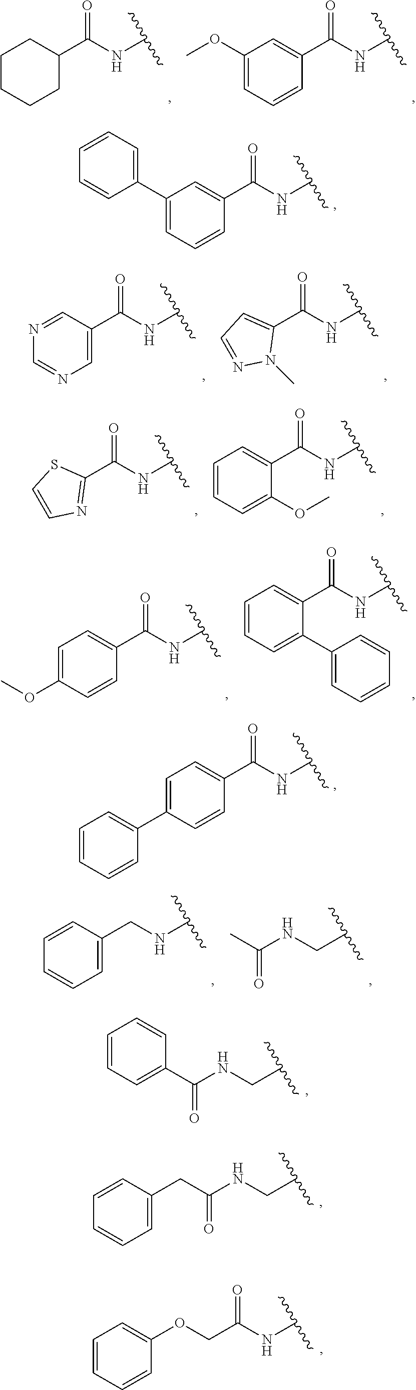

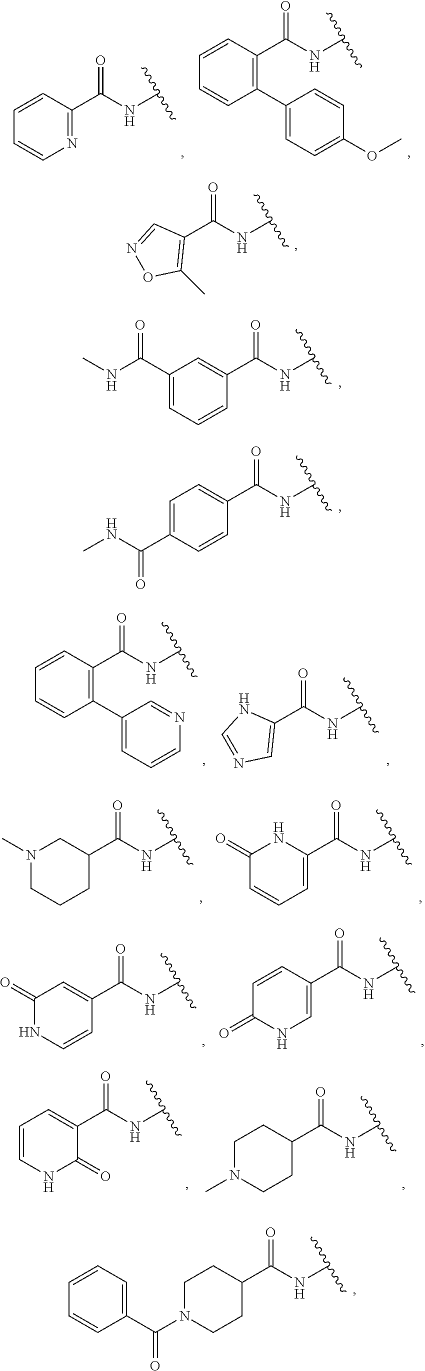

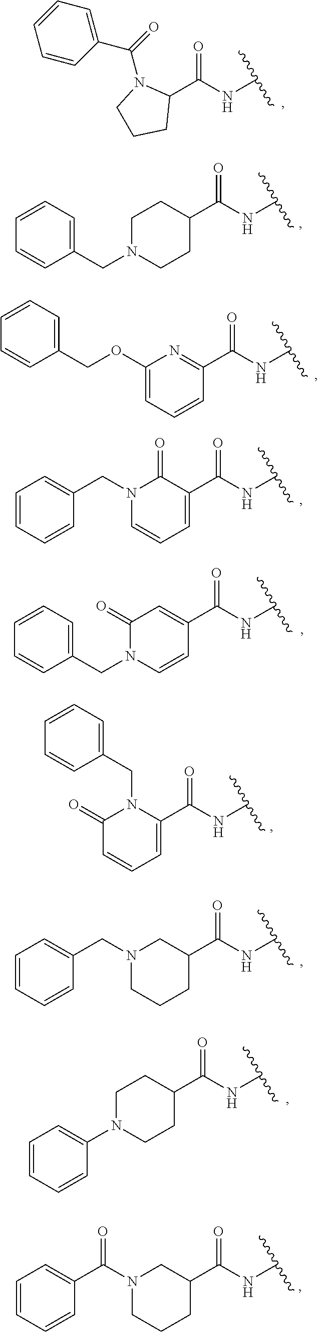

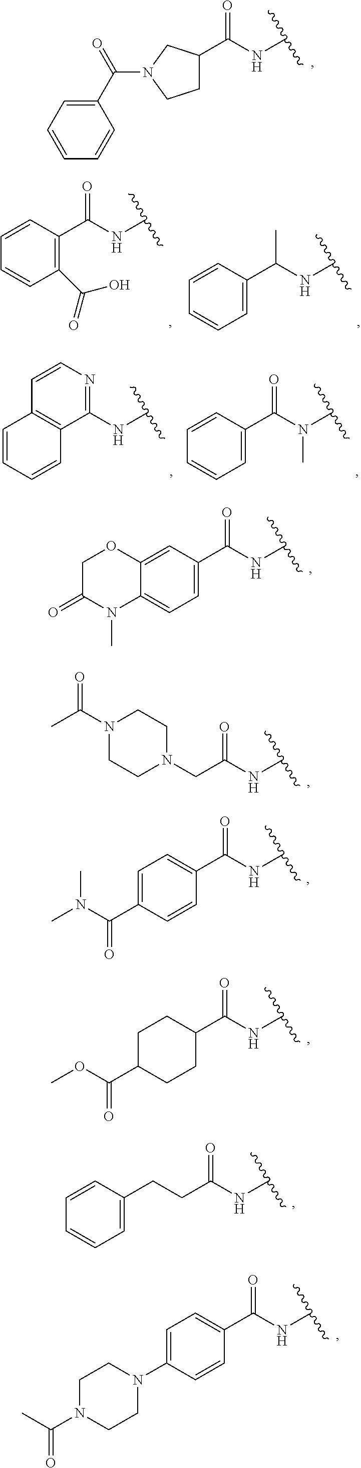

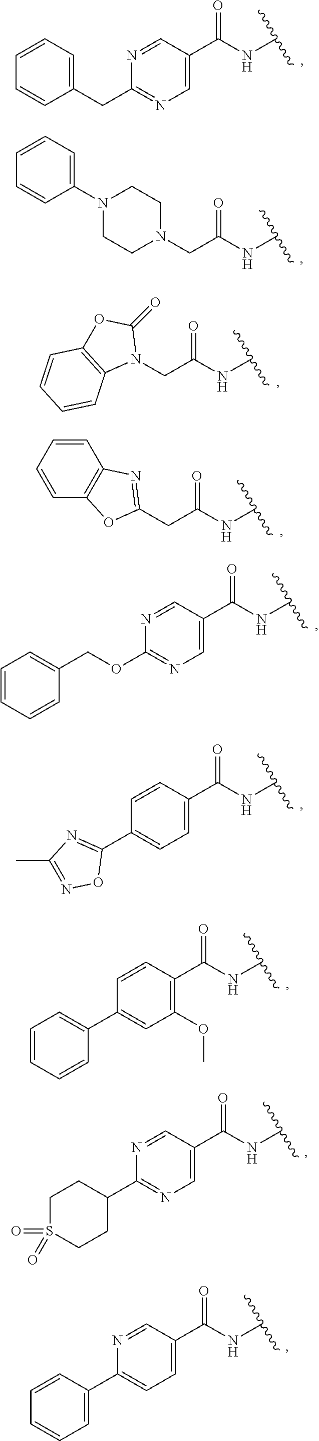

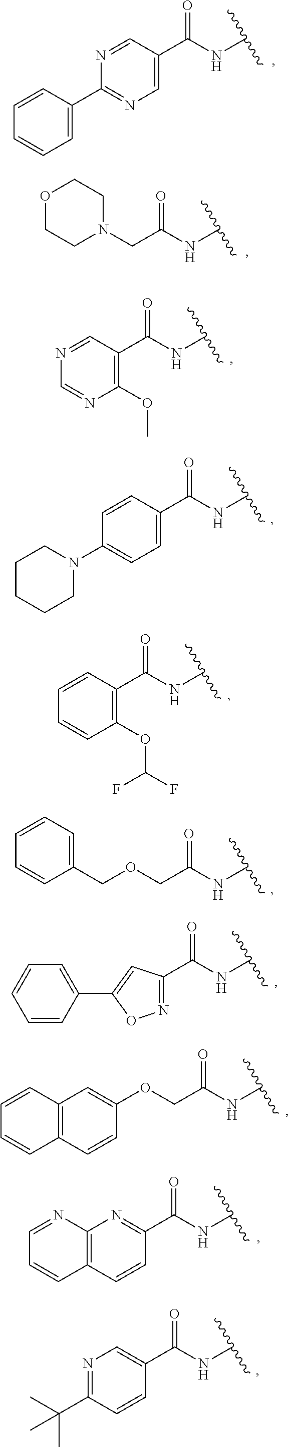

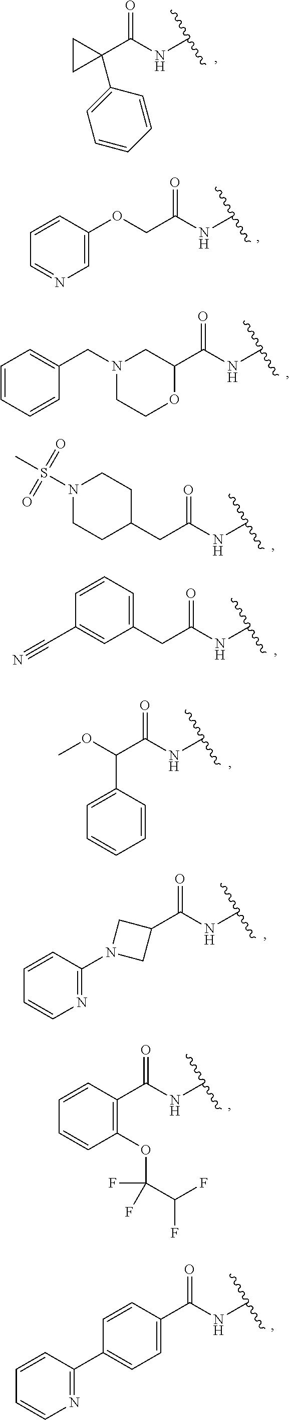

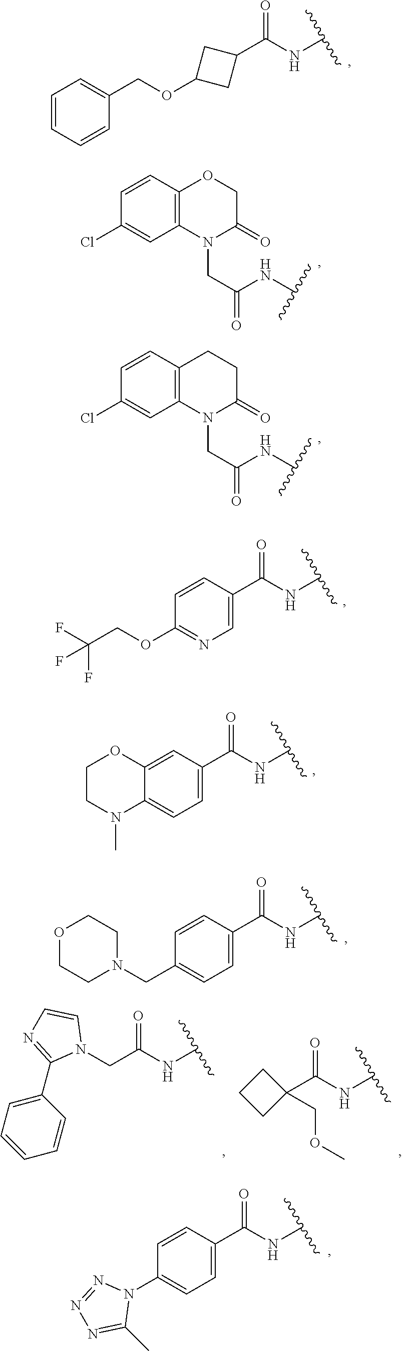

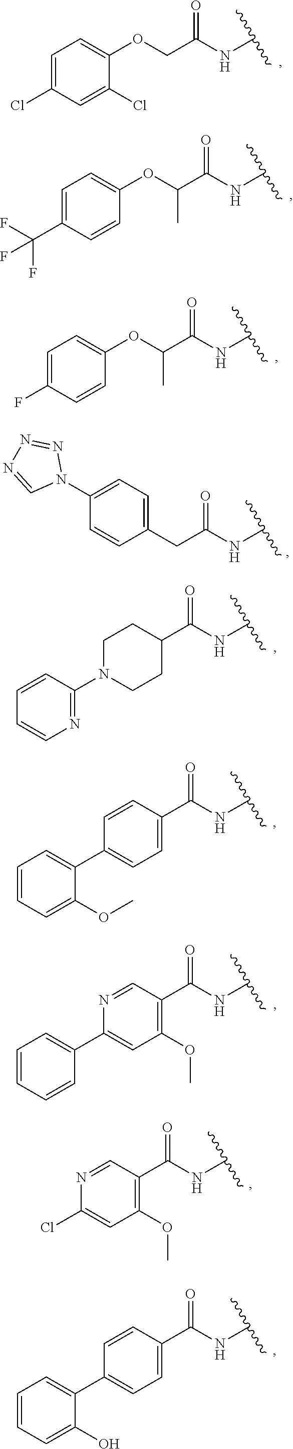

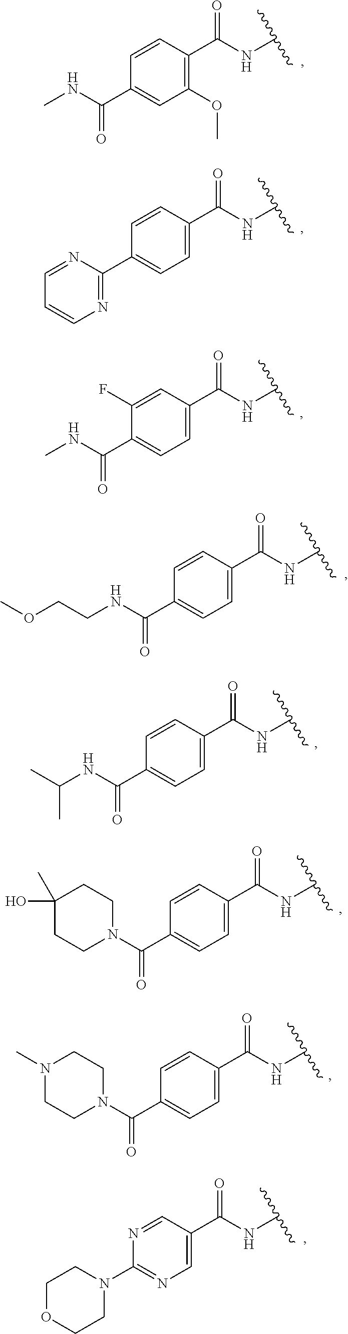

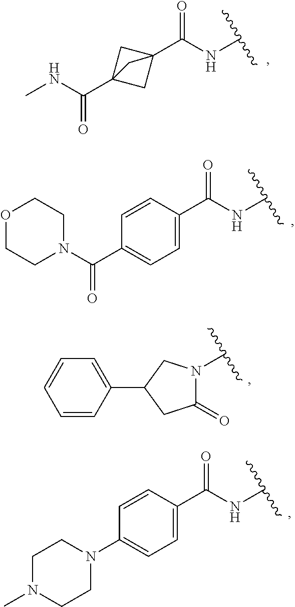

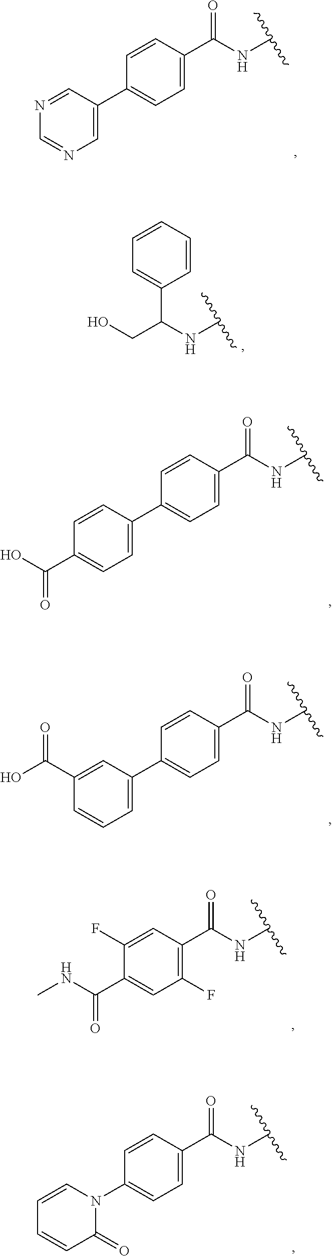 [SMILES: *NC(=O)c1ccc(-c2cncnc2)cc1 ; *NC(CO)c1ccccc1 ; *NC(=O)c1ccc(-c2ccc(C(=O)O)cc2)cc1 ; *NC(=O)c1ccc(-c2cccc(C(=O)O)c2)cc1 ; *NC(=O)c1cc(F)c(C(=O)NC)cc1F ; *NC(=O)c1ccc(-n2ccccc2=O)cc1]

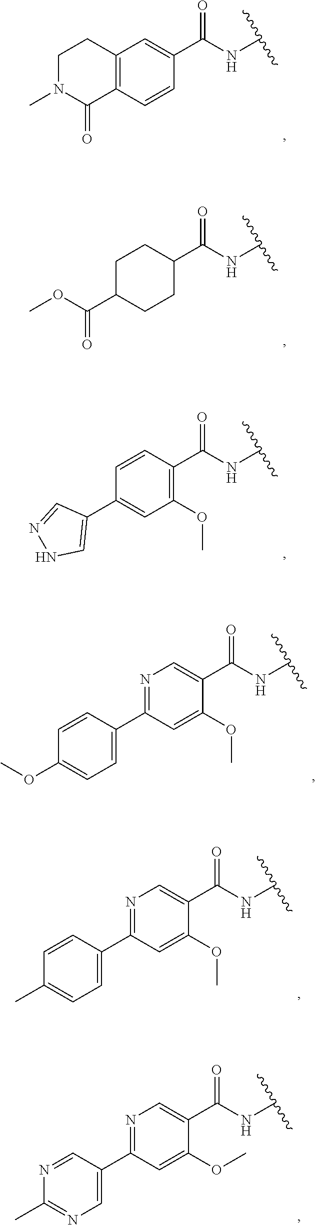 [SMILES: *NC(=O)c1ccc2c(c1)CCN(C)C2=O ; *NC(=O)C1CCC(C(=O)OC)CC1 ; *NC(=O)c1ccc(-c2cn[nH]c2)cc1OC ; *NC(=O)c1cnc(-c2ccc(OC)cc2)cc1OC ; *NC(=O)c1cnc(-c2ccc(C)cc2)cc1OC ; *NC(=O)c1cnc(-c2cnc(C)nc2)cc1OC]

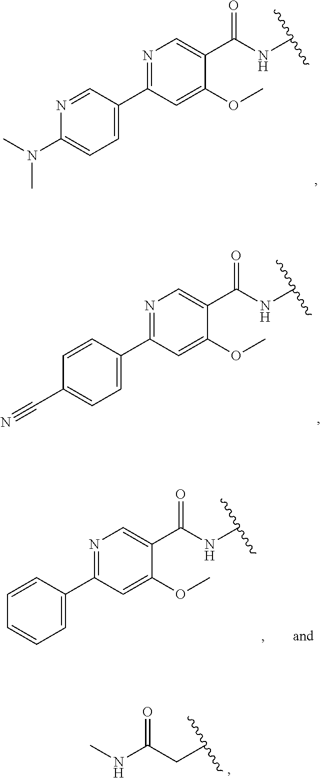

where

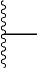

is the point of attachment to the corresponding carbon atom of the structure of Formula (I).

In some embodiments, R³ is selected from the group consisting of

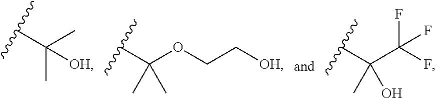

where

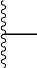

is the point of attachment to the corresponding carbon atom of the structure of Formula (I).

In some embodiments, R¹ is selected from the group consisting of

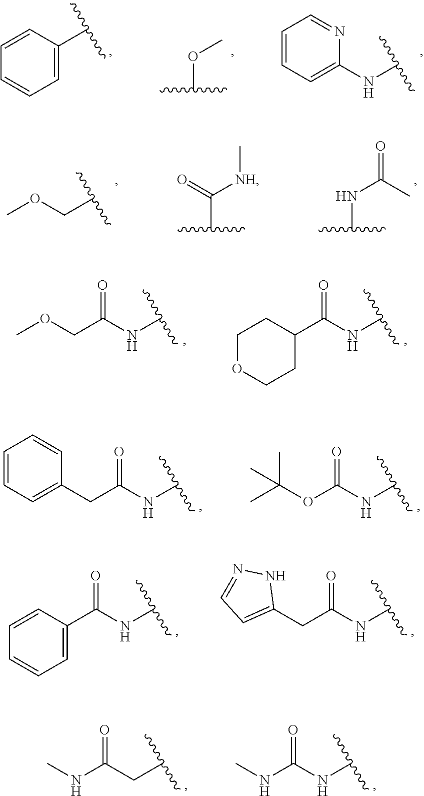

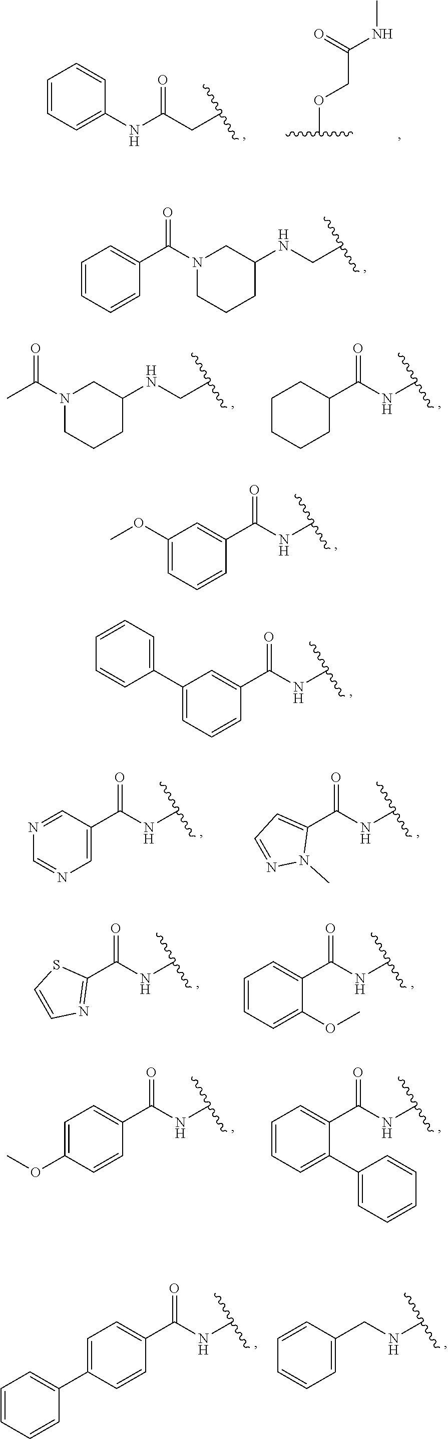

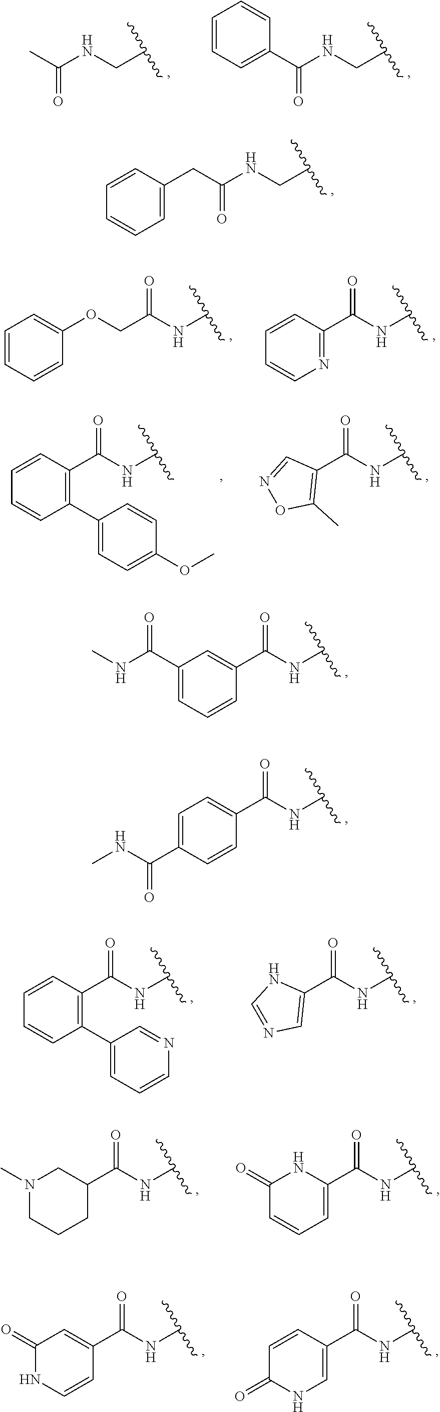

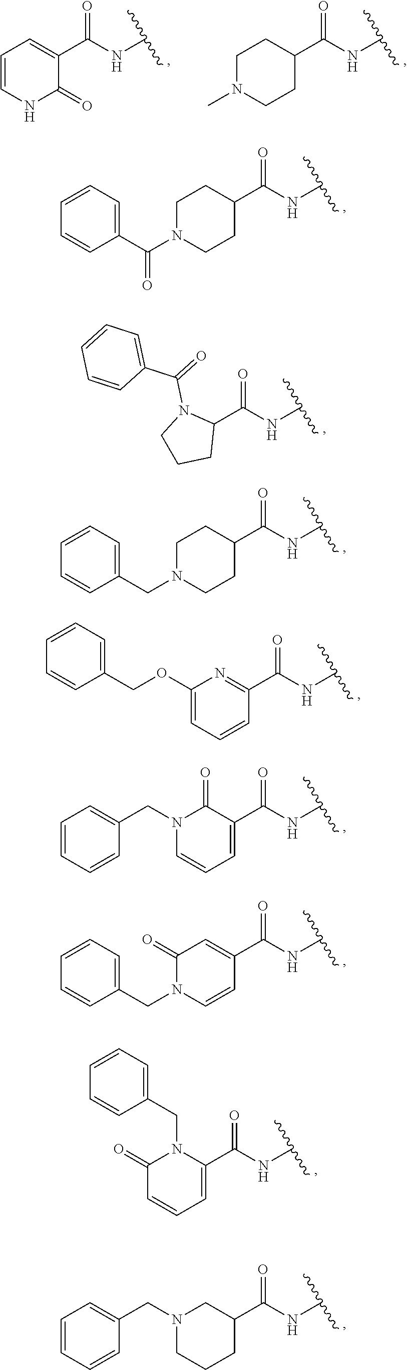

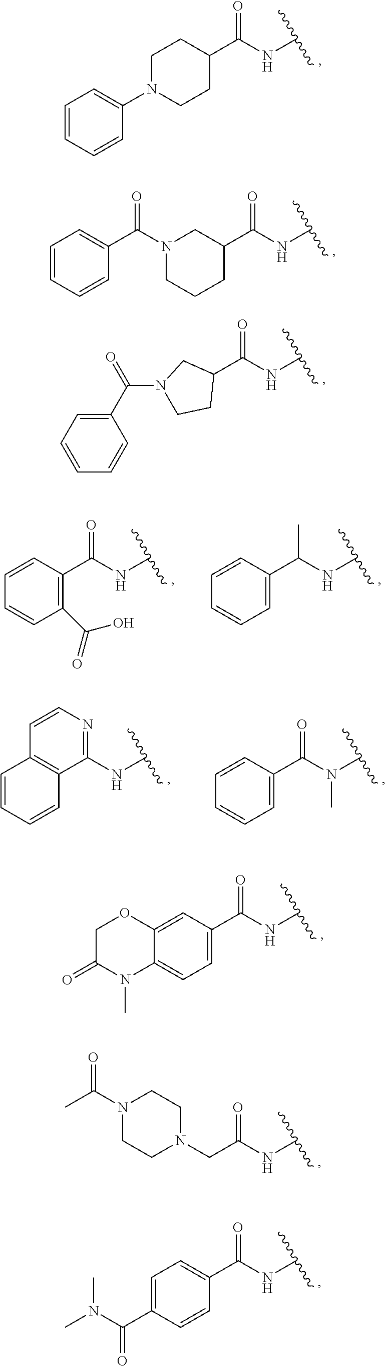

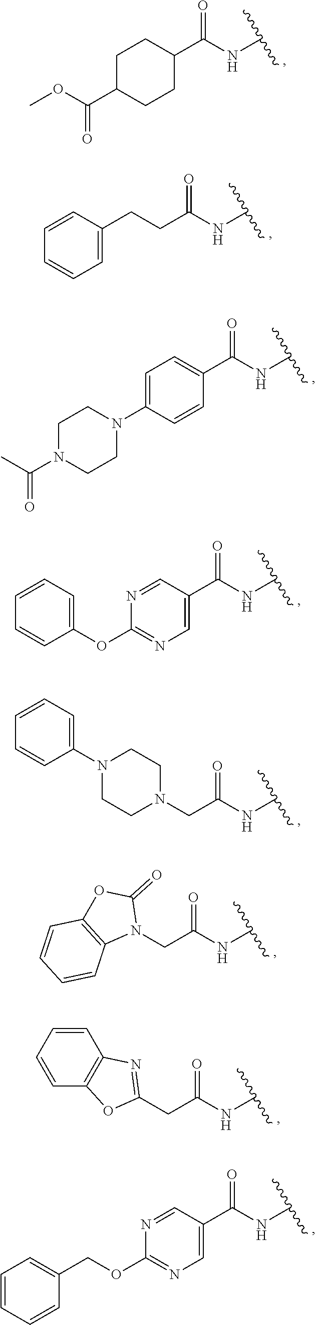

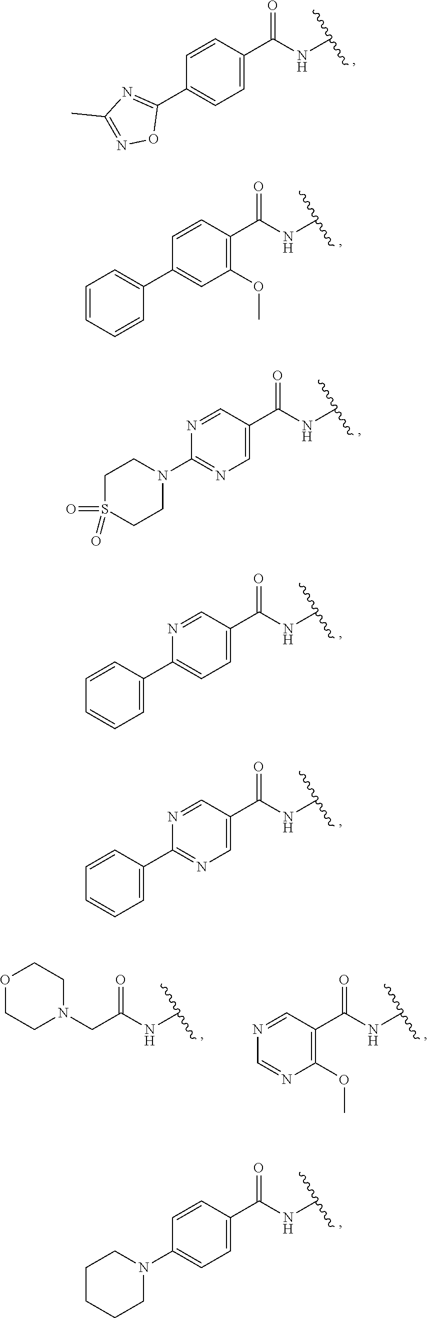

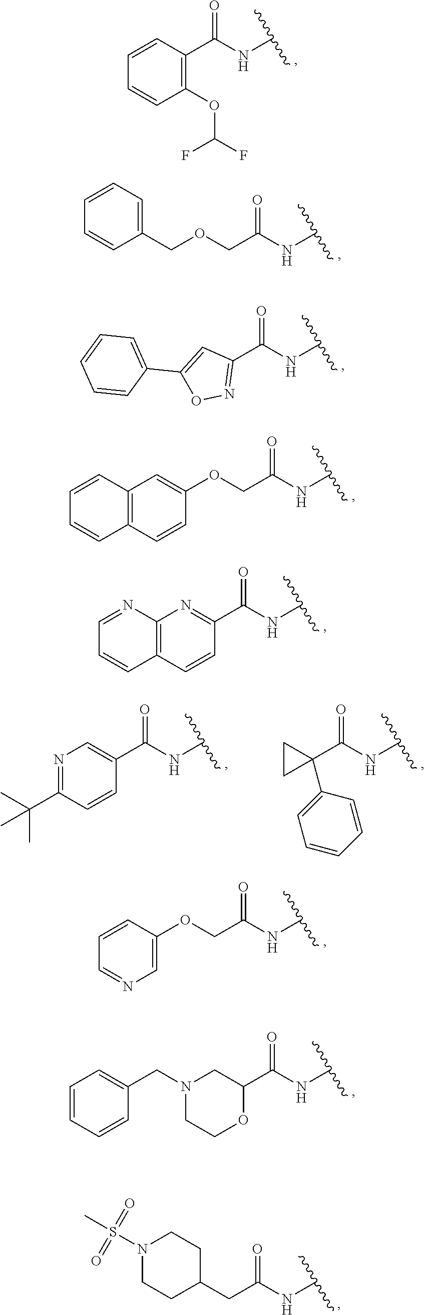

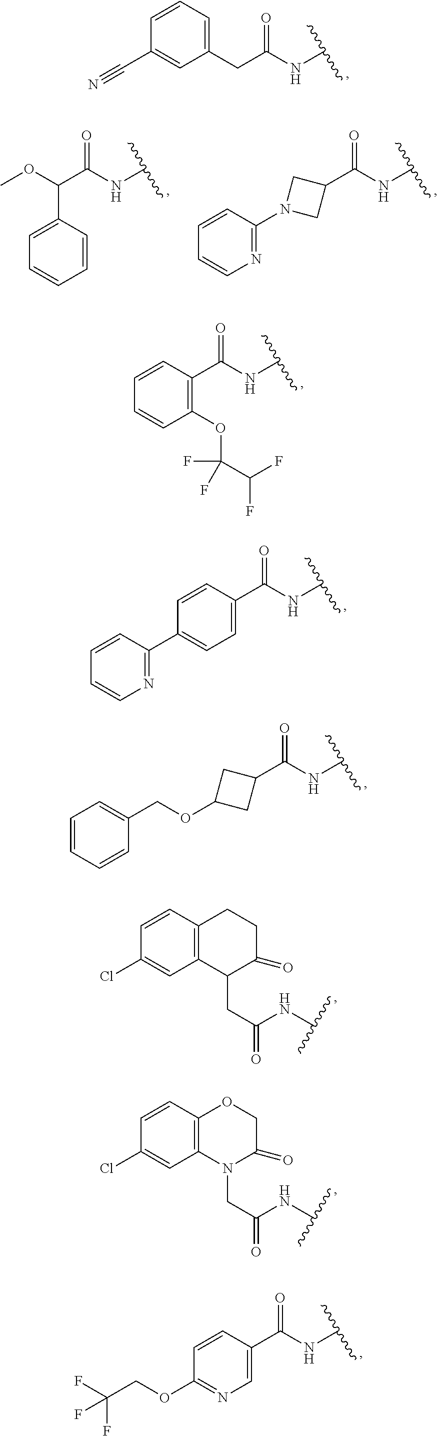

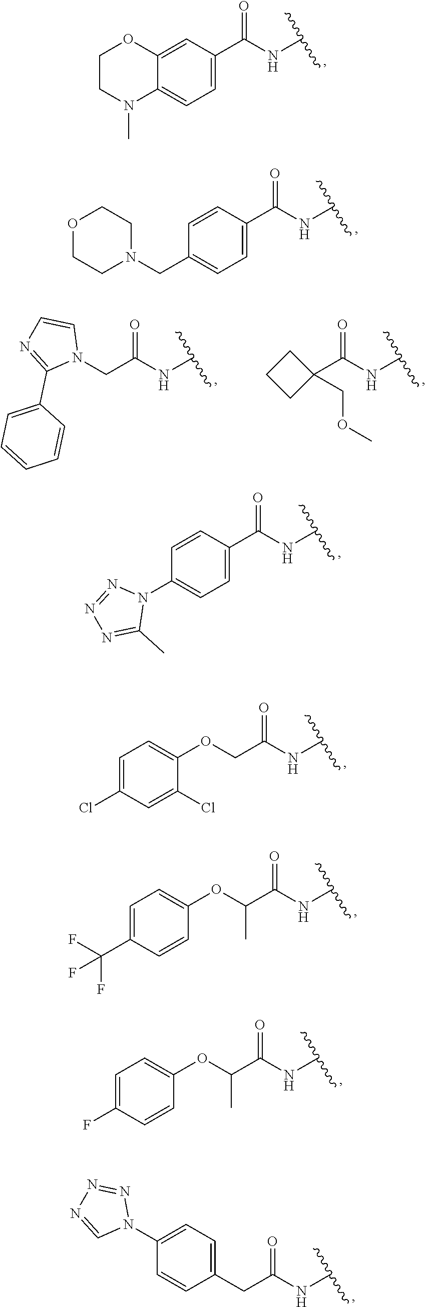

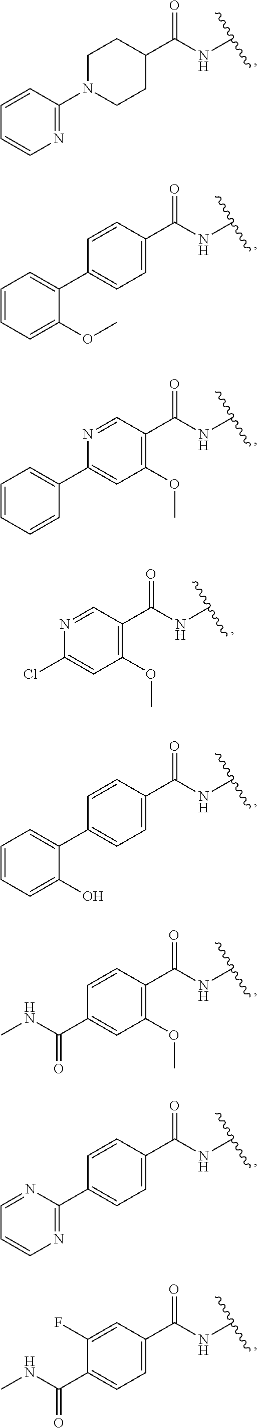

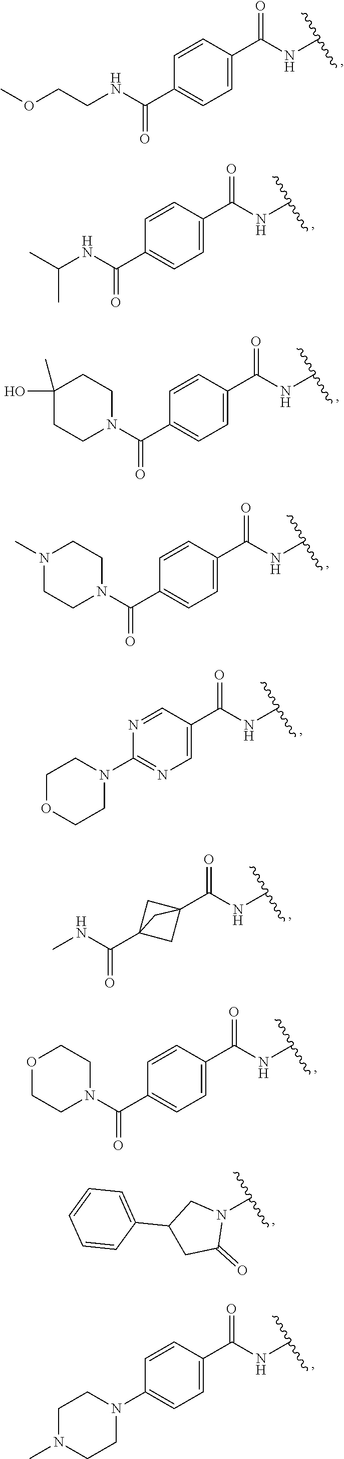

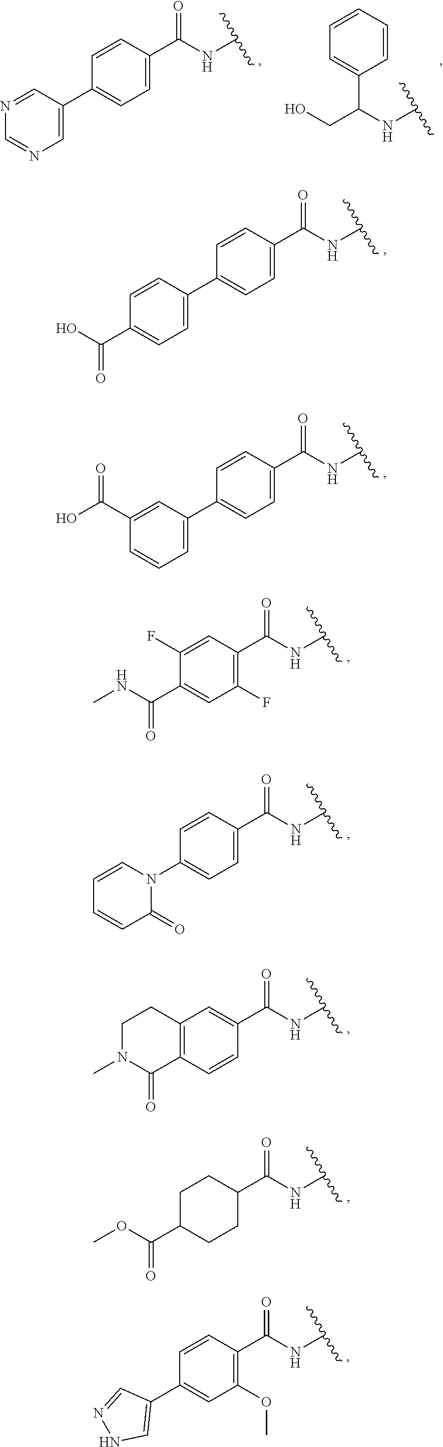

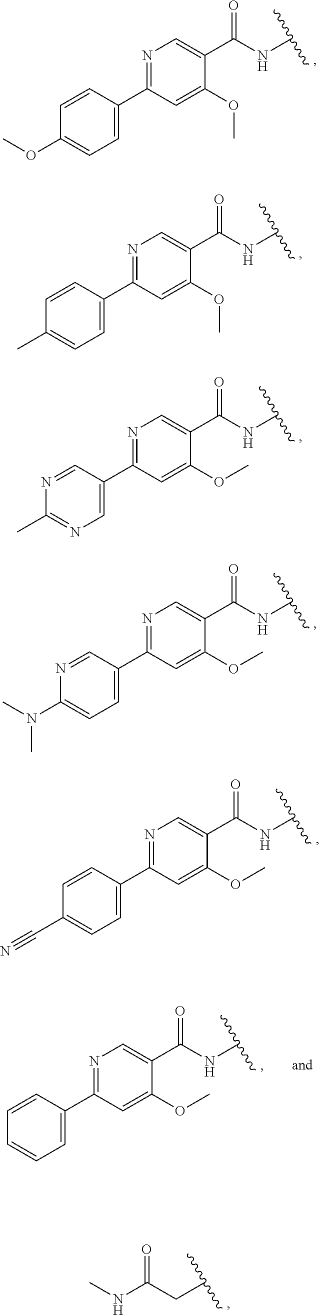

where

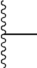

is the point of attachment to the corresponding carbon atom of the structure of Formula (I).

In some embodiments, R² is selected from the group consisting of

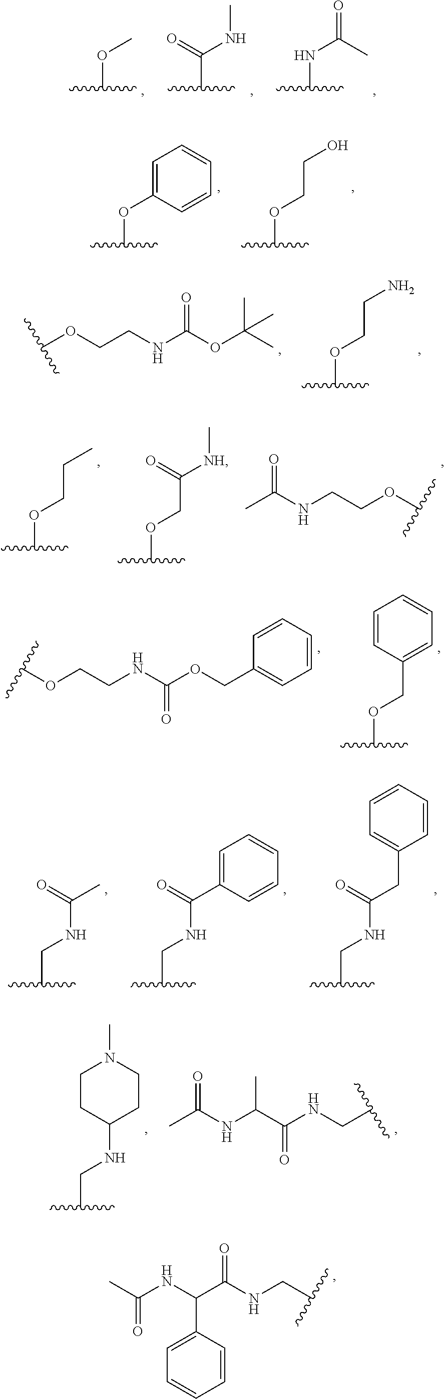

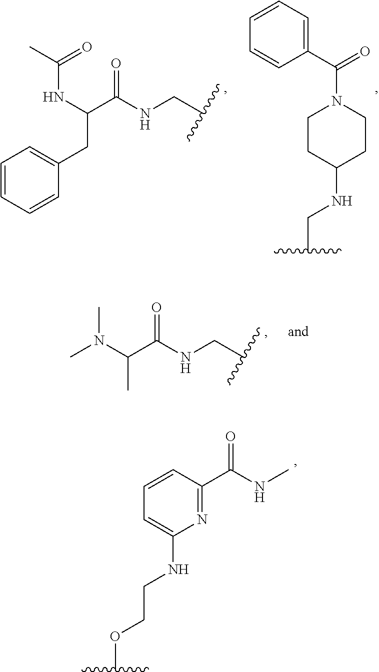

where

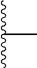

is the point of attachment to the corresponding carbon atom of the structure of Formula (I).

In some embodiments, the compound of Formula (I) is selected from the group consisting of:

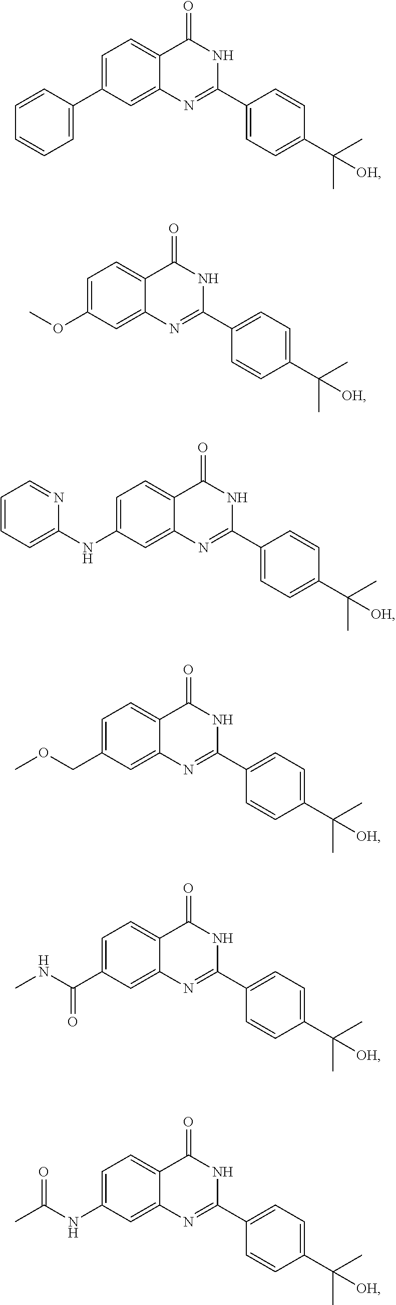 [SMILES: CC(C)(O)c1ccc(-c2nc3cc(-c4ccccc4)ccc3c(=O)[nH]2)cc1 ; COc1ccc2c(=O)[nH]c(-c3ccc(C(C)(C)O)cc3)nc2c1 ; CC(C)(O)c1ccc(-c2nc3cc(Nc4ccccn4)ccc3c(=O)[nH]2)cc1 ; COCc1ccc2c(=O)[nH]c(-c3ccc(C(C)(C)O)cc3)nc2c1 ; CNC(=O)c1ccc2c(=O)[nH]c(-c3ccc(C(C)(C)O)cc3)nc2c1 ; CC(=O)Nc1ccc2c(=O)[nH]c(-c3ccc(C(C)(C)O)cc3)nc2c1]

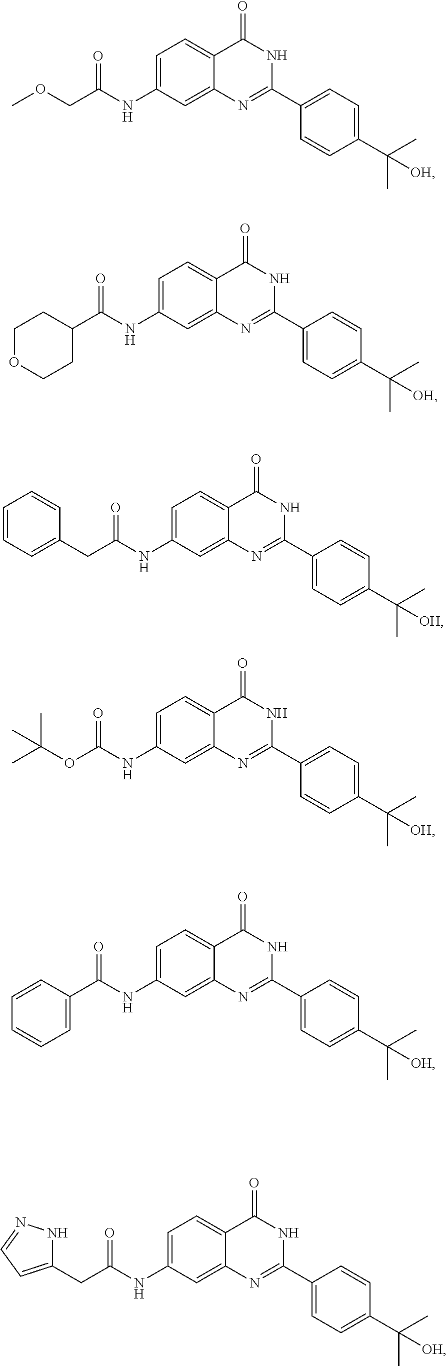 [SMILES: COCC(=O)Nc1ccc2c(=O)[nH]c(-c3ccc(C(C)(C)O)cc3)nc2c1 ; CC(C)(O)c1ccc(-c2nc3cc(NC(=O)C4CCOCC4)ccc3c(=O)[nH]2)cc1 ; CC(C)(O)c1ccc(-c2nc3cc(NC(=O)Cc4ccccc4)ccc3c(=O)[nH]2)cc1 ; CC(C)(C)OC(=O)Nc1ccc2c(=O)[nH]c(-c3ccc(C(C)(C)O)cc3)nc2c1 ; CC(C)(O)c1ccc(-c2nc3cc(NC(=O)c4ccccc4)ccc3c(=O)[nH]2)cc1 ; CC(C)(O)c1ccc(-c2nc3cc(NC(=O)Cc4ccn[nH]4)ccc3c(=O)[nH]2)cc1]

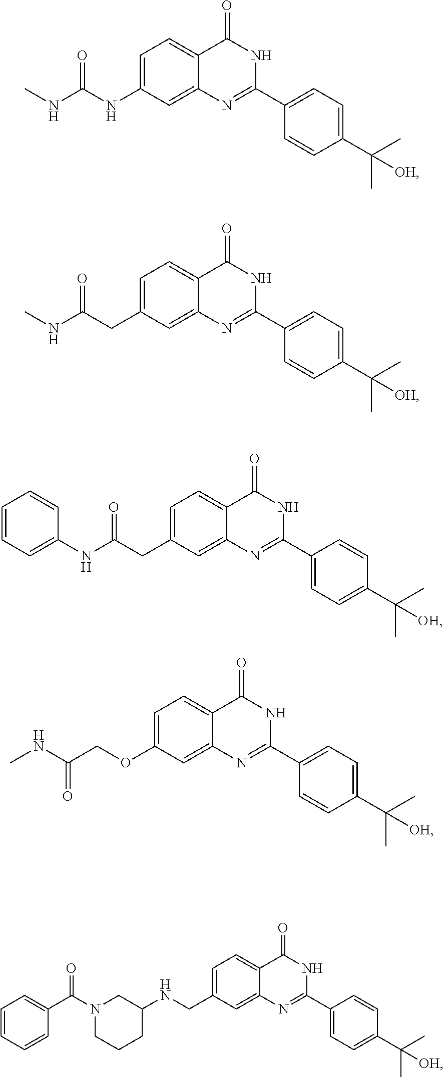 [SMILES: CNC(=O)Nc1ccc2c(=O)[nH]c(-c3ccc(C(C)(C)O)cc3)nc2c1 ; CNC(=O)Cc1ccc2c(=O)[nH]c(-c3ccc(C(C)(C)O)cc3)nc2c1 ; CC(C)(O)c1ccc(-c2nc3cc(CC(=O)Nc4ccccc4)ccc3c(=O)[nH]2)cc1 ; CNC(=O)COc1ccc2c(=O)[nH]c(-c3ccc(C(C)(C)O)cc3)nc2c1 ; CC(C)(O)c1ccc(-c2nc3cc(CNC4CCCN(C(=O)c5ccccc5)C4)ccc3c(=O)[nH]2)cc1]

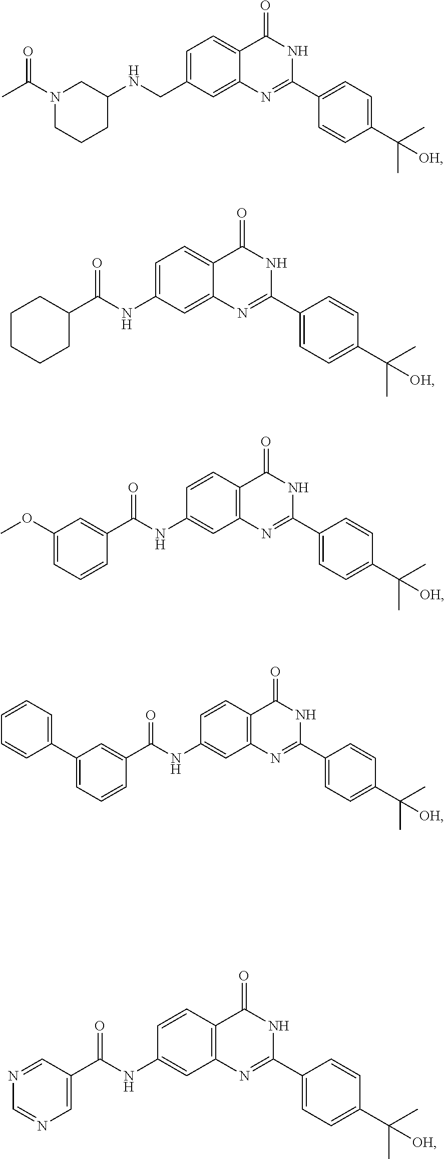 [SMILES: CC(=O)N1CCCC(NCc2ccc3c(=O)[nH]c(-c4ccc(C(C)(C)O)cc4)nc3c2)C1 ; CC(C)(O)c1ccc(-c2nc3cc(NC(=O)C4CCCCC4)ccc3c(=O)[nH]2)cc1 ; COc1cccc(C(=O)Nc2ccc3c(=O)[nH]c(-c4ccc(C(C)(C)O)cc4)nc3c2)c1 ; CC(C)(O)c1ccc(-c2nc3cc(NC(=O)c4cccc(-c5ccccc5)c4)ccc3c(=O)[nH]2)cc1 ; CC(C)(O)c1ccc(-c2nc3cc(NC(=O)c4cncnc4)ccc3c(=O)[nH]2)cc1]

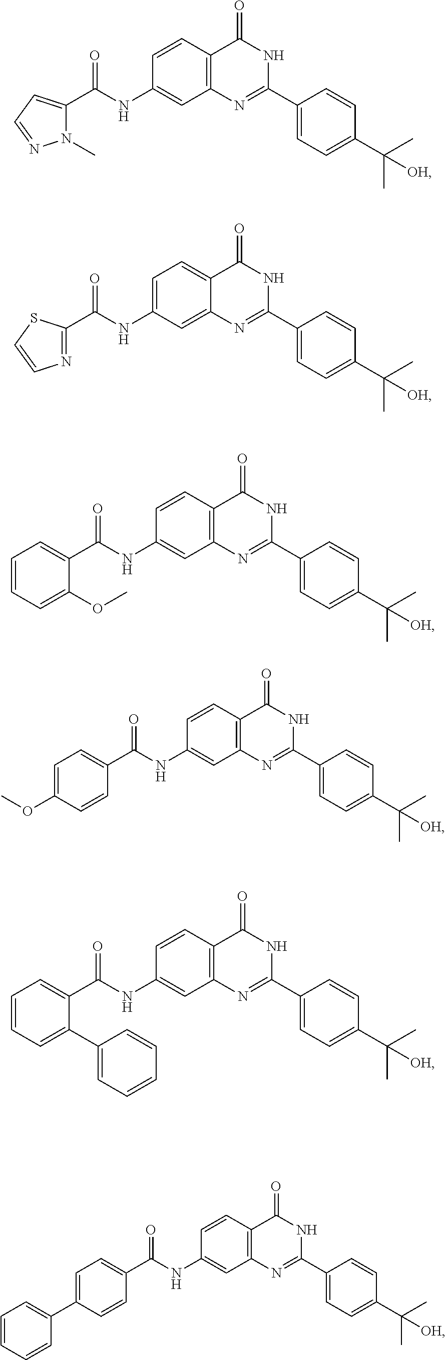 [SMILES: Cn1nccc1C(=O)Nc1ccc2c(=O)[nH]c(-c3ccc(C(C)(C)O)cc3)nc2c1 ; CC(C)(O)c1ccc(-c2nc3cc(NC(=O)c4nccs4)ccc3c(=O)[nH]2)cc1 ; COc1ccccc1C(=O)Nc1ccc2c(=O)[nH]c(-c3ccc(C(C)(C)O)cc3)nc2c1 ; COc1ccc(C(=O)Nc2ccc3c(=O)[nH]c(-c4ccc(C(C)(C)O)cc4)nc3c2)cc1 ; CC(C)(O)c1ccc(-c2nc3cc(NC(=O)c4ccccc4-c4ccccc4)ccc3c(=O)[nH]2)cc1 ; CC(C)(O)c1ccc(-c2nc3cc(NC(=O)c4ccc(-c5ccccc5)cc4)ccc3c(=O)[nH]2)cc1]

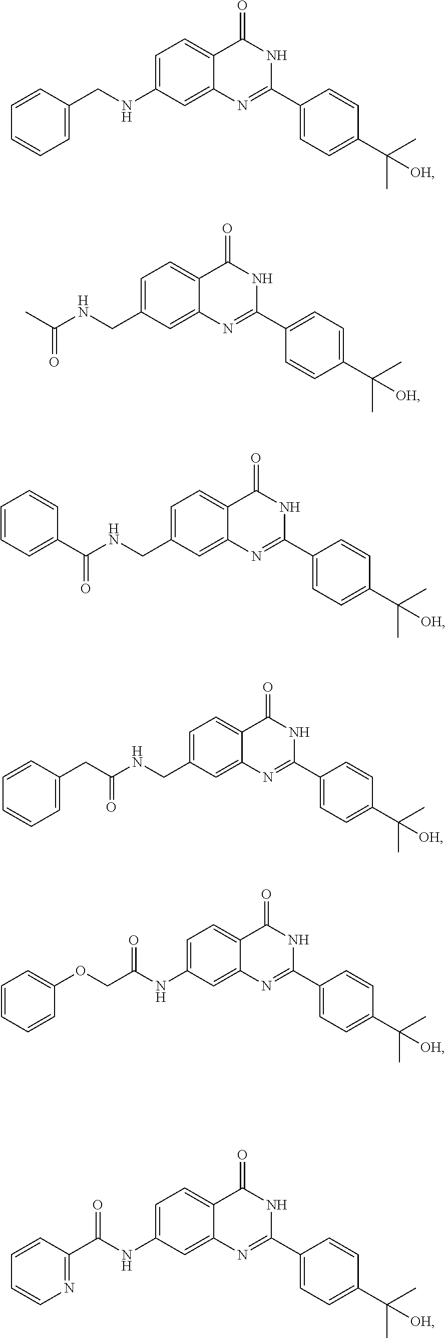 [SMILES: CC(C)(O)c1ccc(-c2nc3cc(NCc4ccccc4)ccc3c(=O)[nH]2)cc1 ; CC(=O)NCc1ccc2c(=O)[nH]c(-c3ccc(C(C)(C)O)cc3)nc2c1 ; CC(C)(O)c1ccc(-c2nc3cc(CNC(=O)c4ccccc4)ccc3c(=O)[nH]2)cc1 ; CC(C)(O)c1ccc(-c2nc3cc(CNC(=O)Cc4ccccc4)ccc3c(=O)[nH]2)cc1 ; CC(C)(O)c1ccc(-c2nc3cc(NC(=O)COc4ccccc4)ccc3c(=O)[nH]2)cc1 ; CC(C)(O)c1ccc(-c2nc3cc(NC(=O)c4ccccn4)ccc3c(=O)[nH]2)cc1]

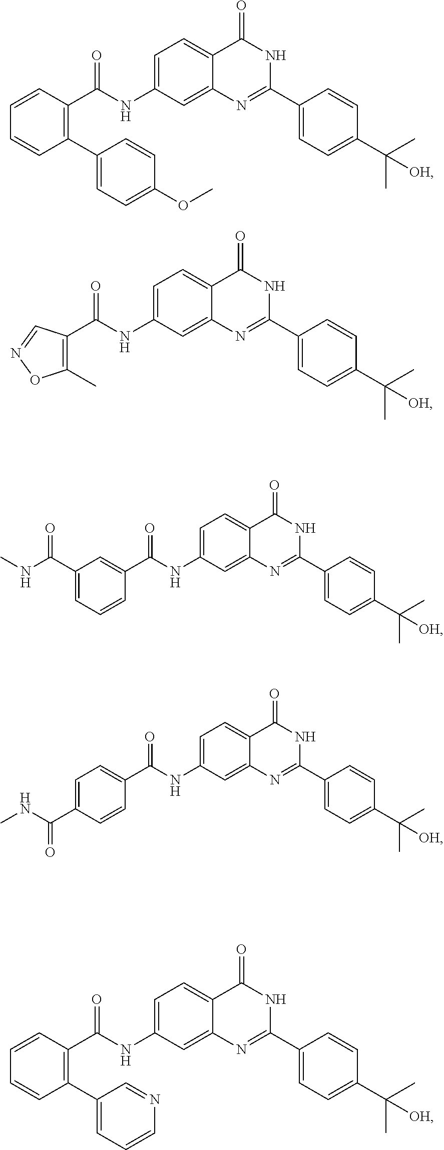 [SMILES: COc1ccc(-c2ccccc2C(=O)Nc2ccc3c(=O)[nH]c(-c4ccc(C(C)(C)O)cc4)nc3c2)cc1 ; Cc1oncc1C(=O)Nc1ccc2c(=O)[nH]c(-c3ccc(C(C)(C)O)cc3)nc2c1 ; CNC(=O)c1cccc(C(=O)Nc2ccc3c(=O)[nH]c(-c4ccc(C(C)(C)O)cc4)nc3c2)c1 ; CNC(=O)c1ccc(C(=O)Nc2ccc3c(=O)[nH]c(-c4ccc(C(C)(C)O)cc4)nc3c2)cc1 ; CC(C)(O)c1ccc(-c2nc3cc(NC(=O)c4ccccc4-c4cccnc4)ccc3c(=O)[nH]2)cc1]

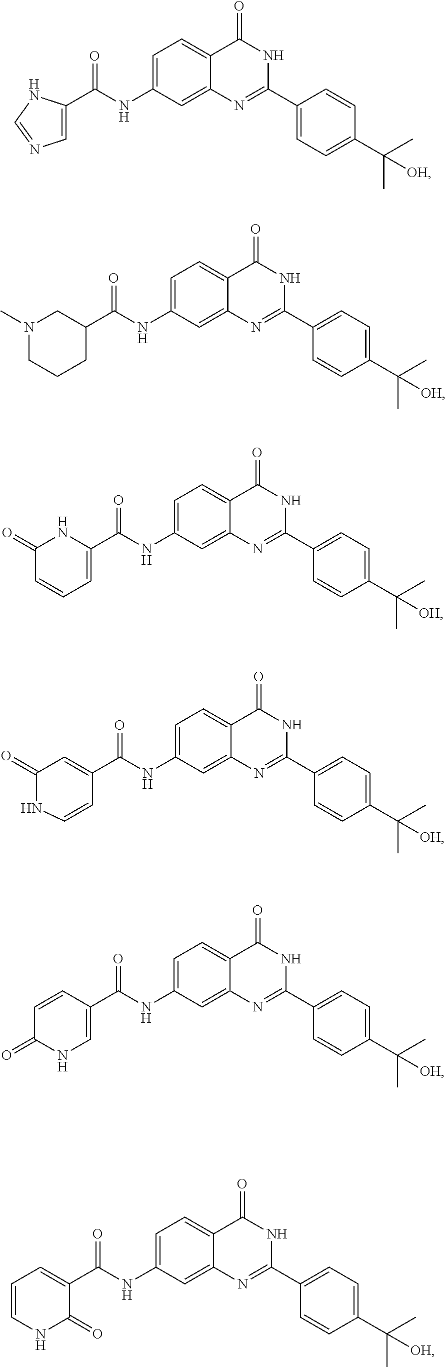 [SMILES: CC(C)(O)c1ccc(-c2nc3cc(NC(=O)c4cnc[nH]4)ccc3c(=O)[nH]2)cc1 ; CN1CCCC(C(=O)Nc2ccc3c(=O)[nH]c(-c4ccc(C(C)(C)O)cc4)nc3c2)C1 ; CC(C)(O)c1ccc(-c2nc3cc(NC(=O)c4cccc(=O)[nH]4)ccc3c(=O)[nH]2)cc1 ; CC(C)(O)c1ccc(-c2nc3cc(NC(=O)c4cc[nH]c(=O)c4)ccc3c(=O)[nH]2)cc1 ; CC(C)(O)c1ccc(-c2nc3cc(NC(=O)c4ccc(=O)[nH]c4)ccc3c(=O)[nH]2)cc1 ; CC(C)(O)c1ccc(-c2nc3cc(NC(=O)c4ccc[nH]c4=O)ccc3c(=O)[nH]2)cc1]

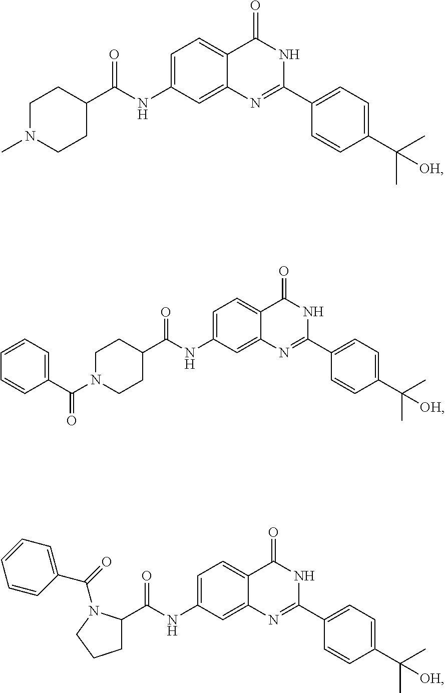 [SMILES: CN1CCC(C(=O)Nc2ccc3c(=O)[nH]c(-c4ccc(C(C)(C)O)cc4)nc3c2)CC1 ; CC(C)(O)c1ccc(-c2nc3cc(NC(=O)C4CCN(C(=O)c5ccccc5)CC4)ccc3c(=O)[nH]2)cc1 ; CC(C)(O)c1ccc(-c2nc3cc(NC(=O)C4CCCN4C(=O)c4ccccc4)ccc3c(=O)[nH]2)cc1]

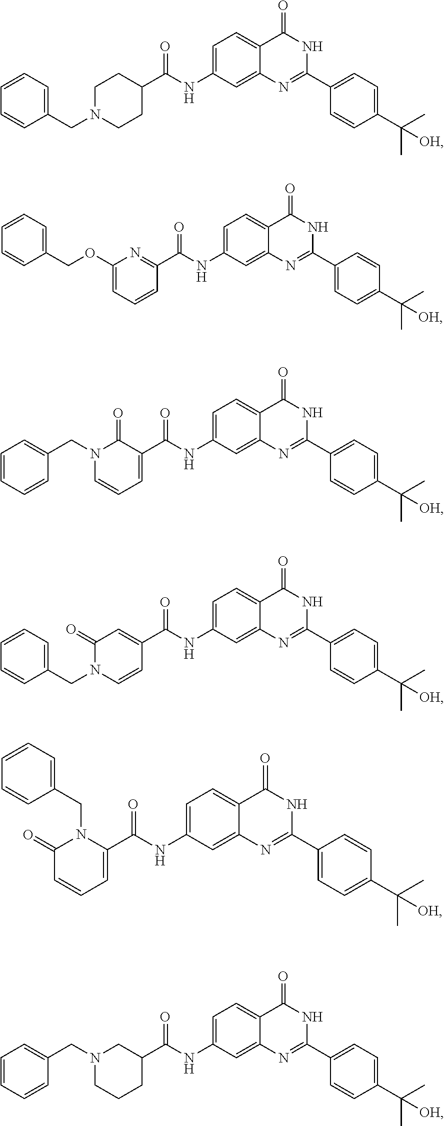 [SMILES: CC(C)(O)c1ccc(-c2nc3cc(NC(=O)C4CCN(Cc5ccccc5)CC4)ccc3c(=O)[nH]2)cc1 ; CC(C)(O)c1ccc(-c2nc3cc(NC(=O)c4cccc(OCc5ccccc5)n4)ccc3c(=O)[nH]2)cc1 ; CC(C)(O)c1ccc(-c2nc3cc(NC(=O)c4cccn(Cc5ccccc5)c4=O)ccc3c(=O)[nH]2)cc1 ; CC(C)(O)c1ccc(-c2nc3cc(NC(=O)c4ccn(Cc5ccccc5)c(=O)c4)ccc3c(=O)[nH]2)cc1 ; CC(C)(O)c1ccc(-c2nc3cc(NC(=O)c4cccc(=O)n4Cc4ccccc4)ccc3c(=O)[nH]2)cc1 ; CC(C)(O)c1ccc(-c2nc3cc(NC(=O)C4CCCN(Cc5ccccc5)C4)ccc3c(=O)[nH]2)cc1]

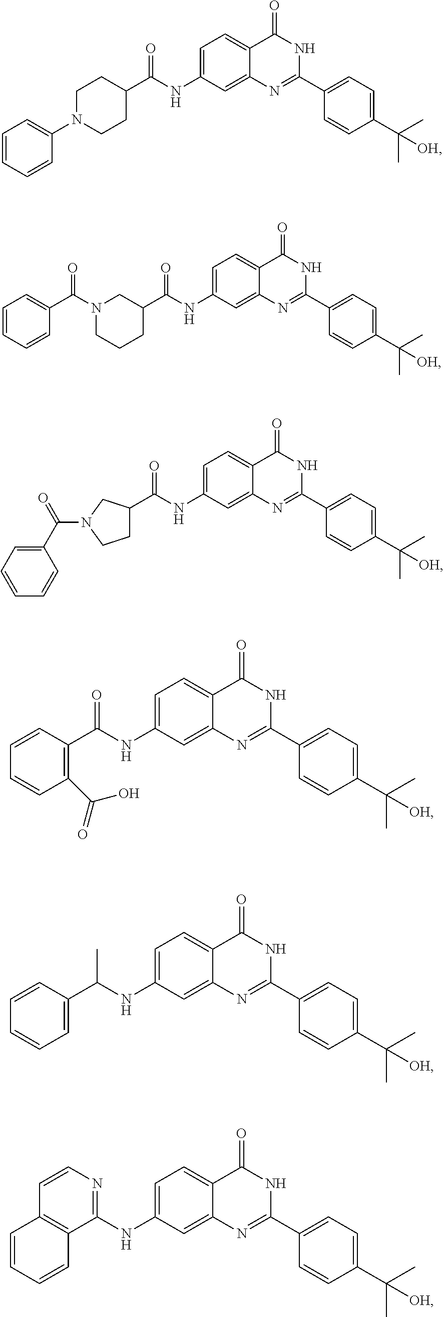 [SMILES: CC(C)(O)c1ccc(-c2nc3cc(NC(=O)C4CCN(c5ccccc5)CC4)ccc3c(=O)[nH]2)cc1 ; CC(C)(O)c1ccc(-c2nc3cc(NC(=O)C4CCCN(C(=O)c5ccccc5)C4)ccc3c(=O)[nH]2)cc1 ; CC(C)(O)c1ccc(-c2nc3cc(NC(=O)C4CCN(C(=O)c5ccccc5)C4)ccc3c(=O)[nH]2)cc1 ; CC(C)(O)c1ccc(-c2nc3cc(NC(=O)c4ccccc4C(=O)O)ccc3c(=O)[nH]2)cc1 ; CC(Nc1ccc2c(=O)[nH]c(-c3ccc(C(C)(C)O)cc3)nc2c1)c1ccccc1 ; CC(C)(O)c1ccc(-c2nc3cc(Nc4nccc5ccccc45)ccc3c(=O)[nH]2)cc1]

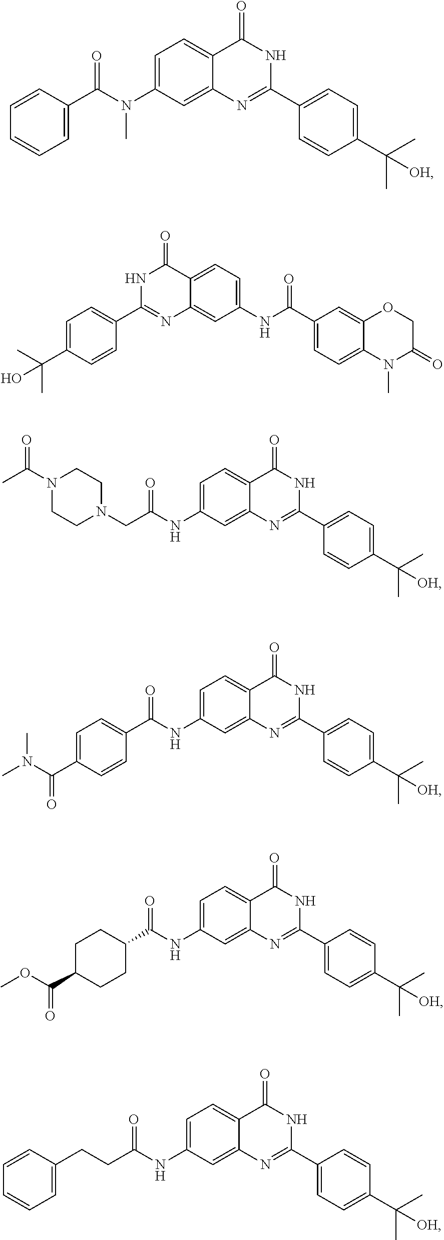 [SMILES: CN(C(=O)c1ccccc1)c1ccc2c(=O)[nH]c(-c3ccc(C(C)(C)O)cc3)nc2c1 ; CN1C(=O)COc2cc(C(=O)Nc3ccc4c(=O)[nH]c(-c5ccc(C(C)(C)O)cc5)nc4c3)ccc21 ; CC(=O)N1CCN(CC(=O)Nc2ccc3c(=O)[nH]c(-c4ccc(C(C)(C)O)cc4)nc3c2)CC1 ; CN(C)C(=O)c1ccc(C(=O)Nc2ccc3c(=O)[nH]c(-c4ccc(C(C)(C)O)cc4)nc3c2)cc1 ; COC(=O)[C@H]1CC[C@H](C(=O)Nc2ccc3c(=O)[nH]c(-c4ccc(C(C)(C)O)cc4)nc3c2)CC1 ; CC(C)(O)c1ccc(-c2nc3cc(NC(=O)CCc4ccccc4)ccc3c(=O)[nH]2)cc1]

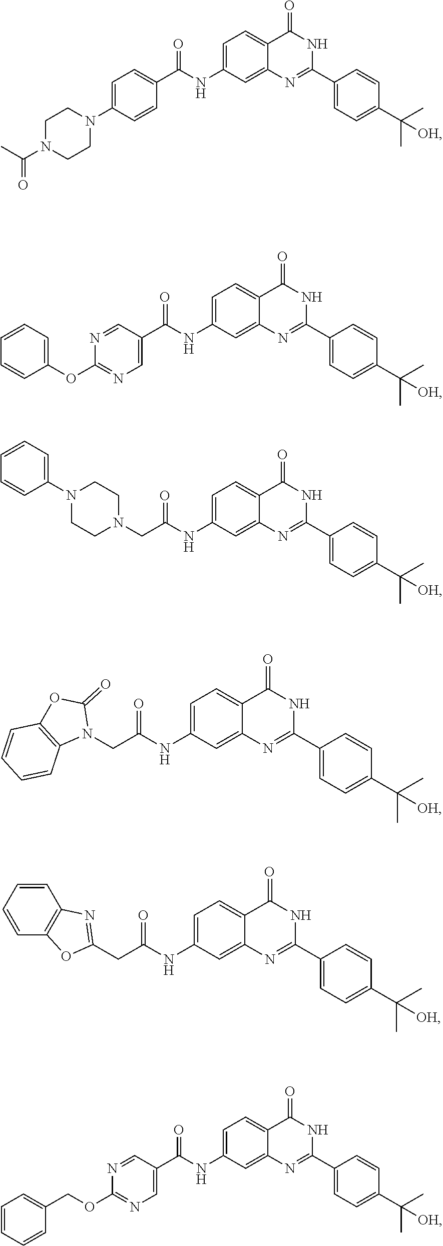 [SMILES: CC(=O)N1CCN(c2ccc(C(=O)Nc3ccc4c(=O)[nH]c(-c5ccc(C(C)(C)O)cc5)nc4c3)cc2)CC1 ; CC(C)(O)c1ccc(-c2nc3cc(NC(=O)c4cnc(Oc5ccccc5)nc4)ccc3c(=O)[nH]2)cc1 ; CC(C)(O)c1ccc(-c2nc3cc(NC(=O)CN4CCN(c5ccccc5)CC4)ccc3c(=O)[nH]2)cc1 ; CC(C)(O)c1ccc(-c2nc3cc(NC(=O)Cn4c(=O)oc5ccccc54)ccc3c(=O)[nH]2)cc1 ; CC(C)(O)c1ccc(-c2nc3cc(NC(=O)Cc4nc5ccccc5o4)ccc3c(=O)[nH]2)cc1 ; CC(C)(O)c1ccc(-c2nc3cc(NC(=O)c4cnc(OCc5ccccc5)nc4)ccc3c(=O)[nH]2)cc1]

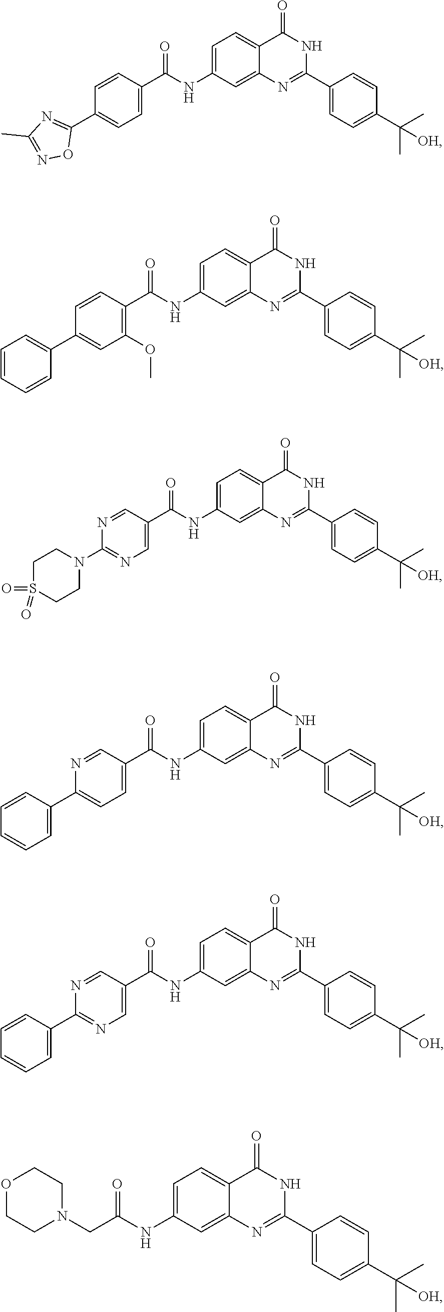 [SMILES: Cc1noc(-c2ccc(C(=O)Nc3ccc4c(=O)[nH]c(-c5ccc(C(C)(C)O)cc5)nc4c3)cc2)n1 ; COc1cc(-c2ccccc2)ccc1C(=O)Nc1ccc2c(=O)[nH]c(-c3ccc(C(C)(C)O)cc3)nc2c1 ; CC(C)(O)c1ccc(-c2nc3cc(NC(=O)c4cnc(N5CCS(=O)(=O)CC5)nc4)ccc3c(=O)[nH]2)cc1 ; CC(C)(O)c1ccc(-c2nc3cc(NC(=O)c4ccc(-c5ccccc5)nc4)ccc3c(=O)[nH]2)cc1 ; CC(C)(O)c1ccc(-c2nc3cc(NC(=O)c4cnc(-c5ccccc5)nc4)ccc3c(=O)[nH]2)cc1 ; CC(C)(O)c1ccc(-c2nc3cc(NC(=O)CN4CCOCC4)ccc3c(=O)[nH]2)cc1]

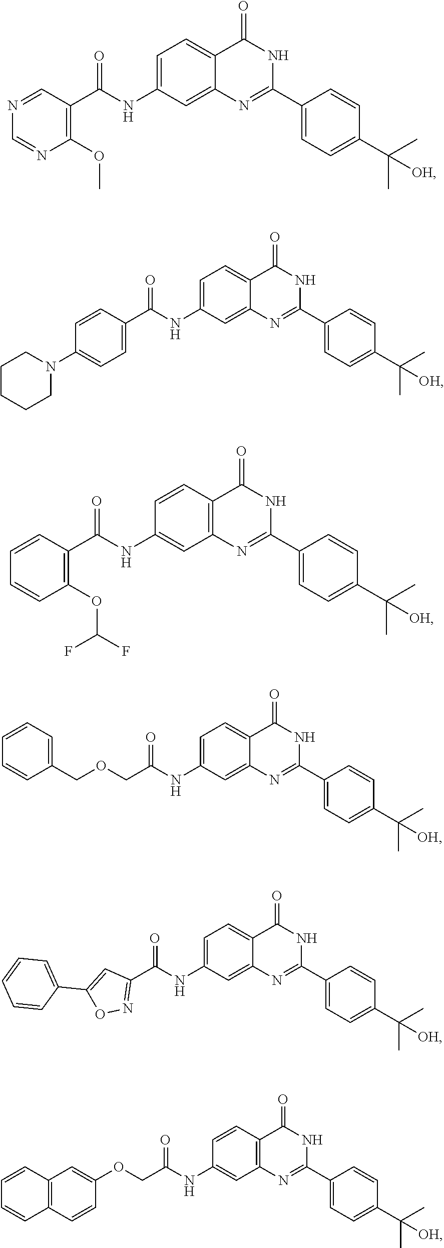 [SMILES: COc1ncncc1C(=O)Nc1ccc2c(=O)[nH]c(-c3ccc(C(C)(C)O)cc3)nc2c1 ; CC(C)(O)c1ccc(-c2nc3cc(NC(=O)c4ccc(N5CCCCC5)cc4)ccc3c(=O)[nH]2)cc1 ; CC(C)(O)c1ccc(-c2nc3cc(NC(=O)c4ccccc4OC(F)F)ccc3c(=O)[nH]2)cc1 ; CC(C)(O)c1ccc(-c2nc3cc(NC(=O)COCc4ccccc4)ccc3c(=O)[nH]2)cc1 ; CC(C)(O)c1ccc(-c2nc3cc(NC(=O)c4cc(-c5ccccc5)on4)ccc3c(=O)[nH]2)cc1 ; CC(C)(O)c1ccc(-c2nc3cc(NC(=O)COc4ccc5ccccc5c4)ccc3c(=O)[nH]2)cc1]

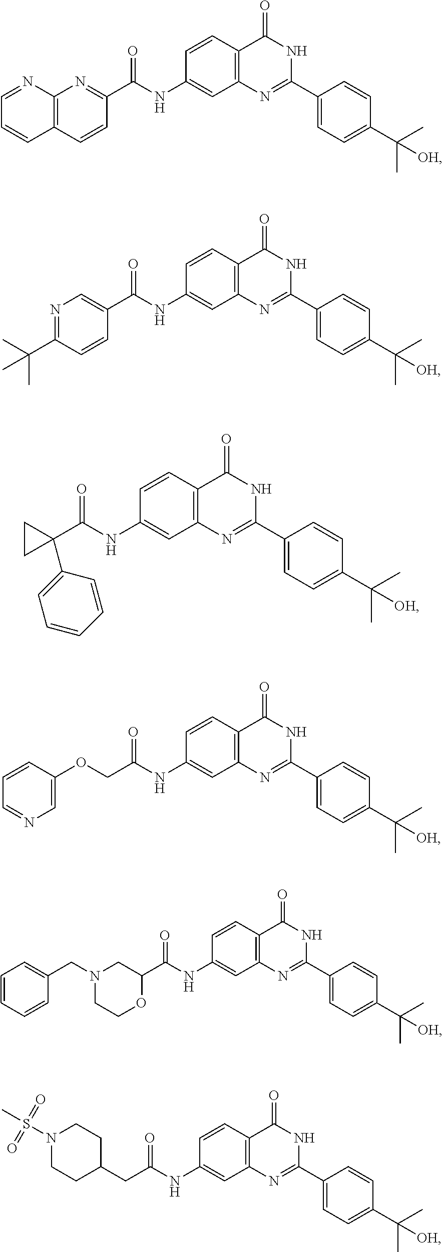 [SMILES: CC(C)(O)c1ccc(-c2nc3cc(NC(=O)c4ccc5cccnc5n4)ccc3c(=O)[nH]2)cc1 ; CC(C)(C)c1ccc(C(=O)Nc2ccc3c(=O)[nH]c(-c4ccc(C(C)(C)O)cc4)nc3c2)cn1 ; CC(C)(O)c1ccc(-c2nc3cc(NC(=O)C4(c5ccccc5)CC4)ccc3c(=O)[nH]2)cc1 ; CC(C)(O)c1ccc(-c2nc3cc(NC(=O)COc4cccnc4)ccc3c(=O)[nH]2)cc1 ; CC(C)(O)c1ccc(-c2nc3cc(NC(=O)C4CN(Cc5ccccc5)CCO4)ccc3c(=O)[nH]2)cc1 ; CC(C)(O)c1ccc(-c2nc3cc(NC(=O)CC4CCN(S(C)(=O)=O)CC4)ccc3c(=O)[nH]2)cc1]

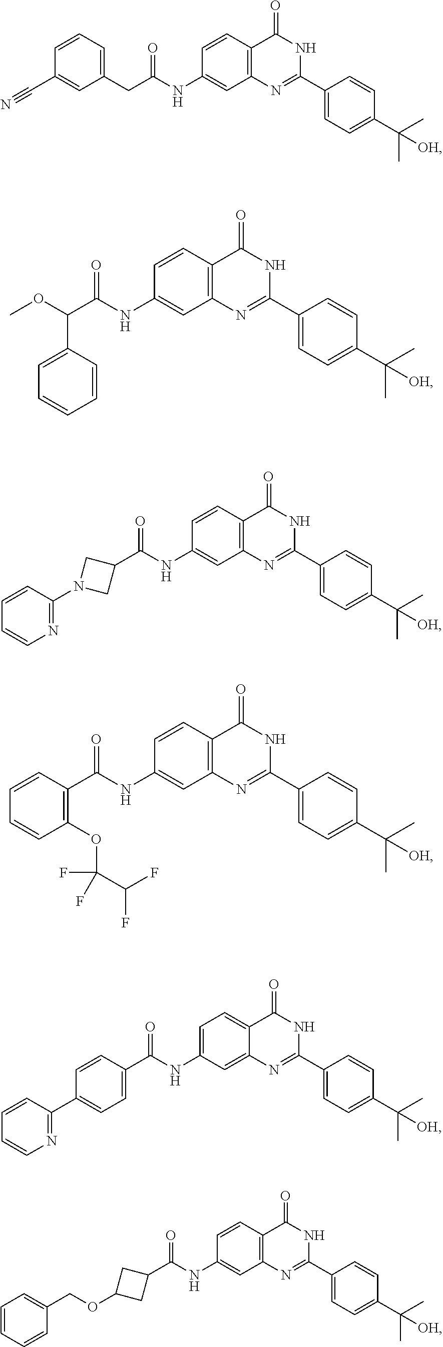 [SMILES: CC(C)(O)c1ccc(-c2nc3cc(NC(=O)Cc4cccc(C#N)c4)ccc3c(=O)[nH]2)cc1 ; COC(C(=O)Nc1ccc2c(=O)[nH]c(-c3ccc(C(C)(C)O)cc3)nc2c1)c1ccccc1 ; CC(C)(O)c1ccc(-c2nc3cc(NC(=O)C4CN(c5ccccn5)C4)ccc3c(=O)[nH]2)cc1 ; CC(C)(O)c1ccc(-c2nc3cc(NC(=O)c4ccccc4OC(F)(F)C(F)F)ccc3c(=O)[nH]2)cc1 ; CC(C)(O)c1ccc(-c2nc3cc(NC(=O)c4ccc(-c5ccccn5)cc4)ccc3c(=O)[nH]2)cc1 ; CC(C)(O)c1ccc(-c2nc3cc(NC(=O)C4CC(OCc5ccccc5)C4)ccc3c(=O)[nH]2)cc1]

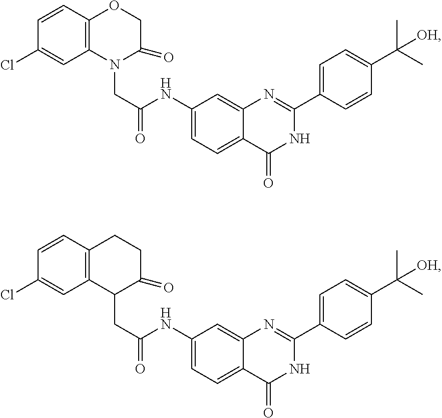 [SMILES: CC(C)(O)c1ccc(-c2nc3cc(NC(=O)CN4C(=O)COc5ccc(Cl)cc54)ccc3c(=O)[nH]2)cc1 ; CC(C)(O)c1ccc(-c2nc3cc(NC(=O)CC4C(=O)CCc5ccc(Cl)cc54)ccc3c(=O)[nH]2)cc1]

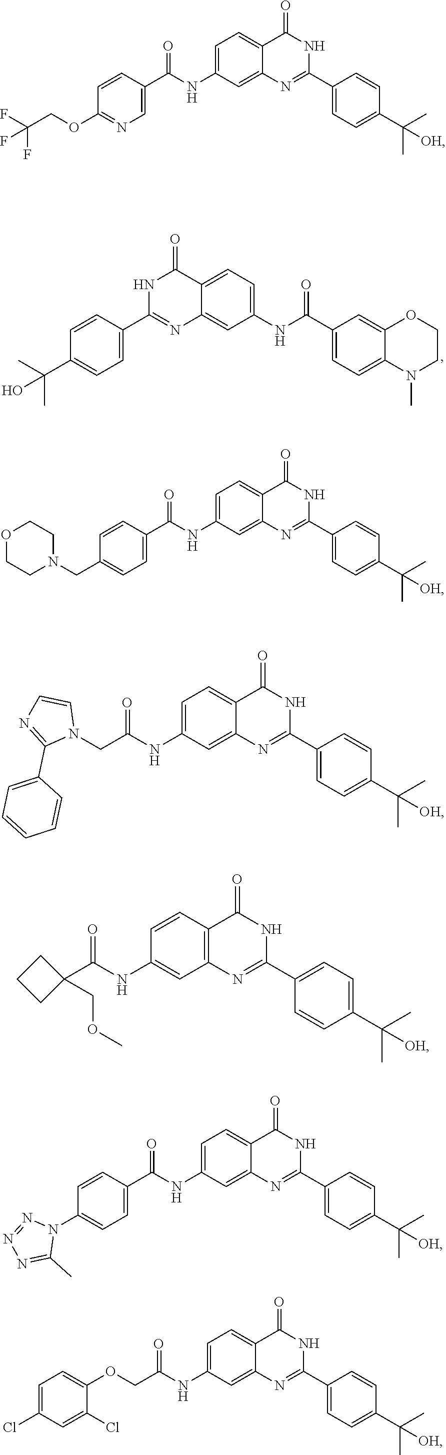

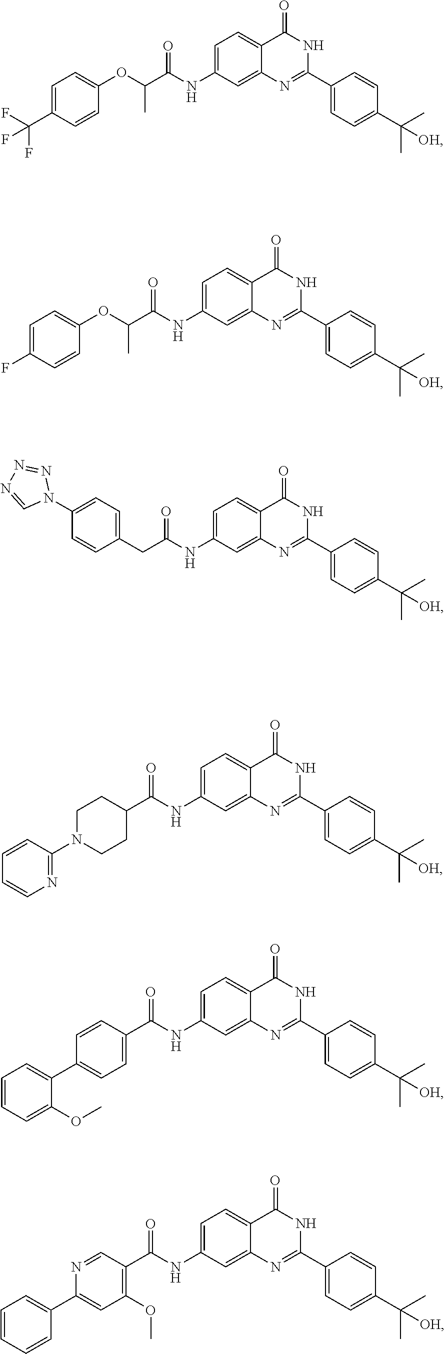 [SMILES: CC(Oc1ccc(C(F)(F)F)cc1)C(=O)Nc1ccc2c(=O)[nH]c(-c3ccc(C(C)(C)O)cc3)nc2c1 ; CC(Oc1ccc(F)cc1)C(=O)Nc1ccc2c(=O)[nH]c(-c3ccc(C(C)(C)O)cc3)nc2c1 ; CC(C)(O)c1ccc(-c2nc3cc(NC(=O)Cc4ccc(-n5cnnn5)cc4)ccc3c(=O)[nH]2)cc1 ; CC(C)(O)c1ccc(-c2nc3cc(NC(=O)C4CCN(c5ccccn5)CC4)ccc3c(=O)[nH]2)cc1 ; COc1ccccc1-c1ccc(C(=O)Nc2ccc3c(=O)[nH]c(-c4ccc(C(C)(C)O)cc4)nc3c2)cc1 ; COc1cc(-c2ccccc2)ncc1C(=O)Nc1ccc2c(=O)[nH]c(-c3ccc(C(C)(C)O)cc3)nc2c1]

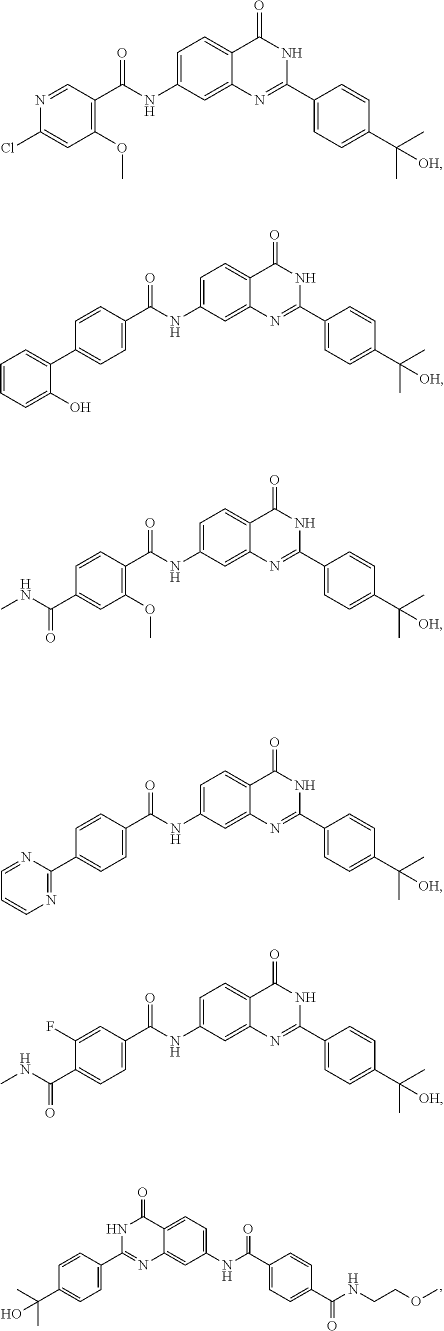 [SMILES: COc1cc(Cl)ncc1C(=O)Nc1ccc2c(=O)[nH]c(-c3ccc(C(C)(C)O)cc3)nc2c1 ; CC(C)(O)c1ccc(-c2nc3cc(NC(=O)c4ccc(-c5ccccc5O)cc4)ccc3c(=O)[nH]2)cc1 ; CNC(=O)c1ccc(C(=O)Nc2ccc3c(=O)[nH]c(-c4ccc(C(C)(C)O)cc4)nc3c2)c(OC)c1 ; CC(C)(O)c1ccc(-c2nc3cc(NC(=O)c4ccc(-c5ncccn5)cc4)ccc3c(=O)[nH]2)cc1 ; CNC(=O)c1ccc(C(=O)Nc2ccc3c(=O)[nH]c(-c4ccc(C(C)(C)O)cc4)nc3c2)cc1F ; COCCNC(=O)c1ccc(C(=O)Nc2ccc3c(=O)[nH]c(-c4ccc(C(C)(C)O)cc4)nc3c2)cc1]

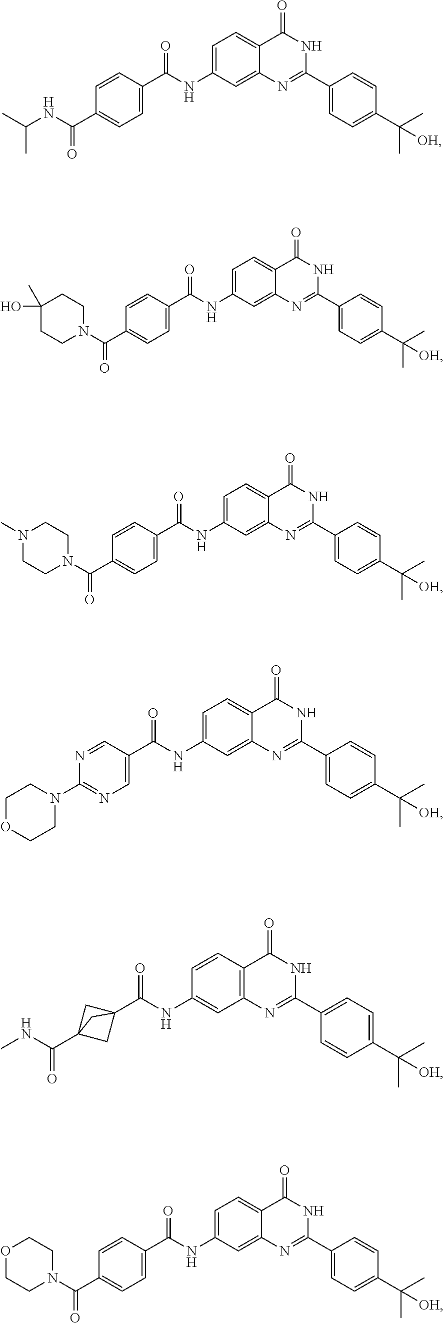

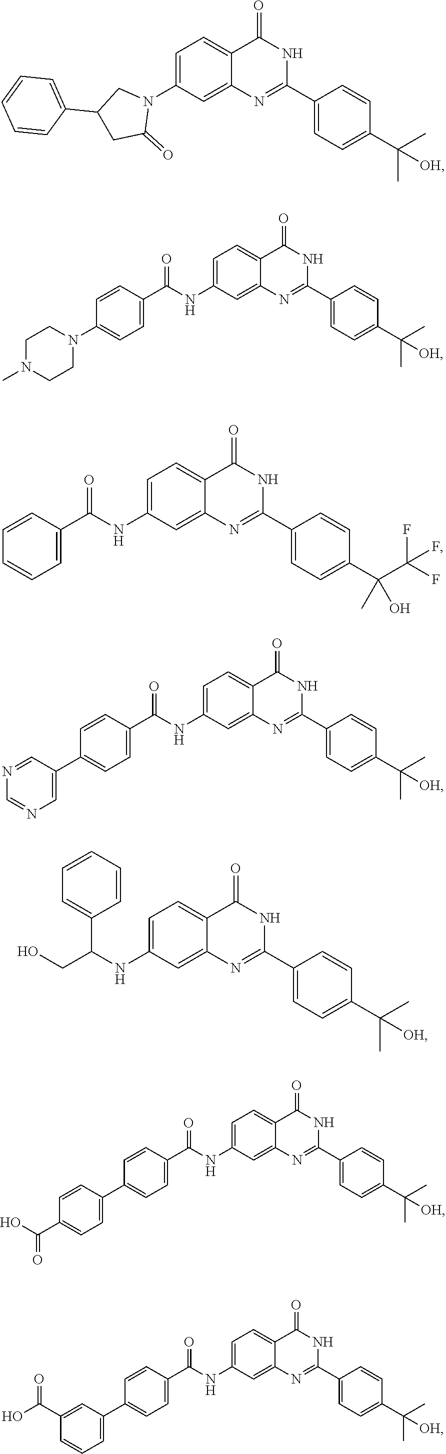

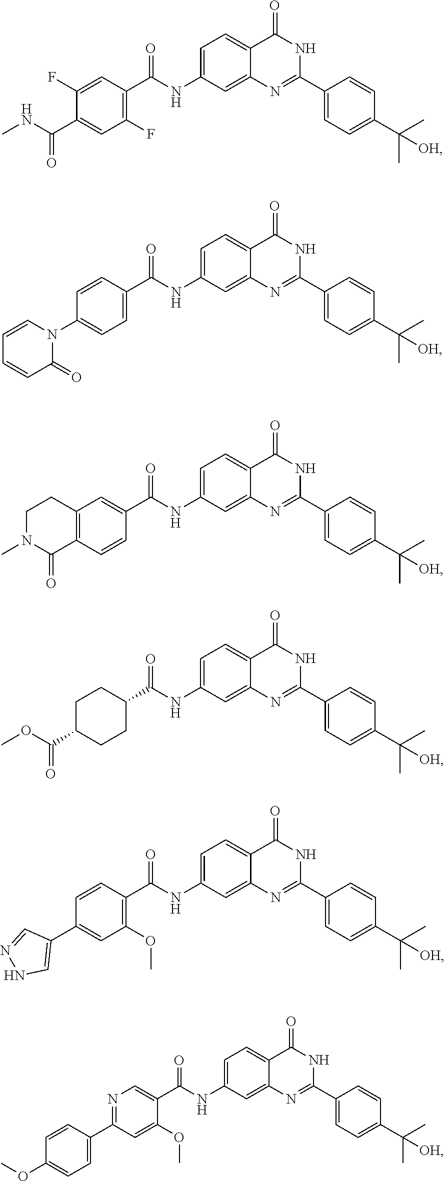 [SMILES: CNC(=O)c1cc(F)c(C(=O)Nc2ccc3c(=O)[nH]c(-c4ccc(C(C)(C)O)cc4)nc3c2)cc1F ; CC(C)(O)c1ccc(-c2nc3cc(NC(=O)c4ccc(-n5ccccc5=O)cc4)ccc3c(=O)[nH]2)cc1 ; CN1CCc2cc(C(=O)Nc3ccc4c(=O)[nH]c(-c5ccc(C(C)(C)O)cc5)nc4c3)ccc2C1=O ; COC(=O)[C@H]1CC[C@@H](C(=O)Nc2ccc3c(=O)[nH]c(-c4ccc(C(C)(C)O)cc4)nc3c2)CC1 ; COc1cc(-c2cn[nH]c2)ccc1C(=O)Nc1ccc2c(=O)[nH]c(-c3ccc(C(C)(C)O)cc3)nc2c1 ; COc1ccc(-c2cc(OC)c(C(=O)Nc3ccc4c(=O)[nH]c(-c5ccc(C(C)(C)O)cc5)nc4c3)cn2)cc1]

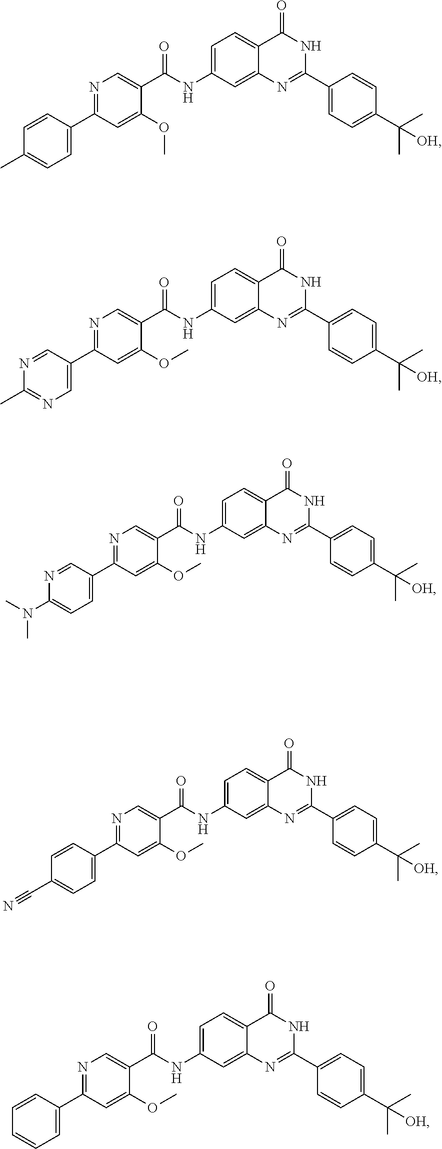 [SMILES: COc1cc(-c2ccc(C)cc2)ncc1C(=O)Nc1ccc2c(=O)[nH]c(-c3ccc(C(C)(C)O)cc3)nc2c1 ; COc1cc(-c2cnc(C)nc2)ncc1C(=O)Nc1ccc2c(=O)[nH]c(-c3ccc(C(C)(C)O)cc3)nc2c1 ; COc1cc(-c2ccc(N(C)C)nc2)ncc1C(=O)Nc1ccc2c(=O)[nH]c(-c3ccc(C(C)(C)O)cc3)nc2c1 ; COc1cc(-c2ccc(C#N)cc2)ncc1C(=O)Nc1ccc2c(=O)[nH]c(-c3ccc(C(C)(C)O)cc3)nc2c1 ; COc1cc(-c2ccccc2)ncc1C(=O)Nc1ccc2c(=O)[nH]c(-c3ccc(C(C)(C)O)cc3)nc2c1]

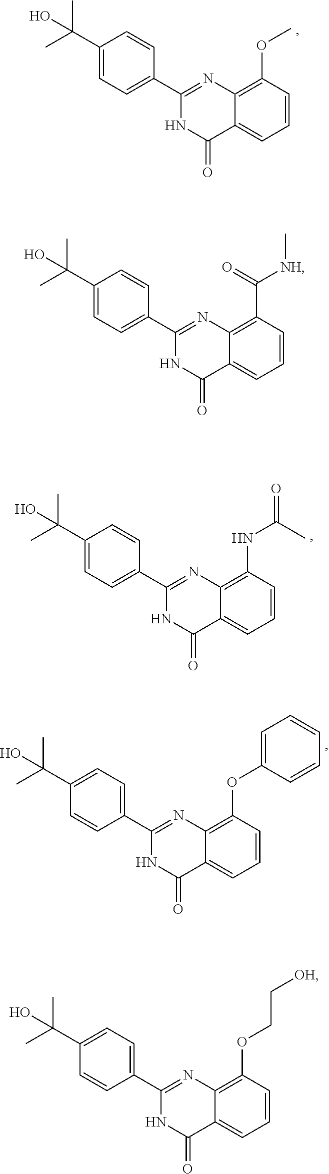

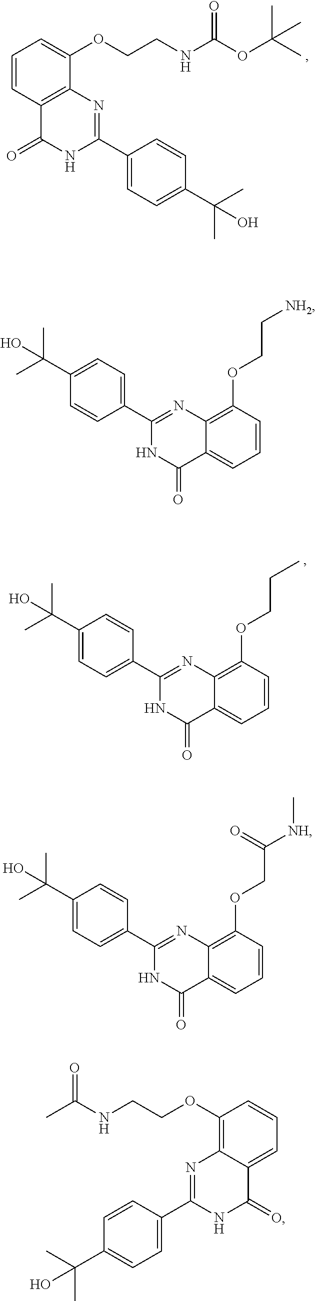

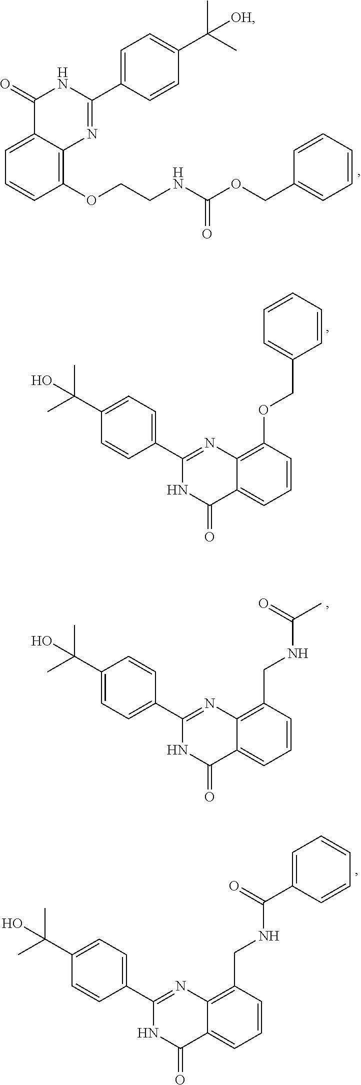

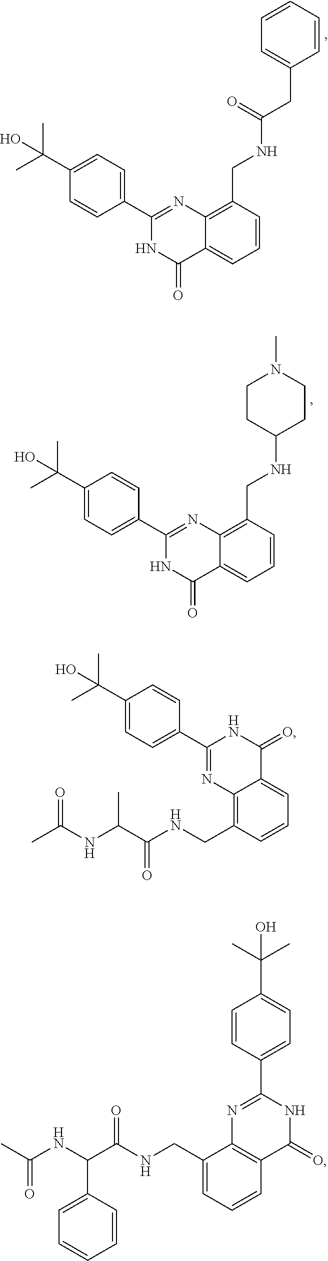 [SMILES: CC(C)(O)c1ccc(-c2nc3c(CNC(=O)Cc4ccccc4)cccc3c(=O)[nH]2)cc1 ; CN1CCC(NCc2cccc3c(=O)[nH]c(-c4ccc(C(C)(C)O)cc4)nc23)CC1 ; CC(=O)NC(C)C(=O)NCc1cccc2c(=O)[nH]c(-c3ccc(C(C)(C)O)cc3)nc12 ; CC(=O)NC(C(=O)NCc1cccc2c(=O)[nH]c(-c3ccc(C(C)(C)O)cc3)nc12)c1ccccc1]

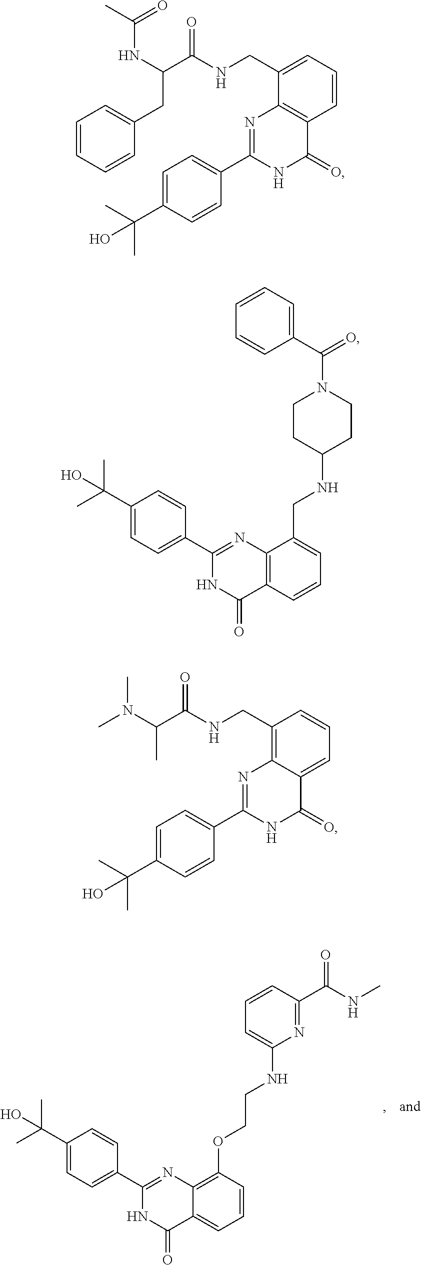 [SMILES: CC(=O)NC(Cc1ccccc1)C(=O)NCc1cccc2c(=O)[nH]c(-c3ccc(C(C)(C)O)cc3)nc12 ; CC(C)(O)c1ccc(-c2nc3c(CNC4CCN(C(=O)c5ccccc5)CC4)cccc3c(=O)[nH]2)cc1 ; CC(C(=O)NCc1cccc2c(=O)[nH]c(-c3ccc(C(C)(C)O)cc3)nc12)N(C)C ; CNC(=O)c1cccc(NCCOc2cccc3c(=O)[nH]c(-c4ccc(C(C)(C)O)cc4)nc23)n1]

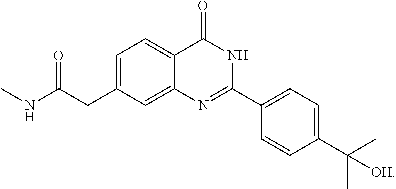 [SMILES: CNC(=O)Cc1ccc2c(=O)[nH]c(-c3ccc(C(C)(C)O)cc3)nc2c1]

Compounds of the present disclosure can be produced according to Scheme 1 below.

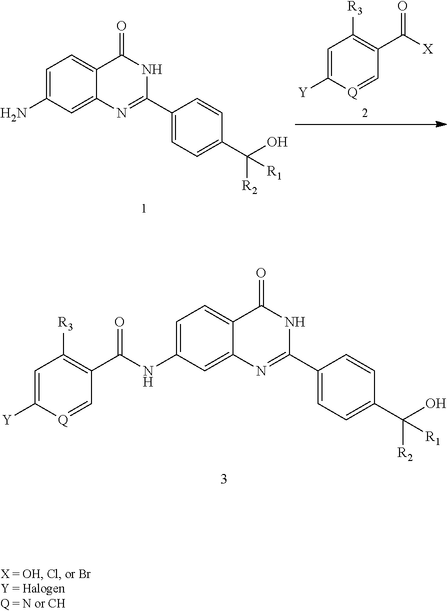

Amine (1) can be reacted with a carboxylic acid or a derivative thereof (2) in the presence of a base to produce compound (3). In some cases (when X is OH) the reaction is carried out in the presence of a base and a coupling agent. Suitable coupling agents that can be used include 2-chloro-1,3-dimethyl-4,5-dihydroimidazol-1-ium chloride (CMPI), N-[(dimethylamino)-1H-1,2,3-triazolo-[4,5-b]pyridin-1-ylmethylene]-N-methylmethanaminium hexafluorophosphate N-oxide (HATU), chloro-N,N,N′,N′-tetramethylformamidinium hexafluorophosphate (TCFH), dicyclohexylcarbodiimide (DCC), 1-Ethyl-3-(3-dimethylaminopropyl) carbodiimide (EDC), and 1-Propanephosphonic anhydride (T₃P). Suitable bases that can be used include pyridine, diisopropylethylamine (DIPEA), triethylamine, and 1-methylimidazole, and. This reaction can be carried out in any suitable solvent, such as pyridine, dichloromethane, and tetrahydrofuran. This reaction can be carried out at room temperature or at elevated temperatures. For example, it can be carried out at a temperature of from about 80° C. to about 120° C. Alternatively, this reaction can be carried out at from about 18° C. to about 27° C. This reaction can be carried out for about 6-20 hours. This reaction can be carried out under inert atmosphere (nitrogen or argon) or open to air.

Another aspect of the present disclosure relates to a pharmaceutical composition comprising a therapeutically effective amount of the compound of Formula (I) and a pharmaceutically acceptable carrier.

The compounds and pharmaceutical compositions of the present invention are particularly useful for the treatment of cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

While it may be possible for compounds of Formula (I), Formula (Ia), and Formula (Ib), to be administered as raw chemicals, it will often be preferable to present them as a part of a pharmaceutical composition. Accordingly, another aspect of the present invention is a pharmaceutical composition containing a therapeutically effective amount of the compound of Formula (I), Formula (Ia), and Formula (Ib), or a pharmaceutically acceptable salt or solvate thereof, and a pharmaceutically acceptable carrier. The carrier must be “acceptable” in the sense of being compatible with the other ingredients of the formulation and not deleterious to the recipient thereof.

Another aspect of the present disclosure relates to a method of treating cancer in a subject. This method comprises administering to the subject in need thereof the compound of Formula (I).

In some embodiments, the cancer is selected from the group consisting of small cell lung carcinoma, non-small cell lung carcinoma, esophageal cancer, melanoma, liver cancer, lung cancer, sarcoma, cholangiocarcinoma, mesothelioma, gastric cancer, hepatocellular cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

Another aspect of the present disclosure relates to a method of inhibiting Tankyrase 2 (TNKS2) activity. This method comprises contacting a TNKS2 with the compound of Formula (I) under conditions effective to inhibit TNKS2 activity.

In some embodiments, the TNKS2 activity is inhibited selectively.

In some embodiments, the compound is at least two-fold more selective for inhibiting TNKS2 activity over Tankyrase 1 (TNKS1) activity.

Another aspect of the present disclosure relates to a method of treating a condition where it is desired to inhibit TNKS2 activity. This method comprises administering to the subject in need thereof the compound of Formula (I).

Another aspect of the present disclosure relates to a method of treating a proliferation-related disorder in a subject. This method comprises administering to the subject in need thereof the compound of Formula (I).

Another aspect of the present disclosure relates to a method of treating abnormal cell growth in a subject. This method comprises administering to the subject in need thereof the compound of Formula (I).

In some embodiments, the abnormal cell growth is a benign growth or a malignant growth.

In some embodiments, the abnormal cell growth is a psoriasis, benign prostatic hypertrophy, or restenosis.

In some embodiments, the abnormal cell growth is carcinoma, sarcoma, lymphoma, or leukemia.

In some embodiments, the abnormal cell growth is a cancer. In some embodiments, the cancer is selected from the group consisting of small cell lung carcinoma, non-small cell lung carcinoma, esophageal cancer, melanoma, liver cancer, lung cancer, sarcoma, cholangiocarcinoma, mesothelioma, gastric cancer, hepatocellular cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

In some embodiments, the methods disclosed herein further comprise administering an additional therapeutic agent. In some embodiments, the additional therapeutic agent is selected from the group consisting of antibiotics, anti-emetic agents, antidepressants, antifungal agents, anti-inflammatory agents, antineoplastic agents, antiviral agents, cytotoxic agents, and other anticancer agents, immunomodulatory agents, alpha-interferons, β-interferons, alkylating agents, hormones, and cytokines.

In some embodiments, the additional therapeutic agent demonstrates anti-cancer activity. In some embodiments, the additional therapeutic agent demonstrates cytotoxic activity.

In practicing the methods of the present disclosure, agents suitable for treating a subject can be administered using any method standard in the art. The agents, in their appropriate delivery form, can be administered orally, intradermally, intramuscularly, intraperitoneally, intravenously, subcutaneously, or intranasally. The compositions of the present disclosure may be administered alone or with suitable pharmaceutical carriers, and can be in solid or liquid form, such as tablets, capsules, powders, solutions, suspensions, or emulsions.

The agents of the present invention may be orally administered, for example, with an inert diluent, or with an assimilable edible carrier, or it may be enclosed in hard or soft shell capsules, or it may be compressed into tablets, or they may be incorporated directly with the food of the diet. Agents of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present invention in such therapeutically useful compositions is such that a suitable dosage will be obtained.

Also specifically contemplated are oral dosage forms of the agents of the present invention. The agents may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, “Soluble Polymer-Enzyme Adducts,” In: Enzymes as Drugs, Hocenberg and Roberts, eds., Wiley-Interscience, New York, N.Y., pp. 367-383 (1981), which are hereby incorporated by reference in their entirety). Other polymers that could be used are poly-1,3-dioxolane and poly-1,3,6-tioxocane. Preferred for pharmaceutical usage, as indicated above, are polyethylene glycol moieties.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, sucralose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The agents of the present invention may also be administered parenterally. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents.

Intraperitoneal or intrathecal administration of the agents of the present invention can also be achieved using infusion pump devices such as those described by Medtronic, Northridge, CA. Such devices allow continuous infusion of desired compounds avoiding multiple injections and multiple manipulations.

In addition to the formulations described previously, the agents may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The agents of the present disclosure may also be administered directly to the airways in the form of an aerosol. For use as aerosols, the agent of the present invention in solution or suspension may be packaged in a pressurized aerosol container together with suitable propellants, for example, hydrocarbon propellants like propane, butane, or isobutane with conventional adjuvants. The agents of the present disclosure also may be administered in a non-pressurized form such as in a nebulizer or atomizer.

Effective doses of the compositions of the present invention, for the treatment of cancer or pathogen infection vary depending upon many different factors, including type and stage of cancer, means of administration, target site, physiological state of the patient, other medications or therapies administered, and physical state of the patient relative to other medical complications. Treatment dosages need to be titrated to optimize safety and efficacy.

The percentage of active ingredient in the compositions of the present invention may be varied, it being necessary that it should constitute a proportion such that a suitable dosage shall be obtained. Obviously, several unit dosage forms may be administered at about the same time. The dose employed will be determined by the physician, and depends upon the desired therapeutic effect, the route of administration and the duration of the treatment, and the condition of the patient. In the adult, the doses are generally from about 0.01 to about 100 mg/kg body weight, preferably about 0.01 to about 10 mg/kg body weight per day by inhalation, from about 0.01 to about 100 mg/kg body weight, preferably 0.1 to 70 mg/kg body weight, more especially 0.1 to 10 mg/kg body weight per day by oral administration, and from about 0.01 to about 50 mg/kg body weight, preferably 0.01 to 10 mg/kg body weight per day by intravenous administration. In each particular case, the doses will be determined in accordance with the factors distinctive to the subject to be treated, such as age, weight, general state of health, and other characteristics which can influence the efficacy of the medicinal product.

The products according to the present disclosure may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active product may be administered orally 1 to 4 times per day. It goes without saying that, for other patients, it will be necessary to prescribe not more than one or two doses per day.

The above disclosure is general. A more specific description is provided below in the following examples. The examples are described solely for the purpose of illustration and are not intended to limit the scope of the present application. Changes in form and substitution of equivalents are contemplated as circumstances suggest or render expedient. Although specific terms have been employed herein, such terms are intended in a descriptive sense and not for purposes of limitation.

### EXAMPLES

The following Examples are presented to illustrate various aspects of the present application, but are not intended to limit the scope of the claimed application.

### Example 1—Materials and Methods

### Cell Lines

DLD1 [CCL-221], SW480 [CCL-228], SW1417 [CCL-238], and 293T [CRL-3216] cell lines were purchased from ATCC and cultured in DMEM supplemented with 1% Penicillin-streptomycin and 10% fetal bovine serum. 3T3 cells [CRL-1658] were purchased from ATCC and cultured DMEM supplemented with 1% Penicillin-streptomycin and 10% fetal calf serum. OVCAR-4 cells were purchased from Sigma-Aldrich [SCC258] and cultured in RPMI-1640 supplemented with 1% Penicillin-streptomycin and 10% fetal bovine serum. SW403 cells were purchased from ATCC

[CCL-230] and cultured in RPMI-1640 supplemented with 1% Penicillin-streptomycin and 10% fetal bovine serum. PC9 and H23 cells (a gift from Dr. Harold Varmus, Weill Cornell Medicine) were cultured in RPMI-1640 supplemented with 1% Penicillin-streptomycin and 10% fetal bovine serum. HCC1171 cells (a gift from Dr. Gina DeNicola, Moffitt Cancer Center, FL) were cultured in RPMI-1640 supplemented with 1% Penicillin-streptomycin and 10% fetal bovine serum. Sf9 (Spodoptera frugiperda) insect cells were obtained from Expression Systems and cultured in ESF 921 supplemented with penicillin, streptomycin, and Amphotericin B. Cells were tested for mycoplasma using a Mycoplasma PCR Detection Kit (ABM #G238).

### Cloning

The shRNA sequences were cloned into the LT3-GEPIR, SGEP, or SiREP vectors using restriction digest cloning. TNKS2 cDNA was purchased from GenScript (OHu10661) and cloned into the SGEP vector with a 3×FLAG tag (gBlock) using Gibson assembly. L1136Y mutation was introduced to the TNKS2 cDNA using overlap PCR, and hygromycin resistance was introduced using restriction digest cloning. TOPFlash sequences were cloned into the SGEN vector using Gibson assembly. AXIN1-GFP reporter sequence was cloned into the SGEN vector, and the Kozak sequence was replaced using Gibson assembly. The SAM-PARP coding region of TNKS2 (amino acids 850-1166) was PCR amplified and cloned into a pFastbac expression vector featuring a N-terminal twin-strep-MBP tag utilizing Gibson assembly. Site directed mutagenesis to generate the L1136Y mutant was performed through Gibson assembly. sgRNAs were cloned into the LRT2B vector or SFFV.NLS-mScarlet vector. shRNA sequences, sgRNA sequences, and cloning primers are listed in Table 1.

TABLE 1
shRNA Sequences, sgRNA Sequences, and Cloning Primers

| | | | SEQ ID |
|---|---|---|---|
| | qPCR Primers | Sequence | NO: |
| | hRPL27_F1 | CATGGGCAAGAAGAAGATCG | 1 |
| | hRPL27_R1 | TCCAAGGGGATATCCACAGA | 2 |
| | Krt20_F1 | CCCAGAAGAACCTGCAAGAG | 3 |
| | Krt20_R1 | CCTCCGTGTTCACTGTGACT | 4 |
| | | | SEQ ID |
| | Cloning Primers | Sequence | NO: |
| shRNAs and | shRenilla.713 | TGCTGTTGACAGTGAGCGCAGGAATTATA | 5 |
| sgRNAs | | ATGCTTATCTATAGTGAAGCCACAGATGTA | |
| | | TAGATAAGCATTATAATTCCTATGCCTACT | |
| | | GCCTCGGA | |
| | shTNKS.2658 | TGCTGTTGACAGTGAGCGCAGCGGCTTTA | 6 |
| | | TTGATAAAATATAGTGAAGCCACAGATGT | |
| | | ATATTTTATCAATAAAGCCGCTATGCCTAC | |
| | | TGCCTCGGA | |
| | shTNKS2.3287 | TGCTGTTGACAGTGAGCGCCAGAAGGTTT | 7 |
| | | GTAACAAGAAATAGTGAAGCCACAGATGT | |
| | | ATTTCTTGTTACAAACCTTCTGATGCCTACT | |
| | | GCCTCGGA | |
| | Tnks_1385 | TGCTGTTGACAGTGAGCGAAAGTCTGTTCT | 8 |
| | | CTGTTACTTATAGTGAAGCCACAGATGTAT | |
| | | AAGTAACAGAGAACAGACTTCTGCCTACT | |
| | | GCCTCGGA | |
| | Tnks_3341 | TGCTGTTGACAGTGAGCGCCCAGAAGATA | 9 |
| | | AAGAATATCAATAGTGAAGCCACAGATGT | |
| | | ATTGATATTCTTTATCTTCTGGATGCCTACT | |
| | | GCCTCGGA | |
| | Tnks2_1328 | TGCTGTTGACAGTGAGCGCGCTGATGTTA | 10 |
| | | CAAGAATCAAATAGTGAAGCCACAGATGT | |
| | | ATTTGATTCTTGTAACATCAGCTTGCCTACT | |
| | | GCCTCGGA | |
| | Tnks2_3004 | TGCTGTTGACAGTGAGCGCACCTTAGATG | 11 |
| | | TCCTAGTTGAATAGTGAAGCCACAGATGT | |
| | | ATTCAACTAGGACATCTAAGGTATGCCTAC | |
| | | TGCCTCGGA | |
| | Tnks_5-2_F | caccgGGGGAGTCCGAAGATGG | 12 |
| | Tnks_5-2_R | aaacCCATCTTCGGACTCCCCC | 13 |
| | Apc.1405_A | CACCGTTCAGAGTGAGCCATGTAG | 14 |
| | Apc.1405_B | AAACCTACATGGCTCACTCTGAAC | 15 |
| | Trp53.Q97_A | CACCGCTTCTCAAAAAACTTACCA | 16 |
| | Trp53.Q97_B | AAACTGGTAAGTTTTTTGAGAAGC | 17 |
| | Tnks 5′ | GTACGGGGGAGTGGGAAGA | 18 |
| | Tnks 3′ | GCTAGAGTGACAGTGCAGAG | 19 |
| TNKS2 cDNA | SFFV_3xFlag_ | AAAGAGCTCACAACCCCTCACTCGGCGCG | 20 |
| expression | TNKS2 | CCAGTCCTCCGACAGGTGGATCCACCATG | |
| plasmids | | GATTATAAGGATCACGATGGCGATTATAA | |
| | | GGACCACGACATCGACTACAAGGACGACG | |
| | | ACGACAAGTCGGGTCGCCGCTGCGCCGGC | |
| | GA_Flag_TNKS2_F | CGACAAGTCGGGTCGCCGCTGCGCC | 21 |
| | GA_Flag_TNKS2_R | AGTAAACAAGATAATTGCTCGAATTTTATC | 22 |
| | | CATCGACCATACCTTCAGGCC | |
| | TNKS2_F | AGTACAGTTCGAGAGCACAGA | 23 |
| | TNKS2_int_Rev | TGCTAGGCCATTTACACTGG | 24 |
| | TNKS2_L1136Y_ | CCAGTGTAAATGGCCTAGCATATGCTGAAT | 25 |
| | For | ATGTTATTTACAG | |
| | TNKS2_Rev | AAGAACGGAGCCGGTTGGCGC | 26 |
| shRNA | miRE-XhoI-Fw_for | TGAACTCGAGAAGGTATATTGCTGTTGAC | 27 |
| plasmids | oligo | AGTGAGCG | |
| | miRE-EcoRI- | TCTCGAATTCTAGCCCCTTGAAGTCCGAGG | 28 |
| | Rev_for oligo | CAGTAGGC | |
| TOPFlash | TOP_FF_Fwd | CGATCACGAGACTAGCCTCGATCGAGCTA | 29 |
| plasmids | | GCTGCCAGAACATTTCTCTATCGAT | |
| | TOP_FF_Rev | TTAGTAAACAAGATAATTGCTCCCGCCCCG | 30 |
| | | TCTCCAATTATTACACGGCGATCTTTCCGC | |
| | Ren-P2A-iRFP_F | TGGTATAAATTAAATCACTTTTTTCAATTGC | 31 |
| | | TCGAGTGGCTCCGGTGCCC | |
| | Ren-P2A-iRFP_R | TGTAATCCAGAGGTTGATTGTTCCAGGCTA | 32 |
| | | GCTCACTCTTCCATCACG | |
| AXIN-GFP | GA_SFFV_Fwd | GATCACGAGACTAGCCTCGATCGAGCTAG | 33 |
| | | CTGCAGTAACGCCATTTT | |
| | GA_SFFV_Rev | CCGGTAGATCCACCGGCCGACTCAGTCTG | 34 |
| | | TCGGAGGACTGGCGCGCC | |
| | GA_BadKozak_ | GTCGGCCGGTGGATCTACCGGTGGATAAC | 35 |
| | GFP_Fwd | AAATGGTGAGCAAGGGCGAGGA | |
| | GA_GFP_Rev | CCACCGATCCCCTCCGGACTTGTACAGCTC | 36 |
| | | GTCCATGCCGAGAGT | |
| TNKS KO | U6-TNKS-F | TCGATCACGAGACTAGCCTCGATCGGAGG | 37 |
| plasmids | | GCCTATTTCCCATGAT | |
| | U6-TNKS-R | CAAAATGGCGTTACTGCAGCTAGCTATACT | 38 |
| | | GCCATTTGTCTCAAG | |
| | NLS-GFP-F | CCGGTGGATCTACCGGTGGATCCACTCGG | 39 |
| | | CTGGCGGTTCCACAGG | |
| | NLS-GFP-R | CTACCCGGTAGAATTACGCGTCGAACCCA | 40 |
| | | ACCCTATAAAACTAGG | |
| | Mouse | | |
| | Genotyping | | SEQ ID |
| Locus | Primers | Sequence | NO: |
| R26 | Rosa_A | AAAGTCGCTCTGAGTTGTTAT | 41 |
| | Rosa_B | GCGAAGAGTTTGTCCTCAACC | 42 |
| | Rosa_C5 | CCTCCAATTTTACACCTGTTC | 43 |
| Col1a1 | Col1a1_F | AATCATCCCAGGTGCACAGCATTGCGG | 44 |
| | Col1a1_R | CTTTGAGGGCTCATGAACCTCCCAGG | 45 |
| | SAdpA_R | ATCAAGGAAACCCTGGACTACTGCG | 46 |
| TGM- | shRen_GT_F | AAGCCACAGATGTATAGATAAGCATT | 47 |
| shRen.713 | RBG_R1 (Rev43) | GAAAGAACAATCAAGGGTCC | 48 |
| TGM- | GT_T2.3004 | AGCCACAGATGTATTCAACTAGGA | 49 |
| shTnks2.3004 | RBG_R1 (Rev43) | GAAAGAACAATCAAGGGTCC | 48 |
| TGM- | GT_T.1385 | AGCCACAGATGTATAAGTAACAGA | 50 |
| shTNKS1/ | RBG_R1 (Rev43) | GAAAGAACAATCAAGGGTCC | 48 |
| 2.13-30 | | | |
| TGM- | GT_T.3341 | AAGCCACAGATGTATTGATATTCTTT | 51 |
| shTNKS1/ | RBG_R2 (Rev45) | CACCCTGAAAACTTTGCCCC | 52 |
| 2.33-13 | | | |
| Tnks | Tnks_3_F | CGGCAGCCATTTCCTCAAAG | 53 |
| | Tnks-5-2_R | CTCCACTACTAAGCCACGCC | 54 |
| | Tnks_5F2_geno | TGGCGGAGCCGAAGCCCCTG | 55 |
| LSL-Kras | LSL-Kras (Y116- | TCCGAATTCAGTGACTACAGATG | 56 |
| | common) | | |
| | LSL-Kras (Y118- | ATGTCTTTCCCCAGCACAGT | 57 |
| | WT) | | |
| | LSL-Kras (Y117- | CTAGCCACCATGGCTTGAGT | 58 |
| | LSL) | | |
| ApcQ1405X | 7a1_miseq_F1 | TGTTGAGTTTTCTTCAGGAGCC | 59 |
| | 7a1_miseq_R2 | TGGTCTGCCCAGGACTATCT | 60 |

### Lentiviral Transduction

HEK-293T cells were seeded to 90% confluency one day prior to transfection. The cells were co-transfected with 5 ug of plasmid DNA, 2.5 ug PAX2 vector, and 1.25 ug VSVg vector. shRNA plasmids were additionally transfected with 1.25 ug pcSUPER-shPasha plasmid. Vectors were combined in 150 ul DMEM with 30 ul PEI and incubated at RT for 10 minutes. Transfection mix was incubated with the 293 Ts overnight. Cells were then transferred to collection media, and lentiviral supernatants were collected at 24 and 48 hours following the change to collection media. Remaining HEK-293T cells were removed through centrifugation. Target cells were seeded to 30-50% confluency. After 24 hours, the target cells were incubated overnight with diluted lentiviral supernatant mixed with 8 ug/mL polybrene. After 24 hours, the viral media was replaced with culture media. Antibiotic selection was initiated 48 hours following transduction.

### Generation of TNKS1 KO Cell Lines

DLD1 cells were transduced with Cas9-p2a-puro and selected with 2 ug/mL puromycin. Cas9-expressing cells were then transduced with an sgRNA sequence targeting TNKS1 (LRT2B or NLS-mScarlet) (sgRNA sequences listed in Table 1) and selected with 5 ug/mL blasticidin or 400 ug/mL neomycin. Transduced cells were plated as single cells by limiting dilution to identify clonal lines.

### Intestinal Organoid Isolation and Culture

Mouse small intestinal organoids were isolated as described previously (O'Rourke et al., “Isolation, Culture, and Maintenance of Mouse Intestinal Stem Cells,” Bio Protoc. 6 (2016), which is hereby incorporated by reference in its entirety). Organoids were plated in basal medium (Advanced DMEM/F12 containing penicillin/streptomycin, glutamine, and HEPES) containing EGF [40 ng/ml], LDN [50 nM LDN]. RSPO1 [5% conditioned media], and Fungin [10 ug/ml]. Organoids were maintained by passaging every 2-3 days. To passage the organoids, the Matrigel containing organoids was collected in PBS. The organoids were mechanically separated using a P1000 pipette and washed with additional PBS. The organoids were spun at 1200 rpm for 4 minutes. The PBS was aspirated, and the organoid pellet was resuspended in Matrigel. After Matrigel polymerization (5 mins at RT, 5 mins at 37C), organoids were cultured in ENR media (basal medium containing EGF [40 ng/ml], LDN [50 nM], RSPO1 [5% conditioned media]).

### Crypt Survival

Mice were treated with doxycycline for 2 or 3 weeks. Small intestinal crypts were isolated as described above and plated in multiple wells of a 48-well plate (40 ul Matrigel/well). Organoids were plated in ENR media (described above) containing 0.5 ug/mL doxycycline when indicated. ENR+dox media was replaced on day 2. Viable organoids were counted 12 hours and 4 days after plating using a brightfield microscope.

### Organoid Transfection

Prior to transfection, organoids were cultured in media containing CHIR99021 (5 uM) and Y-27632 (10 uM) for two days. Organoids were dissociated to single cells by incubating in 200 uL TrypLE at 37 C for 5 minutes, centrifuged, and resuspended in 600 uL culture media. 2 ug of DNA was mixed with 4 uL Lipofectamine 2000 in 200 uL Opti-MEM, and the transfection mix was combined with the organoid suspension in a 24-well plate. The plate was then centrifuged at 600 g for 60 minutes at 32C. Following centrifugation, the plate was incubated at 37 C for an additional 4 hours. The organoids were pelleted and replated in Matrigel in media containing CHIR99021 (5 uM) and Y-27632 (10 uM) for 2 days to facilitate organoid recovery. To select for APC mutations, RSPO1 and LDN were removed from the media 2 days after transfection. To select for p53 mutations, the organoids were treated with Nutlin (10 uM).

### Organoid Proliferation and Viability

Organoids were plated at low density in 40 ul of Matrigel in 24-well plates and cultured in EN media (Basal media+EGF+LDN, described above). Organoids were grown continually for 6 days in media containing DMSO or inhibitor compounds, with compounds replenished by changing media every two days. Organoid area was quantified using the Sartorius Incucyte S3. After 6 days of compound treatment, alamarBlue Cell Viability Reagent (ThermoFisher #DAL1100) was added to each well at a 1:10 dilution and incubated at 37 C for 4 hours. 100 ul of media was transferred to a 96-well plate and fluorescence intensity was quantified using a BMG Labtech Fluostar Omega microplate reader. For serial passaging experiments, organoids were plated at high density in 120 ul of Matrigel each in 2 wells per condition of a 12-well plate in EN media containing DMSO or inhibitor compound. Media was changed every 2 days. Every 4 days, 100 μl of alamarBlue was added to one well per condition and quantified as above. The second well was passaged 1:2 into the same condition in a new 12-well plate.

### Western Blot

700,000 cells were seeded in each well of a 6-well plate and incubated for 24 hours for the cells to adhere. Compounds were then added to the cells and incubated for 24 hours. To isolate protein, cells were washed in PBS and lysed in 200 ul RIPA buffer on ice for 20 minutes. Lysates were centrifuged at 15,000 rpm for 10 minutes and the protein supernatant was stored at −80C. 20 ug of protein was run per sample for western blot analysis. Antibodies used were anti-AXIN1 [Cell Signaling Technology, #2087; 1:1000], anti-TNKS1/2 [Santa Cruz Biotechnology, #sc-365897; 1:1000], anti-GFP [Cell Signaling Technology, #2956; 1:1000], and anti-beta Actin-HRP [Abcam, #ab49900; 1:10,000]. When blotting for TNKS1/2, cells were incubated in 1 uM XAV939 for 24 hours prior to cell lysis unless otherwise indicated. Protein abundance was quantified using Image Lab software.

### Biochemical PARylation Assays

TNKS1 and TNKS2 enzymatic activity was measured using the BPS Bioscience TNKS1 (#80573) and TNKS2 (#80572) Histone Ribosylation assay kit according to the manufacturer's instructions. Chemiluminescence was measured using a BMG Labtech Fluostar Omega microplate reader.

### AXIN1-GFP Flow Cytometry

GFP reporter cell lines were seeded 50,000 cells per well in 24-well plates. The cells were incubated for 24 hours to allow the cells to adhere prior to starting compound treatments. After adhering, the media was replaced with culture media containing inhibitor compounds. The compounds were diluted in DMSO at 1000× final concentration, then diluted 1:1000 in cell culture media. The cells were treated for 24 hours. Following this incubation, the cells were trypsinized in 100 ul at 37 C for 5 minutes, then resuspended in 300 ul culture media containing DAPI. 300 ul of the resuspension was transferred to a round-bottom 96-well plate and fluorescence was measured on a ThermoFisher Attune NxT flow cytometer. Results were analyzed using Flowjo software.

### TOPflash Assay

TOPflash expressing cell lines were seeded 15,000 cells per well in clear-bottom black 96-well plates. The plates were incubated for 24 hours to allow the cells to adhere. Following 24 hours, the compounds were serially diluted in cell culture media using a multichannel pipette and replaced the media in the cell culture plates. Following 48 hours of compound treatment, firefly and renilla luciferase were analyzed using the BPS Bioscience Dual Luciferase (Firefly-Renilla) Assay System (#60683-2). The assay was performed according to the manufacturer's instructions, and luminescence was recorded using a BMG Labtech Fluostar Omega microplate reader.

### Colony Forming Assays

Cells were seeded at 1000 cells per well into 12-well plates and incubated for 24 hours for cells to adhere. Compounds were added to the media and were replenished every two days. Once colonies formed (about 16 days after compounds were added), the media was removed, and the cells were fixed in 4% paraformaldehyde for one hour at RT on an orbital shaker. The PFA was removed, and the fixed cells were stained in a 10% Giemsa solution in PBS overnight on an orbital shaker. After staining, the Giemsa solution was removed, the plates were washed in water, and left to dry overnight. Once dry, the plates were imaged using an Epson Perfection V550 Photo scanner. To quantify cell growth, 1 mL of 10% acetic acid was added to each well. The plate was shaken for 20 minutes, and 100 ul of the acetic acid solution was transferred to a 96-well plate. Absorbance was quantified using a BMG Labtech Fluostar Omega microplate reader.

### RNA Isolation and RNA-seq

One six-well of organoids was plated per condition into EN media (basal medium containing EGF [40 ng/ml] and LDN [50 nM LDN]) containing DMSO, 250 nM G007-LK, or 500 nM TDI-012804. Organoids were cultured for 3 days, with media replenished on the second day. The organoids were collected in 1 mL of TRIzol (Thermo Fisher Scientific, #15596018) and RNA was extracted following the manufacturer's protocol. DNA was removed from the isolated RNA by treating with DNase1 for 15 minutes followed by column purification with the Qiagen RNeasy kit (Qiagen #74106). The RNA quality was confirmed using a 2100 Bioanalyzer (Agilent technologies), the RNA library was prepared using TruSeq Stranded mRNA Sample Library Preparation Kit (Illumina), and RNA-seq was performed on an Illumina NovaSeq 6000 with paired-end 2×100 cycles.

### RNAseq Analysis

Transcript abundance was estimated using Kallisto (Bray et al., “Near-Optimal Probabilistic RNA-Seq Quantification,” Nat. Biotechnol. 34:525-527 (2016), which is herein incorporated by reference in its entirety), aligned to the GRCm38 mouse reference genome. Transcript per million (TPM) data was reported for each gene after mapping gene symbols to ensemble IDs using R packages, “tximport”, tximportData”, “ensembleb”, and “EnsDb.Mmusculus.v79”. Differential gene expression was estimated using DESeq2 (Love et al., “Moderated Estimation of Fold Change and Dispersion for RNA-Seq Data with DESeq2,” Genome Biol. 15:550 (2014), which is herein incorporated by reference in its entirety). For data visualization and gene ranking, log fold changes were adjusted using lfcShrink in DESeq2, to minimize the effect size of poorly expressed genes. GSEA analysis (v3.0) was performed on pre-ranked gene sets from differential expression between control and treated groups. We used R (v3.6.1) and R Studio (v1.2.1335) to create all visualizations, perform hierarchical clustering and principal component analysis. Visualizations of RNAseq data were produced using Enhanced Volcano (volcano plots), pheatmap (heatmaps), and ggplot2 (dotplots).

### Lgr5 ISH

Freshly cut 5-micron paraffin sections were stained using RNAscope 2.5 LS Red kit (ACD, cat #322150) and Bond Polymer Refine Red Detection kit (Leica, cat #DS9390) on Leica Bond RX instrument following routine manufacturer protocol ACD 2.5 Red. RNAscope 2.5 LS probes for Ms-LGR5 (ACD, cat #312178). DapB-negative control (ACD, cat #312038) were used with hybridization at 42C for 2 hours. The sections were pre-treated with Leica Bond ER2 Buffer for 20 min at 95C and Protease III (ACD, cat #322102) for 20 min at 40C. After staining the sections were counterstained with Hematoxylin and 10 ug/ml DAPI for 10 min and mounted with Mowiol mounting media.

### Sf9 Insect Cell Protein Expression

WT TNKS, TNKS2 SAM-PARP and associated mutants were expressed in Sf9 insect cells. Viral bacmids were generated using Tn7 transposition in chemically competent DH10Bac E. coli cells (Thermo Scientific #10361012). Bacmids were purified using a Bacmid DNA miniprep kit (Zymo Research #D4049) and transfected into Sf9 cells using Cellfectin II (Thermo Scientific #10362100) to generate recombinant baculoviruses. Proteins were expressed in 800 ml of Sf9 cells that were infected with the P2 amplified baculovirus at a cell density of 3×10⁶ cells per ml. Infected Sf9 cells were incubated at 28° C. with mild shaking (130 rpm) until viability dropped to approximately 80% (typically 72 hours post infection). Cell pellets were collected via centrifugation at 500 g for 20 minutes and resuspended in lysis buffer (500 mM NaCl, 20 mM HEPES pH 7.5, 2 mM MgCl₂, 5% v/v glycerol and 2 mM 2-mercaptoethanol which was supplemented with complete EDTA free protease inhibitor cocktail (Roche #11697498001) and DNase I (Worthington #LS006342).

### Protein Purification

Cells, resuspended in lysis buffer, were lysed by sonication and centrifuged at 48,000 g to remove insoluble cell debris. Lysates were filtered with a 5-uM filter and loaded onto a 5-ml StrepTrap XT affinity column (Cytiva) using an AKTA PURE. Following loading of the lysate, the column was washed with at least 5 column volumes of wash buffer (lysis buffer without additional supplementation). Proteins were eluted by applying 5 column volumes of elution buffer consisting of the wash buffer supplemented with 50 mM Biotin (IBA #2-1016-002). The eluted fractions, containing tankyrase, were dialyzed overnight against 500 mM NaCl, 20 mM HEPES pH 7.5, 2 mM MgCl₂, 5% v/v glycerol, 0.05% tween-20 and 2 mM 2-mercaptoethanol without the addition of preScission protease. The dialyzed samples were concentrated, supplemented to 20% v/v glycerol, flash-frozen in liquid nitrogen and stored at −80° C.

### Cryo-EM Grid Preparation and Data Collection

3.5 uL of purified protein, at approximately 20 μM, in a buffer containing 150 mM NaCl, 20 mM HEPES pH 7.5, 5% v/v glycerol, 0.05% tween-20 and 2 mM 2-mercaptoethanol were applied onto Quantifoil Au R 1.2/1.3 400 mesh graphene oxide coated grids for 45 seconds in a humidity-controlled vitrobbot Mark IV plunge freezer (Thermo Scientific). Humidity was set to 95% and temperature was set to 10° C. After 45 seconds, 4 μL of a 20 mM HEPES pH 7.5 solution was applied to the grid and removed two times to lower the salt concentration, prior to blotting and vitrification. For all three datasets, grids were imaged using a Titan KRIOS microscope (Thermo Scientific) operated at 300 keV and recorded with a Gatan K3 camera. Micrograph movies were recorded in super-resolution mode (nominal pixel size 0.826 Å), using a total dose of ˜55 electron per Å2, under an applied defocus ranging from −0.8 to −3.0 uM.

### Cryo-EM Image Processing

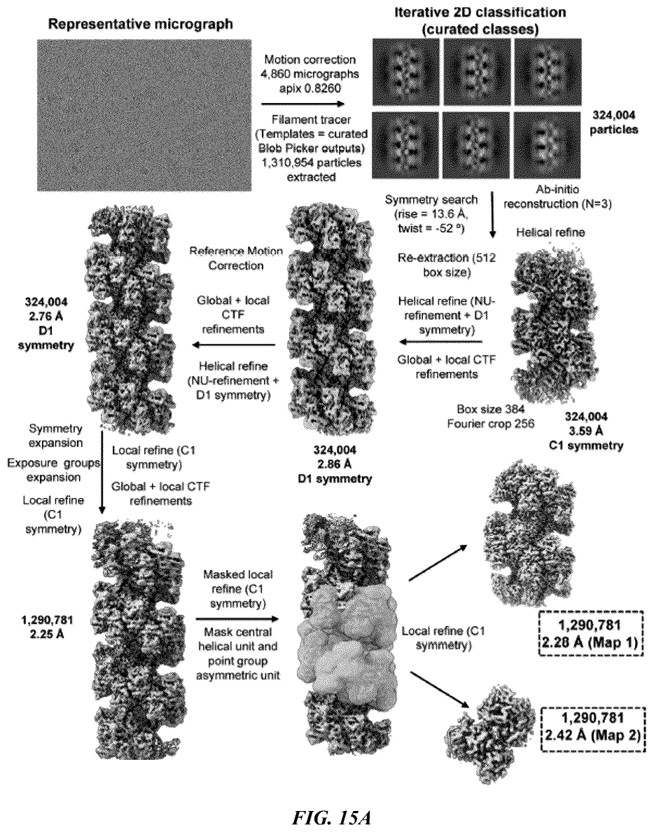

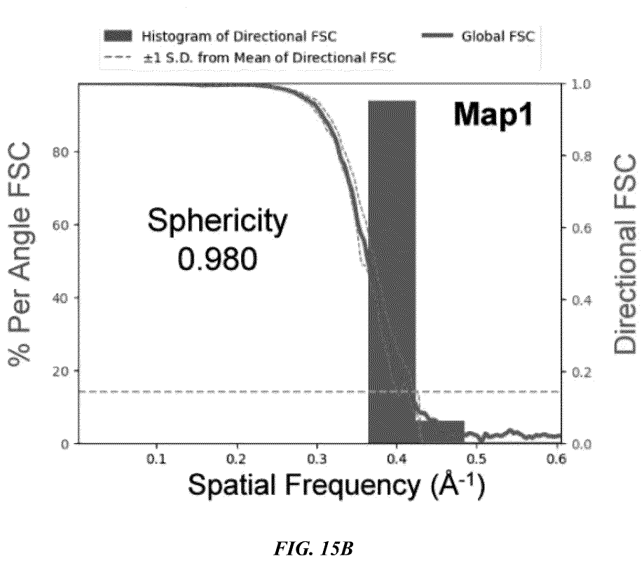

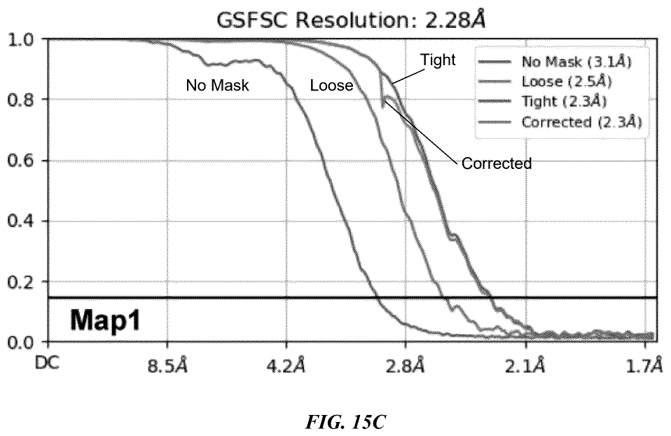

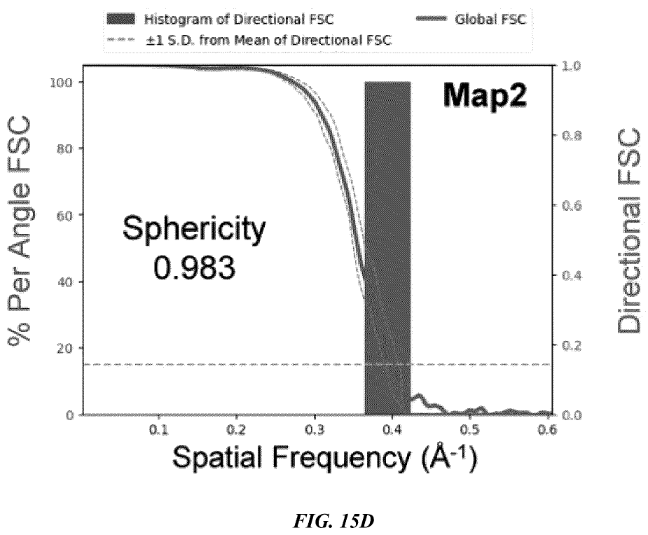

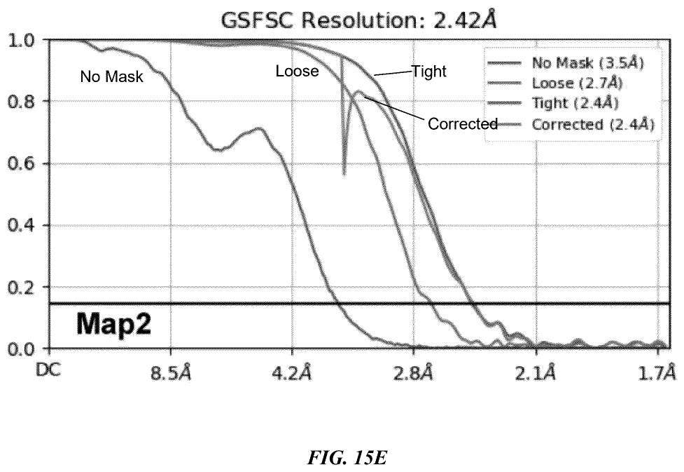

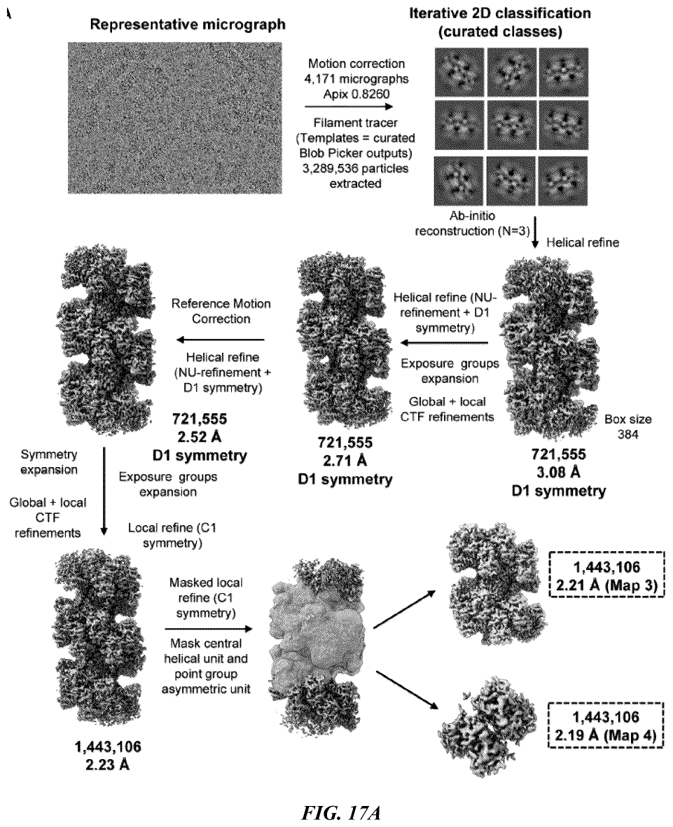

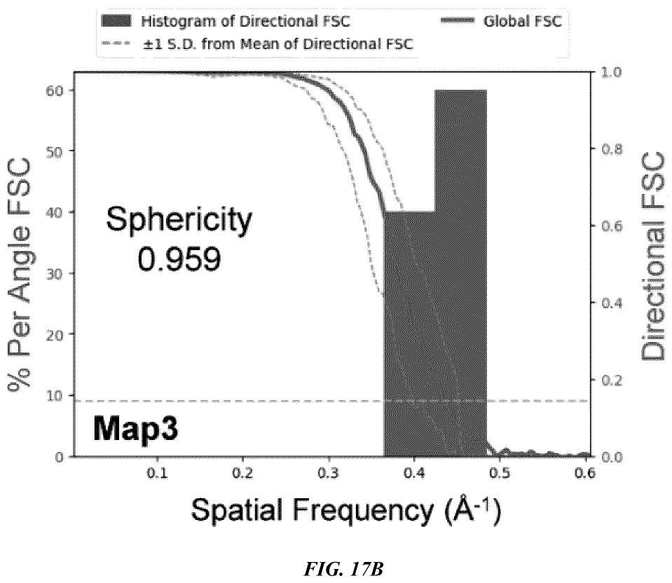

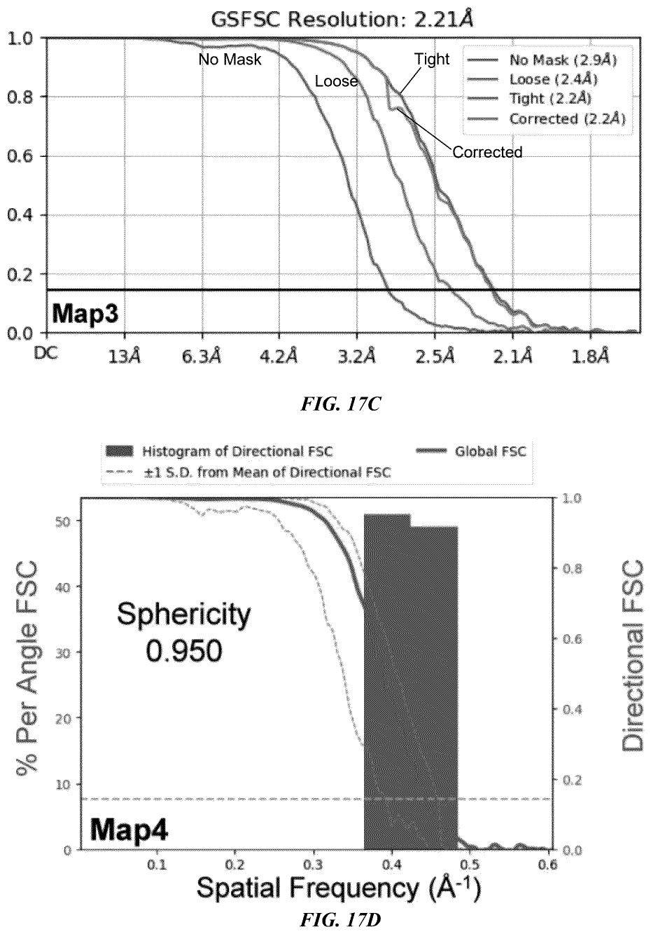

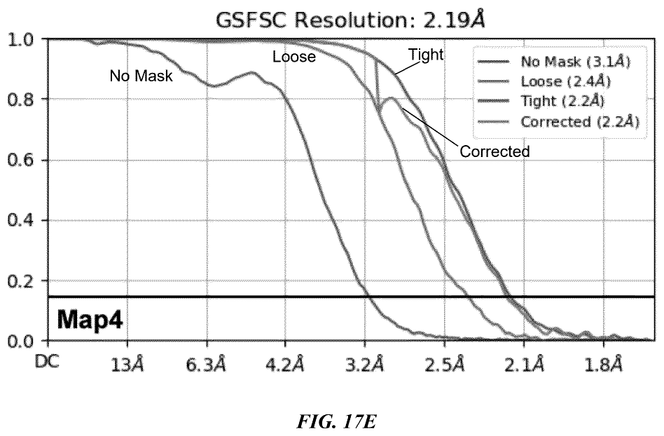

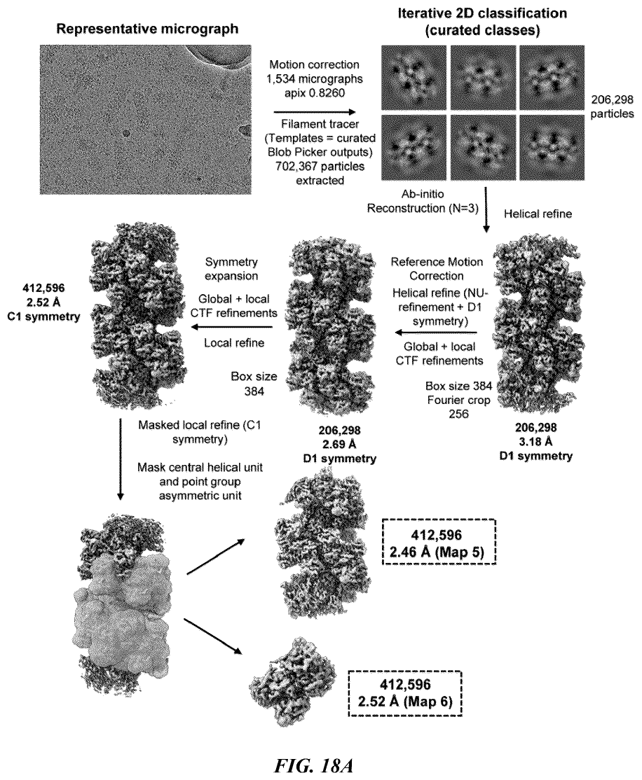

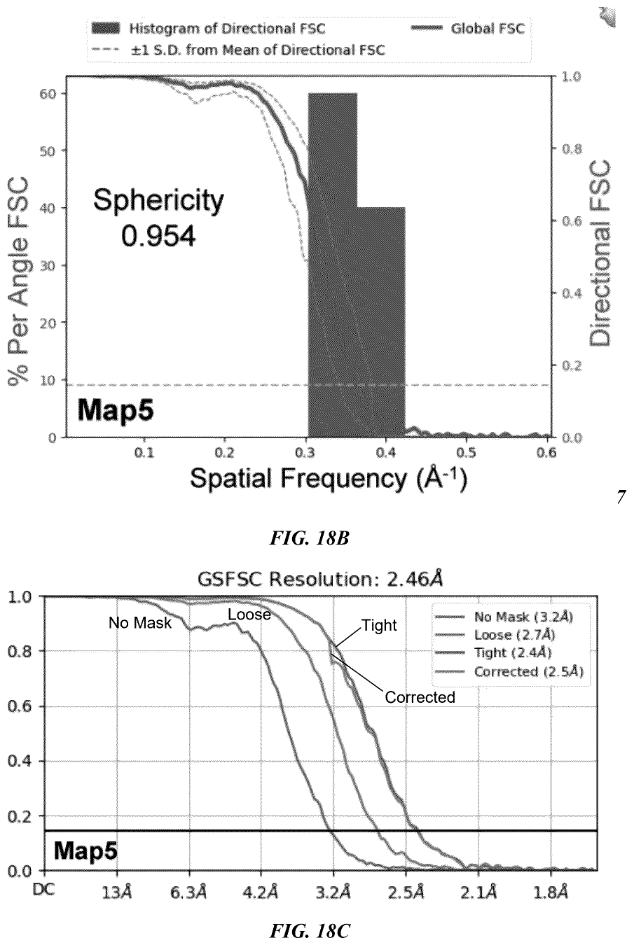

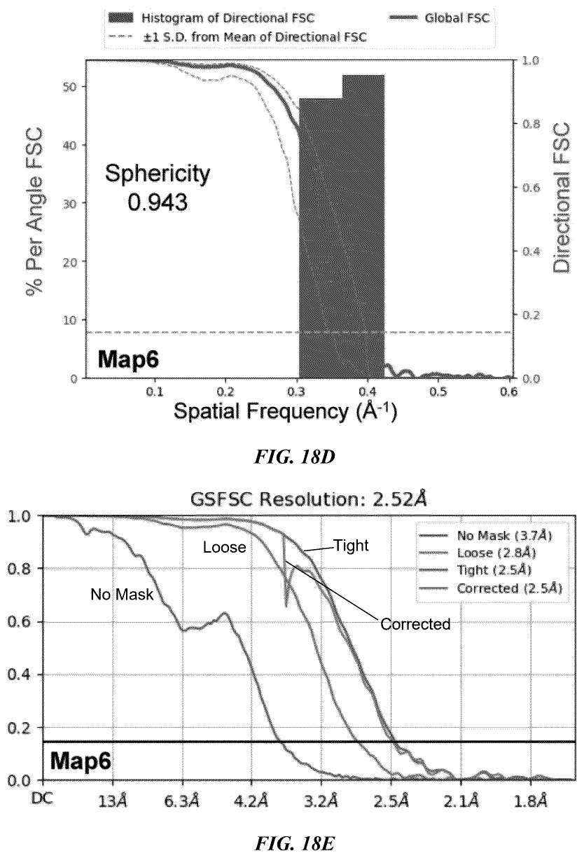

For the three datasets, all processing was conducted using cryosparc (v4.4) (Punjani et al., “CryoSPARC: Algorithms for Rapid Unsupervised Cryo-EM Structure Determination,” Nat. Methods 14:290-296 (2017), which is hereby incorporated by reference in its entirety). Movies were motion-corrected and binned 2× prior to patch CTF refinement. On-the-fly image processing utilized the blob picker particle picking algorithm alongside iterative 2D-classification to identify good segments of the TNKS filaments. Subsequent particle picking was performed using the filament tracer job using the previously selected particles' class averages as templates. The initial volume was reconstructed using cryosparc's ab-initio reconstruction job in which the output was utilized to estimate the helical symmetry parameters using the symmetry search function. Helical reconstructions were conducted using the helical refinement job in which the helical parameters converged to a helical twist of −52.4° and rise of 13.6 Å. The consensus class was further optimized through iterative global and local CTF refinements as well as through reference-based motion correction as implemented in cryosparc v4.4. Prior to the final refinement, the consensus class was symmetry expanded based on the helical and point group D1 symmetry and the exposure optics groups were expanded according to the beam image shift grouping. Subsequently, per-particle CTF refinement was ran prior to local refinement in C1 to yield the final consensus reconstruction. Local refinement jobs utilizing masks around the central helical unit as well as the most visually well-resolved point group D1 symmetry unit were ran to generate the maps used for model building and refinement. The respective maps' local resolution was calculated using Bsoft and subsequently applied to generate a locally filtered map volume for experimental interpretation and model building. For all reconstructions, the global resolution denoted in supplementary pipelines was estimated based on the gold-standard Fourier shell correlation (FSC) 0.143 criterion between two independently refined half maps. Data collection parameters and model statistics are shown in Table 2. The processing steps for the three datasets are described in FIGS. 15A-15E, 16A-16C, 17A-17E, and 18A-18E.

TABLE 2
Cryo-EM Data Analysis

| map1 | map2 | map3 | map4 | map5 | map6 |
|---|---|---|---|---|---|
| 8W23 | 8W25 | 8W27 | 8W28 | 8W2U | 8W2T |
| EMD- | EMD- | EMD- | EMD- | EMD- | EMD- |
| 43738 | 43739 | 43740 | 43471 | 43759 | 43758 |

Data collection and
processing

| Magnification | 29,000 | | 29,000 | | 29,000 | |
|---|---|---|---|---|---|---|
| Voltage (kV) | 300 | | 300 | | 300 | |
| Electron exposure (e−/Å²) | 55.69 | | 55.44 | | 55.33 | |
| Defocus range (μm) | −0.8 uM to −3.0 uM | | −0.8 uM to −3.0 uM | | −0.8 uM to −3.0 uM | |
| Pixel size (Å) | 0.826 | | 0.826 | | 0.826 | |
| Symmetry imposed | Symmetry | | Symmetry | | Symmetry | |
| | expansion via H and | | expansion via H | | expansion via H | |
| | D1, followed by C1 | | and D1, followed by | | and D1, followed by | |
| | refinement | | C1 refinement | | C1 refinement | |
| Initial particle images (no.) | 1,310,954 | | 3,289,536 | | 702,367 | |
| Final particle images (no.) | 1,290,781 | | 1,443,106 | | 412,596 | |
| Map resolution (Å) | 2.28 | 2.42 | 2.21 | 2.19 | 2.46 | 2.52 |
| FSC threshold 0.143 | | | | | | |
| Map resolution range (Å) | 2.01-7.32 | 2.18-6.25 | 2.07-6.05 | 2.06-6.33 | 2.19-9.04 | 2.25-8.06 |
| Refinement | | | | | | |
| Initial models used (PDB | PDB | PDB | PDB | PDB | PDB | PDB |
| code) | 8ALY | 8ALY | 8W23 | 8W23 | 8W23 | 8W23 |
| Model resolution (Å) | 2.4 | 2.4 | 2.4 | 2.4 | 2.6 | 2.6 |
| FSC threshold 0.5 | | | | | | |
| Map sharpening B factor | 70.8 | 93 | 67.9 | 68.4 | 70.6 | 82.6 |
| (Å²) | | | | | | |
| Model composition | | | | | | |
| Non-hydrogen atoms | 45,580 | 4,598 | 45,160 | 4556 | | 4,482 |
| Protein residues | 5,680 | 568 | 5,680 | 568 | 5,680 | 568 |
| Nucleic acid residues | 0 | 0 | 0 | 0 | 0 | 0 |
| (RNA) | | | | | | |
| Ligands | 40 | 4 | 40 | 4 | 20 | 2 |
| B factors (Å²) | | | | | | |
| Protein | 34.53 | 19.32 | 8.97 | 18.54 | 24.5 | 29.69 |
| Nucleic acids | — | — | — | — | — | — |
| Ligand | 39.78 | 16.08 | 10.12 | 10.54 | 90.94 | 72.91 |
| R.m.s. deviations | | | | | | |
| Bond lengths (Å) | 0.004 | 0.003 | 0.004 | 0.002 | 0.007 | 0.003 |
| Bond angles (°) | 0.539 | 0.496 | 0.546 | 0.484 | 0.75 | 0.545 |
| Validation | | | | | | |
| MolProbity score | 0.98 | 0.75 | 1.09 | 0.97 | 1.42 | 1.14 |
| Clashscore | 1.2 | 0.79 | 2.63 | 2.03 | 4.91 | 3.52 |
| Poor rotamers (%) | 1.52 | 0.87 | 1.13 | 0 | 1.09 | 0.87 |
| Ramachandran plot | | | | | | |
| Favored (%) | 98.74 | 98.76 | 98.95 | 98.76 | 97.3 | 98.05 |
| Allowed (%) | 1.26 | 1.24 | 1.05 | 1.24 | 2.7 | 1.95 |
| Disallowed (%) | 0 | 0 | 0 | 0 | 0 | 0 |

### Model Building and Refinement

The starting model for the TNKS2 SAM-PARP protomer was generated by manually fitting PDB 8ALY into the central helical unit local refinement using ChimeraX (v 1.6) and removing all chains other than chain D, corresponding to one of the more well-resolved regions of the map. This fitted protomer was re-built in Coot and Isolde followed by real-space refined in Phenix in an iterative fashion. During this process, the ligand if present, was fitted into the map and refined using a restraints file generated by Phenix eLBow. The final optimized protomer model was symmetry expanded in ChimeraX using either D1 symmetry alone or D1 and helical symmetry to generate models that fitted the local refinement maps and subsequently real-space refined. The respective final models were validated using MolProbity, as implemented in Phenix, in which the output validation statistics are shown in Table 2.

### Computational Model

All calculations were performed using the Schrodinger Suite (version 2019-4 to 2023-3). Free energy calculations were performed using the Schrodinger FEP+method (Wang et al., “Accurate and Reliable Prediction of Relative Ligand Binding Potency in Prospective Drug Discovery by Way of a Modern Free-Energy Calculation Protocol and Force Field,” Journal of the American Chemical Society 137 (7): 2695-2703 (2015), which is hereby incorporated by reference in its entirety) with TNKS2 structure (PDB: 3MHK). The calculations were run for 10 ns or until they reached convergence. The ensemble docking model (Glide SP docking) was set up with 18 grids prepared from TNKS2 crystal structures with complete B-loop (3MHK, 4HYF, 4PML, 4PNL, 4PNM, 4PNN, 4PNQ, 4PNS, 4PNT, 4TJU, 4TJW, 4TJY, 4TK0, 4TK5, 4TKF, 4TKG, 4TKI), together with 2 grids that were generated from the FEP snapshot where the B-loop opened during the simulation. All proteins were prepared using the Protein Preparation Wizard with the default setting. Ligands were prepared using LigPrep.

### Animal Studies

Tnks1 KO mice were produced through zygote electroporation of RNPs in C57Bl/6 mice. Mice were genotyped for Col1a1, R26, TGM-shRen.713, TGM-shTnks2.3004, TGM-shTnks1/2.13-30, TGM-shTnks1/2.33-13, Tnks, Kras, and Apc (1405.X (Genotyping primers listed in Table 1). For crypt survival experiments, mice were administered doxycycline via food pellets (200 mg/kg) for 2 or 3 weeks at 6-9 weeks of age. For Lgr5 ISH, mice were administered doxycycline via food pellets (200 mg/kg) for 3 weeks at 6-9 weeks of age.

### Synthesis of N-[2-[4-(1-Hydroxy-1-methyl-ethyl)phenyl]-4-oxo-3H-quinazolin-7-yl]-2-phenoxy-pyrimidine-5-carboxamide (TDI-012727)

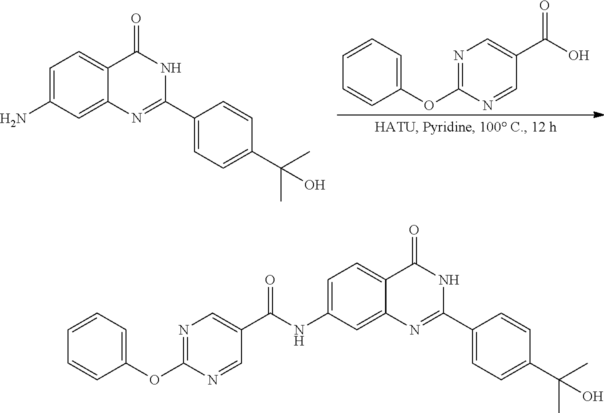 [SMILES: CC(C)(O)c1ccc(-c2nc3cc(N)ccc3c(=O)[nH]2)cc1 ; O=C(O)c1cnc(Oc2ccccc2)nc1 ; CC(C)(O)c1ccc(-c2nc3cc(NC(=O)c4cnc(Oc5ccccc5)nc4)ccc3c(=O)[nH]2)cc1]

A mixture of 7-amino-2-[4-(1-hydroxy-1-methyl-ethyl)phenyl]-3H-quinazolin-4-one (80 mg, 270.88 umol, 1 eq), 2-phenoxypyrimidine-5-carboxylic acid (58.56 mg, 270.88 umol, 1 eq), and HATU (103.00 mg, 270.88 umol, 1 eq) in Py. (1 mL) was stirred at 100° C. for 12 hours. The reaction mixture was concentrated under reduced pressure to give a residue. The residue was purified by prep-HPLC (column: Phenomenex Gemini-NX 150*30 mm*5 um; mobile phase: [water (0.05% NH₃H₂O+10 mM NH₄HCO₃)-ACN]; B %: 30%-70%, 8 min). Compound N-[2-[4-(1-hydroxy-1-methyl-ethyl)phenyl]-4-oxo-3H-quinazolin-7-yl]-2-phenoxy-pyrimidine-5-carboxamide (16.6 mg, purity 96.92%) was obtained as white solid. ¹H NMR: (400 MHz, DMSO-d₆) δ 10.83 (s, 1H), 9.16 (s, 2H), 8.27 (br. s, 1H), 8.16-8.11 (m, 3H), 7.81-7.78 (m, 1H), 7.67-7.61 (m, 2H), 7.55-7.45 (t, 3H), 7.33-7.31 (m, 1H), 7.31-7.25 (m, 2H), 5.17 (br. s, 2H), 1.46 (br. s, 6H). LCMS: (M+H⁺): 494.2@2.242 min (5-95% ACN in H₂O, 6 min).

### Synthesis of N-1-[2-[4-(1-Hydroxy-1-methyl-ethyl)phenyl]-4-oxo-3H-quinazolin-7-yl]-N4-methyl-terephthalamide (TDI-012465)

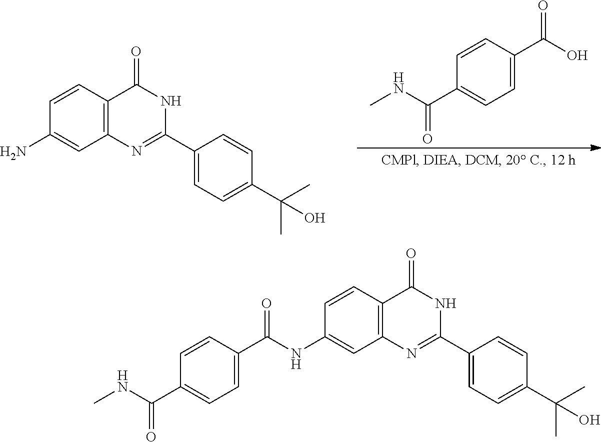 [SMILES: CC(C)(O)c1ccc(-c2nc3cc(N)ccc3c(=O)[nH]2)cc1 ; CNC(=O)c1ccc(C(=O)O)cc1 ; CNC(=O)c1ccc(C(=O)Nc2ccc3c(=O)[nH]c(-c4ccc(C(C)(C)O)cc4)nc3c2)cc1]

A mixture of 7-amino-2-[4-(1-hydroxy-1-methyl-ethyl)phenyl]-3H-quinazolin-4-one (0.1 g, 338.60 umol, 1 eq), 4-(methylcarbamoyl)benzoic acid (91.00 mg, 507.90 umol, 1.5 eq), 2-chloro-1,3-dimethyl-4,5-dihydroimidazol-1-ium chloride (114.48 mg, 677.20 umol, 2 eq), and DIPEA (109.40 mg, 846.50 umol, 147.44 uL, 2.5 eq) in DCM (1 mL) was degassed and purged with N₂ for 3 times, and then the mixture was stirred at 20° C. for 12 hours under N₂ atmosphere. The mixture was concentrated in vacuum. The residue was purified by prep-HPLC (neutral condition: column: Waters Xbridge Prep OBD C18 150*40 mm*10 um; mobile phase: [water (10 mM NH₄HCO₃)-ACN]; B %: 10%-40%, 8 min). Compound N1-[2-[4-(1-hydroxy-1-methyl-ethyl)phenyl]-4-oxo-3H-quinazolin-7-yl]-N4-methyl-terephthalamide (20.6 mg, 40.48 umol, 11.95% yield, 89.7% purity) was obtained as a yellow oil. ¹H NMR: (400 MHz, DMSO-d6) δ 12.41 (br dd, J=1.5, 5.5 Hz, 1H), 10.75 (br s, 1H), 8.70-8.59 (m, 1H), 8.35-8.29 (m, 1H), 8.20-8.11 (m, 3H), 8.09-7.98 (m, 3H), 7.85 (br dd, J=1.6, 8.7 Hz, 1H), 7.71-7.59 (m, 2H), 5.19 (s, 1H), 2.87-2.76 (m, 3H), 1.47 (br s, 6H). LCMS: (M+H⁺): 457.2@1.889 min (5-95% ACN in H₂O, 6 min).

### Synthesis of 2-Benzyloxy-N-[2-[4-(1-hydroxy-1-methyl-ethyl)phenyl]-4-oxo-3H-quinazolin-7-yl]pyrimidine-5-carboxamide (TDI-012747)

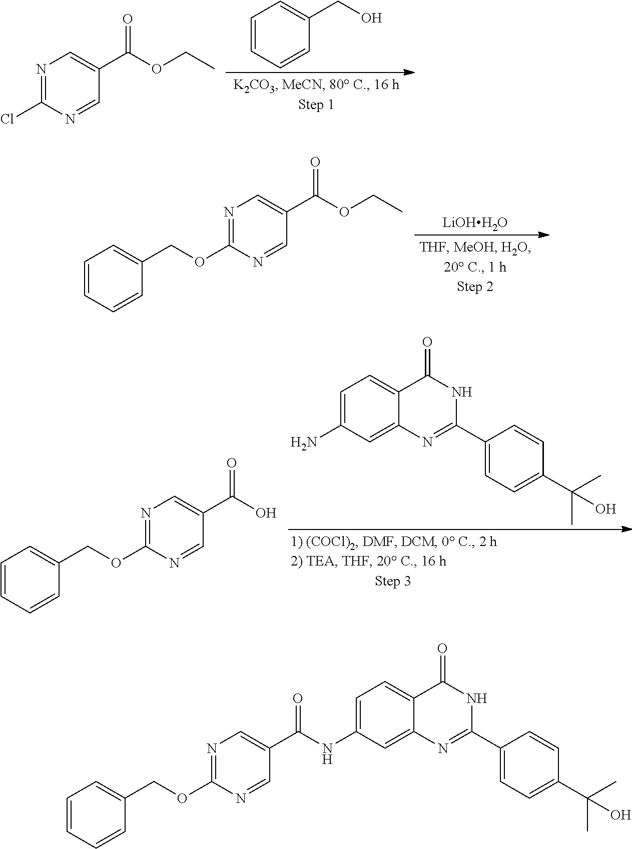

### Step 1: Synthesis of Ethyl 2-benzyloxypyrimidine-5-carboxylate

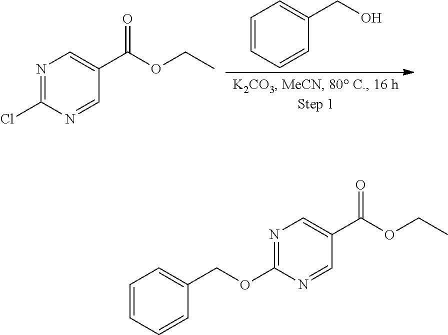

A mixture of phenylmethanol (695.44 mg, 6.43 mmol, 668.69 uL, 1.2 eq), ethyl 2-chloropyrimidine-5-carboxylate (1 g, 5.36 mmol, 1 eq), and K₂CO₃ (1.11 g, 8.04 mmol, 1.5 eq) in MeCN (10 mL) was degassed and purged with N₂ for 3 times, and then the mixture was stirred at 80° C. for 16 hours under N₂ atmosphere. The mixture was poured into H₂O (50 mL) and extracted with EtOAc (20 mL*3). The combined organic layer was washed with brine (50 mL), dried over Na₂SO₄, filtered and concentrated under reduced pressure. The residue was purified by column chromatography (SiO₂, Petroleum ether/Ethyl acetate=I/O to 10/1) to give ethyl 2-benzyloxypyrimidine-5-carboxylate (0.43 g, crude) as a white solid. LCMS: (M+H⁺): 259.1@0.762 min (5_95AB_2 min-220-254, 1.5 min).

### Step 2: Synthesis of 2-Benzyloxypyrimidine-5-carboxylic acid

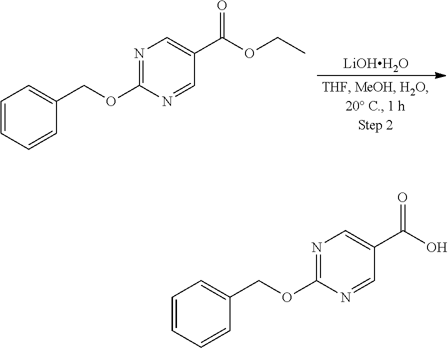

To a solution of ethyl 2-benzyloxypyrimidine-5-carboxylate (230 mg, 890.53 umol, 1 eq) in THF (3 mL), MeOH (1 mL) and H₂O (1 mL) was added LiOH·H₂O (112.10 mg, 2.67 mmol, 3 eq). The mixture was stirred at 20° C. for 1 hour. The mixture was concentrated under reduced pressure, and then the pH of the residue was adjusted to pH=2 with HCl (2 N). The mixture was filtered and the filtered cake was concentrated under reduced pressure to give methyl 2-benzyloxypyrimidine-5-carboxylic acid (0.11 g, crude) as a white solid. LCMS: (M+H⁺): 231.1@0.628 min (5_95AB_2 min-220-254, 1.5 min).

### Step 3: Synthesis of 2-Benzyloxy-N-[2-[4-(1-hydroxy-1-methyl-ethyl)phenyl]-4-oxo-3H-quinazolin-7-yl]pyrimidine-5-carboxamide

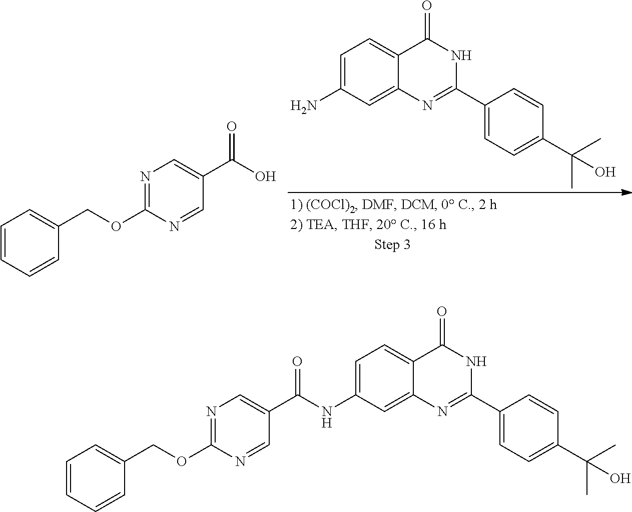 [SMILES: O=C(O)c1cnc(OCc2ccccc2)nc1 ; CC(C)(O)c1ccc(-c2nc3cc(N)ccc3c(=O)[nH]2)cc1 ; CC(C)(O)c1ccc(-c2nc3cc(NC(=O)c4cnc(OCc5ccccc5)nc4)ccc3c(=O)[nH]2)cc1]

To a solution of 2-benzyloxypyrimidine-5-carboxylic acid (0.1 g, 434.37 umol, 1 eq) in DCM (2 mL) was added (COCl)₂ (82.70 mg, 651.55 umol, 57.03 uL, 1.5 eq) and DMF (3.17 mg, 43.44 umol, 3.34 uL, 0.1 eq). The mixture was stirred at 0° C. for 2 hours. The mixture was concentrated under reduced pressure. Compound 2-benzyloxypyrimidine-5-carbonyl chloride (0.1 g, crude) was obtained as a white solid. The crude product was used into the next step without further purification. A mixture of 2-benzyloxypyrimidine-5-carbonyl chloride (0.1 g, 402.15 umol, 1 eq), 7-amino-2-[4-(1-hydroxy-1-methyl-ethyl)phenyl]-3H-quinazolin-4-one (142.52 mg, 482.58 umol, 1.2 eq), and Et₃N (40.69 mg, 402.15 umol, 55.97 uL, 1 eq) in THF (4 mL) was degassed and purged with N₂ for 3 times, and then the mixture was stirred at 20° C. for 16 hours under N₂ atmosphere. The mixture was concentrated under reduced pressure. The residue was purified by prep-HPLC (neutral condition; column: Phenomenex Gemini-NX 80*40 mm*3 um; mobile phase: [water (10 mM NH₄HCO₃)—ACN]; B %: 30%-50%, 8 min) to give 2-benzyloxy-N-[2-[4-(1-hydroxy-1-methyl-ethyl)phenyl]-4-oxo-3H-quinazolin-7-yl]pyrimidine-5-carboxamide (8 mg, 15.51 umol, 3.86% yield, 98.39% purity) as a white solid. ¹H NMR: (400 MHz, DMSO) δ 12.32 (br s, 1H), 10.77 (s, 1H), 9.17 (s, 2H), 8.26 (s, 1H), 8.14 (d, J=8.5 Hz, 3H), 7.84-7.74 (m, 1H), 7.63 (d, J=8.4 Hz, 2H), 7.50 (br d, J=7.0 Hz, 2H), 7.45-7.32 (m, 3H), 5.60-5.44 (m, 2H), 5.18 (s, 1H), 1.47 (s, 6H). LCMS: (M+H⁺): 508.2@2.353 min (5_95AB_6 min-220, 4.5 min).

### Synthesis of N-[2-[4-(1-Hydroxy-1-methyl-ethyl)phenyl]-4-oxo-3H-quinazolin-7-yl]-6-phenyl-pyridine-3-carboxamide (TDI-012756)

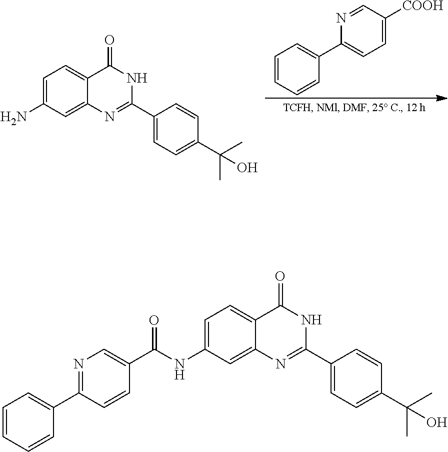 [SMILES: CC(C)(O)c1ccc(-c2nc3cc(N)ccc3c(=O)[nH]2)cc1 ; O=C(O)c1ccc(-c2ccccc2)nc1 ; CC(C)(O)c1ccc(-c2nc3cc(NC(=O)c4ccc(-c5ccccc5)nc4)ccc3c(=O)[nH]2)cc1]

To a mixture of 7-amino-2-[4-(1-hydroxy-1-methyl-ethyl)phenyl]-3H-quinazolin-4-one (0.1 g, 338.60 umol, 1 eq), and 6-phenylpyridine-3-carboxylic acid (80.94 mg, 406.32 umol, 1.2 eq) in DMF (2 mL) was added [chloro(dimethylamino)methylene]-dimethyl-ammonium hexafluorophosphate (104.50 mg, 372.46 umol, 1.1 eq) and 1-methylimidazole (69.50 mg, 846.50 umol, 67.48 uL, 2.5 eq). The mixture was stirred at 25° C. for 12 hours. The mixture was filtered and concentrated in vacuum. The mixture was purified by prep-HPLC (column: Phenomenex Gemini-NX C18 75*30 mm*3 um; mobile phase: [water (0.05% NH₃H₂O+10 mM NH₄HCO₃)-ACN]; B %: 30%-60%, 8 min). Compound N-[2-[4-(1-hydroxy-1-methyl-ethyl)phenyl]-4-oxo-3H-quinazolin-7-yl]-6-phenyl-pyridine-3-carboxamide (28.8 mg, 60.06 umol, 17.74% yield, 99.37% purity) was obtained as white solid. ¹H NMR: (400 MHz, DMSO-d6) δ 12.38 (br. s, 1H), 10.83 (s, 1H), 9.25 (s, 1H), 8.47-8.52 (m, 1H), 8.23 (s, 1H), 8.22-8.16 (m, 3H), 8.16-8.13 (m, 3H), 7.84-7.80 (m, 1H) 7.66-7.64 (m, 2H), 7.63-6.50 (m, 3H), 5.17 (s, 1H), 1.48 (s, 6H). LCMS: (M+H⁺): 477.2@2.296 min (5-95% ACN in H₂O, 4.5 min).

### Synthesis of N-[2-[4-(1-Hydroxy-1-methyl-ethyl)phenyl]-4-oxo-3H-quinazolin-7-yl]-4-phenyl-benzamide (TDI-012424)

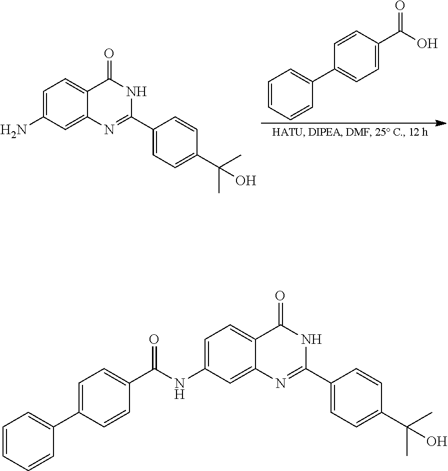 [SMILES: CC(C)(O)c1ccc(-c2nc3cc(N)ccc3c(=O)[nH]2)cc1 ; O=C(O)c1ccc(-c2ccccc2)cc1 ; CC(C)(O)c1ccc(-c2nc3cc(NC(=O)c4ccc(-c5ccccc5)cc4)ccc3c(=O)[nH]2)cc1]

To a solution of 7-amino-2-[4-(1-hydroxy-1-methyl-ethyl)phenyl]-3H-quinazolin-4-one (50 mg, 169.30 umol, 1 eq), 4-phenylbenzoic acid (50.34 mg, 253.95 umol, 1.5 eq), and HATU (96.56 mg, 253.95 umol, 1.5 eq) in DMF (1 mL) was added DIPEA (54.70 mg, 423.25 umol, 73.72 uL, 2.5 eq). The mixture was stirred at 25° C. for 12 hours. The mixture was collected by filtration and the filtrate was concentrated under reduced pressure to give a residue. The residue was purified by prep-HPLC (HCl condition, column: Phenomenex Luna C18 75*30 mm*3 um; mobile phase: [water (0.2% FA)-ACN]; B %: 40%-70%, 8 min). Compound N-[2-[4-(1-hydroxy-1-methyl-ethyl)phenyl]-4-oxo-3H-quinazolin-7-yl]-4-phenyl-benzamide (11.6 mg, 21.76 umol, 12.85% yield, 89.21% purity) was obtained as a white solid. ¹H NMR: (400 MHz, DMSO-d6) δ 10.68 (s, 1H), 8.34 (s, 1H), 8.16-8.09 (m, 5H), 7.88 (br d, J=8.38 Hz, 3H), 7.78 (br d, J=7.25 Hz, 2H), 7.63 (d, J=8.38 Hz, 2H), 7.55-7.49 (m, 2H), 7.46-7.41 (m, 1H), 1.47 (s, 6H). LCMS: (M+H⁺): 476.1@2.611 min (5-95% ACN in H₂O, 6 min).

### Synthesis of N-[2-[4-(1-Hydroxy-1-methyl-ethyl)phenyl]-4-oxo-3H-quinazolin-7-yl]-4-methoxy-6-phenyl-pyridine-3-carboxamide (TDI-012804)

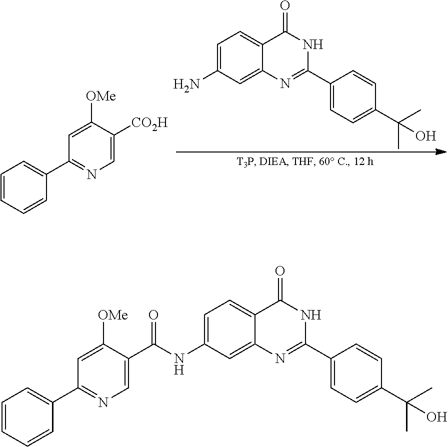 [SMILES: COc1cc(-c2ccccc2)ncc1C(=O)O ; CC(C)(O)c1ccc(-c2nc3cc(N)ccc3c(=O)[nH]2)cc1 ; COc1cc(-c2ccccc2)ncc1C(=O)Nc1ccc2c(=O)[nH]c(-c3ccc(C(C)(C)O)cc3)nc2c1]

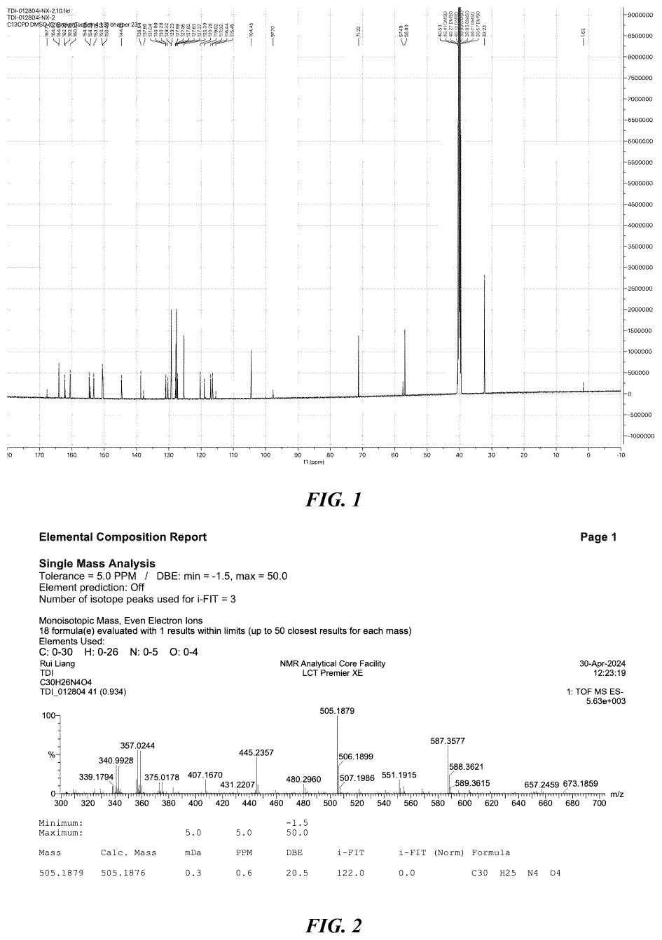

A mixture of 7-amino-2-[4-(1-hydroxy-1-methyl-ethyl)phenyl]-3H-quinazolin-4-one (0.5 g, 1.69 mmol, 1 eq), 4-methoxy-6-phenyl-pyridine-3-carboxylic acid (388.09 mg, 1.69 mmol, 1 eq), T₃P (4.31 g, 6.77 mmol, 4.03 mL, 50% purity, 4 eq), and DIPEA (1.09 g, 8.46 mmol, 1.47 mL, 5 eq) in THF (10 mL) was degassed and purged with N₂ for 3 times, and then the mixture was stirred at 60° C. for 12 hours under N2 atmosphere. The residue was poured into H₂O (100 mL). The aqueous phase was extracted with ethyl acetate (50 mL*3). The combined organic phase was washed with brine (60 mL), dried with anhydrous Na₂SO₄, filtered and concentrated in vacuum. The residue was purified by prep-HPLC (neutral condition, column: Phenomenex C18 75*30 mm*3 um; mobile phase: [water (NH₄HCO₃)-ACN]; B %: 30%-60%, 8 min). Compound N-[2-[4-(1-hydroxy-1-methyl-ethyl)phenyl]-4-oxo-3H-quinazolin-7-yl]-4-methoxy-6-phenyl-pyridine-3-carboxamide (110 mg, 100% purity) was obtained as a white solid. ¹H NMR: (400 MHz, DMSO-d6) δ 10.62 (s, 1H), 8.76 (s, 1H), 8.25 (s, 1H), 8.21 (br d, 2H), 8.16-8.09 (m, 3H), 7.80-7.71 (m, 2H), 7.63 (d, 2H), 7.57-7.49 (m, 3H), 5.17 (s, 1H), 4.14-4.09 (m, 3H), 1.47 (s, 6H). LCMS: (M+H⁺): 507.2 @2.591 min (5-95% ACN in H₂O, 6 min). The 13C NMR is shown in FIG. 1, and the LCMS chromatogram is shown in FIG. 2.

### Example 2—Chromosome 8p Deletions Sensitize Cells to TNKS2-Selective Silencing

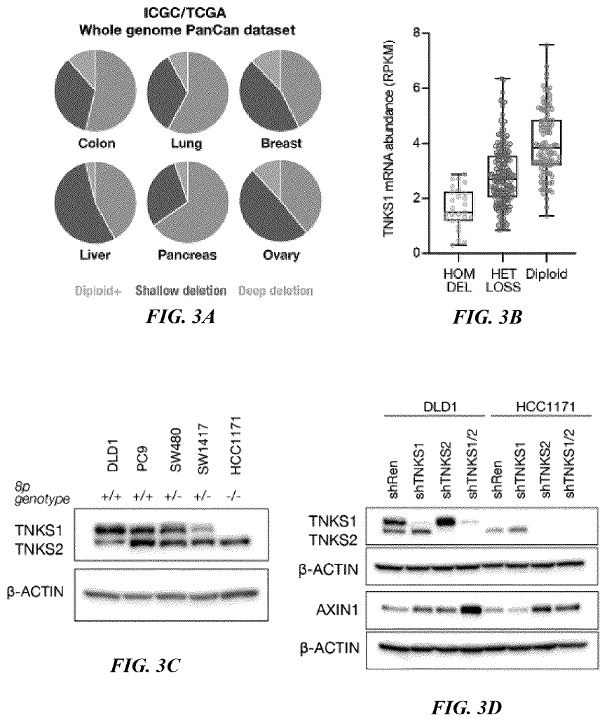

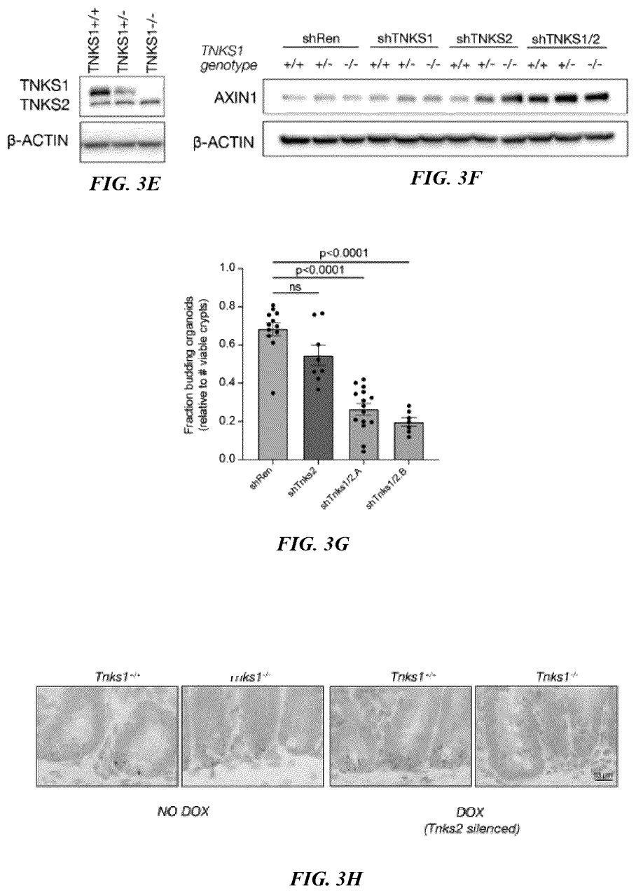

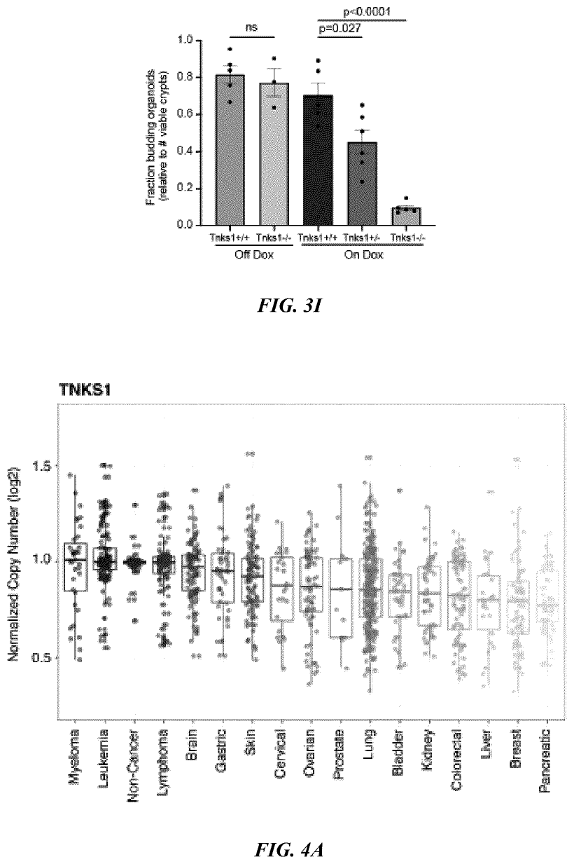

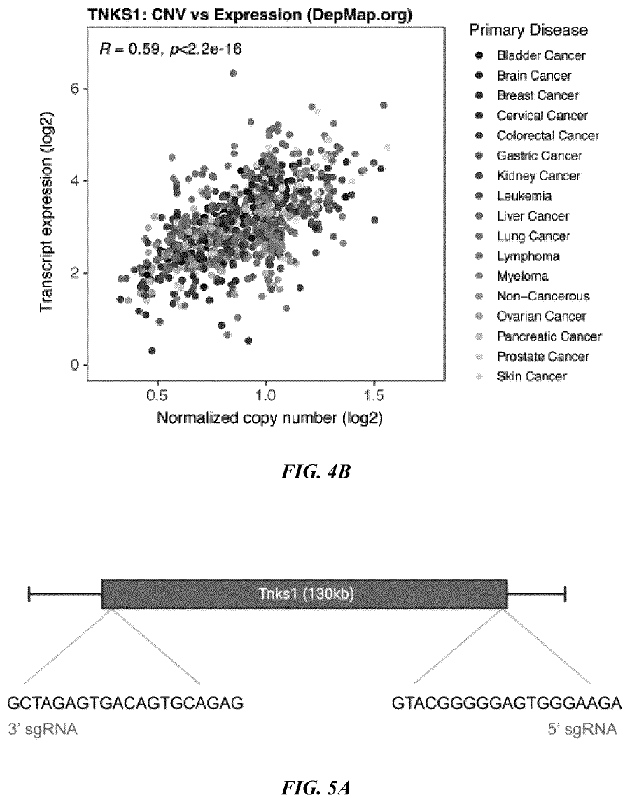

Large genomic deletions in Chromosome 8 are frequently observed in many epithelial cancer types. Analysis of available ICGC and TCGA whole genome sequencing data (I. T. P.-C. A. o. W. G. Consortium, “Pan-Cancer Analysis of Whole Genomes,” Nature 578:82-93 (2020), which is hereby incorporated by reference in its entirety) shows approximately half of colon, lung, breast, liver, and prostate cancers carry heterozygous or homozygous 8p deletions (FIG. 3A). Loss of this genomic segment and reduced TNKS1 gene dosage correlates with a decrease in TNKS1 transcript and protein expression (FIGS. 3B-3C, FIGS. 4A-4B). Consistent with previously published data (Huang et al., “Tankyrase Inhibition Stabilizes Axin and Antagonizes Wnt Signalling,” Nature 461:614-620 (2009); Foronda et al., “Tankyrase Inhibition Sensitizes Cells to CDK4 Blockade,” PLOS One 14: e0226645 (2019), which are hereby incorporated by reference in their entirety), suppression of both TNKS1 and TNKS2 in 8p diploid DLD1 cells was required to stabilize AXIN1; however consistent with an induced dependency on TNKS2 in 8p-deleted cells, silencing of TNKS2 alone in HCC1171 cells was sufficient to stabilize AXIN1 (FIG. 3D).

The 8p chromosome arm contains several potential tumor suppressor genes whose aggregate loss may promote tumorgenicity (Gustafson et al., “Functional Evidence for a Colorectal Cancer Tumor Suppressor Gene at Chromosome 8p22-23 by Monochromosome Transfer,” Cancer Res 56:5238-5245 (1996); Xue et al., “A Cluster of Cooperating Tumor-Suppressor Gene Candidates in Chromosomal Deletions,” Proc Natl Acad Sci USA 109:8212-8217 (2012), which are hereby incorporated by reference in their entirety). To confirm the selective effect of TNKS2 suppression is specifically due to the loss of TNKS1, homozygous knockout (TNKS1^{KO}) and heterozygous (INKS1^{HET}) isogenic DLD1 clones were generated (FIG. 3E). As expected, in TNKS1^{KO} cells, TNKS2 silencing induced AXIN1 stabilization to a level equivalent to suppression of both TNKS enzymes (FIG. 3F). Importantly, AXIN1 stabilization was also apparent at intermediate levels in TNKS1^{HET} cells, suggesting that TNKS1 is haploinsufficient for supporting cellular Tankyrase activity and that heterozygous TNKS1 loss can sensitize cells to TNKS2 inhibition (FIG. 3F).

Homozygous deletion of both Tankyrase enzymes in mice causes embryonic lethality, while TNKS2 knockout mice are viable and fertile (Chiang et al., “Tankyrase 1 and Tankyrase 2 are Essential but Redundant for Mouse Embryonic Development,” PLOS One 3: e2639 (2008), which is hereby incorporated by reference in its entirety). This suggests that systemic, selective TNKS2 targeting would have minimal toxicity to normal tissues. To directly test this idea in vivo, mice were generated harboring an inducible shRNA targeting Tnks2 and it was compared to previously published Tnks1 Thks2 dual knockdown mice (Schatoff et al., “Distinct Colorectal Cancer-Associated APC Mutations Dictate Response to Tankyrase Inhibition,” Cancer Discov 9:1358-1371 (2019), which is hereby incorporated by reference in its entirety). To explore the effect of Tnks2-selective suppression on tissue function with high WNT pathway activity in vivo, the ability of intestinal crypts to form budding organoids in Matrigel culture was measured. Silencing of both Tnks1 and Tnks2 induced a significant decrease in stem-cell driven organoid formation compared to neutral (shRen) controls (FIG. 3G, p<0.01), while Tnks2 suppression showed a mild but not significant decrease in organoid formation (FIG. 3G; p>0.05).

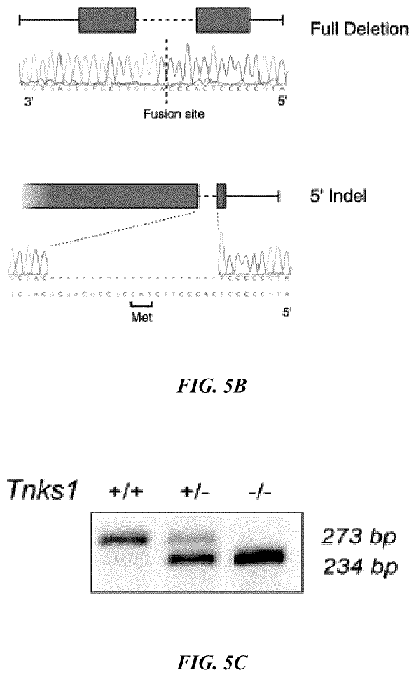

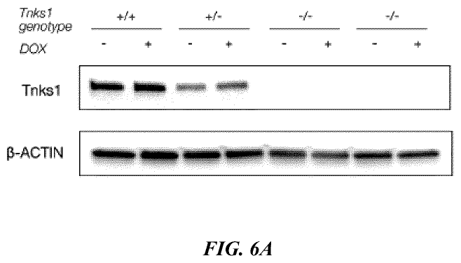

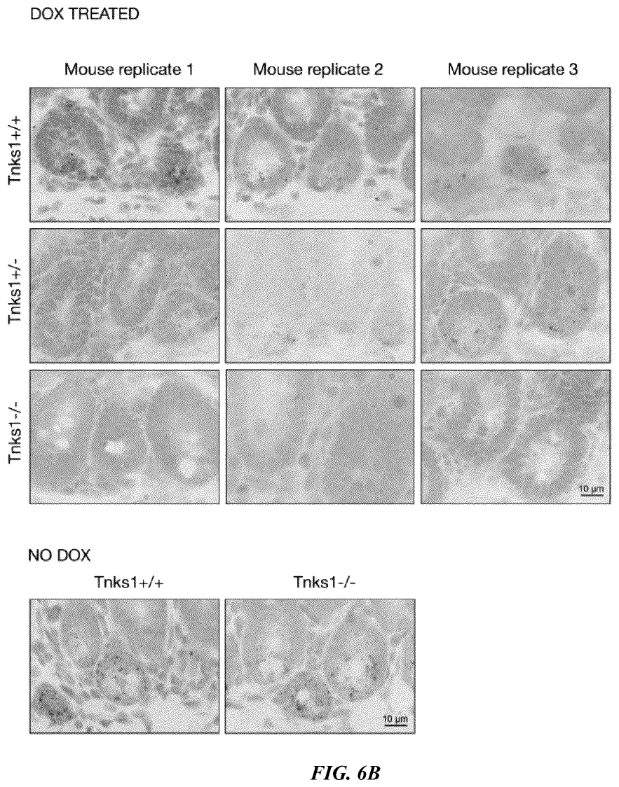

To determine whether loss of one or both copies of Tnks1 would create a Tnks2 dependence in vivo, Cas9 and tandem sgRNAs were used to delete the entire Tnks1 locus in fertilized zygotes and intercrossed the resulting animals to generate wildtype (Tnks1^{WT}), heterozygous (Tnks1^{HET}), and homozygous (Tnks1^{KO}) knockout mice carrying the inducible shTnks2 cassette (FIGS. 5A-5C and FIG. 6A). In the absence of doxycycline (no Tnks2 silencing) there was no difference in organoid forming capacity or the expression of the WNT target Lgr5 between Thks1^{WT} and Thks1^{KO} crypts (FIGS. 3H-3I), implying Tnks1 is largely dispensable for crypt function. Similarly, Tnks1^{WT}/shTnks2 mice treated with dox for 3 weeks showed robust Lgr5 expression and organoid formation comparable to untreated controls (FIGS. 3H-3I and FIG. 6B). In contrast, dox-treated Tnks1^{KO}/shTnks2 mice showed a significant defect in organoid formation and had a marked reduction in Lgr5 expression in crypt stem cells (FIGS. 3H-3I). Further, consistent with the dose dependent AXIN1 stabilization seen in isogenic DLD1 cells, Tnks1^{HET}/shTnks2 animals showed reduced organoid forming capacity and intermediate Lgr5 expression in intestinal crypts (FIGS. 3I, 6B).

Together, these data support the concept that the loss of either one or both copies of TNKS1 following 8p deletions, creates a TNKS2 dependence that could be exploited to enable tumor cell-selective suppression of WNT signaling in 8p-deleted tumor cells while avoiding on-target toxicities associated with WNT blockade in normal tissues.

### Example 3—Rational Design of TNKS2-Selective Small Molecule Inhibitors

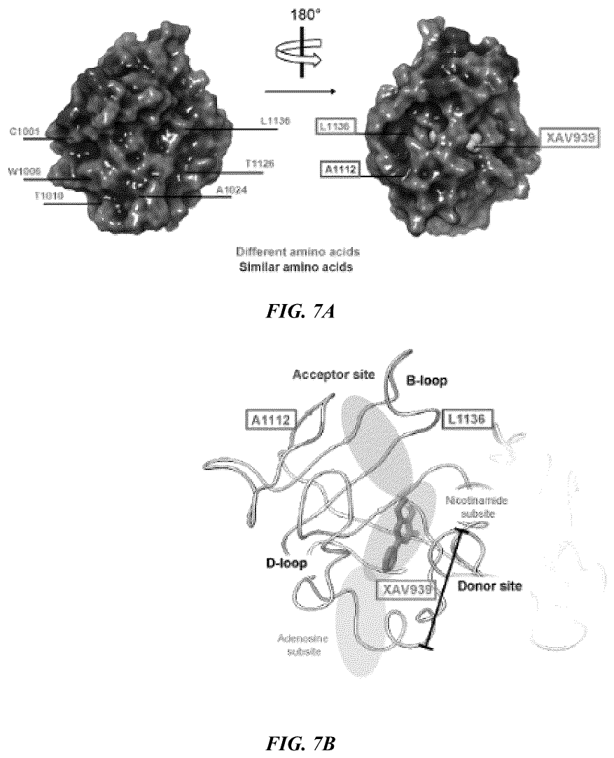

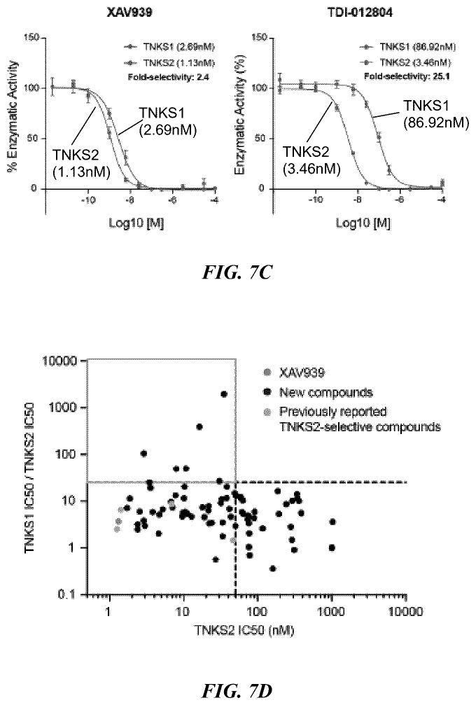

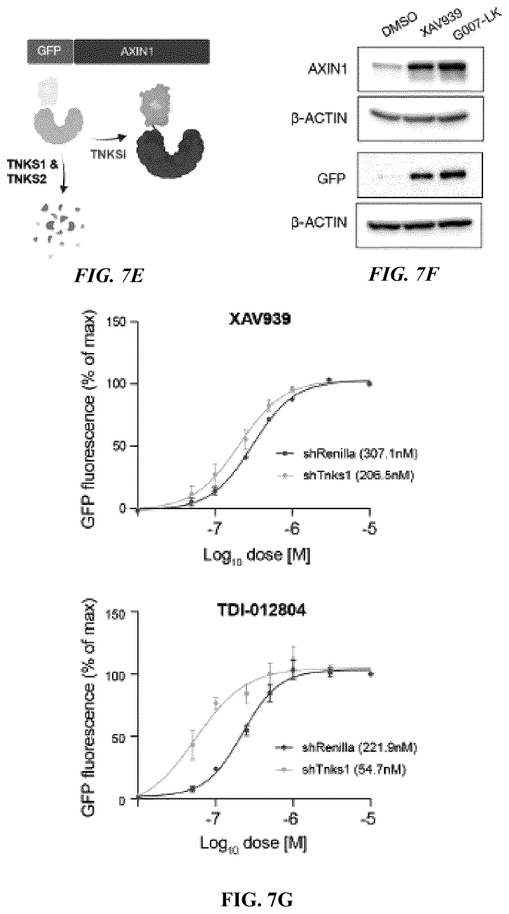

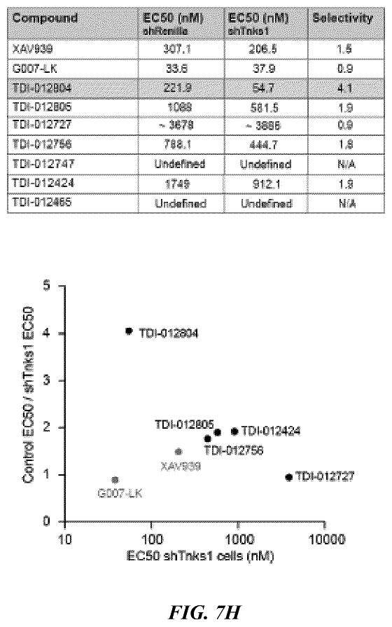

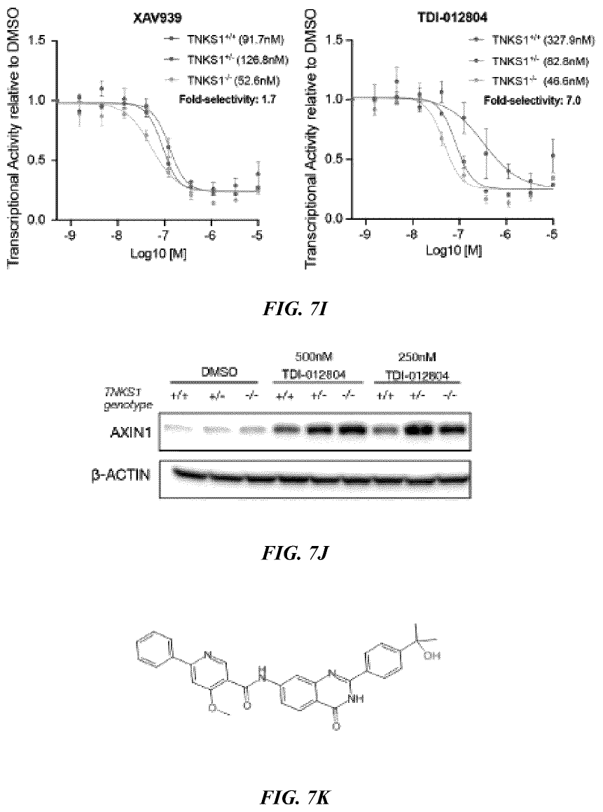

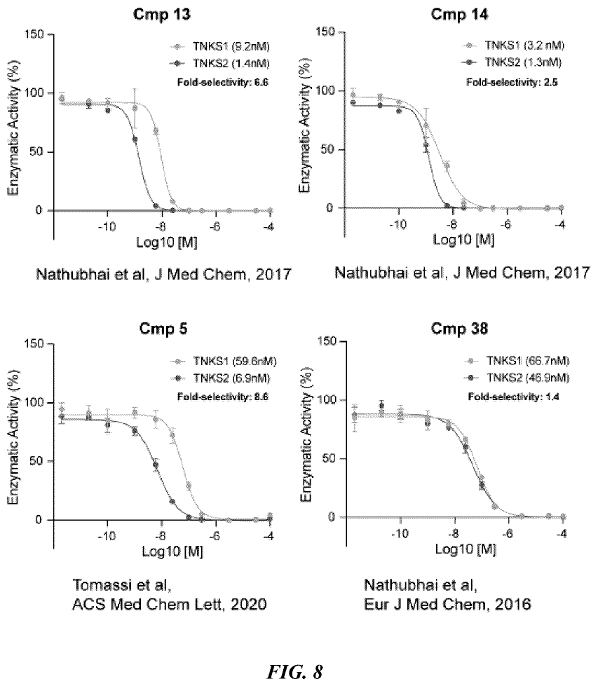

More than 50 small molecule Tankyrase inhibitors have been described in the literature (Lau et al., “A Novel Tankyrase Small-Molecule Inhibitor Suppresses APC Mutation-Driven Colorectal Tumor Growth,” Cancer Res. 73:3132-3144 (2013); Zhong et al., “Tankyrase Inhibition Causes Reversible Intestinal Toxicity in Mice with a Therapeutic Index <1,” Toxicol. Pathol.44:267-278 (2016); Huang et al., “Tankyrase Inhibition Stabilizes Axin and Antagonizes Wnt Signalling,” Nature 461:614-620 (2009); Chen et al., “Small Molecule-Mediated Disruption of Wnt-Dependent Signaling in Tissue Regeneration and Cancer,” Nat Chem Biol 5:100-107 (2009); Waaler et al., “A Novel Tankyrase Inhibitor Decreases Canonical Wnt Signaling in Colon Carcinoma Cells and Reduces Tumor Growth in Conditional Apc Mutant Mice,” Cancer Res. 72:2822-2832 (2012); Shultz et al., “Identification of NVP-TNKS656: the Use of Structure-Efficiency Relationships to Generate a Highly Potent, Selective, and Orally Active Tankyrase Inhibitor,” J. Med. Chem. 56:6495-6511 (2013); C. C. Mehta and H. G. Bhatt, “Tankyrase Inhibitors as Antitumor Agents: A Patent Update (2013-2020),” Expert Opin Ther Pat 31:645-661 (2021); Yu et al., “Small-Molecule Inhibitors of Tankyrases as Prospective Therapeutics for Cancer,” J. Med. Chem. 65:5244-5273 (2022), which are hereby incorporated by reference in their entirety). These compounds bind either the adenosine (e.g. G007-LK) (Lau et al., “A Novel Tankyrase Small-Molecule Inhibitor Suppresses APC Mutation-Driven Colorectal Tumor Growth,” Cancer Res.73:3132-3144 (2013), which is hereby incorporated by reference in its entirety) or nicotinamide (e.g. XAV939) (Huang et al., “Tankyrase Inhibition Stabilizes Axin and Antagonizes Wnt Signalling,” Nature 461:614-620 (2009), which is hereby incorporated by reference in its entirety) subsites of the enzyme (FIG. 7B), or act as dual-binders, contacting both subsites (e.g. TNKS656) (Shultz et al., “Identification of NVP-TNKS656: the Use of Structure-Efficiency Relationships to Generate a Highly Potent, Selective, and Orally Active Tankyrase Inhibitor,” J. Med. Chem. 56:6495-6511 (2013), which is hereby incorporated by reference in its entirety). Most TNKS inhibitors potently inhibit both TNKS1 and TNKS2 enzymes, though some have been reported to have 20-50 fold selectivity for TNKS2 (Nathubhai et al., “Highly Potent and Isoform Selective Dual Site Binding Tankyrase/Wnt Signaling Inhibitors That Increase Cellular Glucose Uptake and Have Antiproliferative Activity,” J. Med. Chem. 60:814-820 (2017); Tomassi et al., “From PARP1 to TNKS2 Inhibition: A Structure-Based Approach,” ACS Med. Chem. Lett. 11:862-868 (2020); Nathubhai et al., “Structure-Activity Relationships of 2-Arylquinazolin-4-Ones as Highly Selective and Potent Inhibitors of the Tankyrases,” Eur J. Med. Chem. 118:316-327 (2016), which are hereby incorporated by reference in their entirety). To directly test whether these existing compounds could provide potent and selective TNKS2 inhibition, four reported TNKS2-selective molecules were resynthesized and tested using a commercial PARylation assay for TNKS1 and TNKS2 (FIG. 8). In contrast to the published data, each of the previously described compounds showed less than 10-fold selectivity toward TNKS2, like reported pan-TNKS inhibitors (FIG. 8).

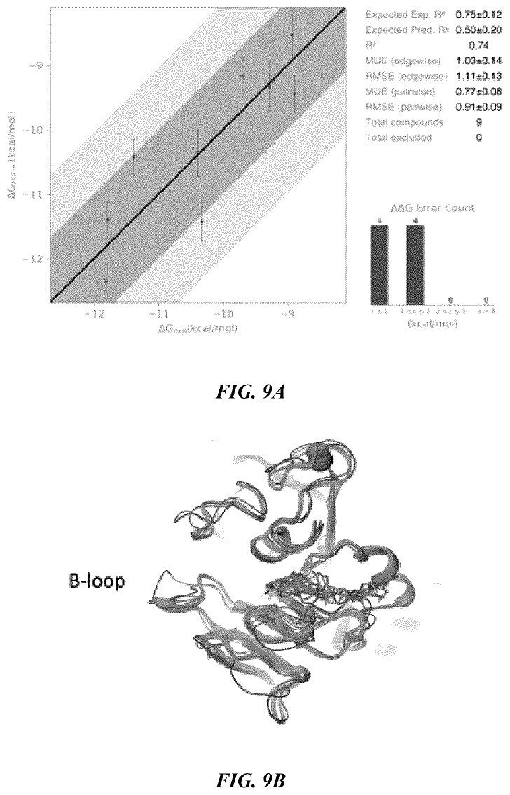

The catalytic PARP domains of TNKS1 and TNKS2 have high sequence identity, particularly in the residues that surround the nicotinamide and adenosine subsites (FIG. 7A), making selective targeting of these regions challenging. Through analysis of available crystal structures of the TNKS1 and TNKS2 enzymatic domains, it was noted that two residues (A1112 and L1136 in TNKS2) surrounding the acceptor site that were not conserved between TNKS1 and TNKS2 (FIG. 7A). It was reasoned that extending a ligand from the adjacent nicotinamide subsite into the acceptor site, could impart selectivity toward TNKS2 (FIG. 7B). An initial set of molecules derived from XAV939 with quinazolin-4 (3H)-one scaffold that effectively inhibited TNKS1/TNKS2 activity was first established and then structure-based design with docking and validated free energy calculations with FEP+ (FIGS. 9A-9B) was used to extend them toward the variant residues through the addition of substituents to quinazolin-4 (3H)-one scaffold (FIG. 7B).

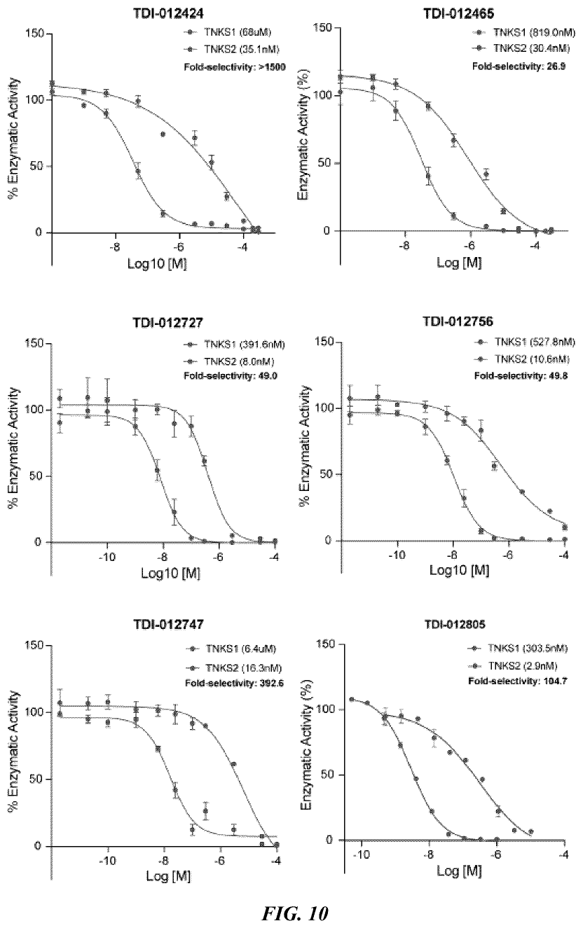

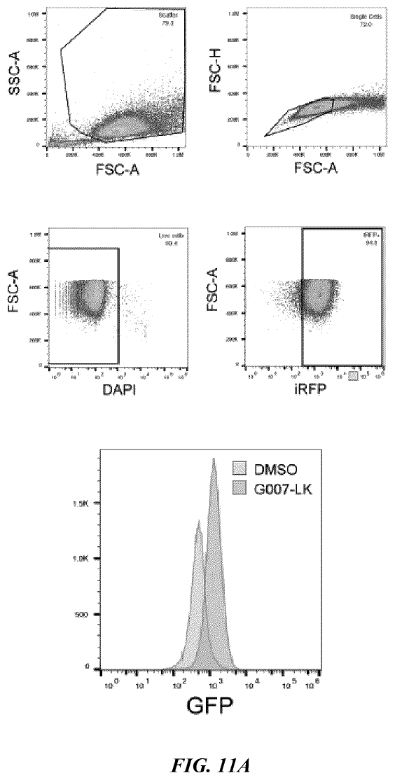

To identify TNKS2 selective inhibitors an SAR campaign exploring different trajectories on the quinazolin-4 (3H)-one core was undertaken with the objective of interacting with the flexible B-loop. In total, over 300 small molecules were synthesized with different chemical moieties and the inhibitory activity of each was measured using biochemical PARylation and cellular assays. From initial screening, 88 small molecules with good potential potency and/or selectivity for full dose response assays against TNKS1 and TNKS2 were identified. Of the 88 small molecules in secondary screening, 7 with high potency against TNKS2 (IC₅₀<50 nM) and greater than 25-fold selectivity toward TNKS2 (TNKS1 IC 50/TNKS2 IC₅₀>25) were identified (FIGS. 7C-7D and 10, Table 3).

TABLE 3
TNKS2 Inhibitors

| | Com- | TNKS2 | |
|---|---|---|---|
| | pound | (IC₅₀, | TNKS1 |
| Compound Structure | Number | nM) | (IC₅₀, nM) |
| 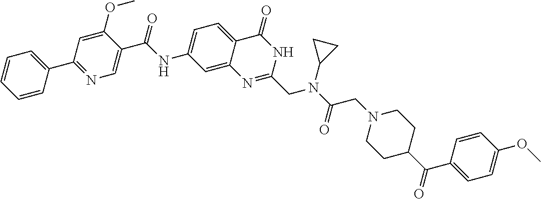 [SMILES: COc1ccc(C(=O)C2CCN(CC(=O)N(Cc3nc4cc(NC(=O)c5cnc(-c6ccccc6)cc5OC)ccc4c(=O)[nH]3)C3CC3)CC2)cc1] | 1 | 1.1 | 1 |
| 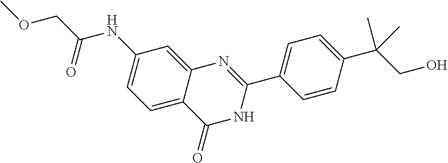 [SMILES: COCC(=O)Nc1ccc2c(=O)[nH]c(-c3ccc(C(C)(C)CO)cc3)nc2c1] | 2 | 440 | >1000 |
| 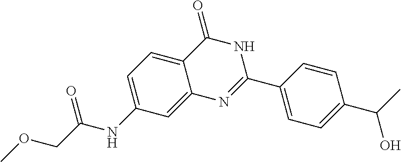 [SMILES: COCC(=O)Nc1ccc2c(=O)[nH]c(-c3ccc(C(C)O)cc3)nc2c1] | 3 | 93 | >1000 |
| 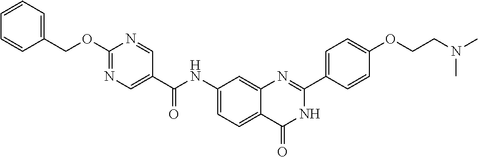 [SMILES: CN(C)CCOc1ccc(-c2nc3cc(NC(=O)c4cnc(OCc5ccccc5)nc4)ccc3c(=O)[nH]2)cc1] | 4 | 390 | >1000 |
| 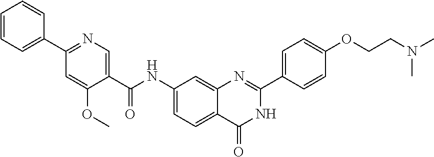 [SMILES: COc1cc(-c2ccccc2)ncc1C(=O)Nc1ccc2c(=O)[nH]c(-c3ccc(OCCN(C)C)cc3)nc2c1] | 5 | 11 | 290 |
| 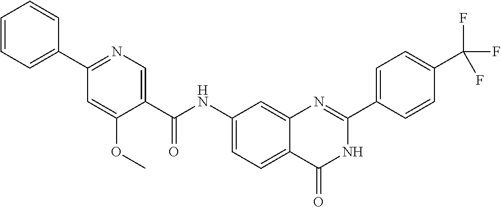 [SMILES: COc1cc(-c2ccccc2)ncc1C(=O)Nc1ccc2c(=O)[nH]c(-c3ccc(C(F)(F)F)cc3)nc2c1] | 6 | 190 | >1000 |
| 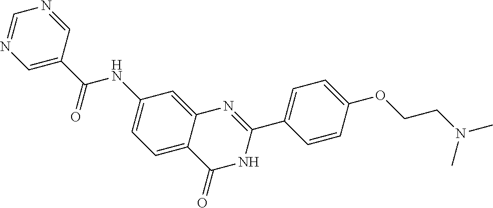 [SMILES: CN(C)CCOc1ccc(-c2nc3cc(NC(=O)c4cncnc4)ccc3c(=O)[nH]2)cc1] | 7 | >1000 | >1000 |
| 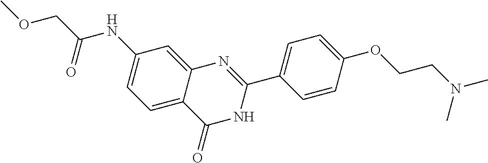 | 8 | >1000 | >1000 |
| 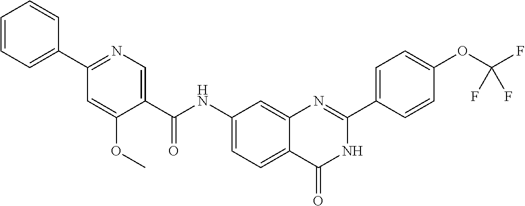 [SMILES: COc1cc(-c2ccccc2)ncc1C(=O)Nc1ccc2c(=O)[nH]c(-c3ccc(OC(F)(F)F)cc3)nc2c1] | 9 | 190 | >1000 |
| 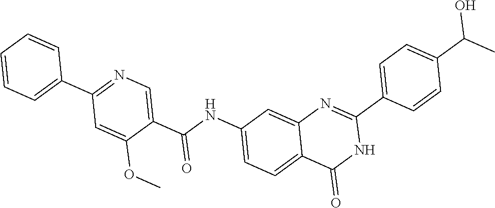 [SMILES: COc1cc(-c2ccccc2)ncc1C(=O)Nc1ccc2c(=O)[nH]c(-c3ccc(C(C)O)cc3)nc2c1] | 10 | 4.1 | 160 |
| 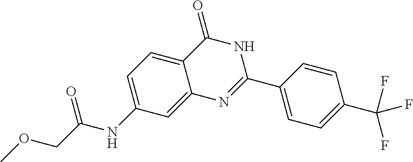 [SMILES: COCC(=O)Nc1ccc2c(=O)[nH]c(-c3ccc(C(F)(F)F)cc3)nc2c1] | 11 | 190 | >1000 |
| 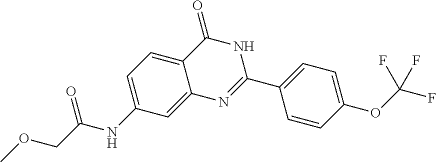 [SMILES: COCC(=O)Nc1ccc2c(=O)[nH]c(-c3ccc(OC(F)(F)F)cc3)nc2c1] | 12 | 190 | >1000 |
| 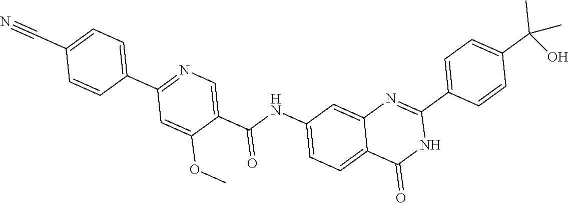 [SMILES: COc1cc(-c2ccc(C#N)cc2)ncc1C(=O)Nc1ccc2c(=O)[nH]c(-c3ccc(C(C)(C)O)cc3)nc2c1] | 13 | 9.8 | >1000 |
| 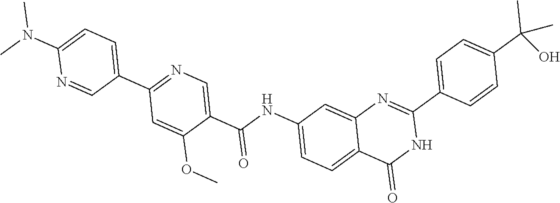 [SMILES: COc1cc(-c2ccc(N(C)C)nc2)ncc1C(=O)Nc1ccc2c(=O)[nH]c(-c3ccc(C(C)(C)O)cc3)nc2c1] | 14 | 3.37 | 4.52705E+12 |
| 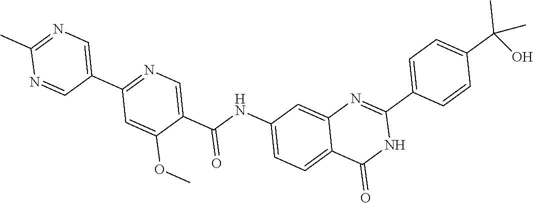 [SMILES: COc1cc(-c2cnc(C)nc2)ncc1C(=O)Nc1ccc2c(=O)[nH]c(-c3ccc(C(C)(C)O)cc3)nc2c1] | 15 | 7.66 | 134.1 |
| 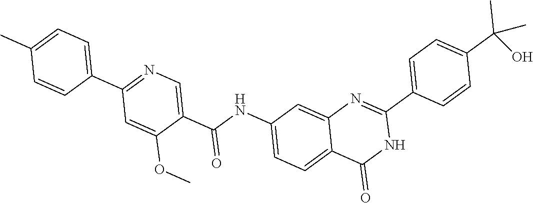 [SMILES: COc1cc(-c2ccc(C)cc2)ncc1C(=O)Nc1ccc2c(=O)[nH]c(-c3ccc(C(C)(C)O)cc3)nc2c1] | 16 | 3.34 | 33.08 |
| 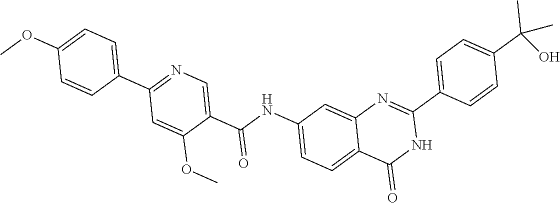 [SMILES: COc1ccc(-c2cc(OC)c(C(=O)Nc3ccc4c(=O)[nH]c(-c5ccc(C(C)(C)O)cc5)nc4c3)cn2)cc1] | 17 | 4.88 | 44.17 |
| 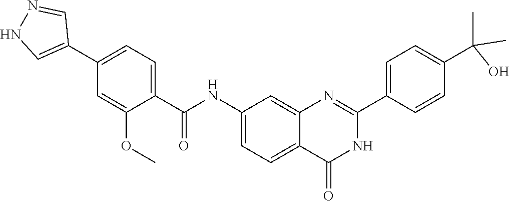 [SMILES: COc1cc(-c2cn[nH]c2)ccc1C(=O)Nc1ccc2c(=O)[nH]c(-c3ccc(C(C)(C)O)cc3)nc2c1] | 18 | 5.99 | 440 |
| 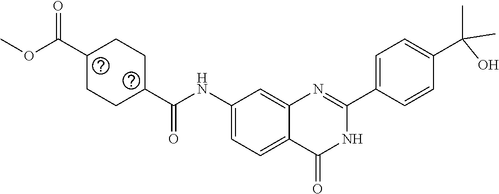 [SMILES: COC(=O)C1CCC(C(=O)Nc2ccc3c(=O)[nH]c(-c4ccc(C(C)(C)O)cc4)nc3c2)CC1] | 19 | 7% inh. at 300 nM | |
| 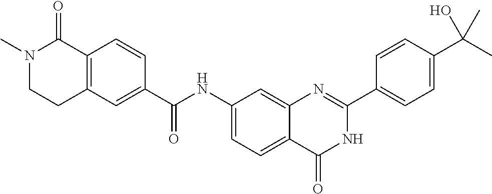 [SMILES: CN1CCc2cc(C(=O)Nc3ccc4c(=O)[nH]c(-c5ccc(C(C)(C)O)cc5)nc4c3)ccc2C1=O] | 20 | | |
| 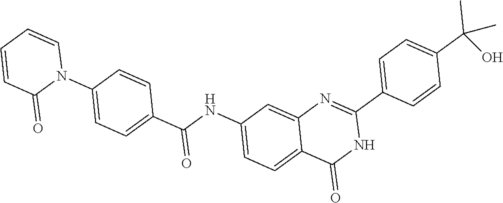 [SMILES: CC(C)(O)c1ccc(-c2nc3cc(NC(=O)c4ccc(-n5ccccc5=O)cc4)ccc3c(=O)[nH]2)cc1] | 21 | | |
| 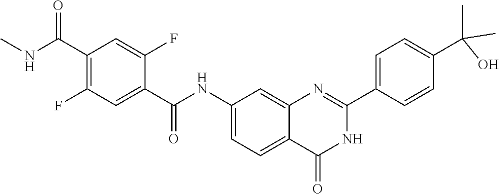 [SMILES: CNC(=O)c1cc(F)c(C(=O)Nc2ccc3c(=O)[nH]c(-c4ccc(C(C)(C)O)cc4)nc3c2)cc1F] | 22 | 3.6 | 77.6 |
| 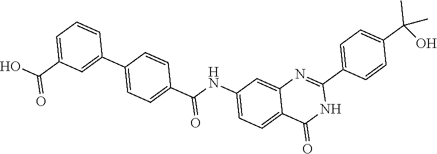 [SMILES: CC(C)(O)c1ccc(-c2nc3cc(NC(=O)c4ccc(-c5cccc(C(=O)O)c5)cc4)ccc3c(=O)[nH]2)cc1] | 23 | | |
| 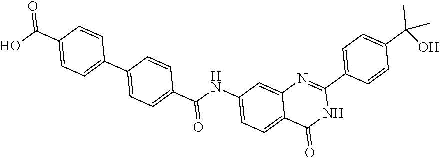 [SMILES: CC(C)(O)c1ccc(-c2nc3cc(NC(=O)c4ccc(-c5ccc(C(=O)O)cc5)cc4)ccc3c(=O)[nH]2)cc1] | 24 | | |
| 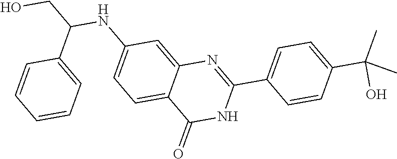 [SMILES: CC(C)(O)c1ccc(-c2nc3cc(NC(CO)c4ccccc4)ccc3c(=O)[nH]2)cc1] | 25 | | |
| 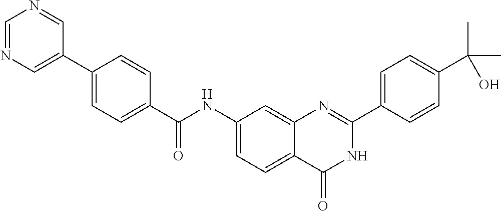 [SMILES: CC(C)(O)c1ccc(-c2nc3cc(NC(=O)c4ccc(-c5cncnc5)cc4)ccc3c(=O)[nH]2)cc1] | 26 | 8 | 108.6 |
| 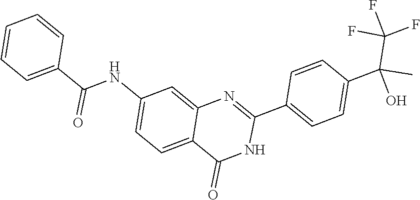 [SMILES: CC(O)(c1ccc(-c2nc3cc(NC(=O)c4ccccc4)ccc3c(=O)[nH]2)cc1)C(F)(F)F] | 27 | | |
| 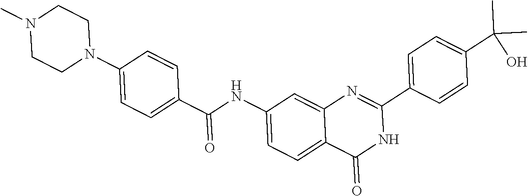 [SMILES: CN1CCN(c2ccc(C(=O)Nc3ccc4c(=O)[nH]c(-c5ccc(C(C)(C)O)cc5)nc4c3)cc2)CC1] | 28 | | |
| 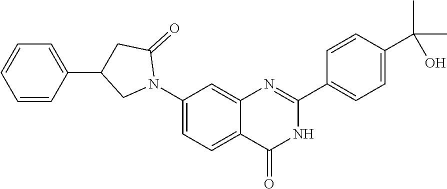 [SMILES: CC(C)(O)c1ccc(-c2nc3cc(N4CC(c5ccccc5)CC4=O)ccc3c(=O)[nH]2)cc1] | 29 | | |
| 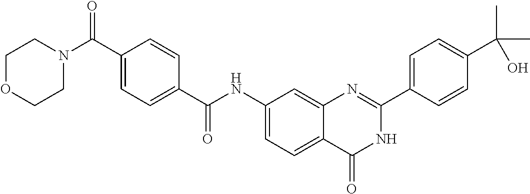 [SMILES: CC(C)(O)c1ccc(-c2nc3cc(NC(=O)c4ccc(C(=O)N5CCOCC5)cc4)ccc3c(=O)[nH]2)cc1] | 30 | | |
| 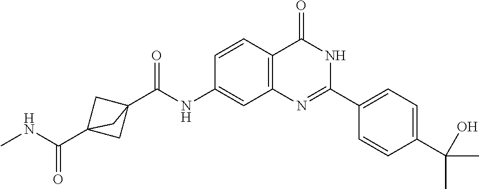 [SMILES: CNC(=O)C12CC(C(=O)Nc3ccc4c(=O)[nH]c(-c5ccc(C(C)(C)O)cc5)nc4c3)(C1)C2] | 31 | | |
| 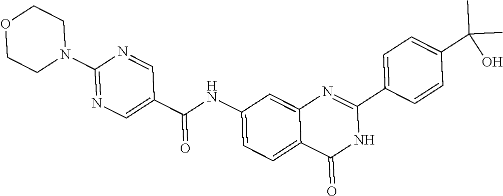 [SMILES: CC(C)(O)c1ccc(-c2nc3cc(NC(=O)c4cnc(N5CCOCC5)nc4)ccc3c(=O)[nH]2)cc1] | 32 | | |
| 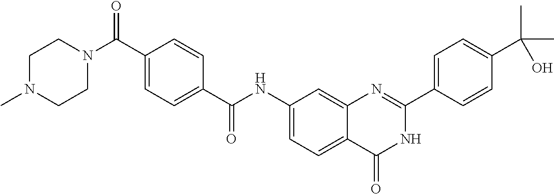 [SMILES: CN1CCN(C(=O)c2ccc(C(=O)Nc3ccc4c(=O)[nH]c(-c5ccc(C(C)(C)O)cc5)nc4c3)cc2)CC1] | 33 | | |
| 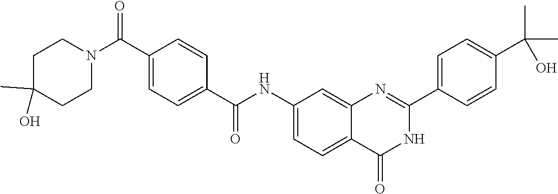 [SMILES: CC1(O)CCN(C(=O)c2ccc(C(=O)Nc3ccc4c(=O)[nH]c(-c5ccc(C(C)(C)O)cc5)nc4c3)cc2)CC1] | 34 | | |
| 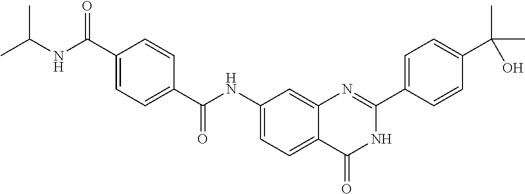 [SMILES: CC(C)NC(=O)c1ccc(C(=O)Nc2ccc3c(=O)[nH]c(-c4ccc(C(C)(C)O)cc4)nc3c2)cc1] | 35 | | |
| 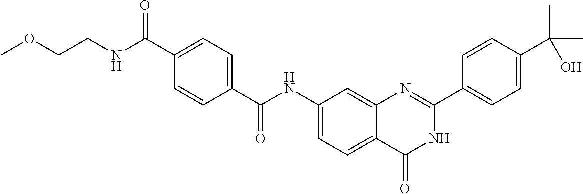 [SMILES: COCCNC(=O)c1ccc(C(=O)Nc2ccc3c(=O)[nH]c(-c4ccc(C(C)(C)O)cc4)nc3c2)cc1] | 36 | | |
| 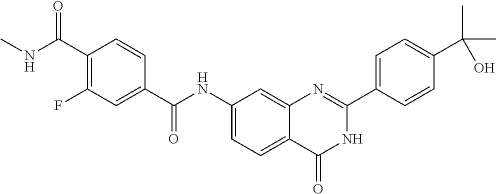 [SMILES: CNC(=O)c1ccc(C(=O)Nc2ccc3c(=O)[nH]c(-c4ccc(C(C)(C)O)cc4)nc3c2)cc1F] | 37 | | |
| 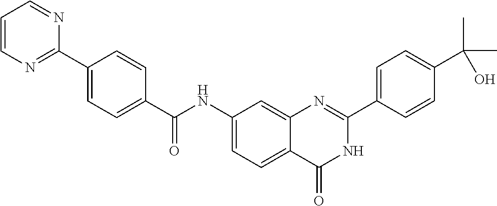 [SMILES: CC(C)(O)c1ccc(-c2nc3cc(NC(=O)c4ccc(-c5ncccn5)cc4)ccc3c(=O)[nH]2)cc1] | 38 | | |
| 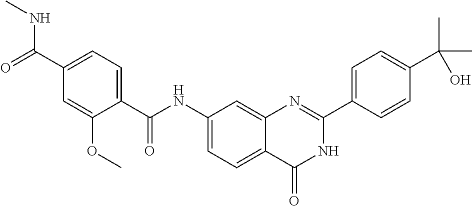 [SMILES: CNC(=O)c1ccc(C(=O)Nc2ccc3c(=O)[nH]c(-c4ccc(C(C)(C)O)cc4)nc3c2)c(OC)c1] | 39 | | |
| 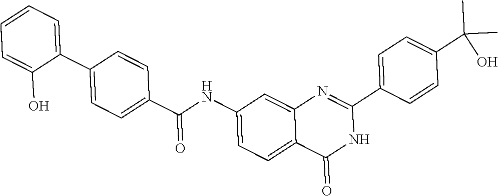 [SMILES: CC(C)(O)c1ccc(-c2nc3cc(NC(=O)c4ccc(-c5ccccc5O)cc4)ccc3c(=O)[nH]2)cc1] | 40 | | |
| 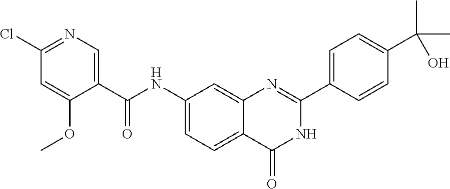 [SMILES: COc1cc(Cl)ncc1C(=O)Nc1ccc2c(=O)[nH]c(-c3ccc(C(C)(C)O)cc3)nc2c1] | 41 | 3.93 | 190 |
| 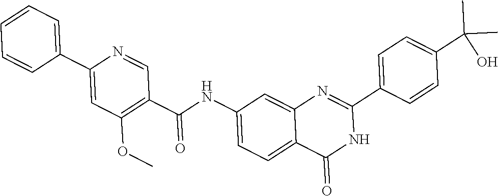 [SMILES: COc1cc(-c2ccccc2)ncc1C(=O)Nc1ccc2c(=O)[nH]c(-c3ccc(C(C)(C)O)cc3)nc2c1] | 42 | 2.9 | 80.81 |
| 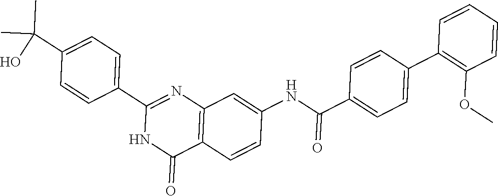 [SMILES: COc1ccccc1-c1ccc(C(=O)Nc2ccc3c(=O)[nH]c(-c4ccc(C(C)(C)O)cc4)nc3c2)cc1] | 43 | 86% inh. at 300 nM | |
| 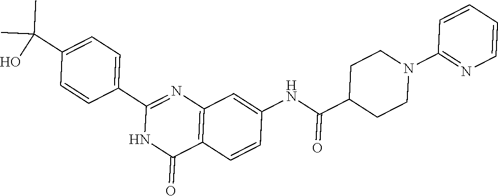 [SMILES: CC(C)(O)c1ccc(-c2nc3cc(NC(=O)C4CCN(c5ccccn5)CC4)ccc3c(=O)[nH]2)cc1] | 44 | 41.44 | 282.9 |
| 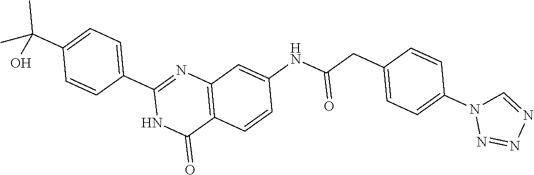 [SMILES: CC(C)(O)c1ccc(-c2nc3cc(NC(=O)Cc4ccc(-n5cnnn5)cc4)ccc3c(=O)[nH]2)cc1] | 45 | 77% inh. at 300 nM | |
| 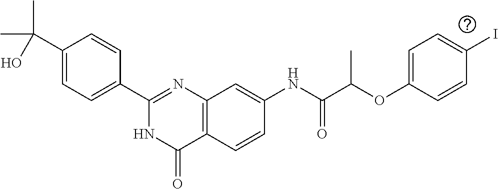 [SMILES: CC(Oc1ccc(I)cc1)C(=O)Nc1ccc2c(=O)[nH]c(-c3ccc(C(C)(C)O)cc3)nc2c1] | 46 | 68% inh. at 300 nM | |
| 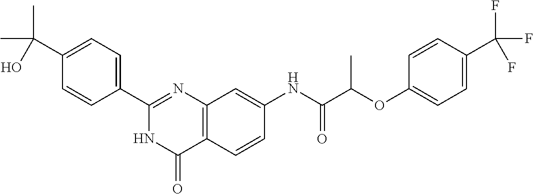 [SMILES: CC(Oc1ccc(C(F)(F)F)cc1)C(=O)Nc1ccc2c(=O)[nH]c(-c3ccc(C(C)(C)O)cc3)nc2c1] | 47 | 24% inh. at 300 nM | |
| 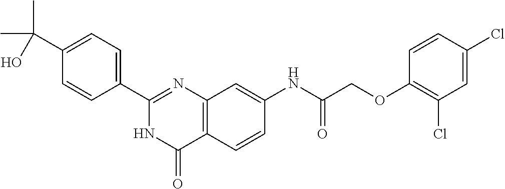 [SMILES: CC(C)(O)c1ccc(-c2nc3cc(NC(=O)COc4ccc(Cl)cc4Cl)ccc3c(=O)[nH]2)cc1] | 48 | 51% inh. at 300 nM | |
| 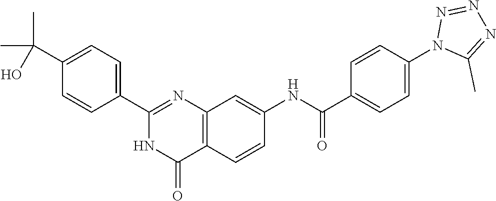 [SMILES: Cc1nnnn1-c1ccc(C(=O)Nc2ccc3c(=O)[nH]c(-c4ccc(C(C)(C)O)cc4)nc3c2)cc1] | 49 | 74.78 | 364.72 |
| 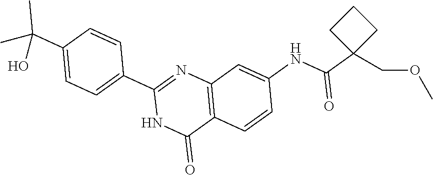 [SMILES: COCC1(C(=O)Nc2ccc3c(=O)[nH]c(-c4ccc(C(C)(C)O)cc4)nc3c2)CCC1] | 50 | 48% inh. at 300 nM | |
| 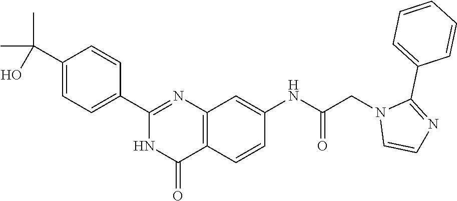 [SMILES: CC(C)(O)c1ccc(-c2nc3cc(NC(=O)Cn4ccnc4-c4ccccc4)ccc3c(=O)[nH]2)cc1] | 51 | 91% inh. at 300 nM | |
| 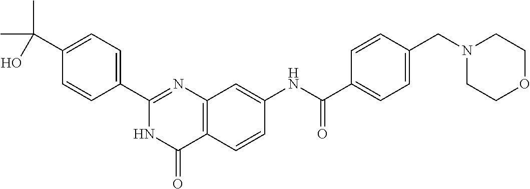 [SMILES: CC(C)(O)c1ccc(-c2nc3cc(NC(=O)c4ccc(CN5CCOCC5)cc4)ccc3c(=O)[nH]2)cc1] | 52 | 193.84 | 1025 |
| 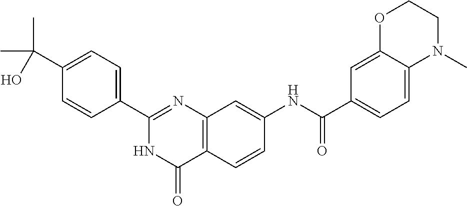 [SMILES: CN1CCOc2cc(C(=O)Nc3ccc4c(=O)[nH]c(-c5ccc(C(C)(C)O)cc5)nc4c3)ccc21] | 53 | 77% inh. at 300 nM | |
| 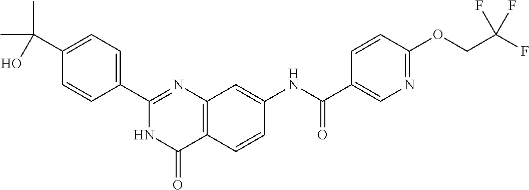 [SMILES: CC(C)(O)c1ccc(-c2nc3cc(NC(=O)c4ccc(OCC(F)(F)F)nc4)ccc3c(=O)[nH]2)cc1] | 54 | 81% inh. at 300 nM | |
| 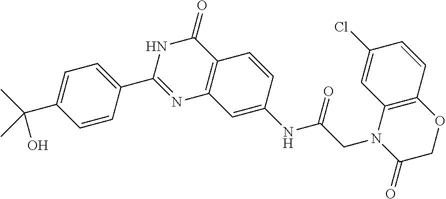 [SMILES: CC(C)(O)c1ccc(-c2nc3cc(NC(=O)CN4C(=O)COc5ccc(Cl)cc54)ccc3c(=O)[nH]2)cc1] | 55 | 83% inh. at 300 nM | |
| 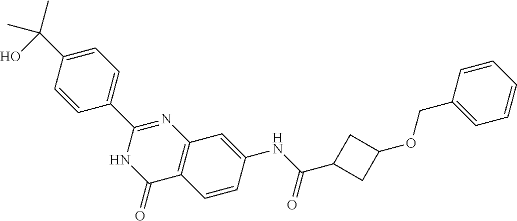 [SMILES: CC(C)(O)c1ccc(-c2nc3cc(NC(=O)C4CC(OCc5ccccc5)C4)ccc3c(=O)[nH]2)cc1] | 56 | 74% inh. at 300 nM | |
| 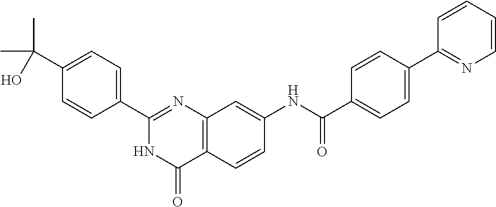 [SMILES: CC(C)(O)c1ccc(-c2nc3cc(NC(=O)c4ccc(-c5ccccn5)cc4)ccc3c(=O)[nH]2)cc1] | 57 | 57.85 | 695.22 |
| 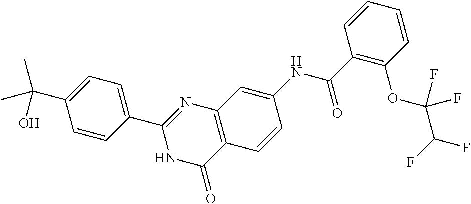 [SMILES: CC(C)(O)c1ccc(-c2nc3cc(NC(=O)c4ccccc4OC(F)(F)C(F)F)ccc3c(=O)[nH]2)cc1] | 58 | 47% inh. at 300 nM | |
| 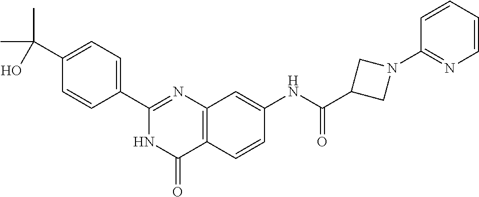 [SMILES: CC(C)(O)c1ccc(-c2nc3cc(NC(=O)C4CN(c5ccccn5)C4)ccc3c(=O)[nH]2)cc1] | 59 | 96% inh. at 300 nM | |
| 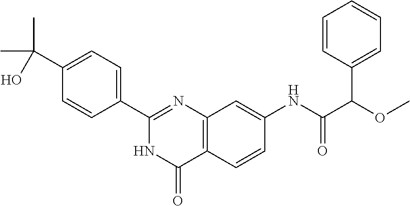 [SMILES: COC(C(=O)Nc1ccc2c(=O)[nH]c(-c3ccc(C(C)(C)O)cc3)nc2c1)c1ccccc1] | 60 | 63% inh. at 300 nM | |
| 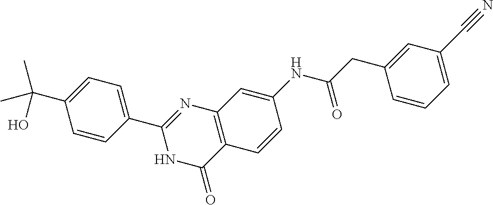 [SMILES: CC(C)(O)c1ccc(-c2nc3cc(NC(=O)Cc4cccc(C#N)c4)ccc3c(=O)[nH]2)cc1] | 61 | 90% inh. at 300 nM | |
| 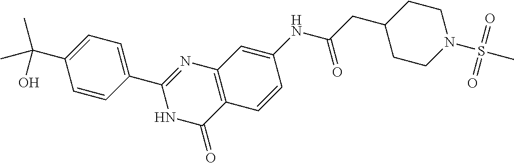 [SMILES: CC(C)(O)c1ccc(-c2nc3cc(NC(=O)CC4CCN(S(C)(=O)=O)CC4)ccc3c(=O)[nH]2)cc1] | 62 | 69% inh. at 300 nM | |
| 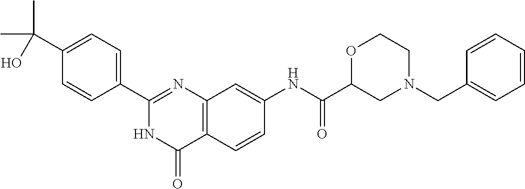 [SMILES: CC(C)(O)c1ccc(-c2nc3cc(NC(=O)C4CN(Cc5ccccc5)CCO4)ccc3c(=O)[nH]2)cc1] | 63 | 67% inh. at 300 nM | |
| 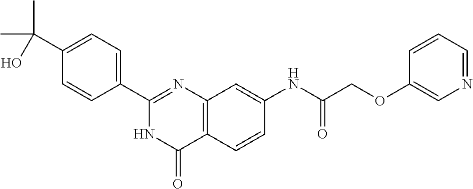 [SMILES: CC(C)(O)c1ccc(-c2nc3cc(NC(=O)COc4cccnc4)ccc3c(=O)[nH]2)cc1] | 64 | 87% inh. at 300 nM | |
| 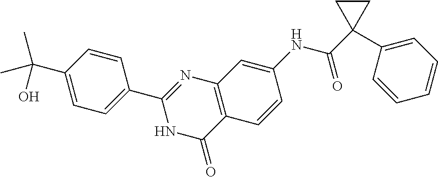 [SMILES: CC(C)(O)c1ccc(-c2nc3cc(NC(=O)C4(c5ccccc5)CC4)ccc3c(=O)[nH]2)cc1] | 65 | 58% inh. at 300 nM | |
| 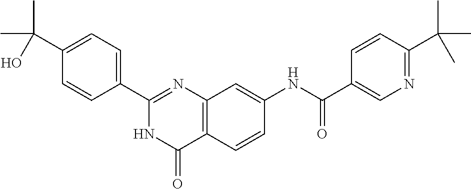 [SMILES: CC(C)(C)c1ccc(C(=O)Nc2ccc3c(=O)[nH]c(-c4ccc(C(C)(C)O)cc4)nc3c2)cn1] | 66 | 84% inh. at 300 nM | |
| 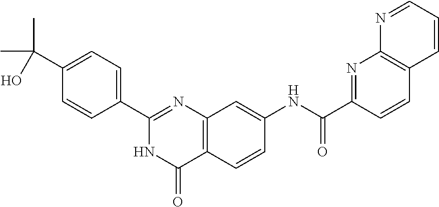 [SMILES: CC(C)(O)c1ccc(-c2nc3cc(NC(=O)c4ccc5cccnc5n4)ccc3c(=O)[nH]2)cc1] | 67 | 68% inh. at 300 nM | |
| 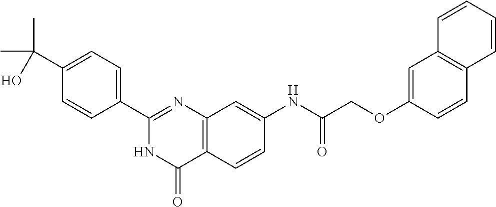 [SMILES: CC(C)(O)c1ccc(-c2nc3cc(NC(=O)COc4ccc5ccccc5c4)ccc3c(=O)[nH]2)cc1] | 68 | 86% inh. at 300 nM | |
| 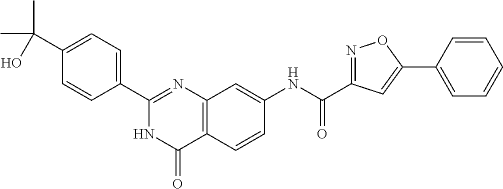 [SMILES: CC(C)(O)c1ccc(-c2nc3cc(NC(=O)c4cc(-c5ccccc5)on4)ccc3c(=O)[nH]2)cc1] | 69 | 142.11 | >25000 |
| 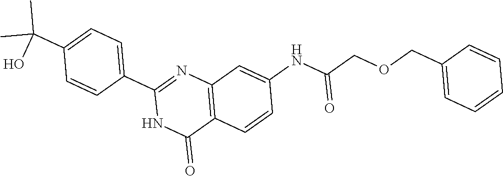 [SMILES: CC(C)(O)c1ccc(-c2nc3cc(NC(=O)COCc4ccccc4)ccc3c(=O)[nH]2)cc1] | 70 | 80% inh. at 300 nM | |
| 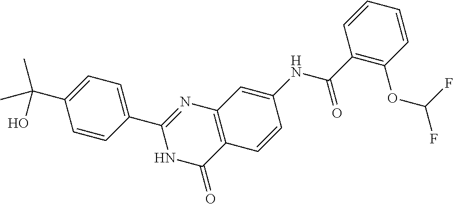 [SMILES: CC(C)(O)c1ccc(-c2nc3cc(NC(=O)c4ccccc4OC(F)F)ccc3c(=O)[nH]2)cc1] | 71 | 32.63 | 359.54 |
| 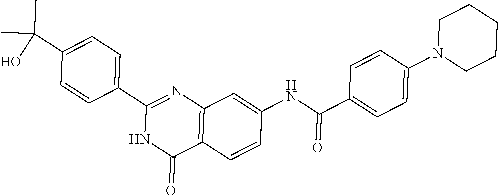 [SMILES: CC(C)(O)c1ccc(-c2nc3cc(NC(=O)c4ccc(N5CCCCC5)cc4)ccc3c(=O)[nH]2)cc1] | 72 | 82% inh. at 300 nM | |
| 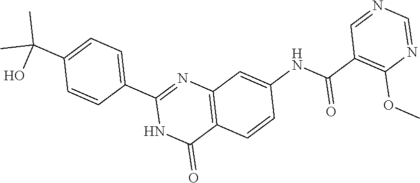 [SMILES: COc1ncncc1C(=O)Nc1ccc2c(=O)[nH]c(-c3ccc(C(C)(C)O)cc3)nc2c1] | 73 | 85% inh. at 30 nM | |
| 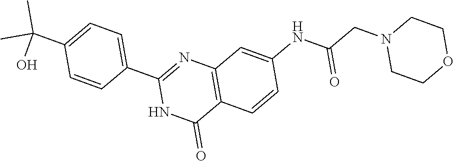 [SMILES: CC(C)(O)c1ccc(-c2nc3cc(NC(=O)CN4CCOCC4)ccc3c(=O)[nH]2)cc1] | 74 | 26% inh. at 300 nM | |
| 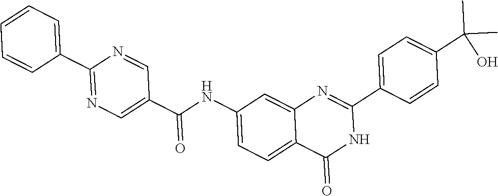 [SMILES: CC(C)(O)c1ccc(-c2nc3cc(NC(=O)c4cnc(-c5ccccc5)nc4)ccc3c(=O)[nH]2)cc1] | 75 | 9.6 | 45 |
| 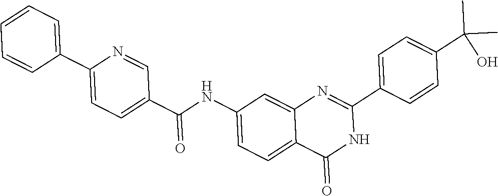 [SMILES: CC(C)(O)c1ccc(-c2nc3cc(NC(=O)c4ccc(-c5ccccc5)nc4)ccc3c(=O)[nH]2)cc1] | 76 | 10.9 | 684.64 |
| 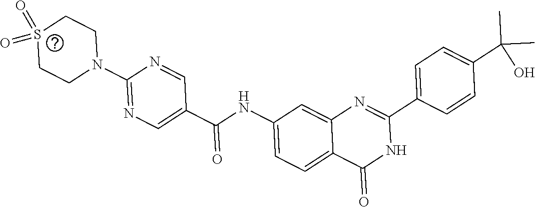 [SMILES: CC(C)(O)c1ccc(-c2nc3cc(NC(=O)c4cnc(N5CCS(=O)(=O)CC5)nc4)ccc3c(=O)[nH]2)cc1] | 77 | 49% inh. at 30 nM | |
| 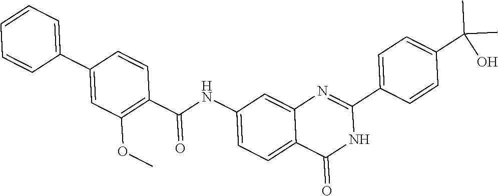 [SMILES: COc1cc(-c2ccccc2)ccc1C(=O)Nc1ccc2c(=O)[nH]c(-c3ccc(C(C)(C)O)cc3)nc2c1] | 78 | 40% inh. at 300 nM | |
| 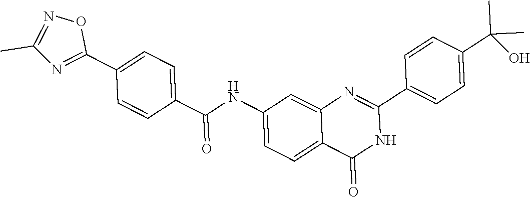 [SMILES: Cc1noc(-c2ccc(C(=O)Nc3ccc4c(=O)[nH]c(-c5ccc(C(C)(C)O)cc5)nc4c3)cc2)n1] | 79 | 3.72 | 37 |
| 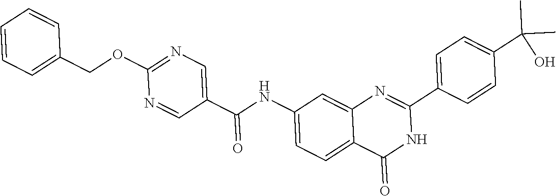 [SMILES: CC(C)(O)c1ccc(-c2nc3cc(NC(=O)c4cnc(OCc5ccccc5)nc4)ccc3c(=O)[nH]2)cc1] | 80 | 16.7 | 5662.38 |
| 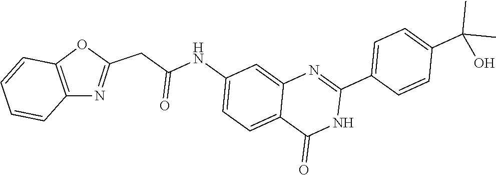 [SMILES: CC(C)(O)c1ccc(-c2nc3cc(NC(=O)Cc4nc5ccccc5o4)ccc3c(=O)[nH]2)cc1] | 81 | 22 | 73 |
| 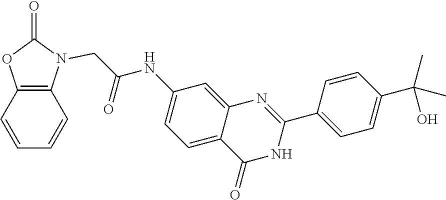 [SMILES: CC(C)(O)c1ccc(-c2nc3cc(NC(=O)Cn4c(=O)oc5ccccc54)ccc3c(=O)[nH]2)cc1] | 82 | 82% inh. at 300 nM | |
| 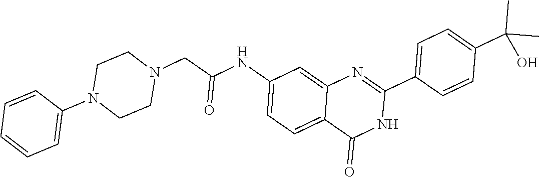 [SMILES: CC(C)(O)c1ccc(-c2nc3cc(NC(=O)CN4CCN(c5ccccc5)CC4)ccc3c(=O)[nH]2)cc1] | 83 | 46% inh. at 300 nM | |
| 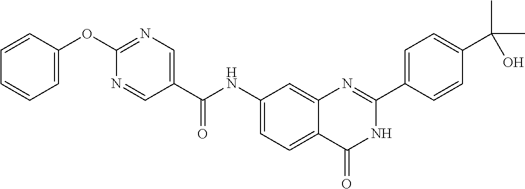 [SMILES: CC(C)(O)c1ccc(-c2nc3cc(NC(=O)c4cnc(Oc5ccccc5)nc4)ccc3c(=O)[nH]2)cc1] | 84 | 8.65 | 419.66 |
| 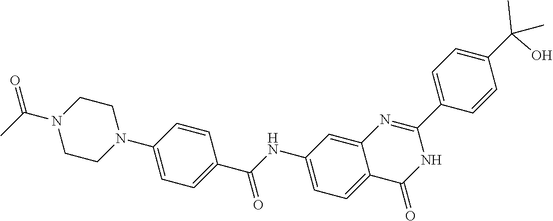 [SMILES: CC(=O)N1CCN(c2ccc(C(=O)Nc3ccc4c(=O)[nH]c(-c5ccc(C(C)(C)O)cc5)nc4c3)cc2)CC1] | 85 | 77 | 156 |
| 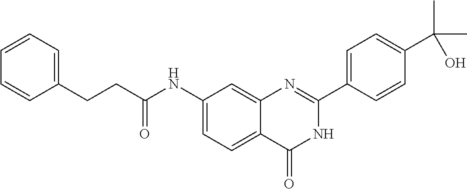 [SMILES: CC(C)(O)c1ccc(-c2nc3cc(NC(=O)CCc4ccccc4)ccc3c(=O)[nH]2)cc1] | 86 | 40% inh. at 300 nM | |
| 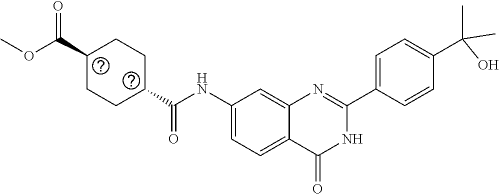 [SMILES: COC(=O)[C@H]1CC[C@H](C(=O)Nc2ccc3c(=O)[nH]c(-c4ccc(C(C)(C)O)cc4)nc3c2)CC1] | 87 | 19 | 86 |
| 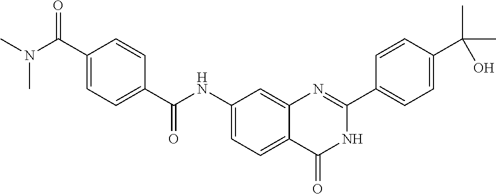 [SMILES: CN(C)C(=O)c1ccc(C(=O)Nc2ccc3c(=O)[nH]c(-c4ccc(C(C)(C)O)cc4)nc3c2)cc1] | 88 | 80% inh. at 300 nM | |
| 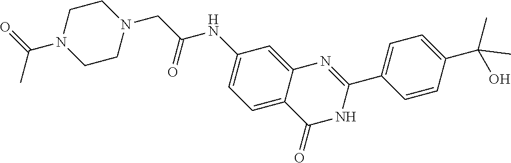 [SMILES: CC(=O)N1CCN(CC(=O)Nc2ccc3c(=O)[nH]c(-c4ccc(C(C)(C)O)cc4)nc3c2)CC1] | 89 | 35% inh. at 300 nM | |
| 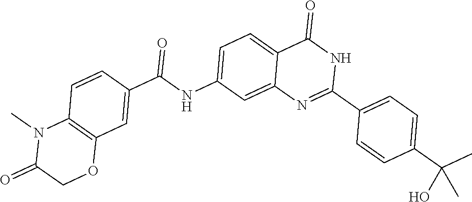 [SMILES: CN1C(=O)COc2cc(C(=O)Nc3ccc4c(=O)[nH]c(-c5ccc(C(C)(C)O)cc5)nc4c3)ccc21] | 90 | 88% inh. at 300 nM | |
| 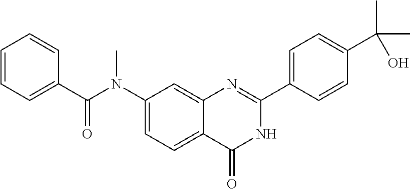 [SMILES: CN(C(=O)c1ccccc1)c1ccc2c(=O)[nH]c(-c3ccc(C(C)(C)O)cc3)nc2c1] | 91 | 2% inh. at 300 nM | |
| 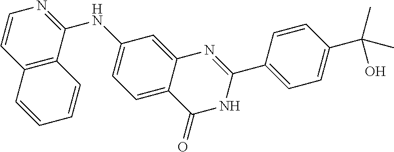 [SMILES: CC(C)(O)c1ccc(-c2nc3cc(Nc4nccc5ccccc45)ccc3c(=O)[nH]2)cc1] | 92 | 29% inh. at 300 nM | |
| 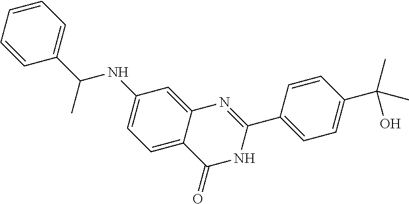 [SMILES: CC(Nc1ccc2c(=O)[nH]c(-c3ccc(C(C)(C)O)cc3)nc2c1)c1ccccc1] | 93 | 31% inh. at 300 nM | |
| 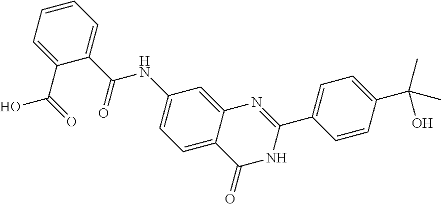 [SMILES: CC(C)(O)c1ccc(-c2nc3cc(NC(=O)c4ccccc4C(=O)O)ccc3c(=O)[nH]2)cc1] | 94 | 62% inh. at 30 nM | |
| 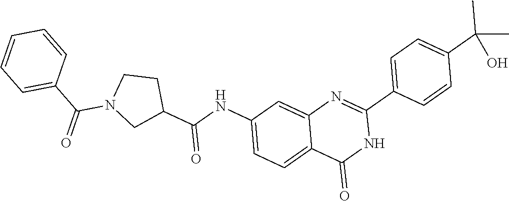 [SMILES: CC(C)(O)c1ccc(-c2nc3cc(NC(=O)C4CCN(C(=O)c5ccccc5)C4)ccc3c(=O)[nH]2)cc1] | 95 | 32.76 | 1000 |
| 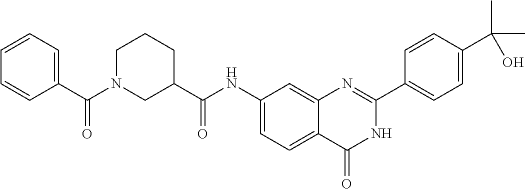 [SMILES: CC(C)(O)c1ccc(-c2nc3cc(NC(=O)C4CCCN(C(=O)c5ccccc5)C4)ccc3c(=O)[nH]2)cc1] | 96 | 21.17 | 100 |
| 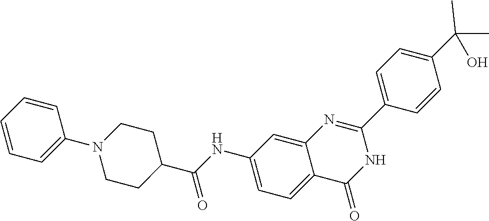 [SMILES: CC(C)(O)c1ccc(-c2nc3cc(NC(=O)C4CCN(c5ccccc5)CC4)ccc3c(=O)[nH]2)cc1] | 97 | 13.86 | 90 |
| 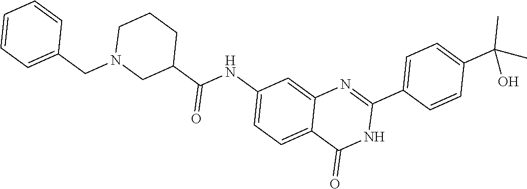 [SMILES: CC(C)(O)c1ccc(-c2nc3cc(NC(=O)C4CCCN(Cc5ccccc5)C4)ccc3c(=O)[nH]2)cc1] | 98 | 149.67 | 2100 |
| 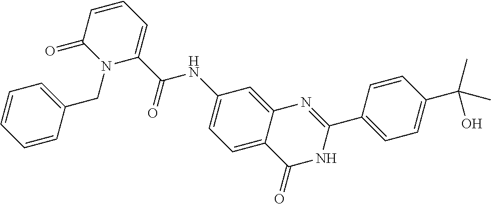 [SMILES: CC(C)(O)c1ccc(-c2nc3cc(NC(=O)c4cccc(=O)n4Cc4ccccc4)ccc3c(=O)[nH]2)cc1] | 99 | 31% inh. at 300 nM | |
| 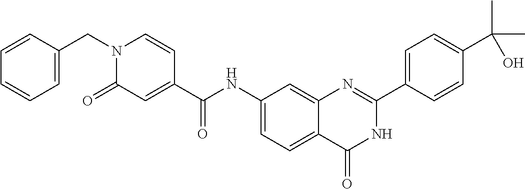 [SMILES: CC(C)(O)c1ccc(-c2nc3cc(NC(=O)c4ccn(Cc5ccccc5)c(=O)c4)ccc3c(=O)[nH]2)cc1] | 100 | 26.83 | 270 |
| 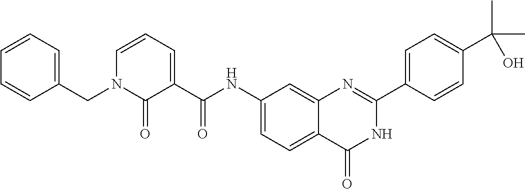 [SMILES: CC(C)(O)c1ccc(-c2nc3cc(NC(=O)c4cccn(Cc5ccccc5)c4=O)ccc3c(=O)[nH]2)cc1] | 101 | 54% inh. at 300 nM | |
| 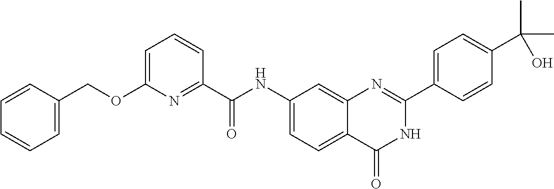 [SMILES: CC(C)(O)c1ccc(-c2nc3cc(NC(=O)c4cccc(OCc5ccccc5)n4)ccc3c(=O)[nH]2)cc1] | 102 | 10% inh. at 300 nM | |
| 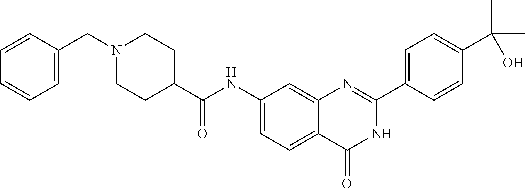 [SMILES: CC(C)(O)c1ccc(-c2nc3cc(NC(=O)C4CCN(Cc5ccccc5)CC4)ccc3c(=O)[nH]2)cc1] | 103 | 83% inh. at 300 nM | |
| 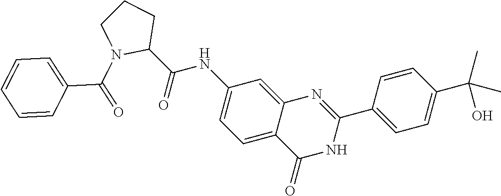 [SMILES: CC(C)(O)c1ccc(-c2nc3cc(NC(=O)C4CCCN4C(=O)c4ccccc4)ccc3c(=O)[nH]2)cc1] | 104 | 36.99 | 120 |
| 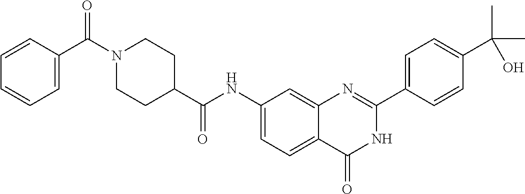 [SMILES: CC(C)(O)c1ccc(-c2nc3cc(NC(=O)C4CCN(C(=O)c5ccccc5)CC4)ccc3c(=O)[nH]2)cc1] | 105 | 92% inh. at 300 nM | |
| 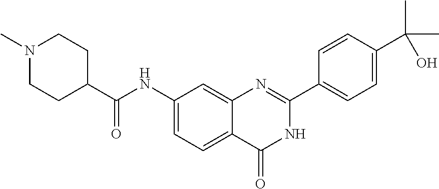 [SMILES: CN1CCC(C(=O)Nc2ccc3c(=O)[nH]c(-c4ccc(C(C)(C)O)cc4)nc3c2)CC1] | 106 | 32% inh. at 300 nM | |
| 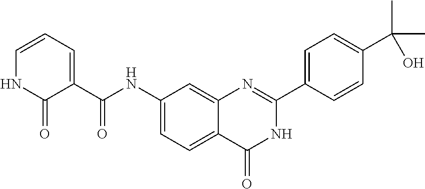 [SMILES: CC(C)(O)c1ccc(-c2nc3cc(NC(=O)c4ccc[nH]c4=O)ccc3c(=O)[nH]2)cc1] | 107 | >100000 | >100000 |
| 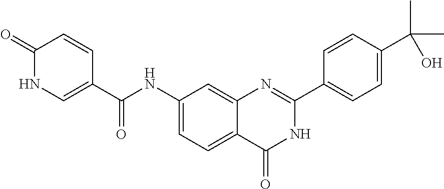 [SMILES: CC(C)(O)c1ccc(-c2nc3cc(NC(=O)c4ccc(=O)[nH]c4)ccc3c(=O)[nH]2)cc1] | 108 | 290.59 | 2830.32 |
| 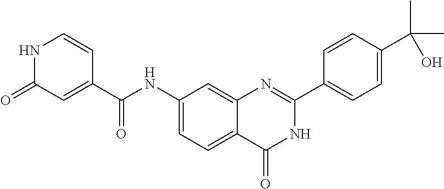 [SMILES: CC(C)(O)c1ccc(-c2nc3cc(NC(=O)c4cc[nH]c(=O)c4)ccc3c(=O)[nH]2)cc1] | 109 | 50.17 | 656.24 |
| 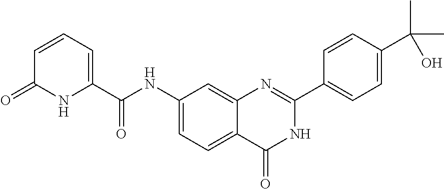 [SMILES: CC(C)(O)c1ccc(-c2nc3cc(NC(=O)c4cccc(=O)[nH]4)ccc3c(=O)[nH]2)cc1] | 110 | 42.75 | 199.4 |
| 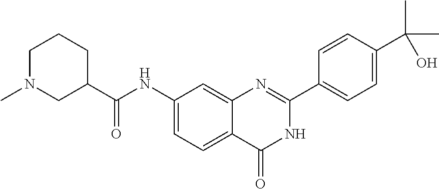 [SMILES: CN1CCCC(C(=O)Nc2ccc3c(=O)[nH]c(-c4ccc(C(C)(C)O)cc4)nc3c2)C1] | 111 | 234.87 | 2093.39 |
| 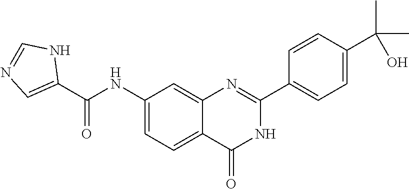 [SMILES: CC(C)(O)c1ccc(-c2nc3cc(NC(=O)c4cnc[nH]4)ccc3c(=O)[nH]2)cc1] | 112 | 359.8 | 3571.67 |
| 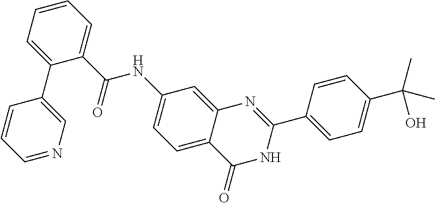 [SMILES: CC(C)(O)c1ccc(-c2nc3cc(NC(=O)c4ccccc4-c4cccnc4)ccc3c(=O)[nH]2)cc1] | 113 | 73.02 | 296.11 |
| 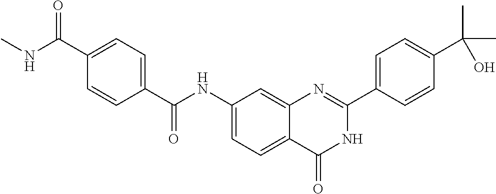 [SMILES: CNC(=O)c1ccc(C(=O)Nc2ccc3c(=O)[nH]c(-c4ccc(C(C)(C)O)cc4)nc3c2)cc1] | 114 | 22.18 | 1273.09 |
| 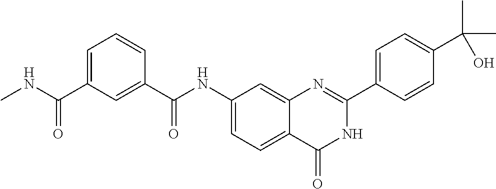 [SMILES: CNC(=O)c1cccc(C(=O)Nc2ccc3c(=O)[nH]c(-c4ccc(C(C)(C)O)cc4)nc3c2)c1] | 115 | 48.97 | 717.39 |
| 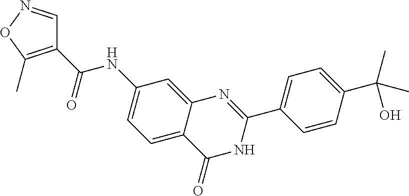 [SMILES: Cc1oncc1C(=O)Nc1ccc2c(=O)[nH]c(-c3ccc(C(C)(C)O)cc3)nc2c1] | 116 | 978.35 | 14472 |
| 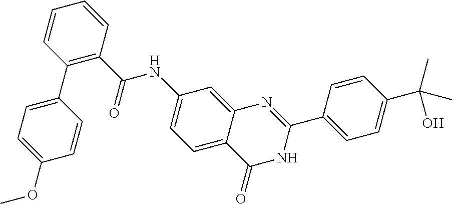 [SMILES: COc1ccc(-c2ccccc2C(=O)Nc2ccc3c(=O)[nH]c(-c4ccc(C(C)(C)O)cc4)nc3c2)cc1] | 117 | >100000 | >100000 |
| 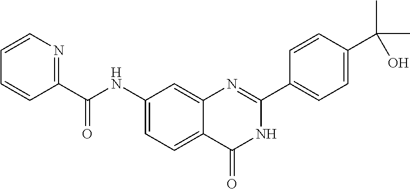 [SMILES: CC(C)(O)c1ccc(-c2nc3cc(NC(=O)c4ccccn4)ccc3c(=O)[nH]2)cc1] | 118 | 387.04 | 2121.32 |
| 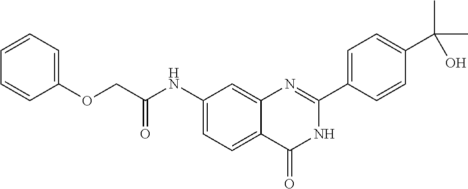 [SMILES: CC(C)(O)c1ccc(-c2nc3cc(NC(=O)COc4ccccc4)ccc3c(=O)[nH]2)cc1] | 119 | 52.45 | 953.94 |
| 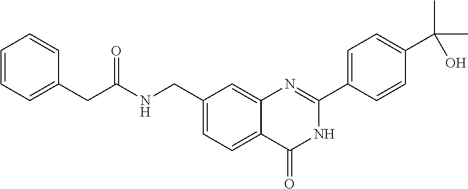 [SMILES: CC(C)(O)c1ccc(-c2nc3cc(CNC(=O)Cc4ccccc4)ccc3c(=O)[nH]2)cc1] | 120 | 1240.97 | ~5000 |
| 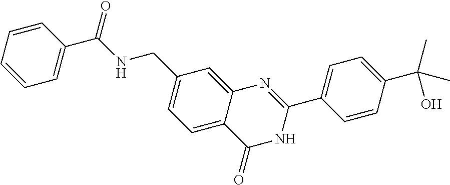 [SMILES: CC(C)(O)c1ccc(-c2nc3cc(CNC(=O)c4ccccc4)ccc3c(=O)[nH]2)cc1] | 121 | 2078.46 | ~10000 |
| 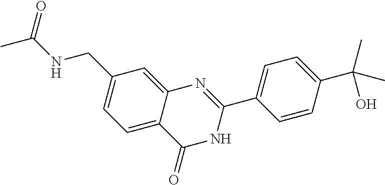 [SMILES: CC(=O)NCc1ccc2c(=O)[nH]c(-c3ccc(C(C)(C)O)cc3)nc2c1] | 122 | 2900 | 5500 |
| 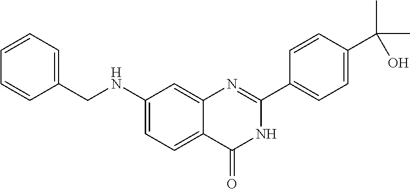 [SMILES: CC(C)(O)c1ccc(-c2nc3cc(NCc4ccccc4)ccc3c(=O)[nH]2)cc1] | 123 | 275.44 | 753.79 |
| 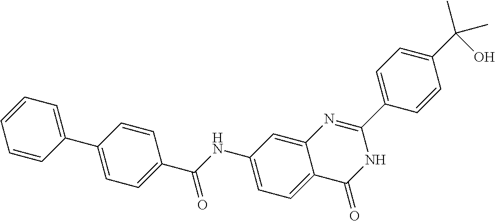 [SMILES: CC(C)(O)c1ccc(-c2nc3cc(NC(=O)c4ccc(-c5ccccc5)cc4)ccc3c(=O)[nH]2)cc1] | 124 | 33.3 | 13890 |
| 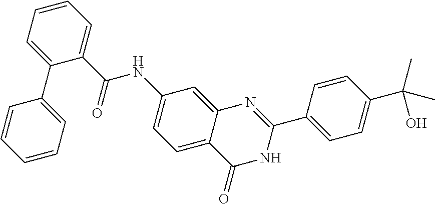 [SMILES: CC(C)(O)c1ccc(-c2nc3cc(NC(=O)c4ccccc4-c4ccccc4)ccc3c(=O)[nH]2)cc1] | 125 | 333.15 | 4800 |
| 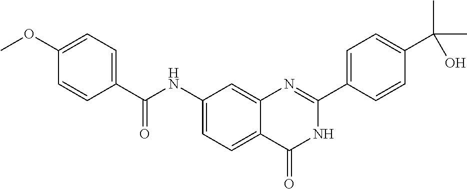 [SMILES: COc1ccc(C(=O)Nc2ccc3c(=O)[nH]c(-c4ccc(C(C)(C)O)cc4)nc3c2)cc1] | 126 | 58.22 | 703.63 |
| 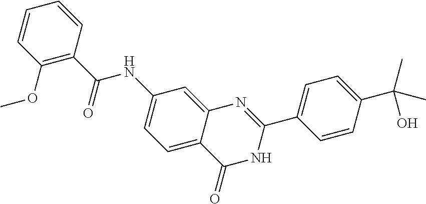 [SMILES: COc1ccccc1C(=O)Nc1ccc2c(=O)[nH]c(-c3ccc(C(C)(C)O)cc3)nc2c1] | 127 | 10.3 | 203.38 |
| 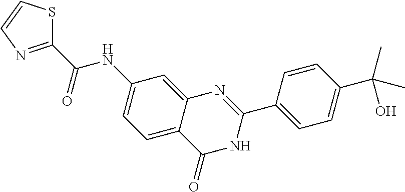 [SMILES: CC(C)(O)c1ccc(-c2nc3cc(NC(=O)c4nccs4)ccc3c(=O)[nH]2)cc1] | 128 | 88.8 | 362.6 |
| 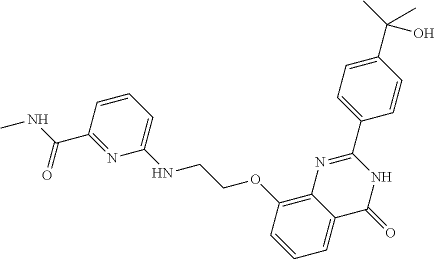 [SMILES: CNC(=O)c1cccc(NCCOc2cccc3c(=O)[nH]c(-c4ccc(C(C)(C)O)cc4)nc23)n1] | 129 | 21.55 | 132.25 |
| 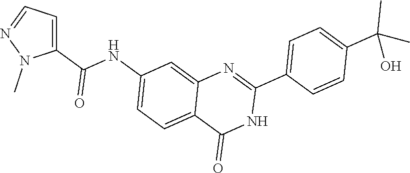 [SMILES: Cn1nccc1C(=O)Nc1ccc2c(=O)[nH]c(-c3ccc(C(C)(C)O)cc3)nc2c1] | 130 | 343.89 | 3934.46 |
| 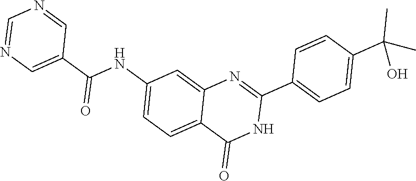 [SMILES: CC(C)(O)c1ccc(-c2nc3cc(NC(=O)c4cncnc4)ccc3c(=O)[nH]2)cc1] | 131 | 38.33 | 770.68 |
| 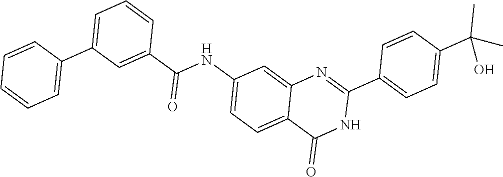 [SMILES: CC(C)(O)c1ccc(-c2nc3cc(NC(=O)c4cccc(-c5ccccc5)c4)ccc3c(=O)[nH]2)cc1] | 132 | 246.95 | >1000 |
| 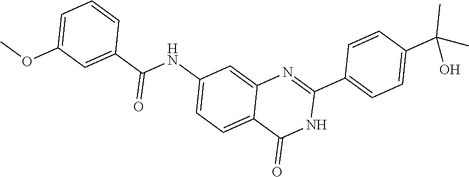 [SMILES: COc1cccc(C(=O)Nc2ccc3c(=O)[nH]c(-c4ccc(C(C)(C)O)cc4)nc3c2)c1] | 133 | 61.01 | 561.16 |
| 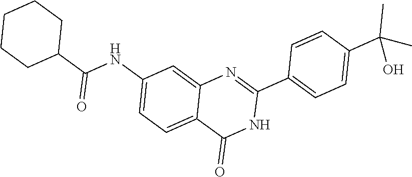 [SMILES: CC(C)(O)c1ccc(-c2nc3cc(NC(=O)C4CCCCC4)ccc3c(=O)[nH]2)cc1] | 134 | 58.19 | 349.63 |
| 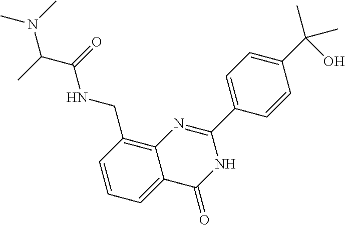 [SMILES: CC(C(=O)NCc1cccc2c(=O)[nH]c(-c3ccc(C(C)(C)O)cc3)nc12)N(C)C] | 135 | 116.9 | 302.1 |
| 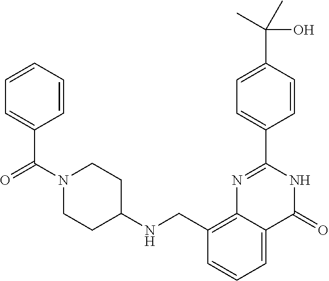 [SMILES: CC(C)(O)c1ccc(-c2nc3c(CNC4CCN(C(=O)c5ccccc5)CC4)cccc3c(=O)[nH]2)cc1] | 136 | 33.5 | 59.1 |
| 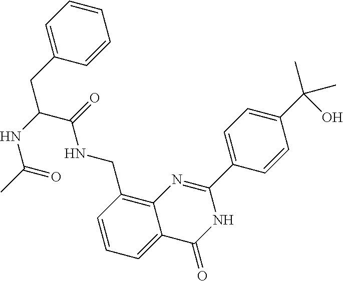 [SMILES: CC(=O)NC(Cc1ccccc1)C(=O)NCc1cccc2c(=O)[nH]c(-c3ccc(C(C)(C)O)cc3)nc12] | 137 | 76.13 | 78.07 |
| 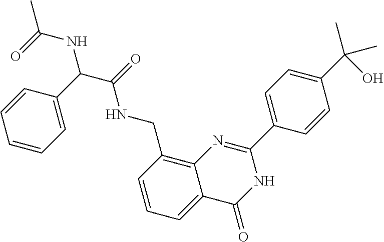 [SMILES: CC(=O)NC(C(=O)NCc1cccc2c(=O)[nH]c(-c3ccc(C(C)(C)O)cc3)nc12)c1ccccc1] | 138 | 70% inh. at 300 nM | |
| 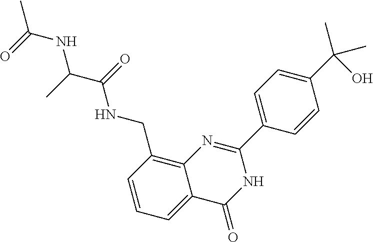 [SMILES: CC(=O)NC(C)C(=O)NCc1cccc2c(=O)[nH]c(-c3ccc(C(C)(C)O)cc3)nc12] | 139 | 91% inh. at 300 nM | 95% inh. at 300 nM |
| 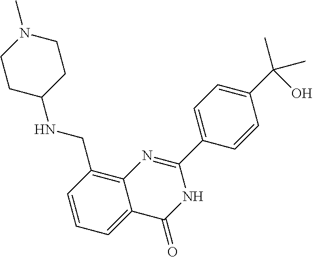 [SMILES: CN1CCC(NCc2cccc3c(=O)[nH]c(-c4ccc(C(C)(C)O)cc4)nc23)CC1] | 140 | 34% inh. at 300 nM | |
| 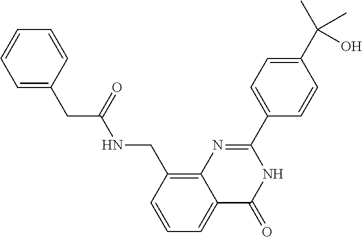 [SMILES: CC(C)(O)c1ccc(-c2nc3c(CNC(=O)Cc4ccccc4)cccc3c(=O)[nH]2)cc1] | 141 | 87% inh. at 300 nM | 72% inh. at 300 nM |
| 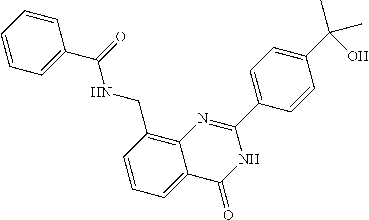 [SMILES: CC(C)(O)c1ccc(-c2nc3c(CNC(=O)c4ccccc4)cccc3c(=O)[nH]2)cc1] | 142 | 8.91 | 52.85 |
| 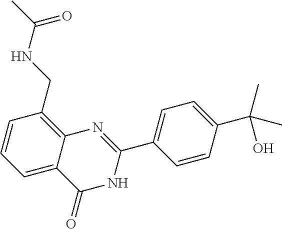 [SMILES: CC(=O)NCc1cccc2c(=O)[nH]c(-c3ccc(C(C)(C)O)cc3)nc12] | 143 | 86% inh. at 300 nM | 97% inh. at 300 nM |
| 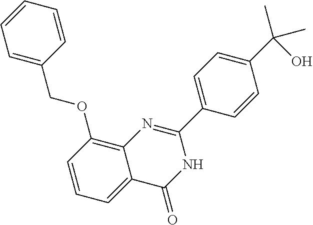 [SMILES: CC(C)(O)c1ccc(-c2nc3c(OCc4ccccc4)cccc3c(=O)[nH]2)cc1] | 144 | 46% inh. at 30 nM | 92% inh. at 300 nM |
| 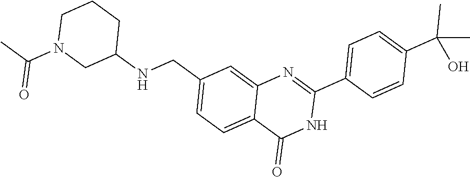 [SMILES: CC(=O)N1CCCC(NCc2ccc3c(=O)[nH]c(-c4ccc(C(C)(C)O)cc4)nc3c2)C1] | 145 | 2% inh. at 300 nM | |
| 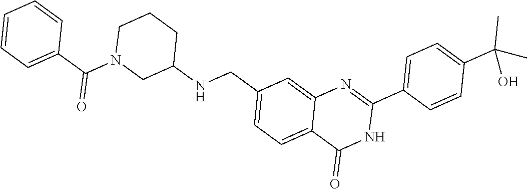 [SMILES: CC(C)(O)c1ccc(-c2nc3cc(CNC4CCCN(C(=O)c5ccccc5)C4)ccc3c(=O)[nH]2)cc1] | 146 | 4% inh. at 300 nM | |
| 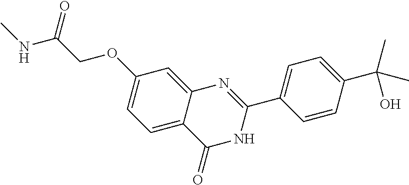 [SMILES: CNC(=O)COc1ccc2c(=O)[nH]c(-c3ccc(C(C)(C)O)cc3)nc2c1] | 147 | 35.89 | 457.98 |
| 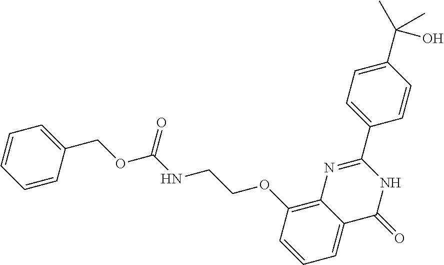 [SMILES: CC(C)(O)c1ccc(-c2nc3c(OCCNC(=O)OCc4ccccc4)cccc3c(=O)[nH]2)cc1] | 148 | 85% inh. at 300 nM | 72% inh. at 300 nM |
| 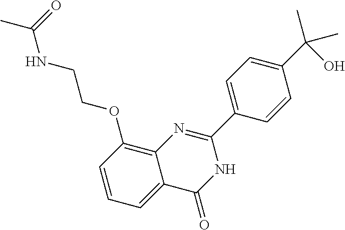 [SMILES: CC(=O)NCCOc1cccc2c(=O)[nH]c(-c3ccc(C(C)(C)O)cc3)nc12] | 149 | 69% inh. at 300 nM | 68% inh. at 300 nM |
| 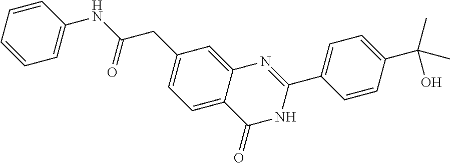 [SMILES: CC(C)(O)c1ccc(-c2nc3cc(CC(=O)Nc4ccccc4)ccc3c(=O)[nH]2)cc1] | 150 | 41% inh. at 300 nM | |
| 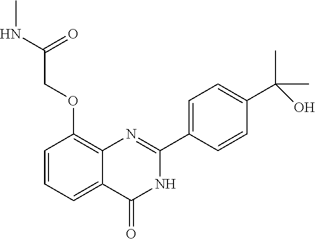 [SMILES: CNC(=O)COc1cccc2c(=O)[nH]c(-c3ccc(C(C)(C)O)cc3)nc12] | 151 | 85% inh. at 300 nM | 92% inh. at 300 nM |
| 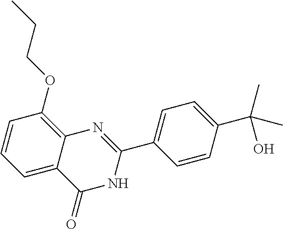 [SMILES: CCCOc1cccc2c(=O)[nH]c(-c3ccc(C(C)(C)O)cc3)nc12] | 152 | 68% inh. at 300 nM | |
| 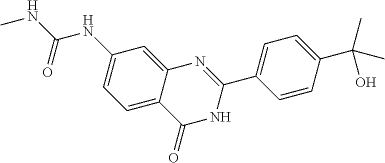 [SMILES: CNC(=O)Nc1ccc2c(=O)[nH]c(-c3ccc(C(C)(C)O)cc3)nc2c1] | 153 | 20.72 | 166.72 |
| 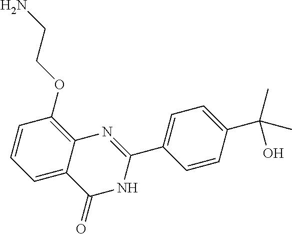 [SMILES: CC(C)(O)c1ccc(-c2nc3c(OCCN)cccc3c(=O)[nH]2)cc1] | 154 | 49% inh. at 30 nM | 91% inh. at 300 nM |
| 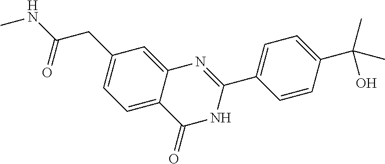 [SMILES: CNC(=O)Cc1ccc2c(=O)[nH]c(-c3ccc(C(C)(C)O)cc3)nc2c1] | 155 | 30% inh. at 300 nM | |
| 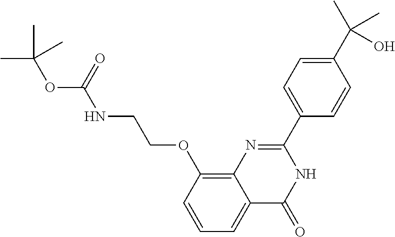 [SMILES: CC(C)(C)OC(=O)NCCOc1cccc2c(=O)[nH]c(-c3ccc(C(C)(C)O)cc3)nc12] | 156 | 79% inh. at 300 nM | 55% inh. at 300 nM |
| 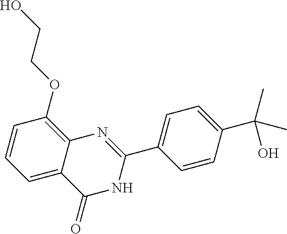 [SMILES: CC(C)(O)c1ccc(-c2nc3c(OCCO)cccc3c(=O)[nH]2)cc1] | 157 | 77% inh. at 30 nM | 51% inh. at 30 nM |
| 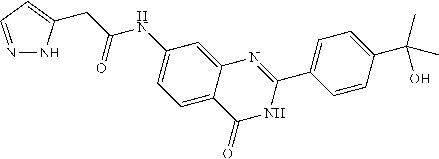 [SMILES: CC(C)(O)c1ccc(-c2nc3cc(NC(=O)Cc4ccn[nH]4)ccc3c(=O)[nH]2)cc1] | 158 | 90% inh. at 300 nM | 37% inh. at 300 nM |
| 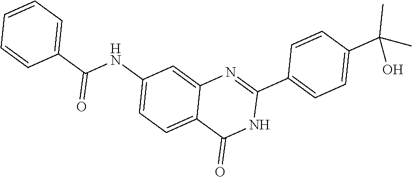 [SMILES: CC(C)(O)c1ccc(-c2nc3cc(NC(=O)c4ccccc4)ccc3c(=O)[nH]2)cc1] | 159 | 31.8 | 288.3 |
| 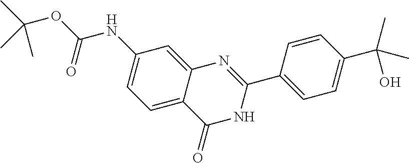 [SMILES: CC(C)(C)OC(=O)Nc1ccc2c(=O)[nH]c(-c3ccc(C(C)(C)O)cc3)nc2c1] | 160 | 17% inh. at 300 nM | |
| 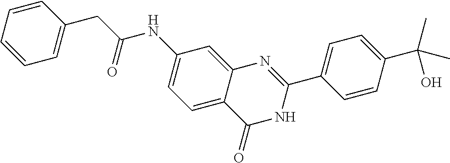 [SMILES: CC(C)(O)c1ccc(-c2nc3cc(NC(=O)Cc4ccccc4)ccc3c(=O)[nH]2)cc1] | 161 | 84% inh. at 300 nM | |
| 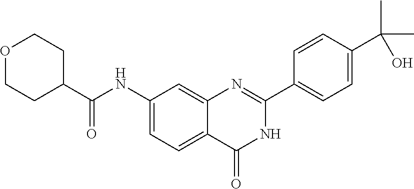 [SMILES: CC(C)(O)c1ccc(-c2nc3cc(NC(=O)C4CCOCC4)ccc3c(=O)[nH]2)cc1] | 162 | 69% inh. at 300 nM | |
| 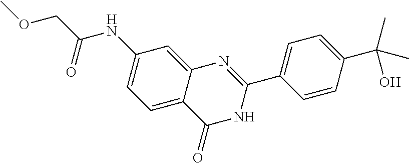 [SMILES: COCC(=O)Nc1ccc2c(=O)[nH]c(-c3ccc(C(C)(C)O)cc3)nc2c1] | 163 | 50.7 | 675.6 |
| 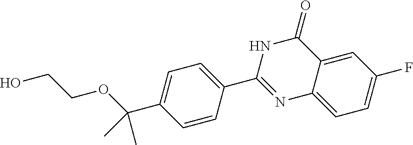 [SMILES: CC(C)(OCCO)c1ccc(-c2nc3ccc(F)cc3c(=O)[nH]2)cc1] | 164 | 96% inh. at 30 nM | |
| 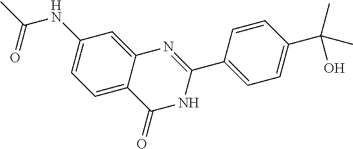 [SMILES: CC(=O)Nc1ccc2c(=O)[nH]c(-c3ccc(C(C)(C)O)cc3)nc2c1] | 165 | 24 | 82 |
| 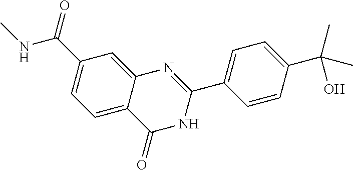 [SMILES: CNC(=O)c1ccc2c(=O)[nH]c(-c3ccc(C(C)(C)O)cc3)nc2c1] | 166 | >1000 | >1000 |
| 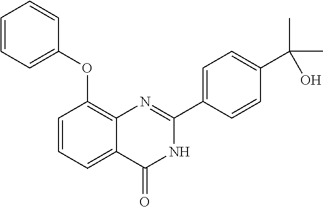 [SMILES: CC(C)(O)c1ccc(-c2nc3c(Oc4ccccc4)cccc3c(=O)[nH]2)cc1] | 167 | >1000 | >1000 |
| 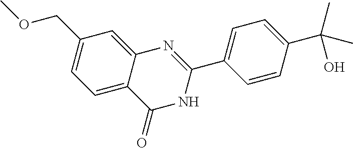 [SMILES: COCc1ccc2c(=O)[nH]c(-c3ccc(C(C)(C)O)cc3)nc2c1] | 168 | 290 | 430 |
| 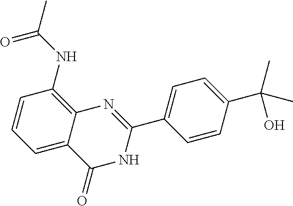 [SMILES: CC(=O)Nc1cccc2c(=O)[nH]c(-c3ccc(C(C)(C)O)cc3)nc12] | 169 | 160 | 57 |
| 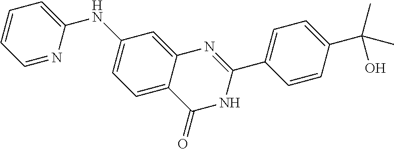 [SMILES: CC(C)(O)c1ccc(-c2nc3cc(Nc4ccccn4)ccc3c(=O)[nH]2)cc1] | 170 | 310 | 280 |
| 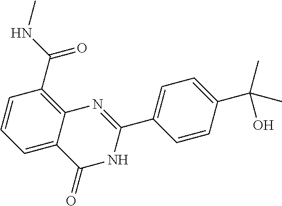 [SMILES: CNC(=O)c1cccc2c(=O)[nH]c(-c3ccc(C(C)(C)O)cc3)nc12] | 171 | 78 | 54 |
| 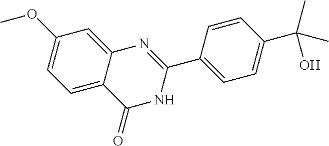 [SMILES: COc1ccc2c(=O)[nH]c(-c3ccc(C(C)(C)O)cc3)nc2c1] | 172 | 27 | 15 |
| 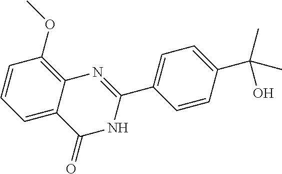 [SMILES: COc1cccc2c(=O)[nH]c(-c3ccc(C(C)(C)O)cc3)nc12] | 173 | 4.6 | 9.3 |
| 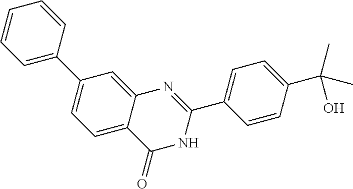 [SMILES: CC(C)(O)c1ccc(-c2nc3cc(-c4ccccc4)ccc3c(=O)[nH]2)cc1] | 174 | >1000 | >1000 |

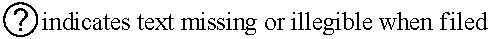

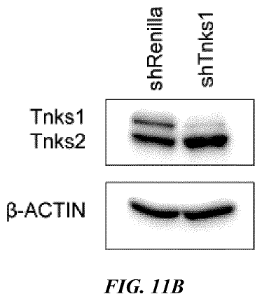

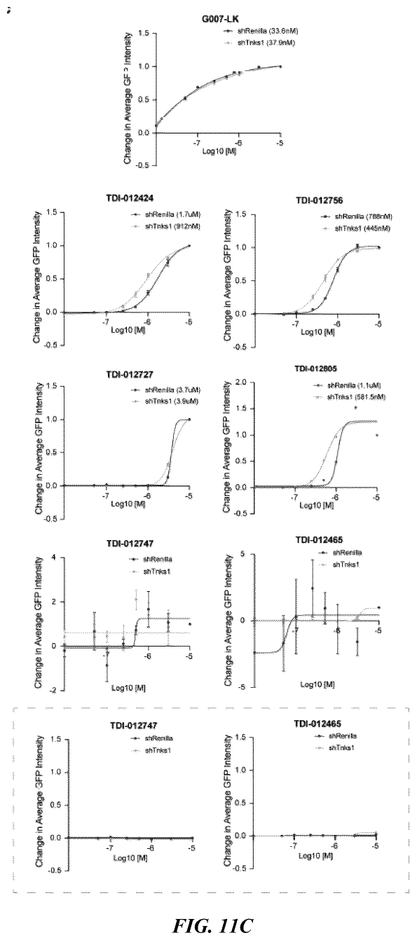

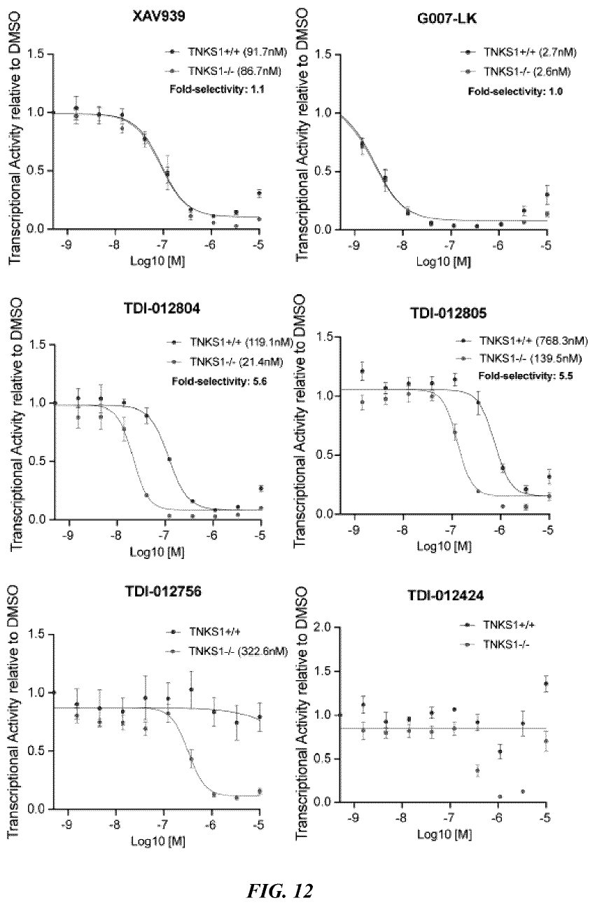

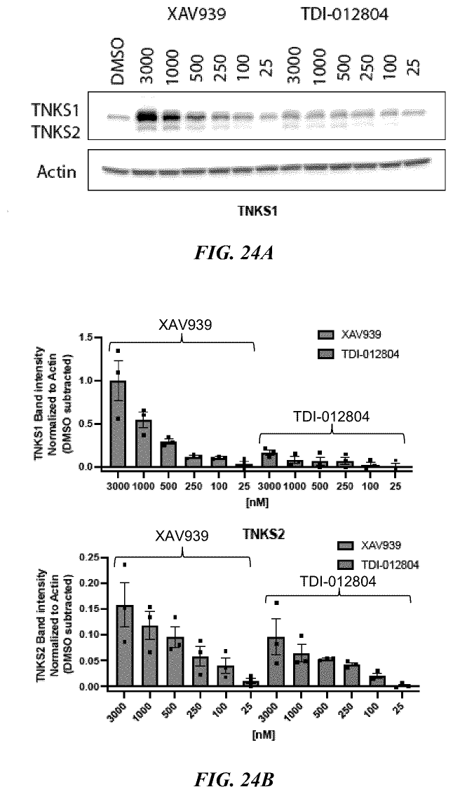

To determine whether the seven identified compounds showed selective inhibition of endogenous TNKS2 protein in cells, the stability of a GFP-AXIN1 reporter was quantified in both wildtype (TNKS1 WT) and TNKS1 knockdown (TNKS1^{KD}), NIH3T3 cells (FIGS. 7E-7F, FIGS. 11A-11B). This reporter allows direct and simple quantification of AXIN1 protein stabilization while avoiding confounding changes in AXIN1 transcription. In this assay, TDI-012804 was most potent and showed the clearest selectivity profile (FIGS. 7G-7H, FIG. 11C). To further evaluate TDI-012804 activity in cells, the impact on downstream WNT-mediated transcription was quantified in TNKS1 isogenic DLD1 cells using an integrated TOP-Flash reporter. Consistent with the GFP-AXIN1 data, TDI-012804 was potent (EC₅₀<100 nM) and showed TNKS2-selectivity in these cells as compared to the nonselective XAV939 (FIG. 7I, FIG. 12). As observed in TNKS2 shRNA experiments (FIG. 3F), TNKS1^{HET} cells showed increased response to TDI-012804 compared to TNKS1^{WT} cells (FIG. 7I), further supporting the notion that a single allele of TNKS1 is not sufficient to support cellular Tankyrase function. FIGS. 24A-24B show that TDI-012804 preferentially stabilizes TNKS2 over TNKS1. FIG. 7J shows that the downstream biochemical effect (AXIN1 stabilization) is only apparent in TNKS1 KO and HET cells.

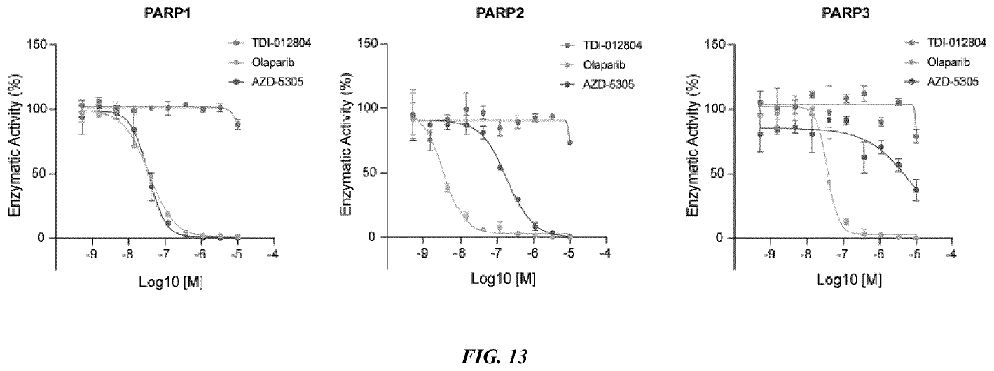

Consistent with the GFP-AXIN1 reporter, TDI-012804 induced marked stabilization of endogenous AXIN1 in both TNKS1^{HET} and TNKS1^{KO} cells but induced only a minimal increase in AXIN1 protein in TNKS1 diploid controls (FIG. 7J). Importantly, TDI-012804 (FIG. 7K) showed no inhibition of related PARP-family enzymes (PARP1, PARP2, and PARP3), highlighting it as a potent and selective TNKS2 inhibitor (FIG. 13).

### Example 4—Structural Basis for TNKS2 Selective Inhibition

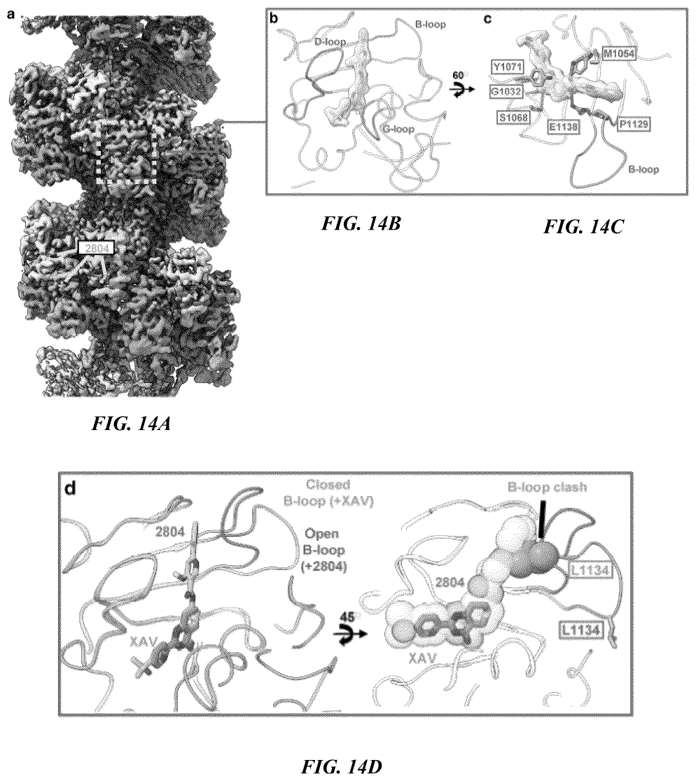

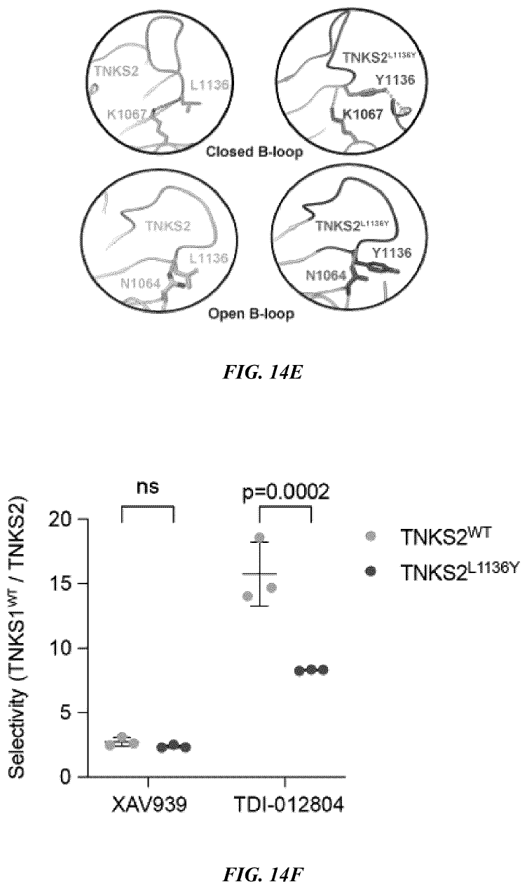

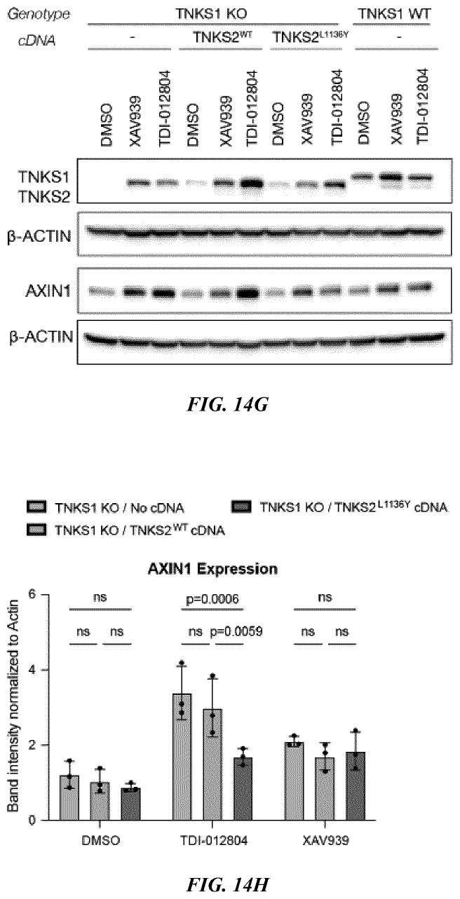

To define the mechanism underlying TNKS2 inhibition by TDI-012804, structural characterization of the ligand bound state to the TNKS2 catalytic domain was sought. As previously described (Pillay et al., “Structural Basis of Tankyrase Activation by Polymerization,” Nature 612:162-169 (2022); Mariotti et al., “Tankyrase Requires SAM Domain-Dependent Polymerization to Support Wnt-β-Catenin Signaling,” Mol. Cell 63:498-513 (2016), which are hereby incorporated by reference in their entirety), the minimal active ADP-ribosyl transferase unit of TNKS2, the SAM-PARP domains, adopts a helical filamentous architecture, aiding the determination of a high-resolution structure (FIG. 14A). Using a combination of helical reconstruction and focused refinements, it was determined that a ˜2.2 Å nominal resolution cryo-electron microscopy (cryo-EM) map of TDI-012804 bound to TNKS2 (FIGS. 15A-E, 16A-C, Table 2). This structure illustrated that the ligand occupied both the nicotinamide donor site and the acceptor site (FIG. 14B), consistent with the computational design. TDI-012804 has 2-phenyl-3,4-dihydroquinazoline-4-one core, which is anchored in the nicotinamide subsites by similar interactions as previously reported for this scaffold. The lactam portion of the quinazolinone ring forms hydrogen bond networks with Ser1068 side chain and the Gly 1032 backbone amide. The quinazolinone core forms a T-x stacking interaction with Tyr1071, while the phenyl group at 2-position fits into a hydrophobic pocket. In the acceptor subsite, the amide NH forms a hydrogen bond with Glu1138, while the pyridine nitrogen interacts with Met1054 through water-mediated interaction. (FIG. 14C).

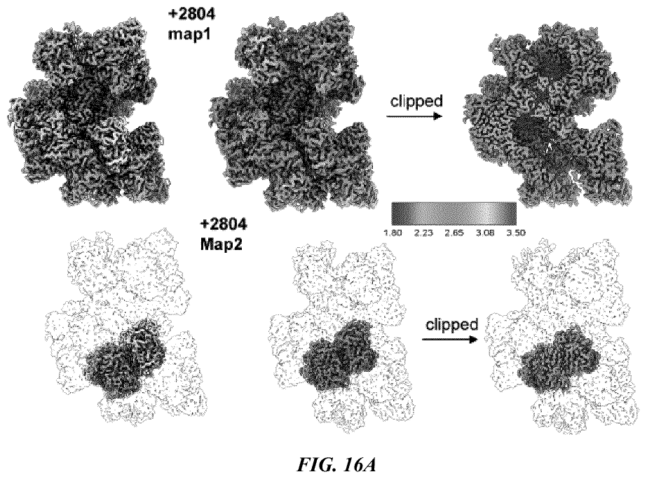

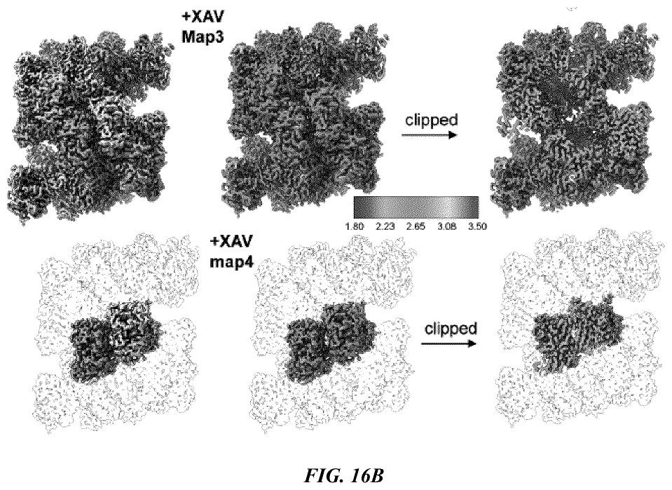

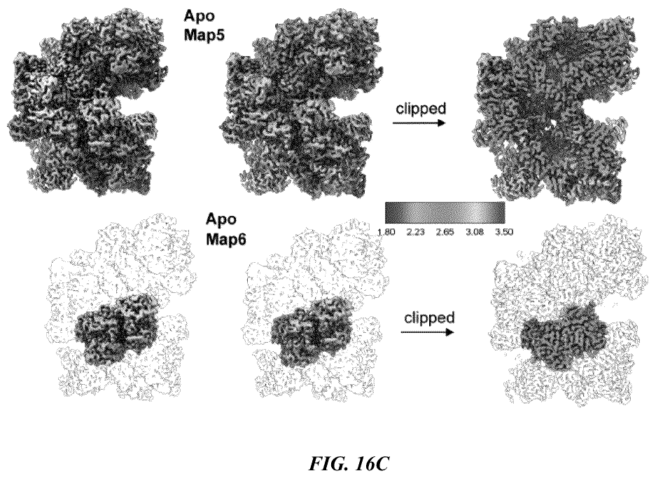

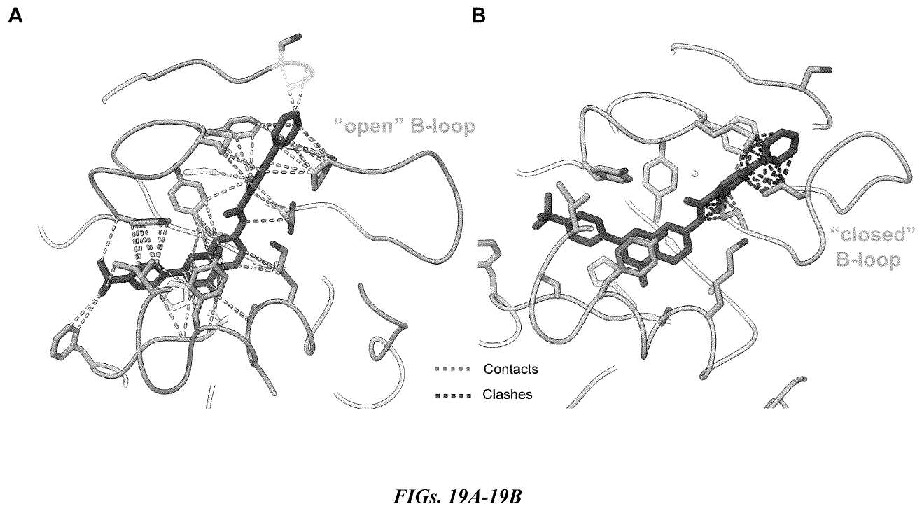

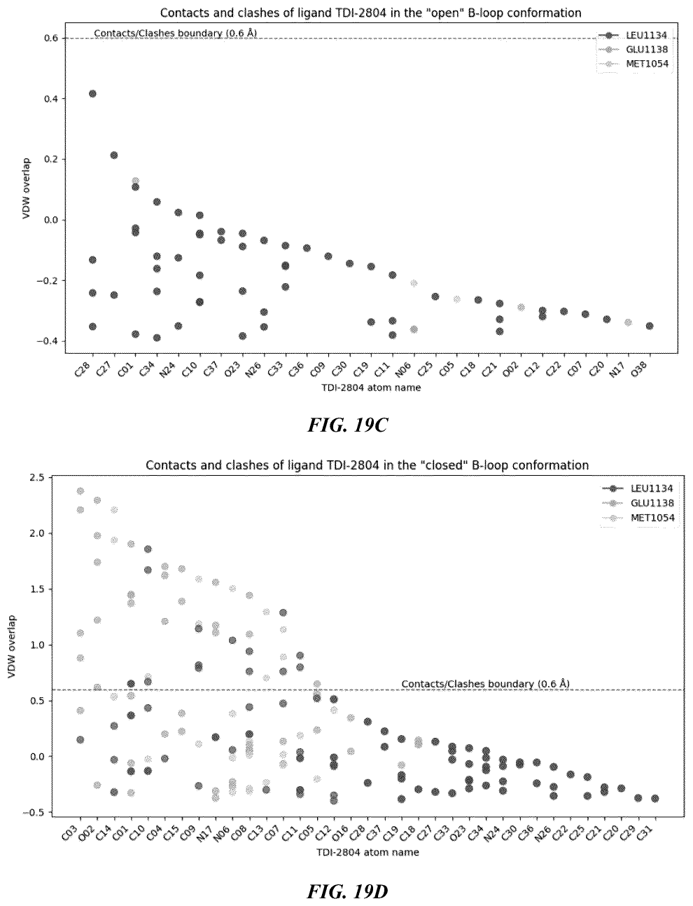

To gain insight into potential mechanisms of TNKS2 selectivity, cryo-EM structures of TNKS2 in the absence of ligand (apo) and bound to the non-selective inhibitor XAV939 were additionally resolved (FIGS. 16A-16C, 17A-17F, 18A-18E). Analysis of these structures revealed that the terminal phenyl ring of TDI-012804 forces the B-loop in the acceptor subsite to adopt an “open” conformation in contrast to the “closed” conformation observed in the presence of XAV939 or without ligand (FIG. 14D). While the TNKS2 B-loop is notably dynamic in all conditions, the closed loop conformation of Leucine 1134 resolved in the presence of XAV939 or in the absence of ligand would clash with the phenyl ring in TDI-012804, reducing ligand association (FIGS. 19A-19D). However, rearrangement of the B-loop into the open conformation when bound to TDI-012804 re-positions L1134 away from the active site and allows the phenyl ring to form a pi-pi stacking interaction with P1129. An analysis of all known crystal structures of both TNKS1 and TNKS2 indicates that the TNKS1 B-loop is modeled in the closed conformation near exclusively whereas the TNKS2 B-loop has been modeled in a continuum of conformations between the open and closed conformations.

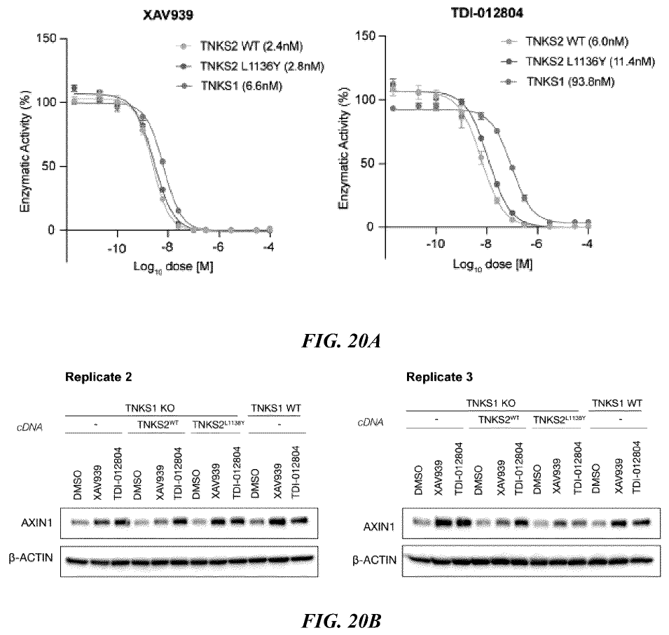

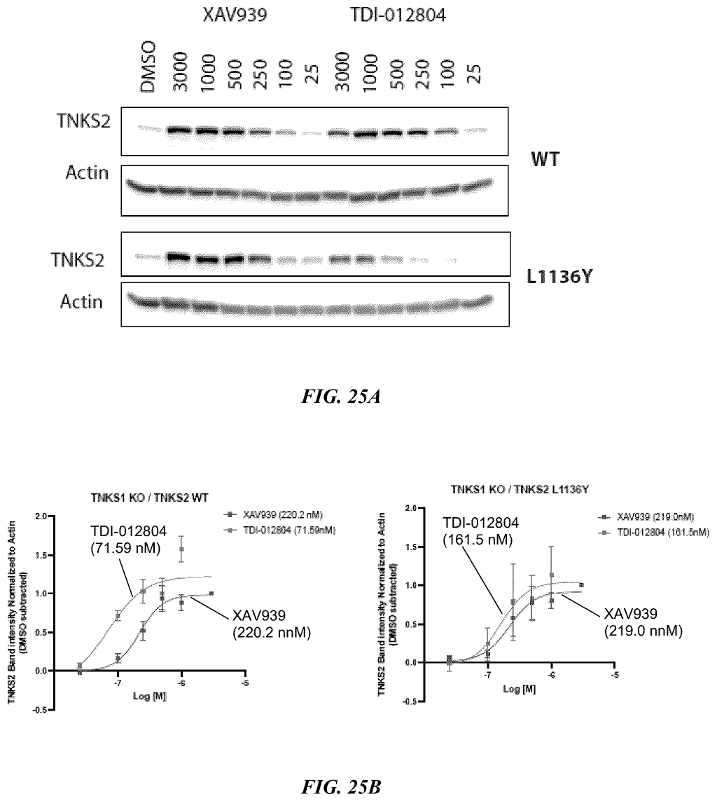

Together, these data indicate that the B-loop is more dynamic in TNKS2 than in TNKS1. A single amino acid (L1136 of TNKS2; Y1289 in TNKS1) distinguishes the B-loops of TNKS1 and TNKS2. It was reasoned that the enhanced intramolecular hydrogen bonding network provided by a tyrosine in this position may increase the rigidity of the B-loop of TNKS1 and thus impede binding of TDI-012804 (FIG. 14E). To directly test this, TNKS2^{WT}, TNKS2^{L1136Y} and TNKS1 SAM-PARP domains were purified and the IC₅₀ for TDI-012804 and XAV939 was measured by in vitro PARylation assay. Consistent with the model, mutation of L1136 to tyrosine (L1136Y) significantly reduced the potency of TDI-012804 against TNKS2 but had no effect on the activity of XAV939 (FIG. 14F, FIG. 20A). These data suggest that L1136 in TNKS2 contributes to the enhanced flexibility of the TNKS2 B-loop, thereby increasing TDI-012804 affinity. To investigate the effect of the L1136Y substitution on the selectivity of TDI-012804 in cells, TNKS1^{KO} DLD1 cell lines that exogenously expressed TNKS2^{WT} or TNKS2^{L1136Y} were generated and AXIN1 stabilization was measured 24 hours following treatment. Consistent with the biochemical assay, TDI-012804 increased AXIN1 protein in TNKS1^{KO} and TNKS1^{KO}/TNKS2^{WT} cells, but not in TNKS1^{WT} or TNKS1^{KO}/TNKS2^{L1136Y} cells (FIGS. 14G-14H, FIG. 20B). Further, L1136Y mutant TNKS2 protein showed reduced stabilization following treatment with TDI-012804, consistent with the notion that mutation of L1136Y reduces the engagement of TDI-012804 (FIGS. 25A-25B). Consistent with the PARylation assay, the L1136Y substitution did not alter the response to the pan-TNKS inhibitor XAV939 (FIGS. 14G-14H, FIG. 20B).

### Example 5—TNKS2-Selective Inhibitors Suppress WNT Signaling in TNKS-Depleted Cells

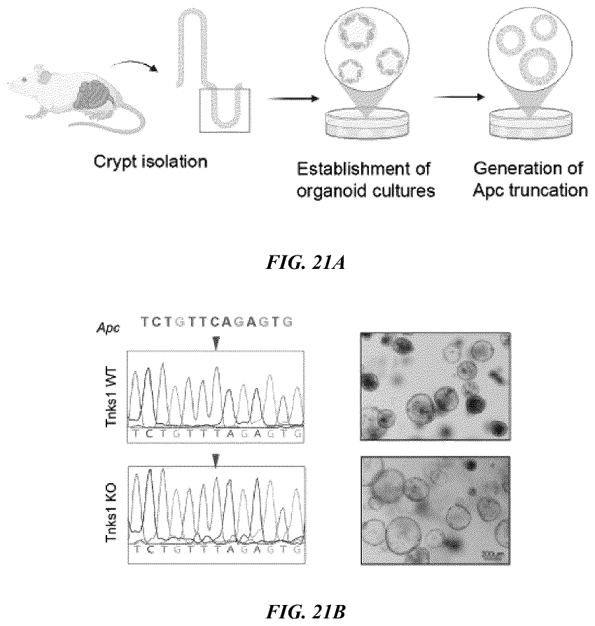

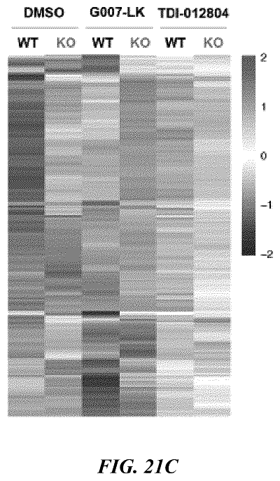

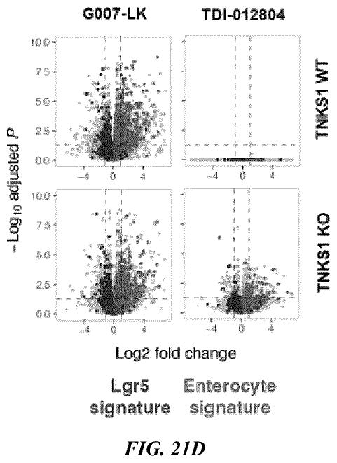

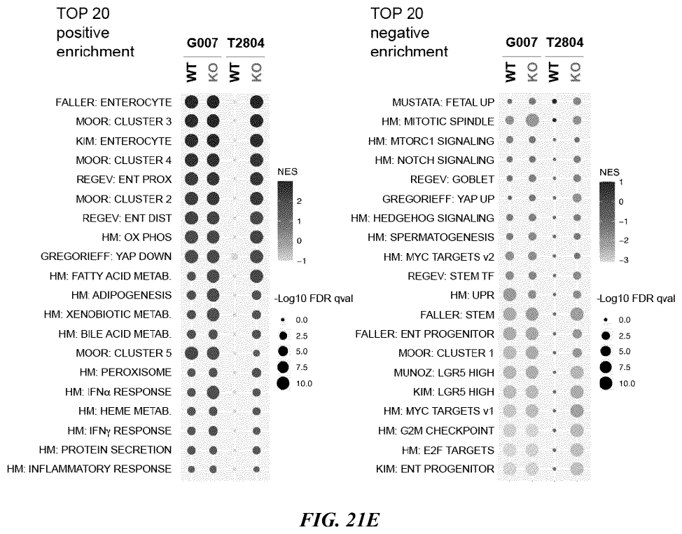

Tankyrase inhibition in DLD1 cells induces WNT pathway suppression but does not impact cell proliferation or viability (Lau et al., “A Novel Tankyrase Small-Molecule Inhibitor Suppresses APC Mutation-Driven Colorectal Tumor Growth,” Cancer Res. 73:3132-3144 (2013); Foronda et al., “Tankyrase Inhibition Sensitizes Cells to CDK4 Blockade,” PLOS One 14: e0226645 (2019), which are hereby incorporated by reference in their entirety). To test the effect of TDI-012804 in a WNT-dependent system that closely mimics normal intestinal biology, Tnks1^{WT}, Tnks1^{HET} and Tnks1^{KO} intestinal organoid cultures were generated harboring a homozygous nonsense mutation (Q1405X) in the APC tumor suppressor (FIGS. 21A-21B). This mutation models common truncating events within the mutation cluster region of APC observed in both familial and sporadic CRC. Consistent with previously published strong WNT and TNKS dependence of APC mutant organoids (Schatoff et al., “Distinct Colorectal Cancer-Associated APC Mutations Dictate Response to Tankyrase Inhibition,” Cancer Discov. 9:1358-1371 (2019), which is hereby incorporated by reference in its entirety), RNA-sequencing of G007-LK treated Apc^{Q1405X}/Tnks1^{WT} and Apc^{Q1405X}/Tnks1^{KO} small intestinal organoids revealed broad transcriptional changes including reduced stem and progenitor cell signatures and upregulation of intestinal differentiation markers across multiple cell lineages (FIGS. 21C-21E). Apc^{Q1405X} Tnks1^{KO} organoids treated with TDI-012804 showed a near-identical overall transcriptional response to G007-LK treated cultures, while Apc^{Q1405X}/Tnks1^{WT} organoids showed no significant gene expression changes compared to DMSO controls (FIGS. 21C-21E). Notably, there were only two significantly deregulated genes in the RNAseq analysis comparing G007-LK and TDI-012804 in Tnks1^{KO} organoids, implying that TDI-012804 does not have measurable non-Tankyrase (off-target) activity at effective doses.

### Example 6—TNKS2-Selective Inhibitors Block Proliferation in TNKS-Depleted Cancer Cells and Organoids

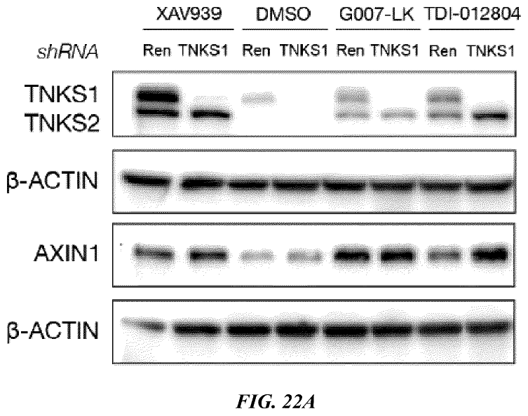

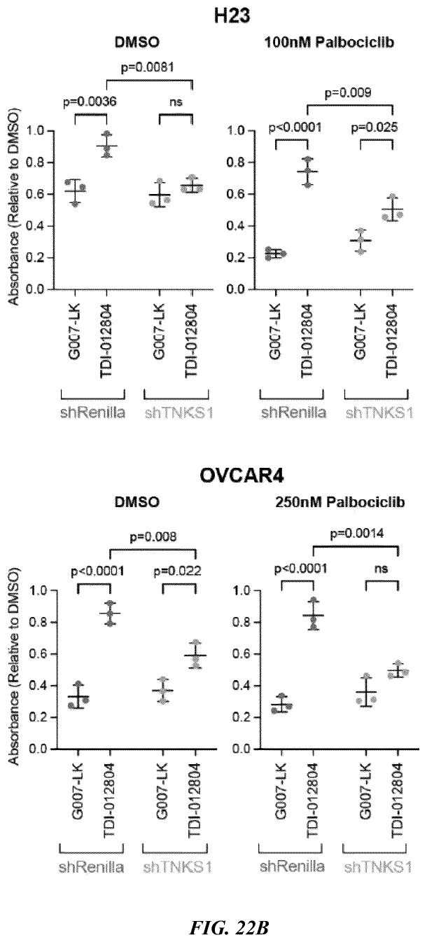

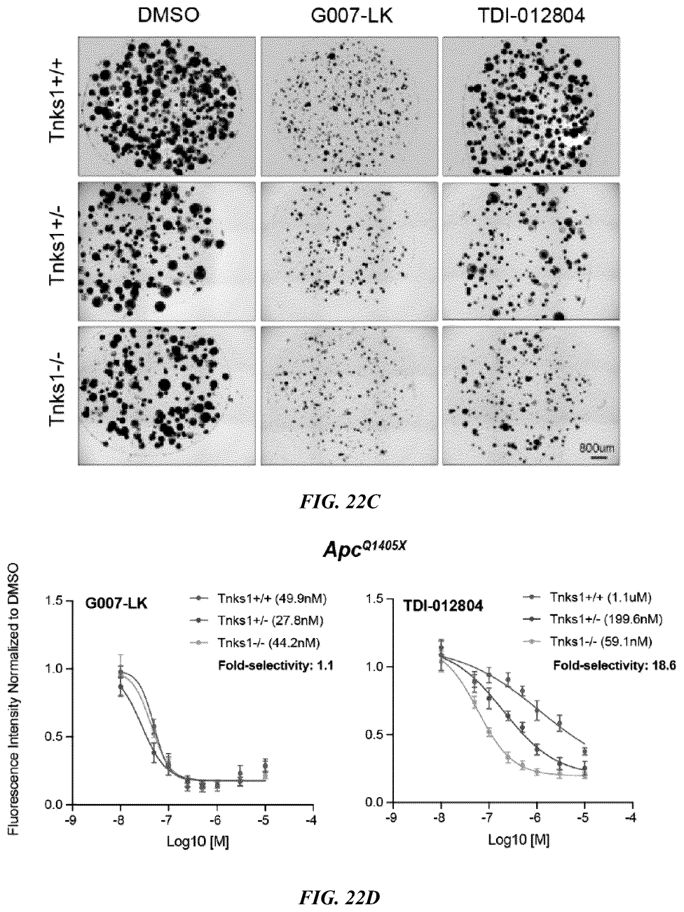

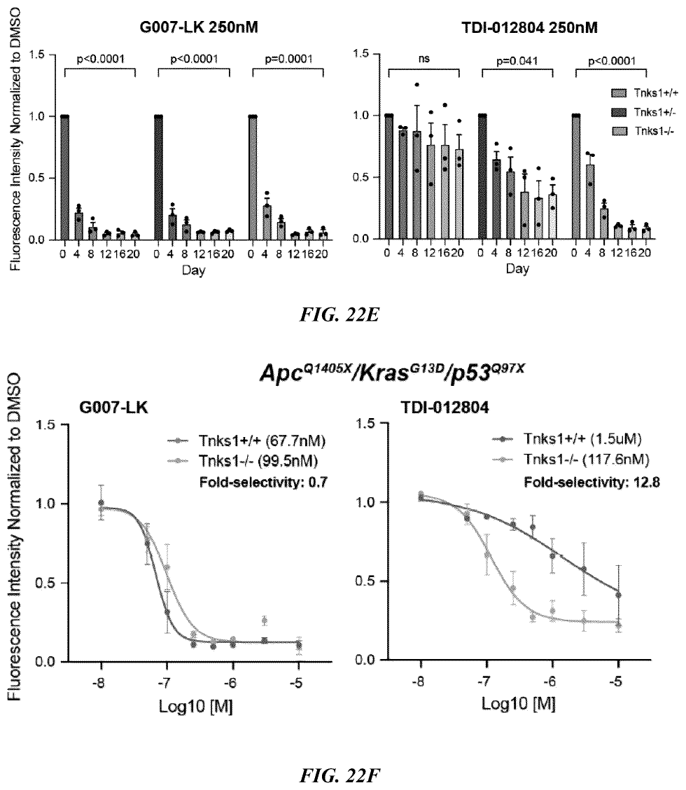

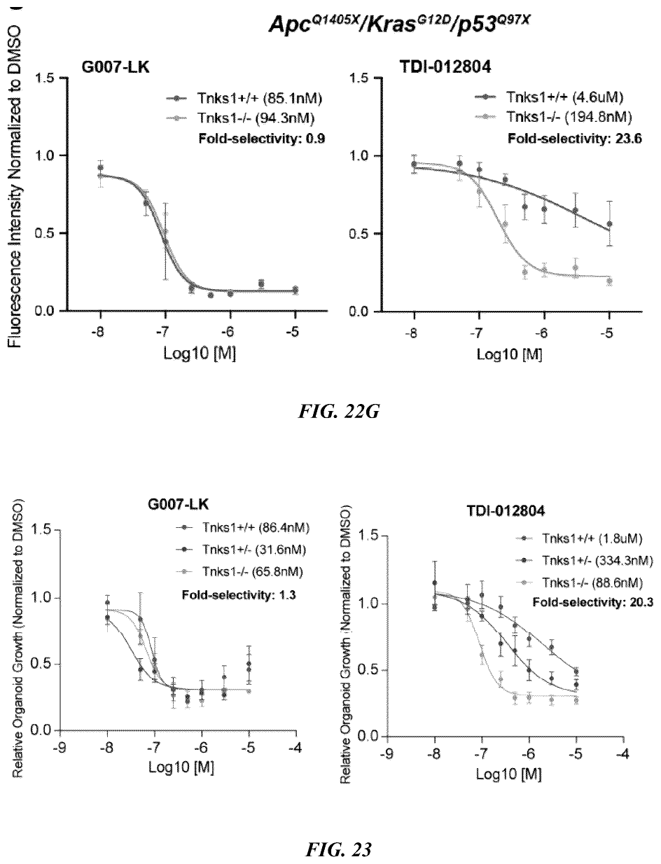

To further explore the effects of TNKS2 inhibition on cell survival and proliferation, several human cancer cell lines that are responsive to TNKS inhibition either alone or in combination with other targeted therapies were identified (Lau et al., “A Novel Tankyrase Small-Molecule Inhibitor Suppresses APC Mutation-Driven Colorectal Tumor Growth,” Cancer Res. 73:3132-3144 (2013); Foronda et al., “Tankyrase Inhibition Sensitizes Cells to CDK4 Blockade,” PLOS One 14: e0226645 (2019); Mygland et al., “Identification of Response Signatures for Tankyrase Inhibitor Treatment in Tumor Cell Lines,” iScience 24:102807 (2021), which are hereby incorporated by reference in their entirety). These included one 8p heterozygous cell line, SW403 (CRC), and two 8p diploid lines, OVCAR4 (Ovarian) and H23 (lung). Consistent with the response seen in TNKS1 heterozygous DLD1 cells, TDI-012804 treatment of SW403 cells induced AXIN1 stabilization to similar levels as the pan-TNKS inhibitor G007-LK (FIG. 22A). Consequently, treatment with either TDI-012804 or G007-LK resulted in a similar reduction in colony formation (FIG. 22B). As expected, treatment of 8p diploid H23 cells with TDI-012804 did not induce AXIN1 stabilization nor suppress colony formation like G007-LK (FIG. 23); however, suppression of TNKS1 in these cells induced a significant sensitization to TDI-012804 that was further enhanced in the presence of Palbociclib (FIG. 22B, FIG. 23). OVCAR4 cells were sensitive to pan-TNKS inhibition by G007-LK with or without Palbociclib (FIG. 22B, FIGS. 23A-23B), and consistent with the response seen in H23 cells, TDI-012804 significantly reduced OVCAR4 colony formation only in TNKS1-depleted cells (FIG. 22B).

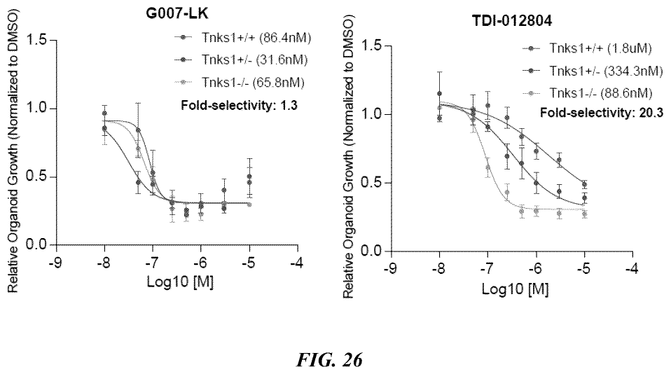

Cell proliferation in Apc^{Q1405T} mutant organoids were next measured over 6 days of treatment. As expected, G007-LK showed robust suppression of proliferation across all Tnks1 genotypes, as measured by a marked reduction in total organoid area (FIG. 22C, FIG. 26) and viability/metabolic activity (Alamar blue; FIG. 22D). Likewise, TDI-012804 strongly suppressed Apc^{Q1405X}/Tnks1^{KO} organoid proliferation (EC₅₀ 59.1 nM), while Tnks1 diploid organoids were significantly less sensitive, showing a 19-fold increase in EC₅₀ (1.1 uM) (FIG. 22D, FIG. 26). Again, Tnks1^{HET} organoids were sensitive to TNKS2 inhibition, but showed a 3-fold higher EC50 (199.6 nM) compared to Tnks1^{KO} organoids (FIG. 22D, FIG. 26). To confirm that prolonged treatment with TDI-012804 does not have a cumulative detrimental effect in Tnks1^{WT} cells, organoids were treated for 20 days (refreshing compound every 2 days), measuring metabolic viability and passaging every 4 days. Consistent with the short-term analysis, G007-LK showed robust suppression of organoid growth, while TDI-021804 induced a significant reduction in proliferation in Tnks1^{KO} and Tnks1^{HET} cells, but not Tnks1^{WT} organoids (FIG. 22E). Finally, to determine whether TDI-012804 could suppress proliferation in the context of common CRC oncogenic mutations, Apc^{Q1405X}/Kras^{G13D}/p53^{Q97X} (AKP-G13D) and Apc^{Q1405X} Kras^{G12D}/p53^{Q97X} (AKP-G12D) mutant intestinal organoids were generated with or without Tnks1 loss. Mirroring data in Apc^{Q1405X} mutant cultures, TDI-012804 was selectively toxic to Tnks1^{KO} AKP-G12D and AKP-G13D organoids, compared to Thks1^{WT} lines (FIGS. 22F-22G).

### Example 7—Discussion of Examples 1-6

WNT hyperactivation is a potent driver in many types of cancer and there is substantial interest in targeting the WNT pathway as a therapeutic strategy. However, multiple studies have identified significant on-target toxicity in the gut and bone from systemic WNT inhibition, highlighting the need for more tumor-selective therapies. Here, is shown that loss of TNKS1 expression through cancer-associated chromosome 8p deletions creates a functional dependency on TNKS2 activity, which sensitizes cancer cells to TNKS2-selective inhibition. Further, through rational design, a first-in-class TNKS2-selective small molecule inhibitor was developed and this compound was shown to selectively target TNKS1-depleted human cancer cells and murine organoids with APC, KRAS and p53 mutations.

Collateral vulnerability or ‘collateral lethality’ is the emergence of cancer-specific dependency due to loss of large chromosomal regions that delete a redundant ortholog of a key pathway regulator (Muller et al., “Passenger Deletions Generate Therapeutic Vulnerabilities in Cancer,” Nature 488:337-342 (2012); Muller et al., “Collateral Lethality: A New Therapeutic Strategy in Oncology,” Trends Cancer 1:161-173 (2015), which are hereby incorporated by reference in their entirety). The first example of this concept was described by DePinho and colleagues, who showed that the homozygous loss of the distal region of chromosome 1p (1p36) containing Enolase1 (ENO1), makes glioblastomas selectively dependent on ENO2 (Muller et al., “Passenger Deletions Generate Therapeutic Vulnerabilities in Cancer,” Nature 488:337-342 (2012), which is hereby incorporated by reference in its entirety). Recent development of an ENO2-selective inhibitor supports the therapeutic potential of these strategies for developing cancer-selective treatment approaches (Muller et al., “Passenger Deletions Generate Therapeutic Vulnerabilities in Cancer,” Nature 488:337-342 (2012); Lin et al., “An Enolase Inhibitor for the Targeted Treatment of ENO1-Deleted Cancers,” Nat, Metab, 2:1413-1426 (2020), which are hereby incorporated by reference in their entirety). Importantly, though the concept of collateral vulnerability was proposed based on homozygous deletion of a redundant ortholog, heterozygous ENO1 cells do show increased sensitivity to ENO2 inhibition (Muller et al., “Passenger Deletions Generate Therapeutic Vulnerabilities in Cancer,” Nature 488:337-342 (2012); Lin et al., “An Enolase Inhibitor for the Targeted Treatment of ENO1-Deleted Cancers,” Nat. Metab. 2:1413-1426 (2020), which are hereby incorporated by reference in their entirety). Similarly, while TNKS2 blockade is most effective in cells carrying homozygous TNKS1 deletions, heterozygous TNKS1 loss also sensitizes cells to TNKS2-selective inhibition. This is consistent with data from earlier mouse genetic studies showing that while TNKS2 knockout mice are viable and fertile, loss of a single TNKS1 allele in this background is embryonic lethal (Chiang et al., “Tankyrase 1 and Tankyrase 2 are Essential but Redundant for Mouse Embryonic Development,” PLOS One 3: e2639 (2008), which is hereby incorporated by reference in its entirety). Together with data described here, these observations suggest that TNKS1 is haploinsufficient for maintaining Tankyrase activity in the context of TNKS2 disruption and expands the potential scope of TNKS2 inhibition to cancers with 8p homozygous and heterozygous loss.

More than 50 Tankyrase inhibitors have been reported in the literature (Lau et al., “A Novel Tankyrase Small-Molecule Inhibitor Suppresses APC Mutation-Driven Colorectal Tumor Growth,” Cancer Res 73:3132-3144 (2013; Zhong et al., “Tankyrase Inhibition Causes Reversible Intestinal Toxicity in Mice with a Therapeutic Index<1,” Toxicol. Pathol. 44:267-278 (2016); Huang et al., “Tankyrase Inhibition Stabilizes Axin and Antagonizes Wnt Signalling,” Nature 461:614-620 (2009); Chen et al., “Small Molecule-Mediated Disruption of Wnt-Dependent Signaling in Tissue Regeneration and Cancer,” Nat. Chem. Biol. 5:100-107 (2009); Waaler et al., “A Novel Tankyrase Inhibitor Decreases Canonical Wnt Signaling in Colon Carcinoma Cells and Reduces Tumor Growth in Conditional Apc Mutant Mice,” Cancer Res. 72:2822-2832 (2012); Shultz et al., “Identification of NVP-TNKS656: the Use of Structure-Efficiency Relationships to Generate a Highly Potent, Selective, and Orally Active Tankyrase Inhibitor,” J. Med. Chem. 56:6495-6511 (2013); C. C. Mehta and H. G. Bhatt, “Tankyrase Inhibitors as Antitumor Agents: A Patent Update (2013-2020),” Expert Opin. Ther. Pat. 31:645-661 (2021), Yu et al., “Small-Molecule Inhibitors of Tankyrases as Prospective Therapeutics for Cancer,” J. Med. Chem. 65:5244-5273 (2022), which are hereby incorporated by reference in their entirety), and in almost all cases, they inhibit both TNKS1 and TNKS2. This is in part due to the high degree of structural similarity in the enzymatic domain of TNKS1 and TNKS2 (FIG. 7A). Through computational modeling, the B-loop motif adjacent to the ‘acceptor’ site in TNKS2 was predicted to be conformationally flexible, assuming structurally ordered ‘open’ and ‘closed’ loop conformations. Specifically, it was predicted that presence of a Leucine residue in the TNKS2 B-loop enhances the dynamics of this loop relative to a tyrosine at this position in TNKS1 which possesses stronger intramolecular interactions in the ‘closed’ B-loop conformation. By extending a non-selective inhibitor scaffold through the active site to target the open B-loop, TDI-012804 that selectively inhibits TNKS2 was developed. Using cryoEM, it was shows that binding of TDI-012804 forces movement of this loop to an open conformation. This conformational change is unfavorable in the more rigid TNKS1 B-loop, allowing for selective TNKS2 binding. A similar type of allosteric selectivity has previously been used successfully in the development of mutant KRAS inhibitors, where targeting the S-IIP allosteric pocket in KRAS^{G12C} was able to disrupt functional binding interactions (Ostrem et al., “K-Ras (G12C) Inhibitors Allosterically Control GTP Affinity and Effector interactions” Nature 503:548-551 (2013), which is hereby incorporated by reference in its entirety). This method of allosteric binding could open many more opportunities to target disease-related proteins that are difficult to target or were previously thought undruggable.

Previous studies have shown that WNT suppression can induce tumor regression, indicating a significant therapeutic potential. However, WNT suppression is limited by dose-limiting toxicities due to the key role of WNT signaling in maintaining healthy tissue (Lau et al., “A Novel Tankyrase Small-Molecule Inhibitor Suppresses APC Mutation-Driven Colorectal Tumor Growth,” Cancer Res 73:3132-3144 (2013); Zhong et al., “Tankyrase Inhibition Causes Reversible Intestinal Toxicity in Mice with a Therapeutic Index <1,” Toxicol. Pathol. 44:267-278 (2016), which are hereby incorporated by reference in their entirety). While WNT targeted therapies have made little clinical progress, identifying safe and effective strategies to inhibit WNT would have a significant clinical impact. In addition to the direct targeting of WNT-hyperactive cancers, it has been shown that modulating WNT signaling can improve immune-based therapies (Waaler et al., “Tankyrase Inhibition Sensitizes Melanoma to PD-1 Immune Checkpoint Blockade in Syngeneic Mouse Models,” Commun. Biol. 3:196 (2020); DeVito et al., “Pharmacological Wnt Ligand Inhibition Overcomes Key Tumor-Mediated Resistance Pathways to Anti-PD-1 Immunotherapy” Cell Rep. 35:109071 (2021); Huang et al., “Wnt Inhibition Sensitizes PD-L1 Blockade Therapy by Overcoming Bone Marrow-Derived Myofibroblasts-Mediated Immune Resistance in Tumors” Front. Immunol. 12:619209 (2021), which are hereby incorporated by reference in their entirety). The development of safe WNT therapies would also allow for WNT targeting to be combined with existing treatments, enabling the development of more precise and effective combination therapies. The concept and compounds described here represent a new step towards developing targeted WNT therapies.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A compound of Formula (I): 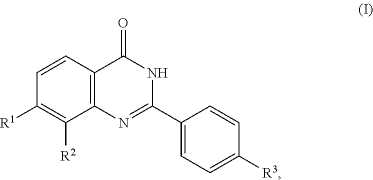
wherein
R¹ is selected from the group consisting of H, C₁₋₆ alkyl, C₁₋₆ alkoxy, Oaryl, aryl, heterocyclyl, —NR⁴R⁵, —NHC(O)NHR⁵, —CONR⁶R⁷, and —NR⁸CO(CH₂)ₘR⁹, wherein the C₁₋₆ alkyl, C₁₋₆ alkoxy, —Oaryl, aryl, and heterocyclyl can be optionally substituted from 1 to 3 times with R′;
R² is selected from the group consisting of H, C₁₋₆ alkyl, C₁₋₆ alkoxy, —Oaryl, aryl, heterocyclyl, —NR⁴R⁵, —NHC(O)NHR⁵, —CONR⁶R⁷, and —NR⁸CO(CH₂)ₘR⁹, wherein the C₁₋₆ alkyl, C₁₋₆ alkoxy, —Oaryl, aryl, and heterocyclyl can be optionally substituted from 1 to 3 times with R′;
R′ is independently selected at each occurrence thereof from the group consisting of —OH, ═O, —NH₂, halogen, —NHCO₂R¹⁰, —CONR¹¹R¹², —NHCOR¹³, —NHR¹⁴, C₁₋₆ alkoxy, C₁₋₆ alkyl, aryl, biaryl, heteroaryl, —Oaryl, —Oheteroaryl, and heterocyclyl, wherein heteroaryl and heterocyclyl can be optionally substituted from 1 to 3 times with ═O;
R³ is C₁₋₆ alkyl optionally substituted from 1 to 3 times with a substituent independently selected at each occurrence thereof from the group consisting of OH, CF₃, and —O—(CH₂)ₘOH;
R⁴ is selected from the group consisting of H and C₁₋₆ alkyl;
R⁵ is selected from the group consisting of H, C₁₋₆ alkyl, aryl, benzyl, and heteroaryl, wherein C₁₋₆ alkyl can be optionally substituted from 1 to 3 times with a substituent selected independently at each occurrence thereof from the group consisting of —OH and aryl;
R⁶ is H or C₁₋₆ alkyl;
R⁷ is H or C₁₋₆ alkyl;
R⁸ is H or C₁₋₆ alkyl;
R⁹ is selected from the group consisting of C₁₋₆ alkyl, C₁₋₆ alkoxy, —Oaryl, —Oheteroaryl, C₃₋₁₂ cycloalkyl, aryl, biaryl, heterocyclyl, arylalkyl, heteroaryl, and —NHC₁₋₆ alkyl, wherein C₁₋₆ alkyl, —Oaryl, C₃₋₁₂ cycloalkyl, heterocyclyl, aryl, biaryl, arylalkyl, and heteroaryl can be optionally substituted from 1 to 3 times with R¹⁵;
R¹⁰ is selected from the group consisting of H, C₁₋₆ alkyl, and benzyl;
R¹¹ is H or C₁₋₆ alkyl;
R¹² is selected from the group consisting of H, C₁₋₆ alkyl, and aryl, wherein C₁₋₆ alkyl can be optionally substituted with C₁₋₆ alkoxy;
R¹³ is selected from the group consisting of H, C₁₋₆ alkyl, aryl, and —Obenzyl, wherein C₁₋₆ alkyl can be optionally substituted from 1 to 3 times with a substituent independently selected from —NHCOR¹⁶, aryl, benzyl, and —NR¹⁷R¹⁸,
R¹⁴ is selected from the group consisting of H, C₁₋₆ alkyl, heterocyclyl, and heteroaryl, wherein heterocyclyl and heteroaryl can be optionally substituted with C₁₋₆ alkyl or —COR¹⁹;
R¹⁵ is independently selected at each occurrence from the group consisting of ═O, —OH, halogen, CN, —COC₁₋₆ alkyl, —COOH, —COOC₁₋₆ alkyl, aryl, C₁₋₆ alkyl, C₁₋₆ alkoxy, —CONHR¹⁶, —C(O)R²⁰, —C(O)aryl, benzyl, —Oaryl, —Obenzyl, —SO₂Me, heterocyclyl, heteroaryl, —OCHF₂, —OCF₂CHF₂, and —OCH₂CF₃, wherein C₁₋₆ alkyl, —Oaryl, aryl, heterocyclyl, and heteroaryl can be optionally substituted from 1 to 3 times with a substituent independently selected at each occurrence thereof from the group consisting of —OH, halogen, ═O, —C(O) CH₃, C₁₋₆ alkyl, —CF₃, heterocyclyl, C₁₋₆ alkoxy, —NR⁴R⁵, and —CN;
R¹⁶ is C₁₋₆ alkyl optionally substituted with C₁₋₆ alkoxy;
R¹⁷ is H or C₁₋₆ alkyl;
R¹⁸ is H or C₁₋₆ alkyl;
R¹⁹ is selected from the group consisting of C₁₋₆ alkyl, aryl, and —NHC₁₋₆ alkyl;
R²⁰ is —NMe₂ or heterocyclyl, wherein heterocyclyl can be optionally substituted 1 to 3 times with OH or C₁₋₆ alkyl;
n is 1, 2, 3, 4, or 5; and
m is 0 or 1;
with the proviso that both R¹ and R² cannot be H,
or an oxide thereof, a pharmaceutically acceptable salt thereof, a solvate thereof, or a prodrug thereof.

2. The compound according to claim 1, which has the Formula (Ia):

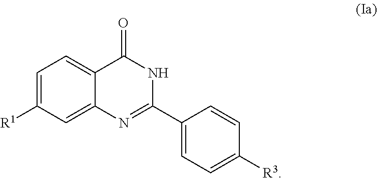

3. The compound according to claim 1, which has the Formula (Ib):

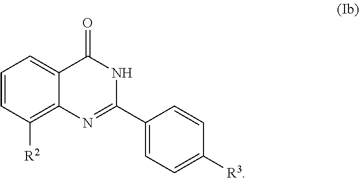

4. The compound according to claim 1, wherein R¹ is selected from the group consisting of H, 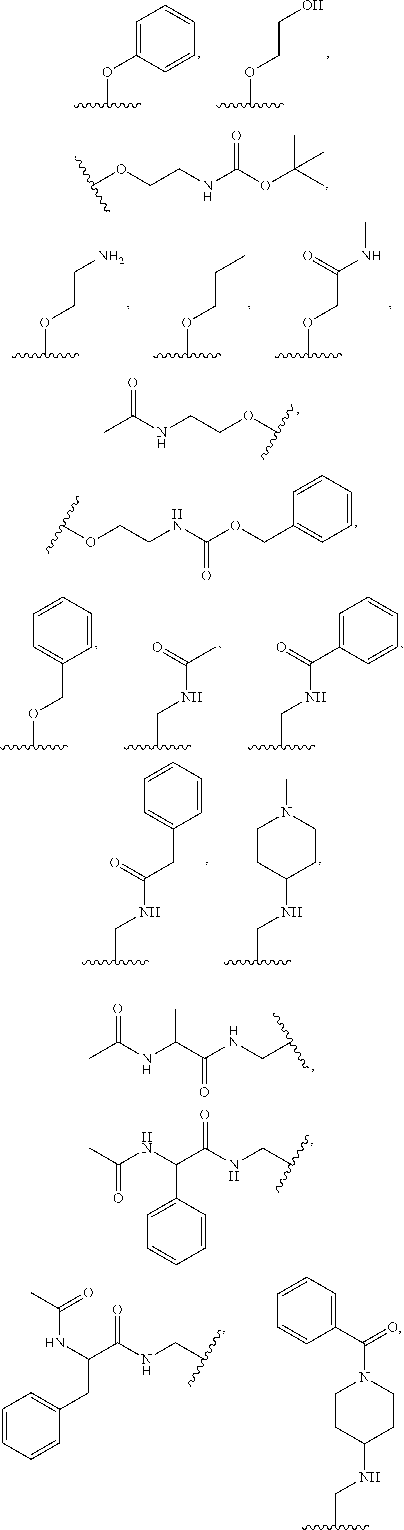 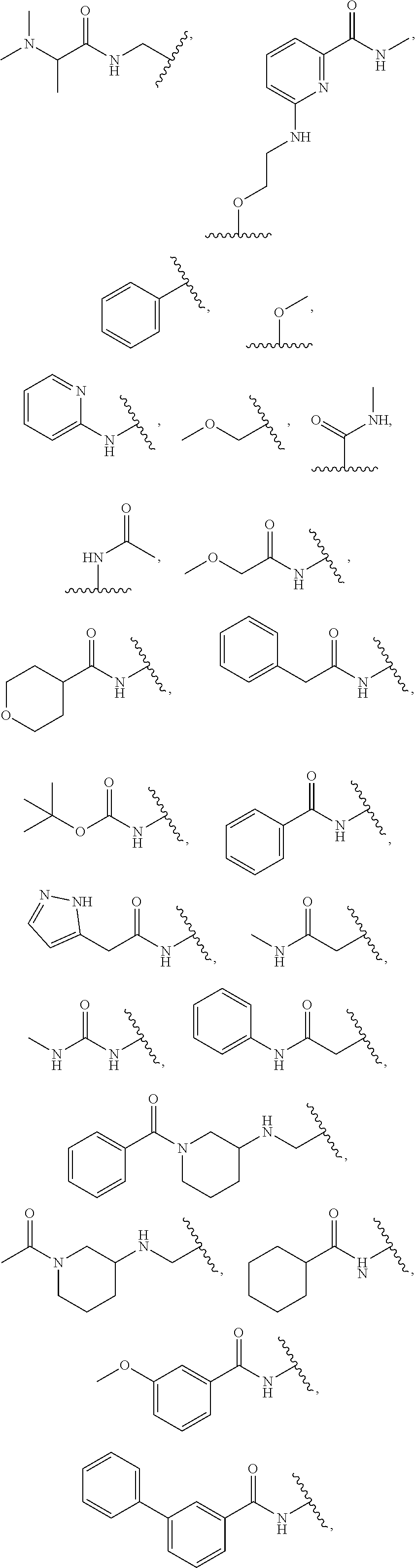 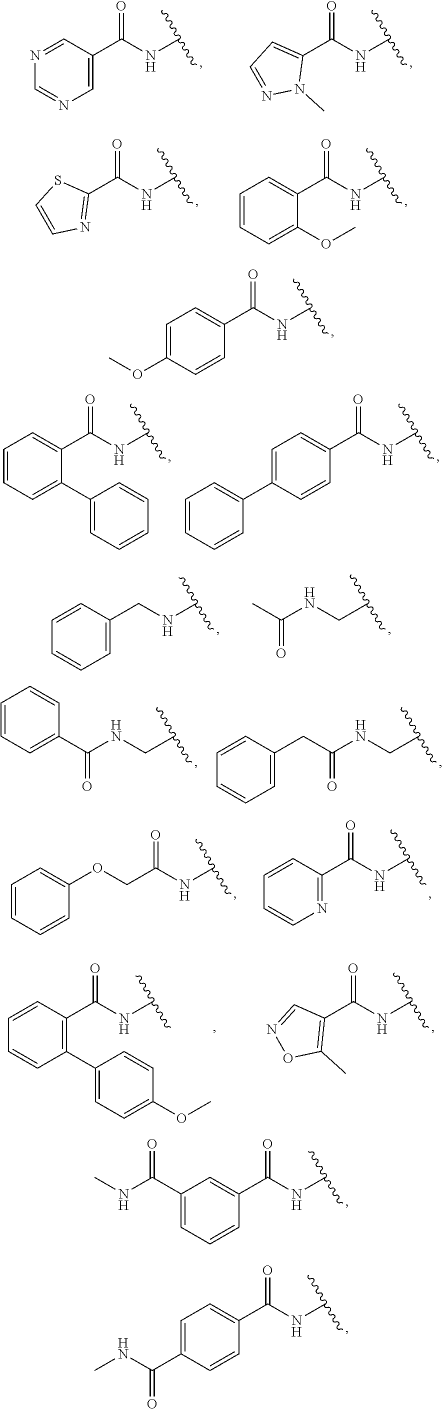 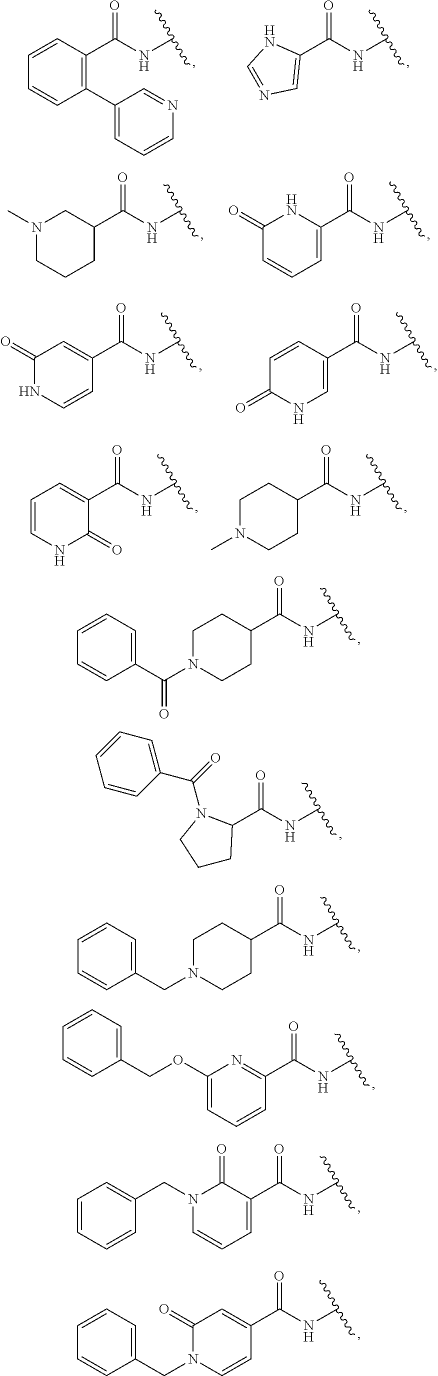 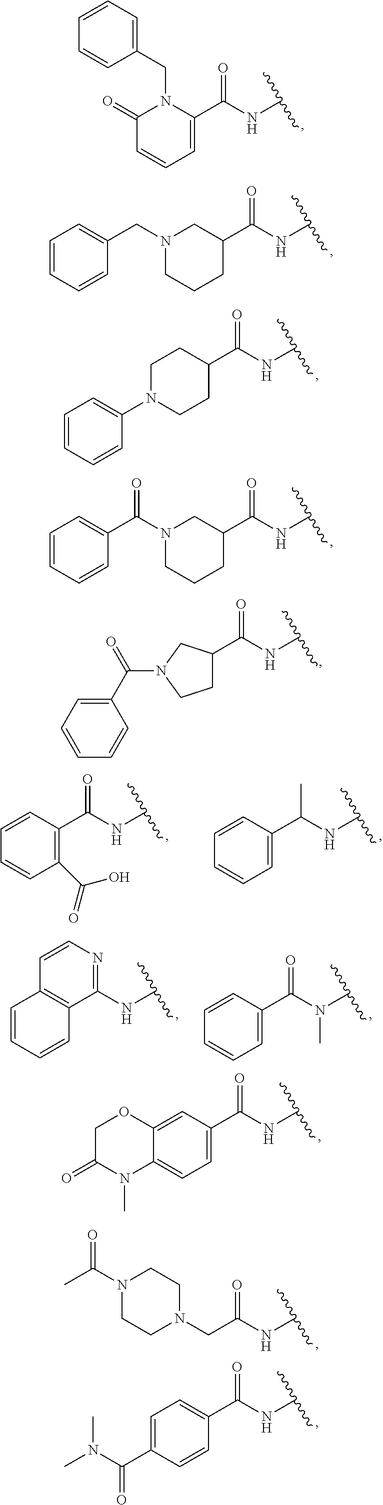 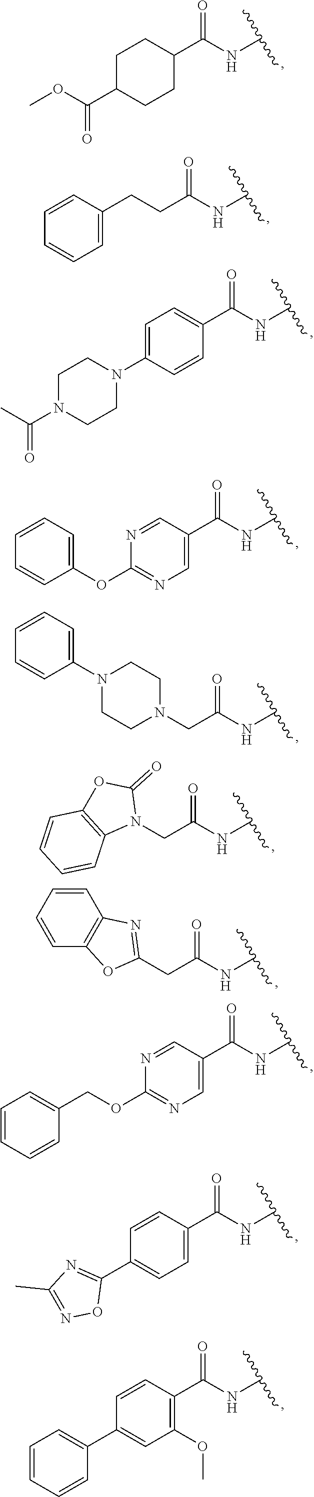 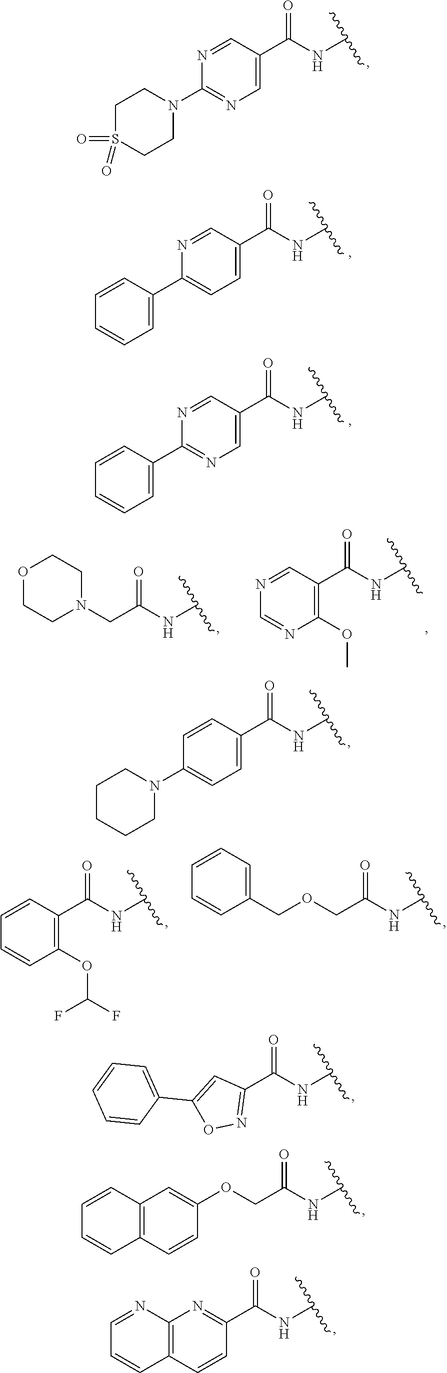 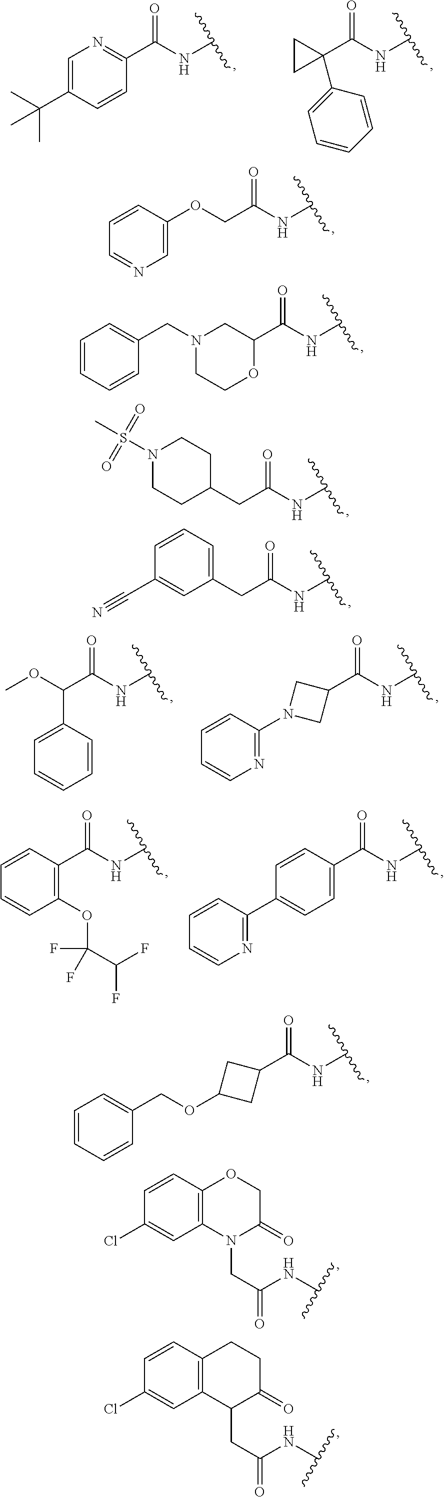 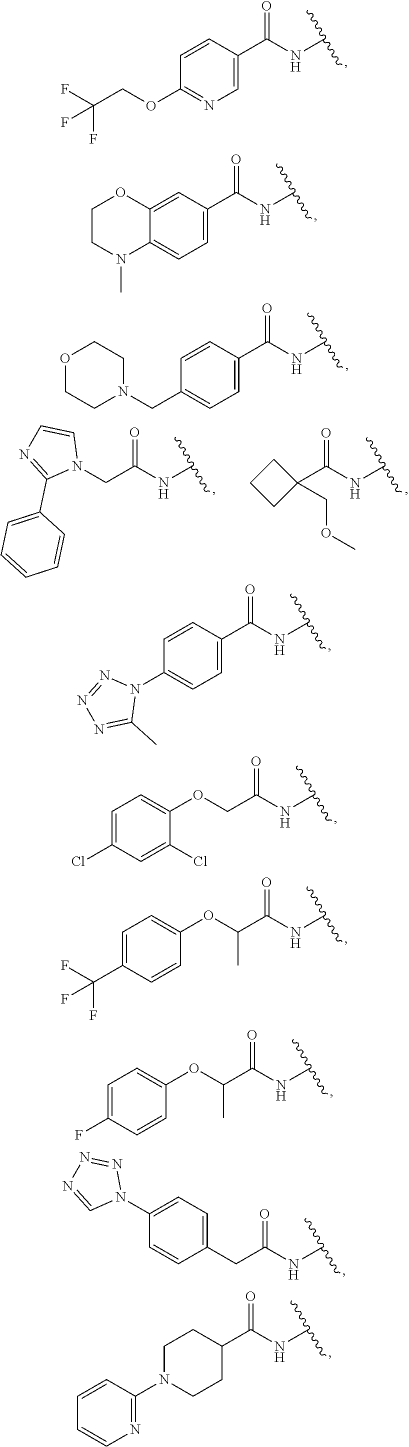 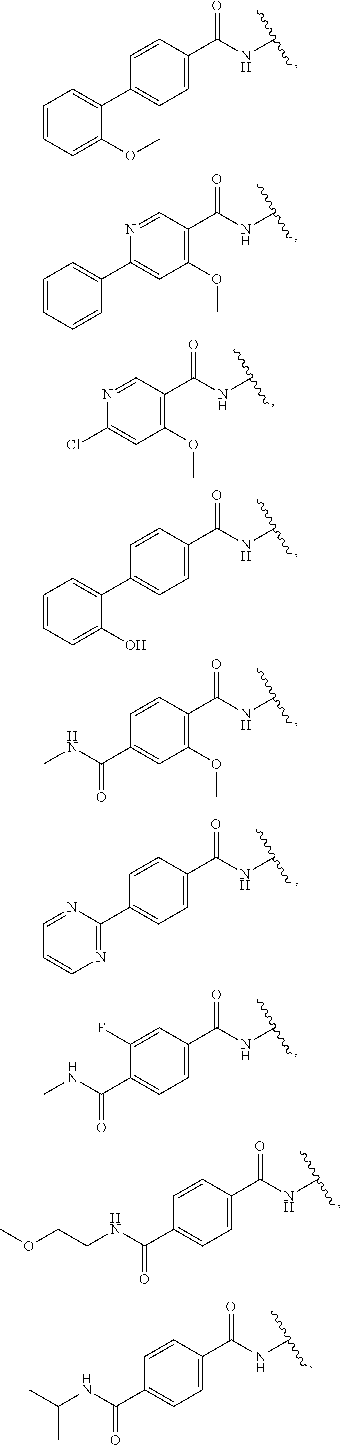 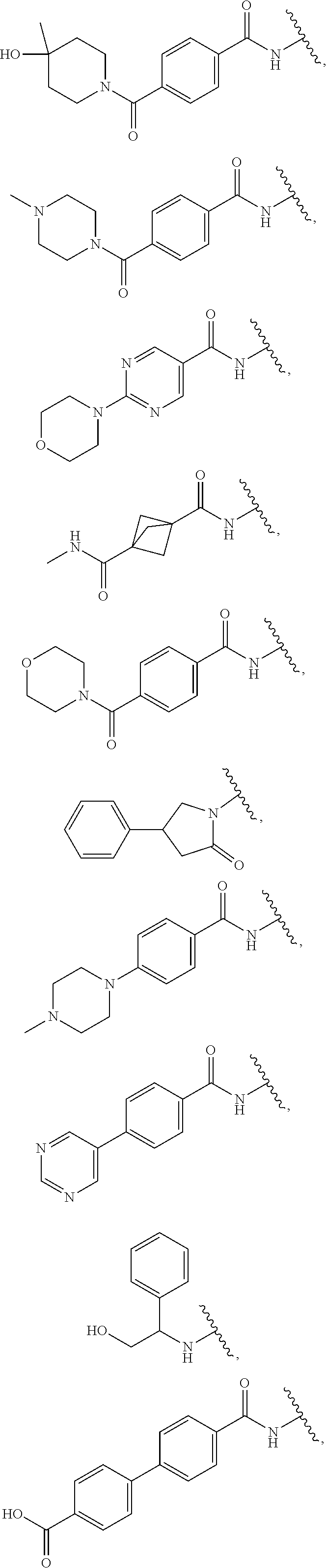 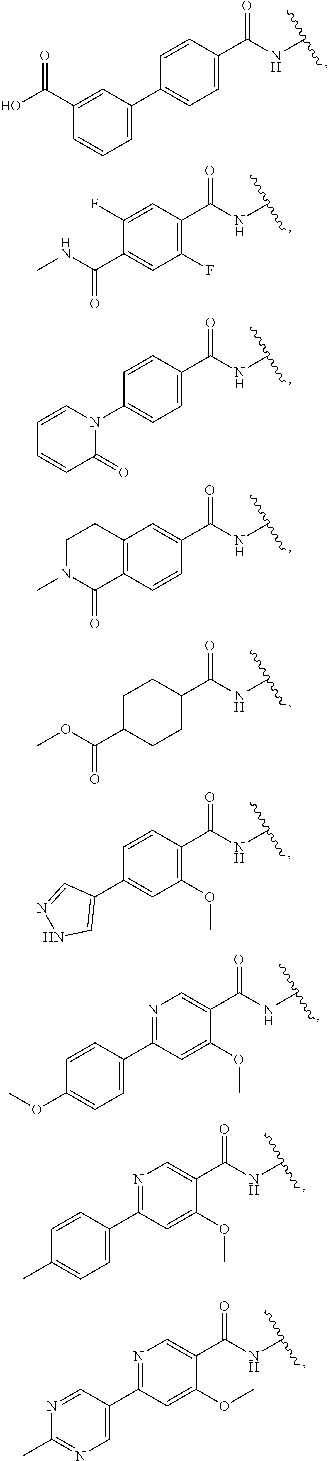 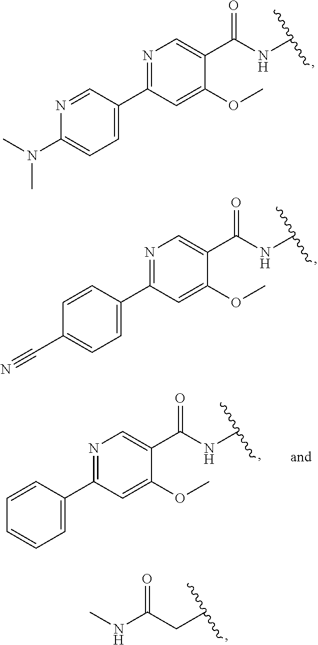
wherein 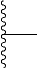
is the point of attachment to the corresponding carbon atom of the structure of Formula (I).

5. The compound according to claim 1, wherein R² is selected from the group consisting of H, 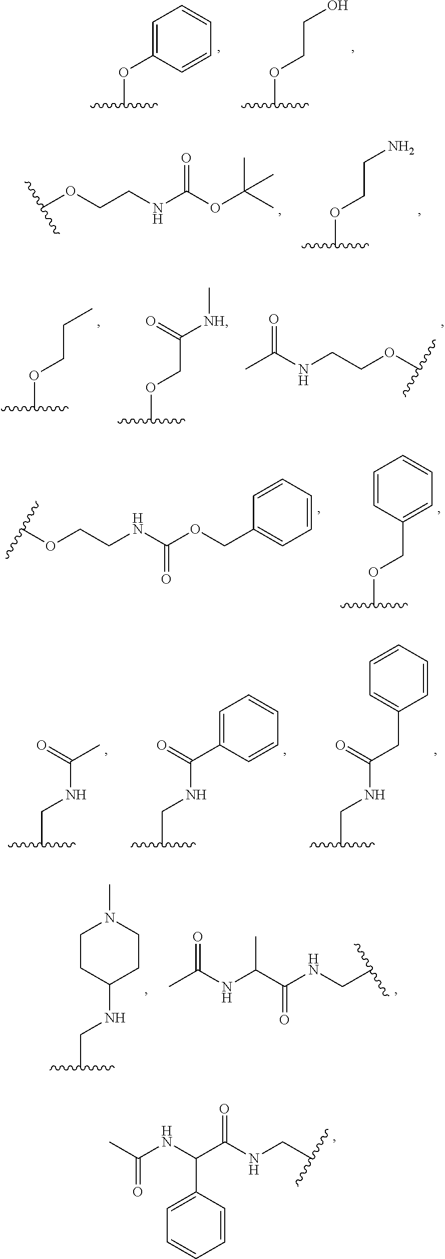 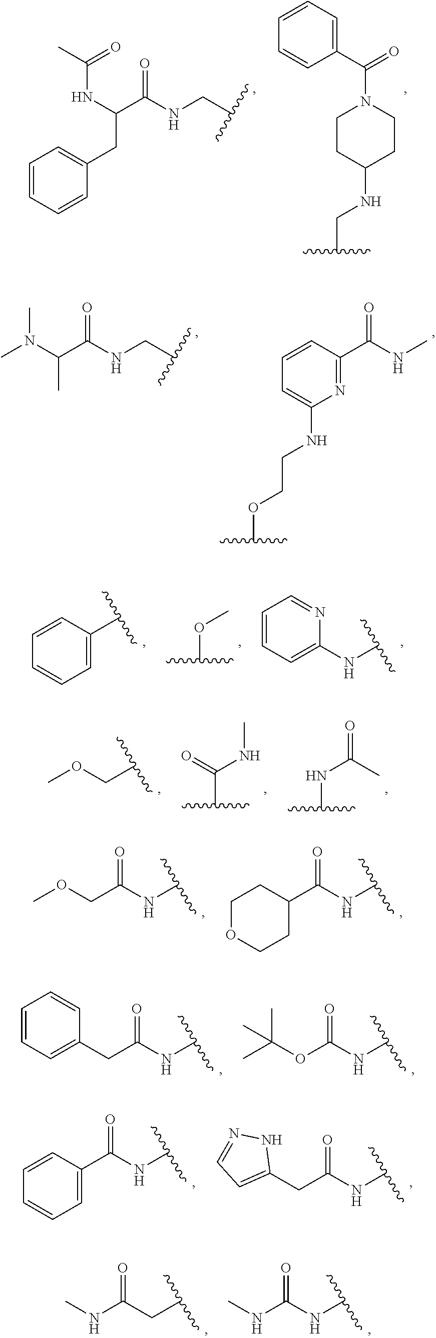 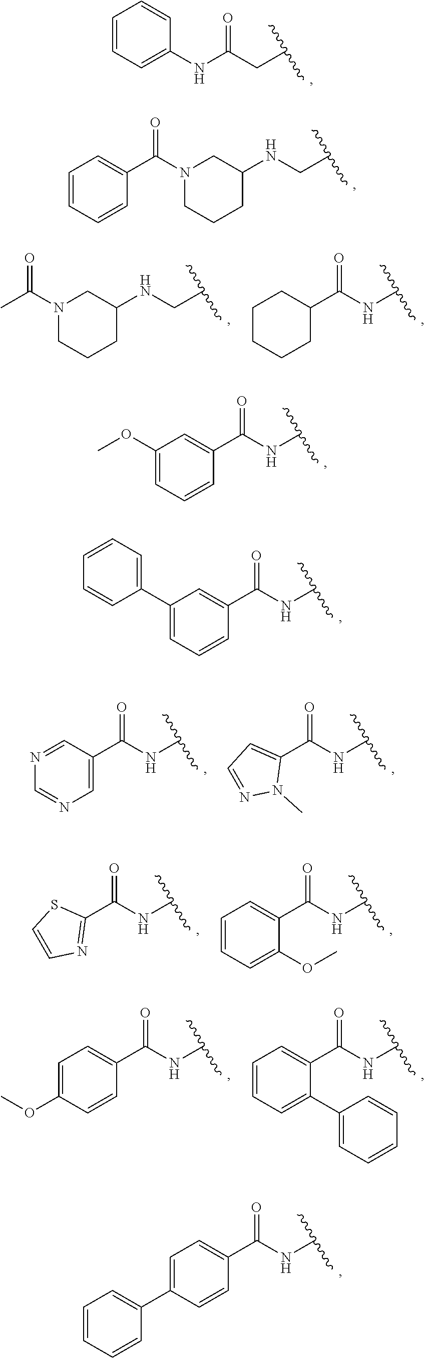 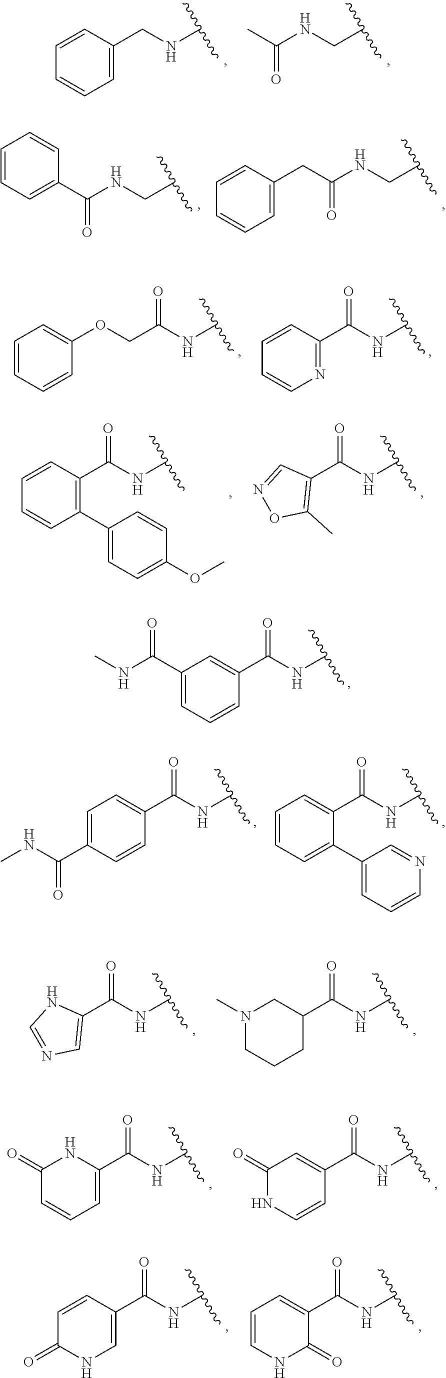 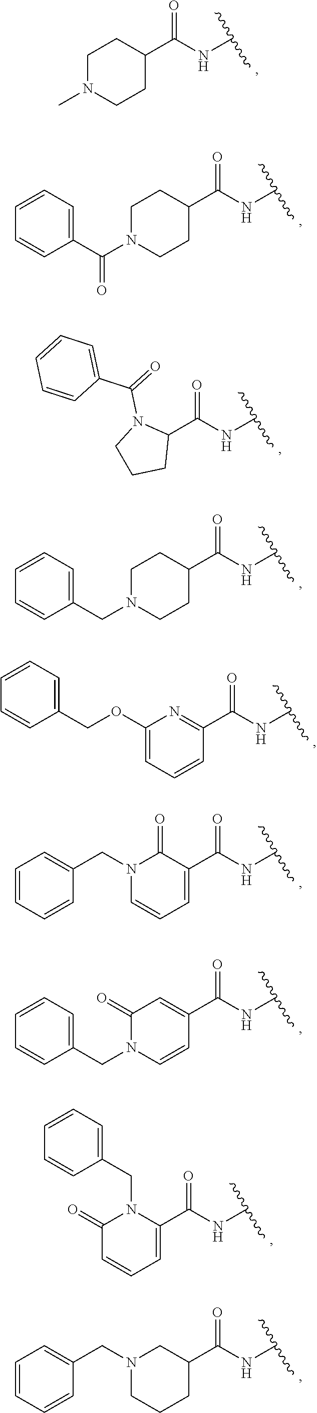 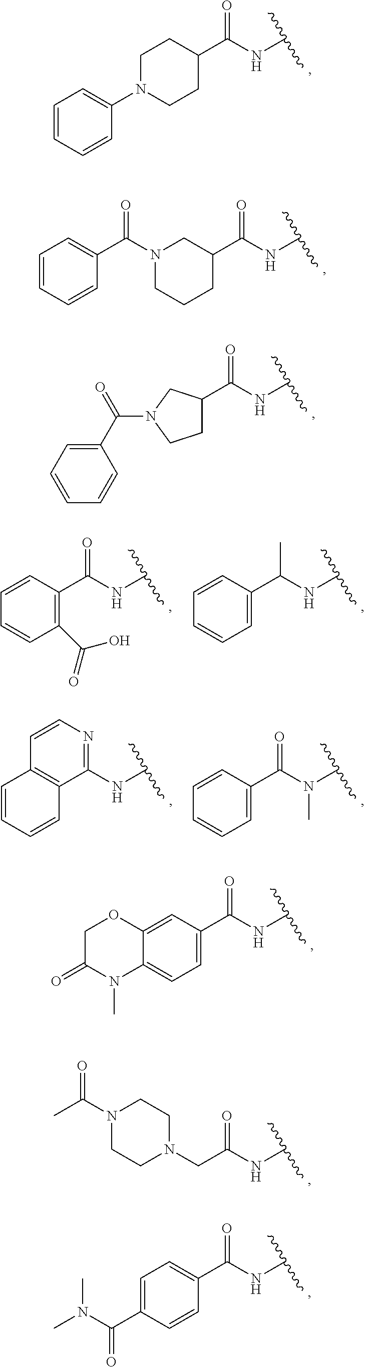 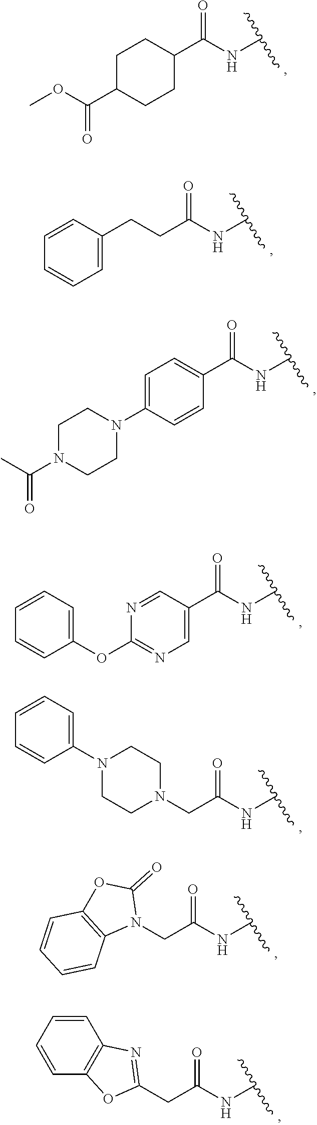 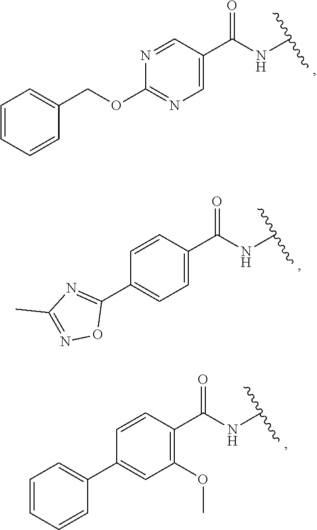 [SMILES: *NC(=O)c1cnc(OCc2ccccc2)nc1 ; *NC(=O)c1ccc(-c2nc(C)no2)cc1 ; *NC(=O)c1ccc(-c2ccccc2)cc1OC] 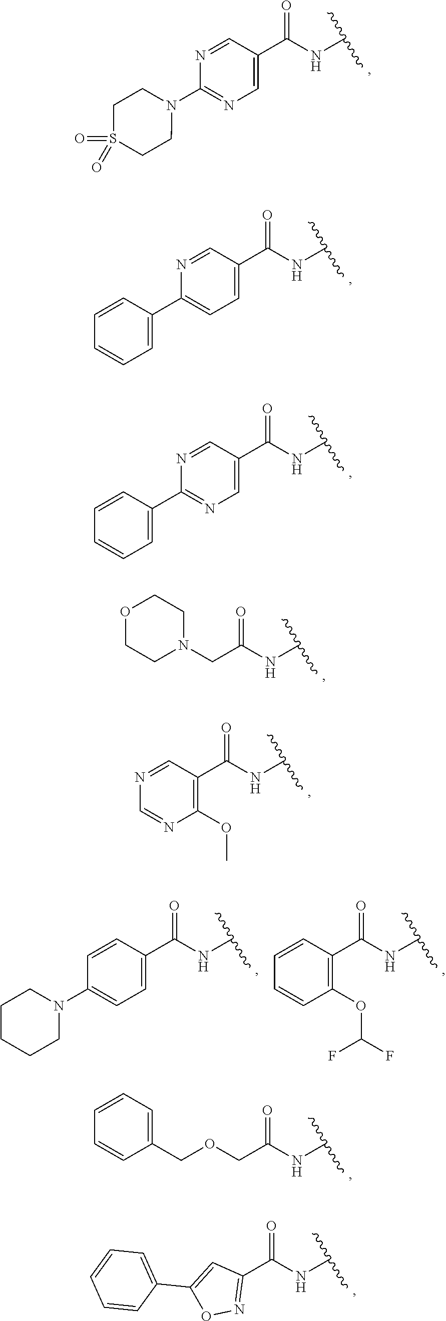 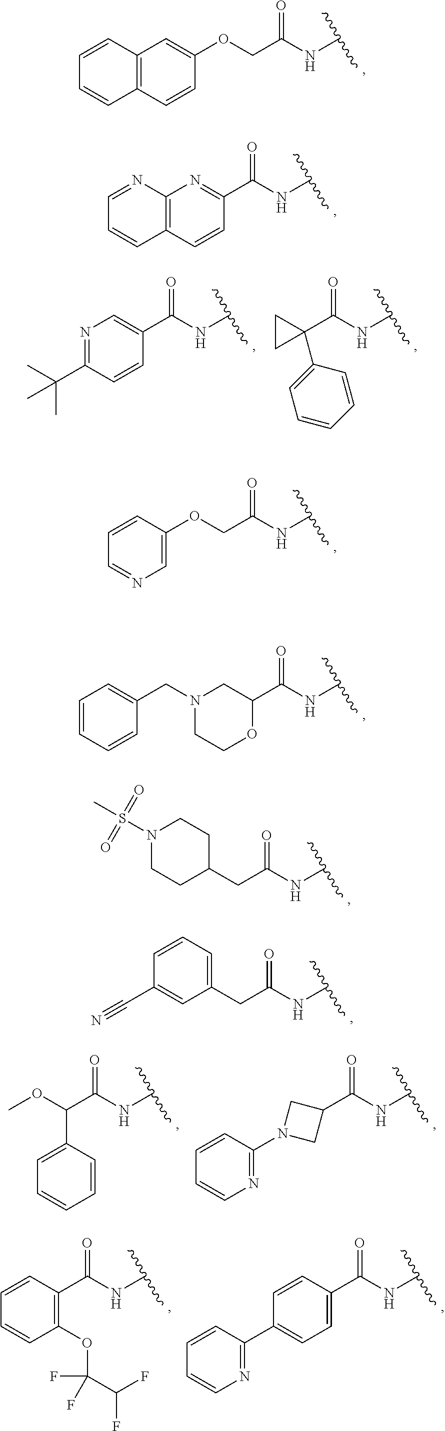 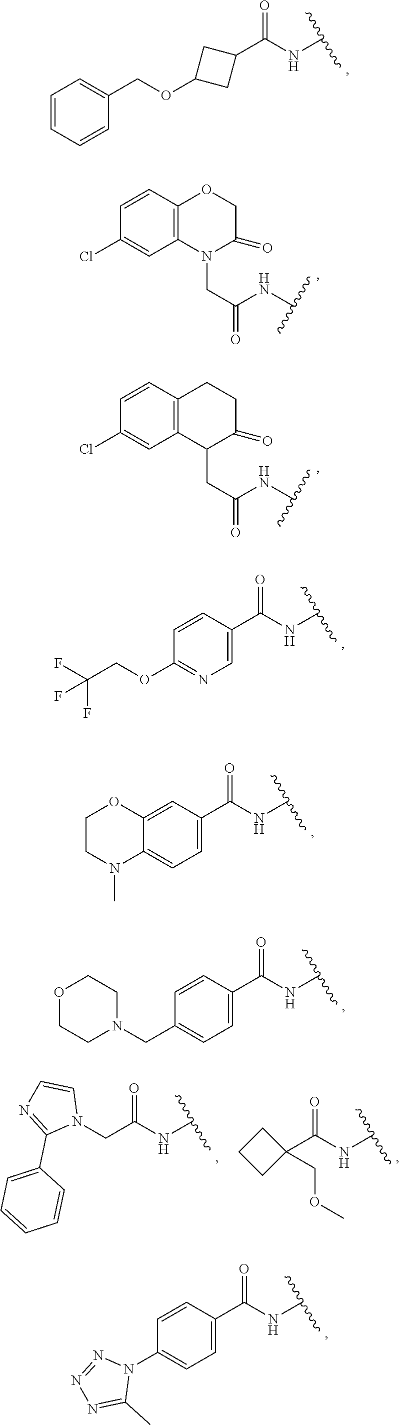 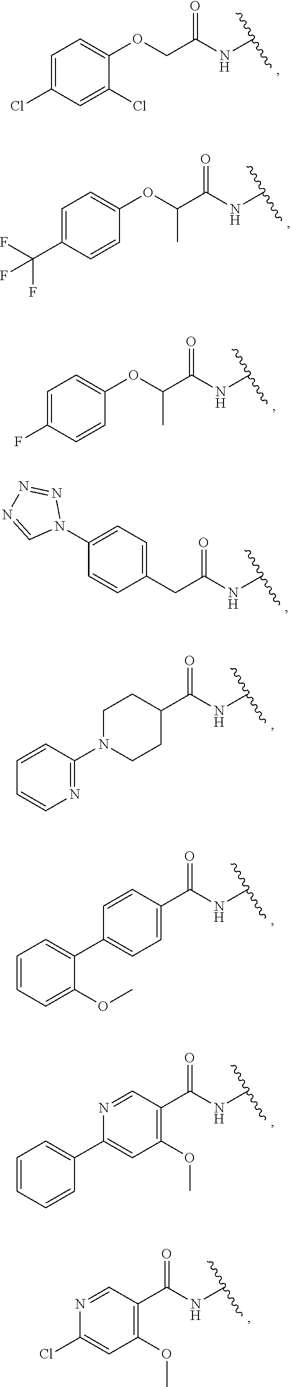 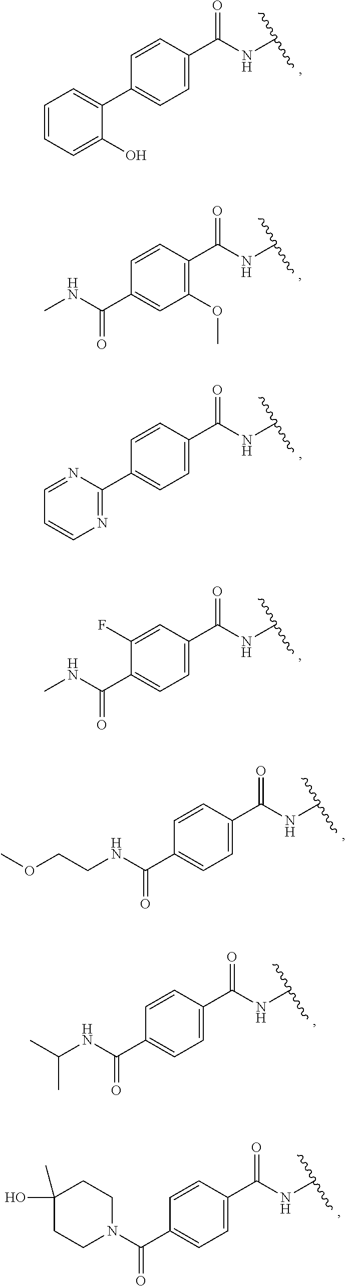 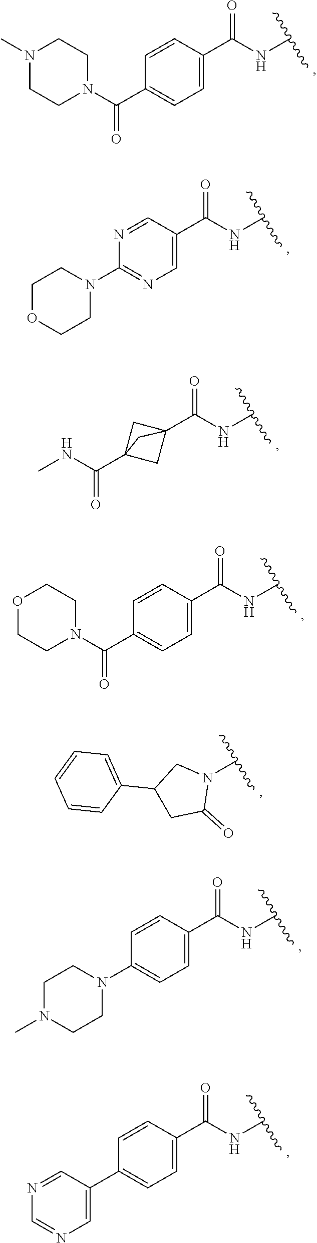 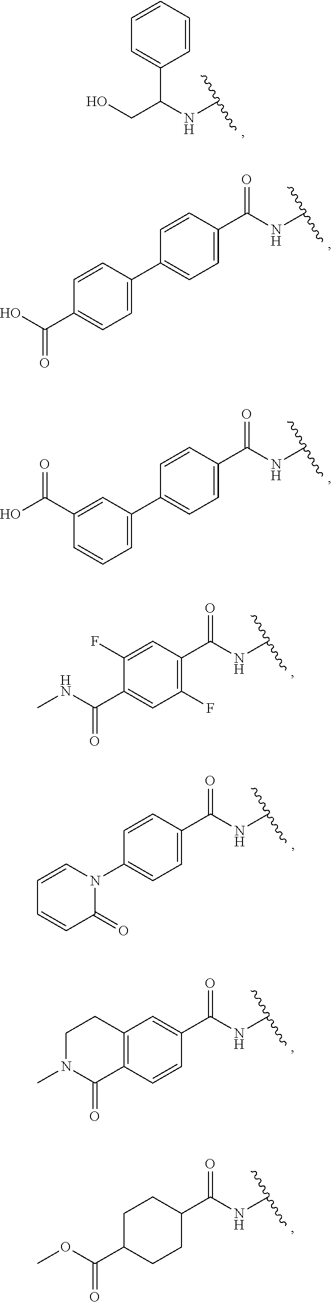 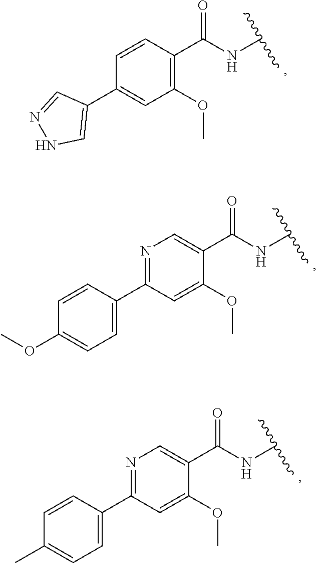 [SMILES: *NC(=O)c1ccc(-c2cn[nH]c2)cc1OC ; *NC(=O)c1cnc(-c2ccc(OC)cc2)cc1OC ; *NC(=O)c1cnc(-c2ccc(C)cc2)cc1OC] 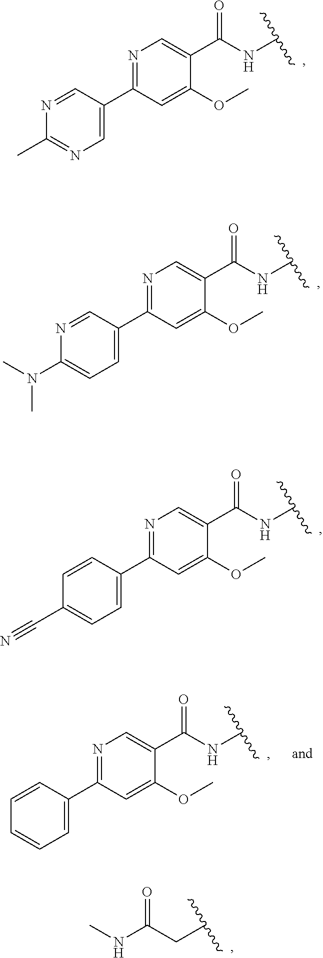
wherein 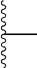
is the point of attachment to the corresponding carbon atom of the structure of Formula (I).

6. The compound according to claim 1, wherein R³ is selected from the group consisting of 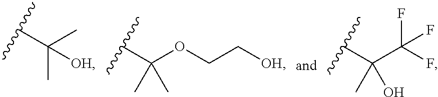
wherein 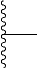
is the point of attachment to the corresponding carbon atom of the structure of Formula (I).

7. The compound according to claim 2, wherein R¹ is selected from the group consisting of 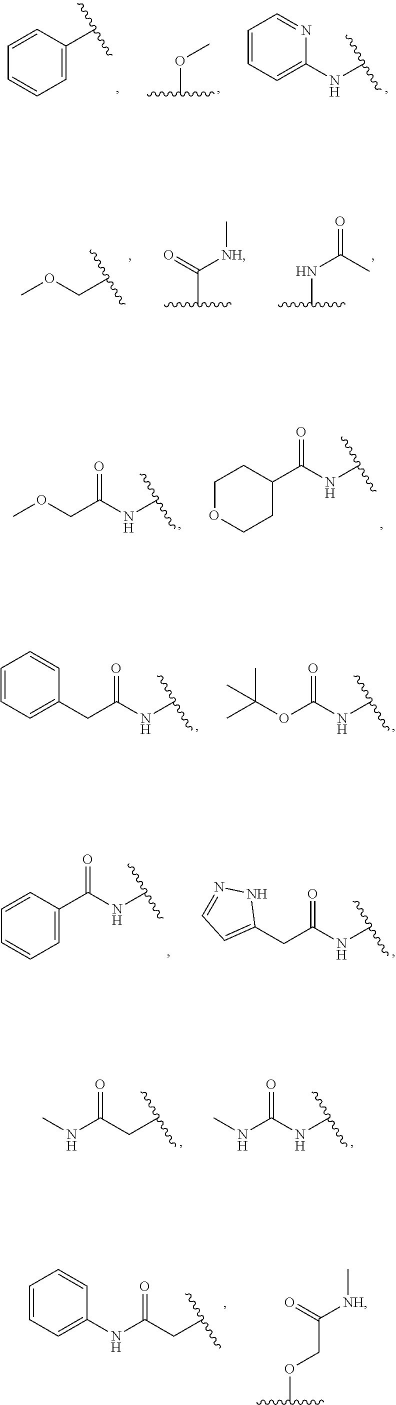 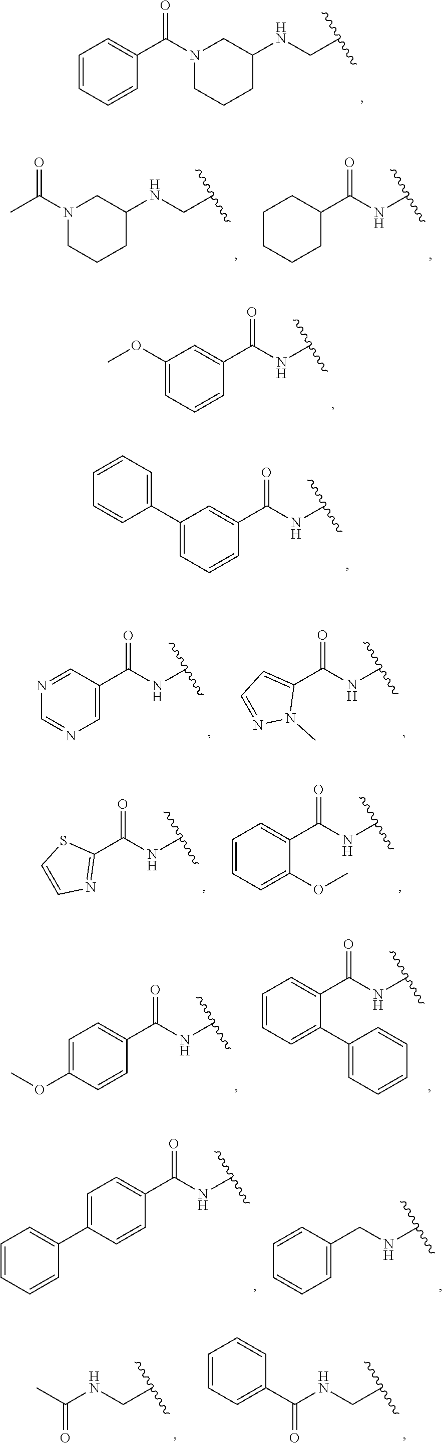 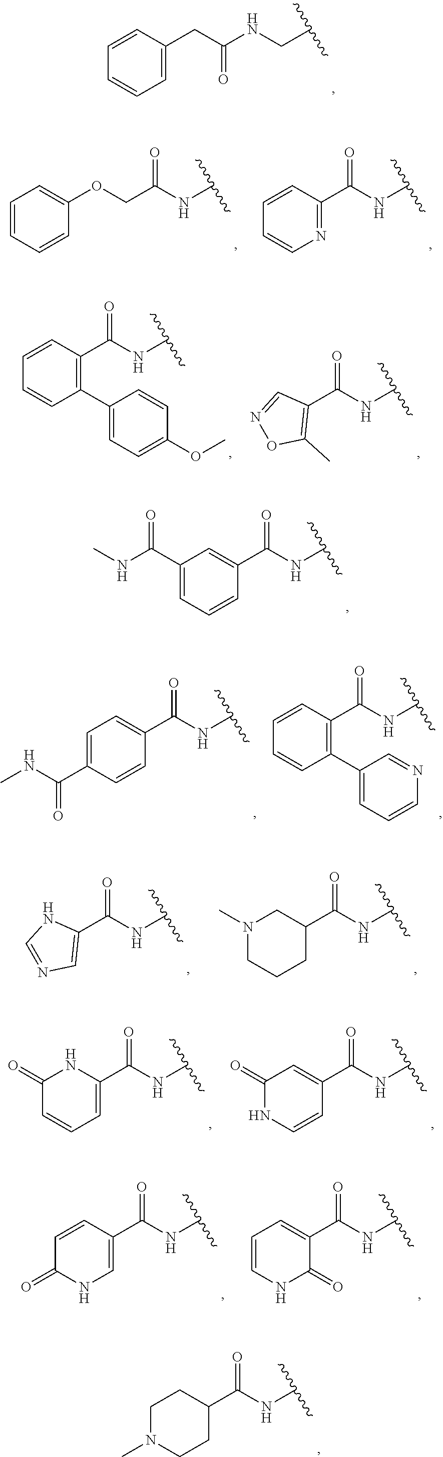 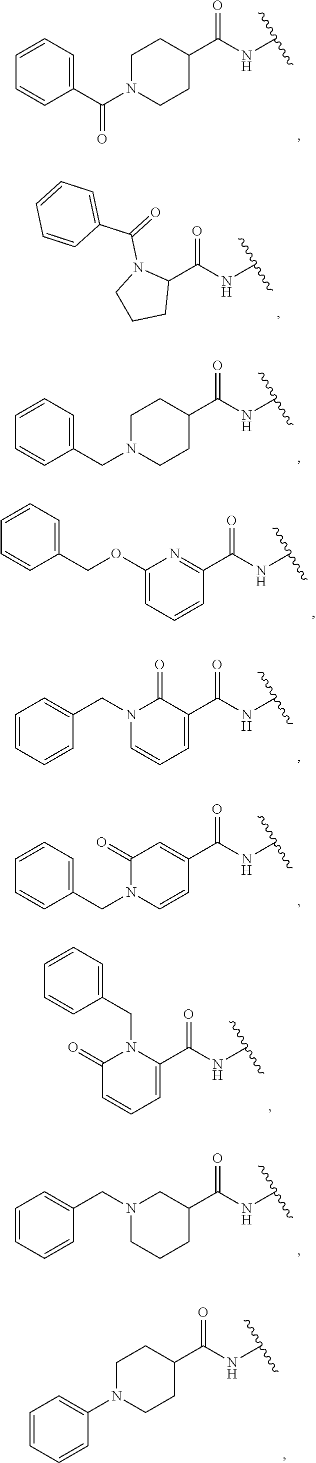 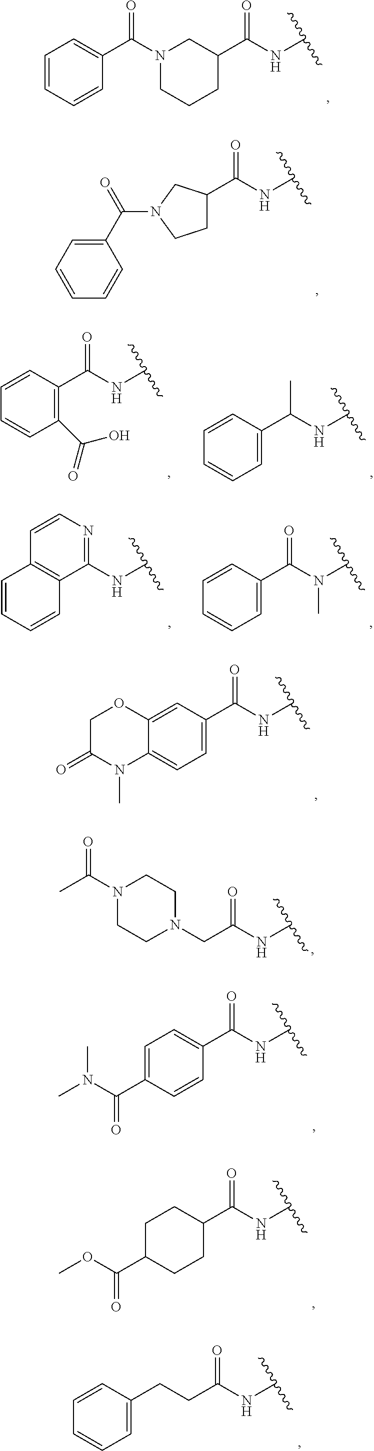 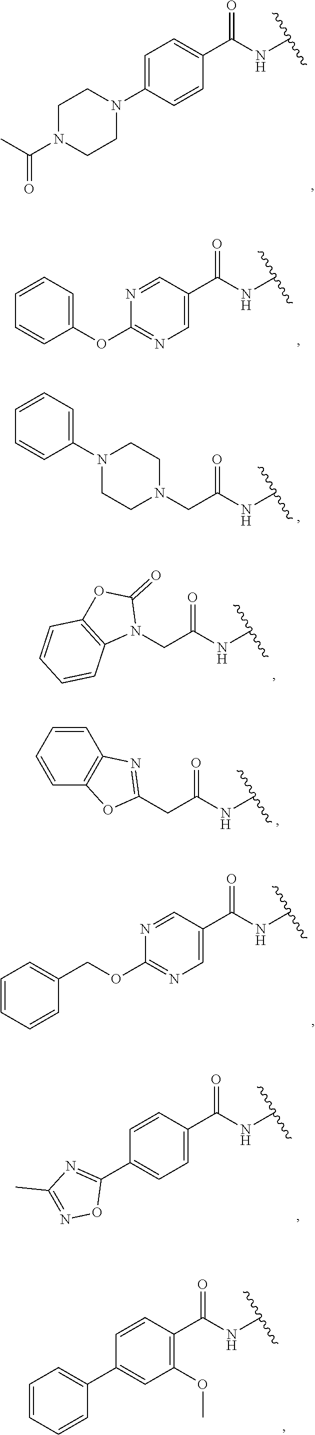 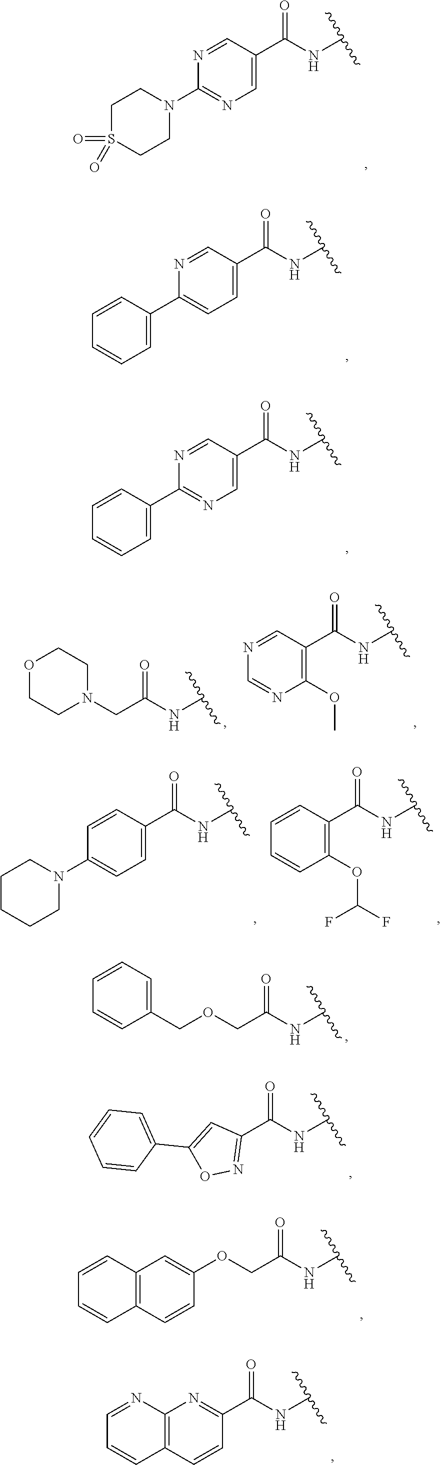 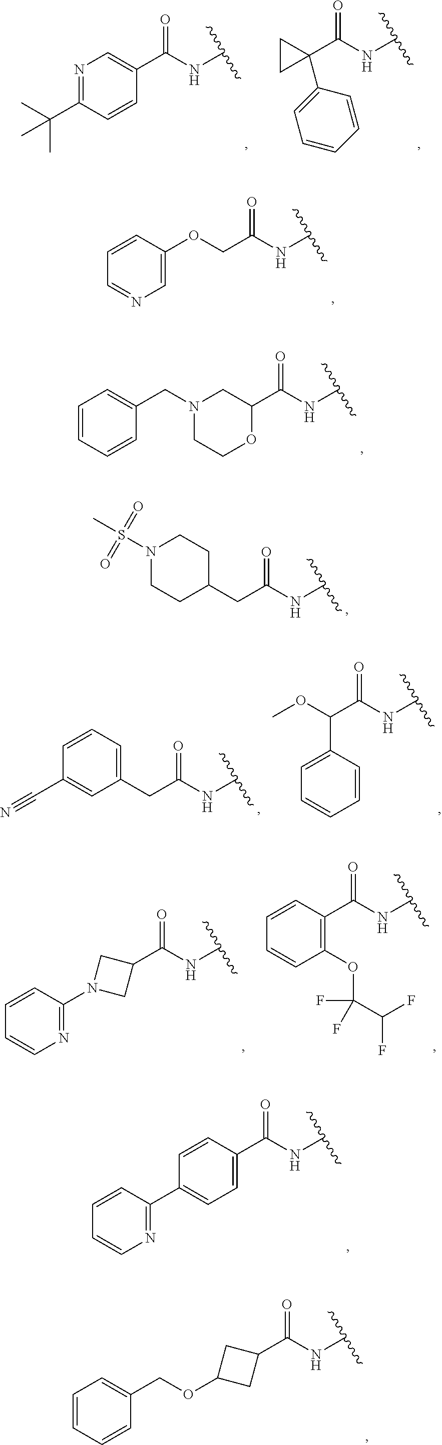 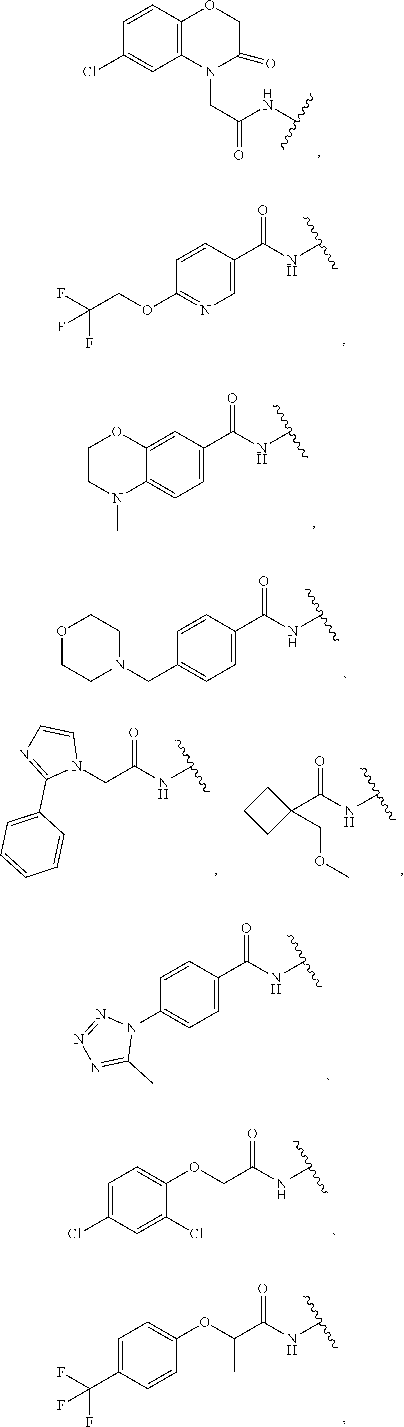 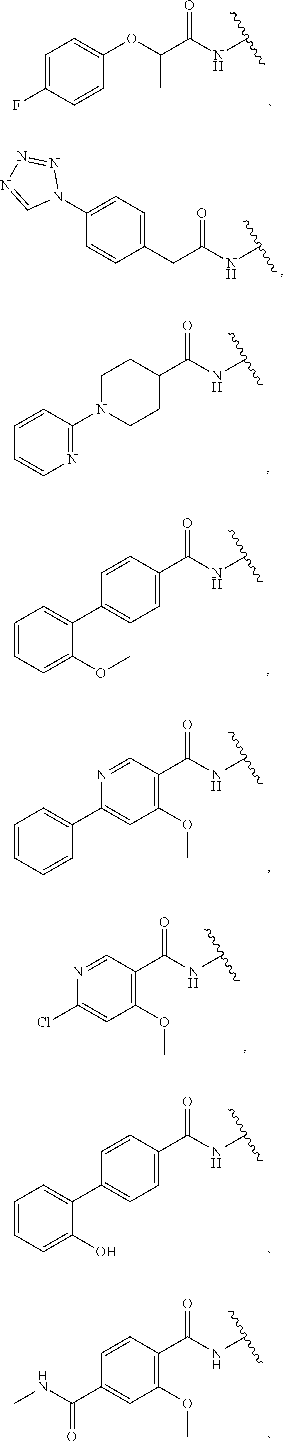 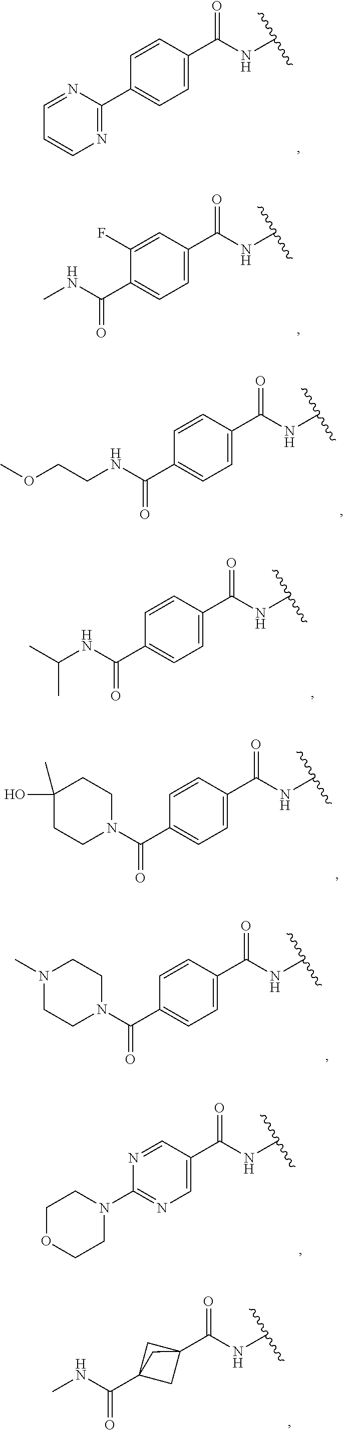 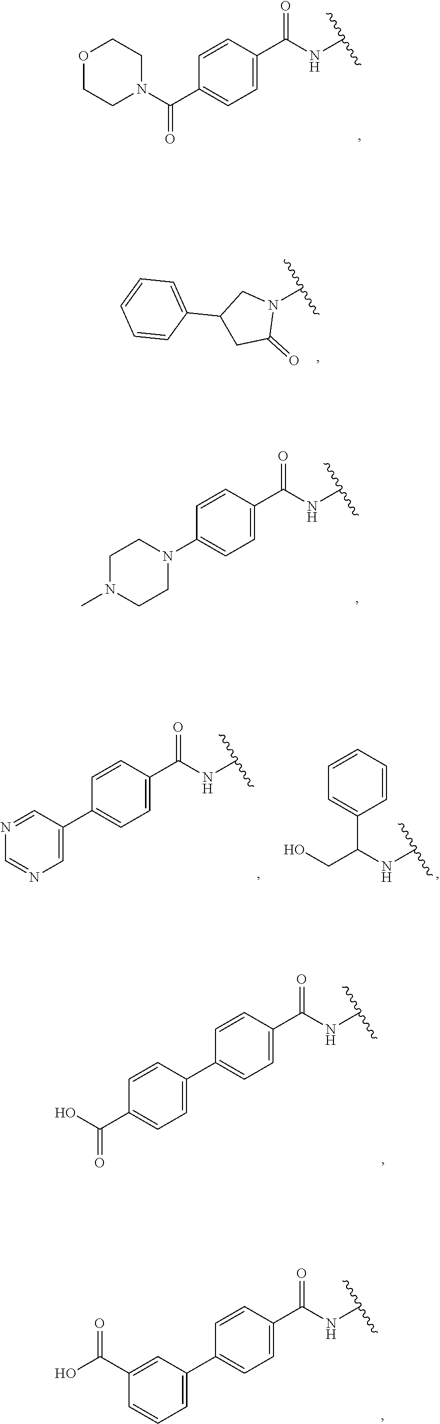 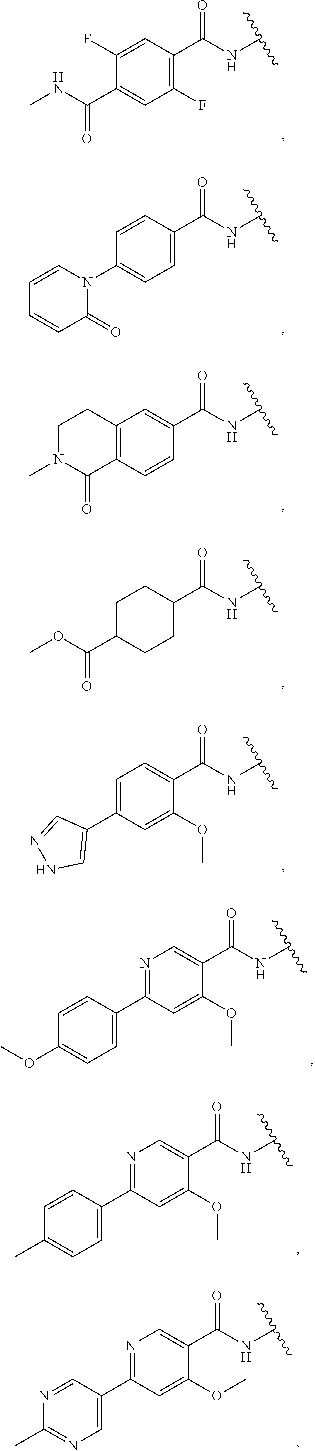 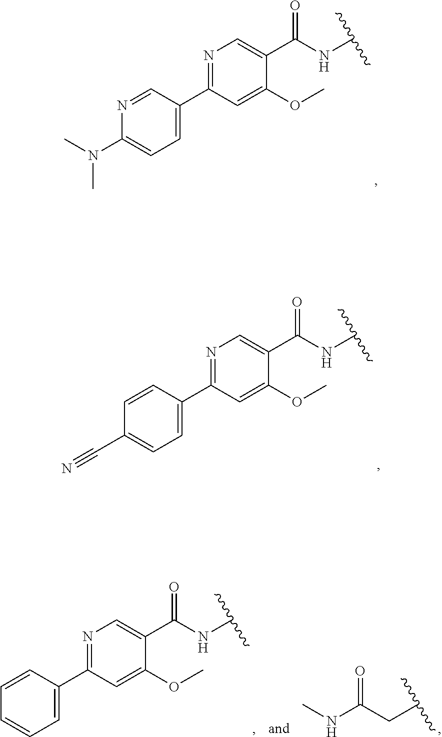
wherein 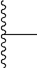
is the point of attachment to the corresponding carbon atom of the structure of Formula (Ia).

8. The compound according to claim 3, wherein R² is selected from the group consisting of 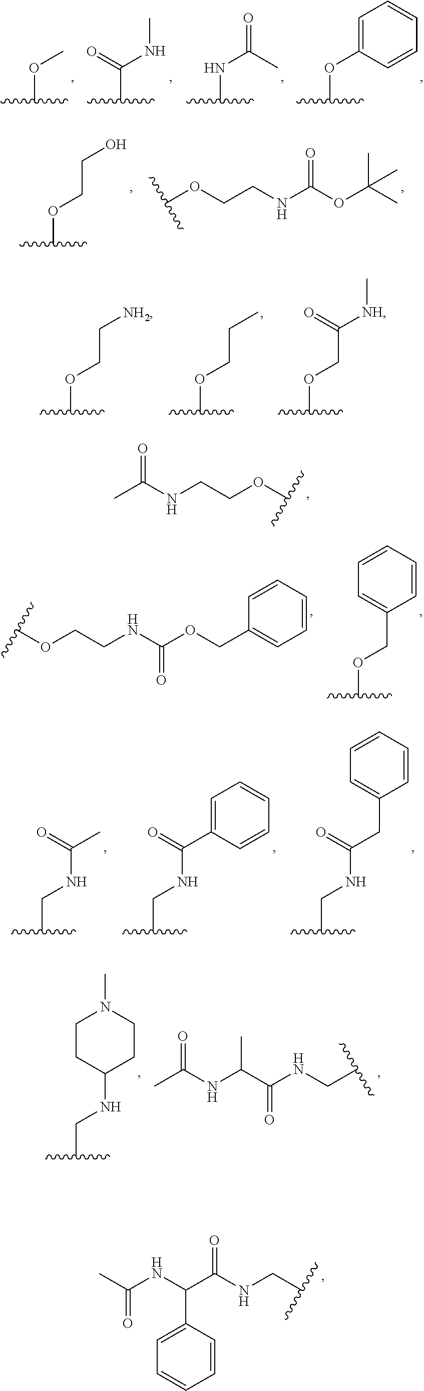 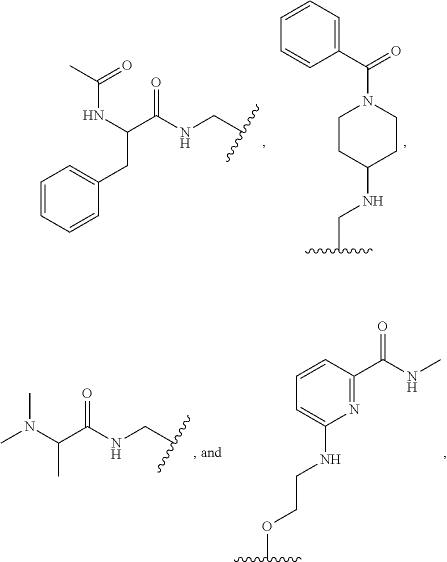
wherein 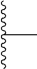
is the point of attachment to the corresponding carbon atom of the structure of Formula (Ib).

9. The compound according to claim 1, wherein the compound of Formula (I) is selected from the group consisting of:

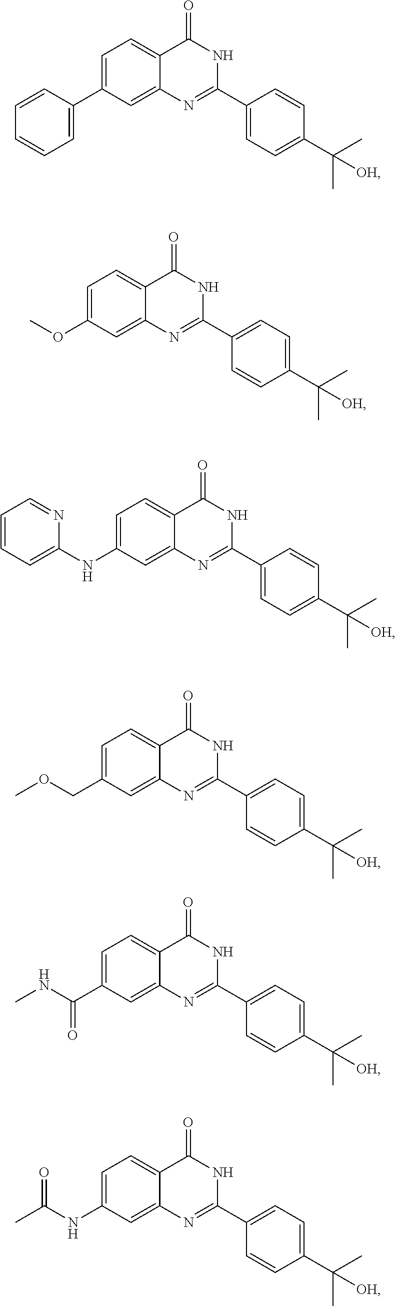 [SMILES: CC(C)(O)c1ccc(-c2nc3cc(-c4ccccc4)ccc3c(=O)[nH]2)cc1 ; COc1ccc2c(=O)[nH]c(-c3ccc(C(C)(C)O)cc3)nc2c1 ; CC(C)(O)c1ccc(-c2nc3cc(Nc4ccccn4)ccc3c(=O)[nH]2)cc1 ; COCc1ccc2c(=O)[nH]c(-c3ccc(C(C)(C)O)cc3)nc2c1 ; CNC(=O)c1ccc2c(=O)[nH]c(-c3ccc(C(C)(C)O)cc3)nc2c1 ; CC(=O)Nc1ccc2c(=O)[nH]c(-c3ccc(C(C)(C)O)cc3)nc2c1]

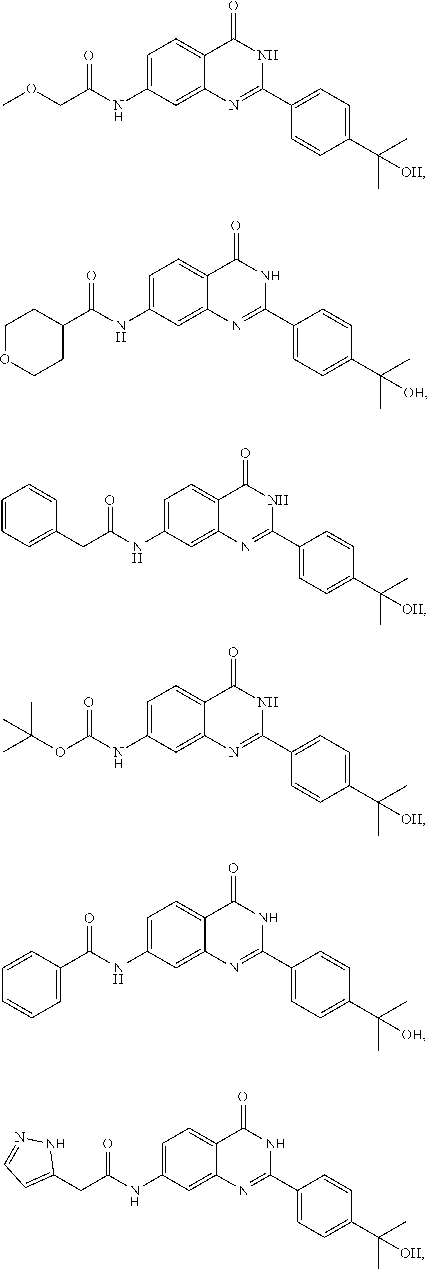 [SMILES: COCC(=O)Nc1ccc2c(=O)[nH]c(-c3ccc(C(C)(C)O)cc3)nc2c1 ; CC(C)(O)c1ccc(-c2nc3cc(NC(=O)C4CCOCC4)ccc3c(=O)[nH]2)cc1 ; CC(C)(O)c1ccc(-c2nc3cc(NC(=O)Cc4ccccc4)ccc3c(=O)[nH]2)cc1 ; CC(C)(C)OC(=O)Nc1ccc2c(=O)[nH]c(-c3ccc(C(C)(C)O)cc3)nc2c1 ; CC(C)(O)c1ccc(-c2nc3cc(NC(=O)c4ccccc4)ccc3c(=O)[nH]2)cc1 ; CC(C)(O)c1ccc(-c2nc3cc(NC(=O)Cc4ccn[nH]4)ccc3c(=O)[nH]2)cc1]

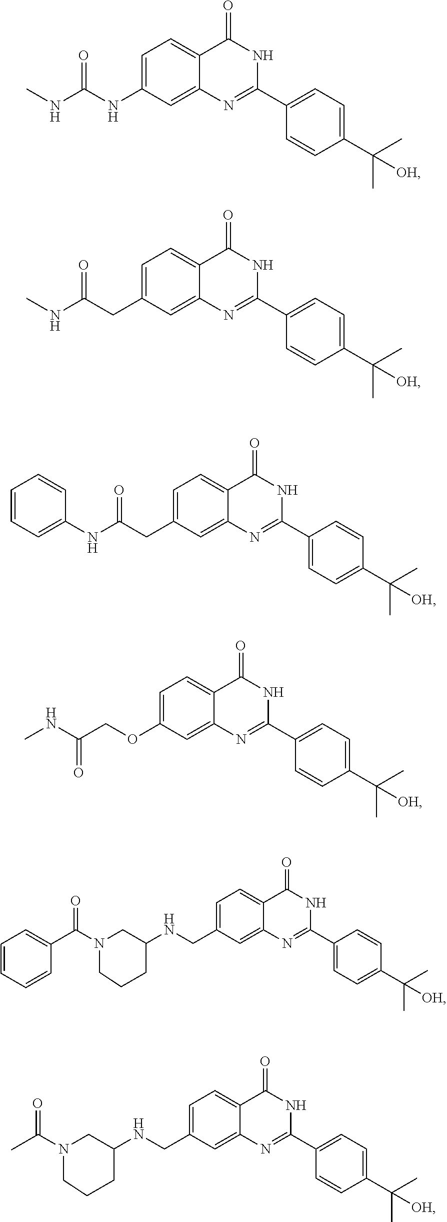 [SMILES: CNC(=O)Nc1ccc2c(=O)[nH]c(-c3ccc(C(C)(C)O)cc3)nc2c1 ; CNC(=O)Cc1ccc2c(=O)[nH]c(-c3ccc(C(C)(C)O)cc3)nc2c1 ; CC(C)(O)c1ccc(-c2nc3cc(CC(=O)Nc4ccccc4)ccc3c(=O)[nH]2)cc1 ; CNC(=O)COc1ccc2c(=O)[nH]c(-c3ccc(C(C)(C)O)cc3)nc2c1 ; CC(C)(O)c1ccc(-c2nc3cc(CNC4CCCN(C(=O)c5ccccc5)C4)ccc3c(=O)[nH]2)cc1 ; CC(=O)N1CCCC(NCc2ccc3c(=O)[nH]c(-c4ccc(C(C)(C)O)cc4)nc3c2)C1]

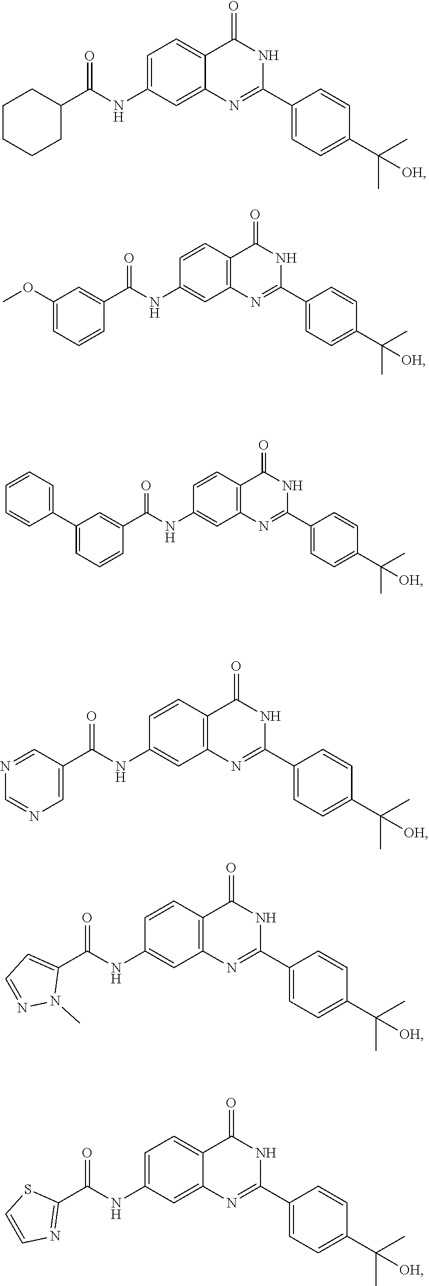 [SMILES: CC(C)(O)c1ccc(-c2nc3cc(NC(=O)C4CCCCC4)ccc3c(=O)[nH]2)cc1 ; COc1cccc(C(=O)Nc2ccc3c(=O)[nH]c(-c4ccc(C(C)(C)O)cc4)nc3c2)c1 ; CC(C)(O)c1ccc(-c2nc3cc(NC(=O)c4cccc(-c5ccccc5)c4)ccc3c(=O)[nH]2)cc1 ; CC(C)(O)c1ccc(-c2nc3cc(NC(=O)c4cncnc4)ccc3c(=O)[nH]2)cc1 ; Cn1nccc1C(=O)Nc1ccc2c(=O)[nH]c(-c3ccc(C(C)(C)O)cc3)nc2c1 ; CC(C)(O)c1ccc(-c2nc3cc(NC(=O)c4nccs4)ccc3c(=O)[nH]2)cc1]

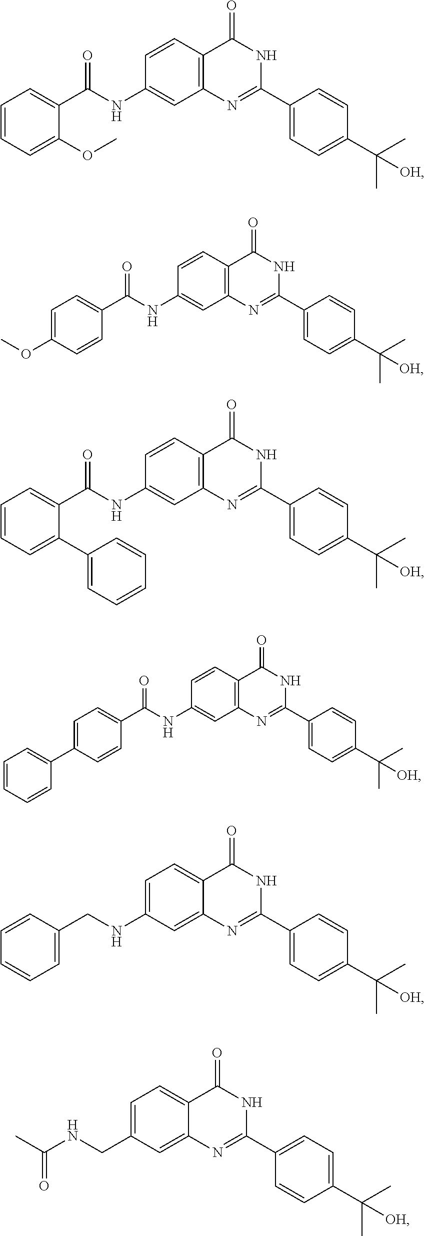 [SMILES: COc1ccccc1C(=O)Nc1ccc2c(=O)[nH]c(-c3ccc(C(C)(C)O)cc3)nc2c1 ; COc1ccc(C(=O)Nc2ccc3c(=O)[nH]c(-c4ccc(C(C)(C)O)cc4)nc3c2)cc1 ; CC(C)(O)c1ccc(-c2nc3cc(NC(=O)c4ccccc4-c4ccccc4)ccc3c(=O)[nH]2)cc1 ; CC(C)(O)c1ccc(-c2nc3cc(NC(=O)c4ccc(-c5ccccc5)cc4)ccc3c(=O)[nH]2)cc1 ; CC(C)(O)c1ccc(-c2nc3cc(NCc4ccccc4)ccc3c(=O)[nH]2)cc1 ; CC(=O)NCc1ccc2c(=O)[nH]c(-c3ccc(C(C)(C)O)cc3)nc2c1]

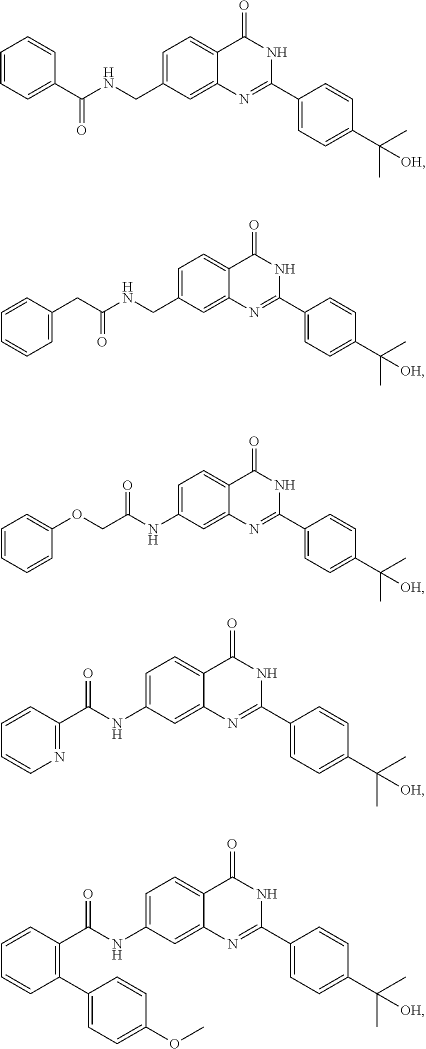 [SMILES: CC(C)(O)c1ccc(-c2nc3cc(CNC(=O)c4ccccc4)ccc3c(=O)[nH]2)cc1 ; CC(C)(O)c1ccc(-c2nc3cc(CNC(=O)Cc4ccccc4)ccc3c(=O)[nH]2)cc1 ; CC(C)(O)c1ccc(-c2nc3cc(NC(=O)COc4ccccc4)ccc3c(=O)[nH]2)cc1 ; CC(C)(O)c1ccc(-c2nc3cc(NC(=O)c4ccccn4)ccc3c(=O)[nH]2)cc1 ; COc1ccc(-c2ccccc2C(=O)Nc2ccc3c(=O)[nH]c(-c4ccc(C(C)(C)O)cc4)nc3c2)cc1]

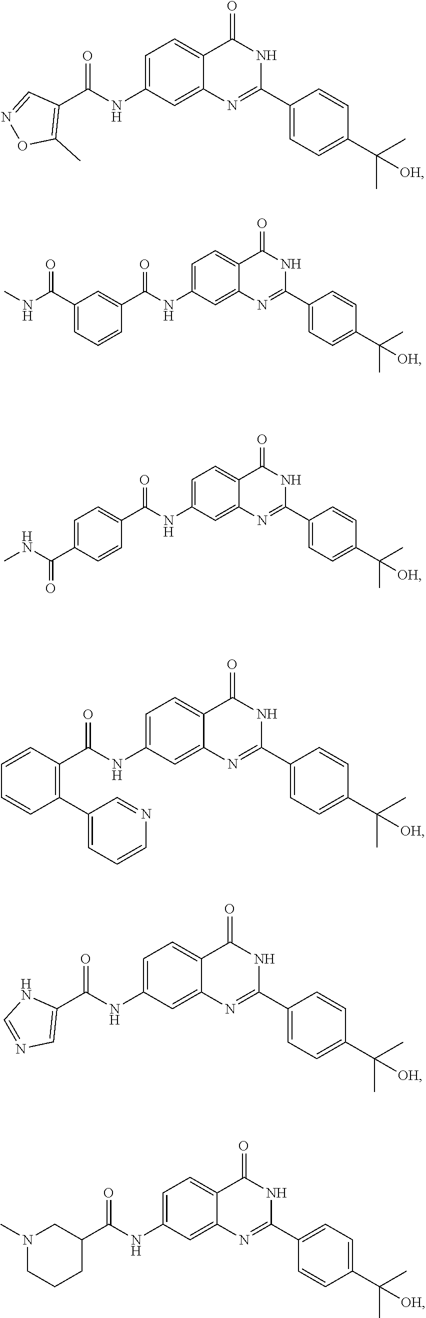 [SMILES: Cc1oncc1C(=O)Nc1ccc2c(=O)[nH]c(-c3ccc(C(C)(C)O)cc3)nc2c1 ; CNC(=O)c1cccc(C(=O)Nc2ccc3c(=O)[nH]c(-c4ccc(C(C)(C)O)cc4)nc3c2)c1 ; CNC(=O)c1ccc(C(=O)Nc2ccc3c(=O)[nH]c(-c4ccc(C(C)(C)O)cc4)nc3c2)cc1 ; CC(C)(O)c1ccc(-c2nc3cc(NC(=O)c4ccccc4-c4cccnc4)ccc3c(=O)[nH]2)cc1 ; CC(C)(O)c1ccc(-c2nc3cc(NC(=O)c4cnc[nH]4)ccc3c(=O)[nH]2)cc1 ; CN1CCCC(C(=O)Nc2ccc3c(=O)[nH]c(-c4ccc(C(C)(C)O)cc4)nc3c2)C1]

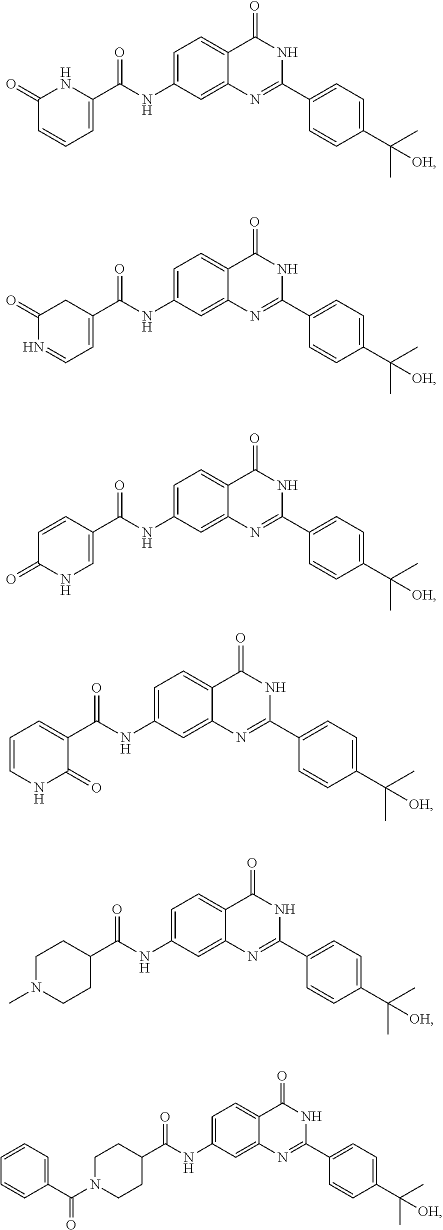 [SMILES: CC(C)(O)c1ccc(-c2nc3cc(NC(=O)c4cccc(=O)[nH]4)ccc3c(=O)[nH]2)cc1 ; CC(C)(O)c1ccc(-c2nc3cc(NC(=O)C4=CC=NC(=O)C4)ccc3c(=O)[nH]2)cc1 ; CC(C)(O)c1ccc(-c2nc3cc(NC(=O)c4ccc(=O)[nH]c4)ccc3c(=O)[nH]2)cc1 ; CC(C)(O)c1ccc(-c2nc3cc(NC(=O)c4ccc[nH]c4=O)ccc3c(=O)[nH]2)cc1 ; CN1CCC(C(=O)Nc2ccc3c(=O)[nH]c(-c4ccc(C(C)(C)O)cc4)nc3c2)CC1 ; CC(C)(O)c1ccc(-c2nc3cc(NC(=O)C4CCN(C(=O)c5ccccc5)CC4)ccc3c(=O)[nH]2)cc1]

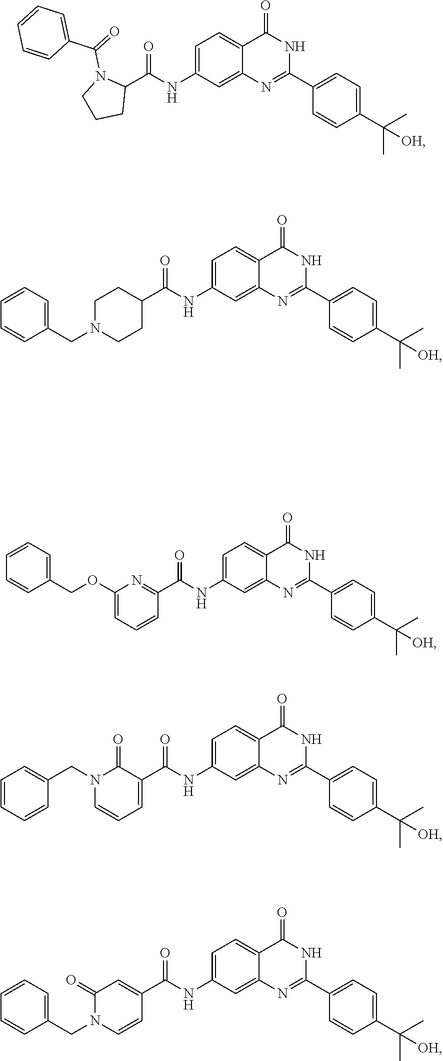 [SMILES: CC(C)(O)c1ccc(-c2nc3cc(NC(=O)C4CCCN4C(=O)c4ccccc4)ccc3c(=O)[nH]2)cc1 ; CC(C)(O)c1ccc(-c2nc3cc(NC(=O)C4CCN(Cc5ccccc5)CC4)ccc3c(=O)[nH]2)cc1 ; CC(C)(O)c1ccc(-c2nc3cc(NC(=O)c4cccc(OCc5ccccc5)n4)ccc3c(=O)[nH]2)cc1 ; CC(C)(O)c1ccc(-c2nc3cc(NC(=O)c4cccn(Cc5ccccc5)c4=O)ccc3c(=O)[nH]2)cc1 ; CC(C)(O)c1ccc(-c2nc3cc(NC(=O)c4ccn(Cc5ccccc5)c(=O)c4)ccc3c(=O)[nH]2)cc1]

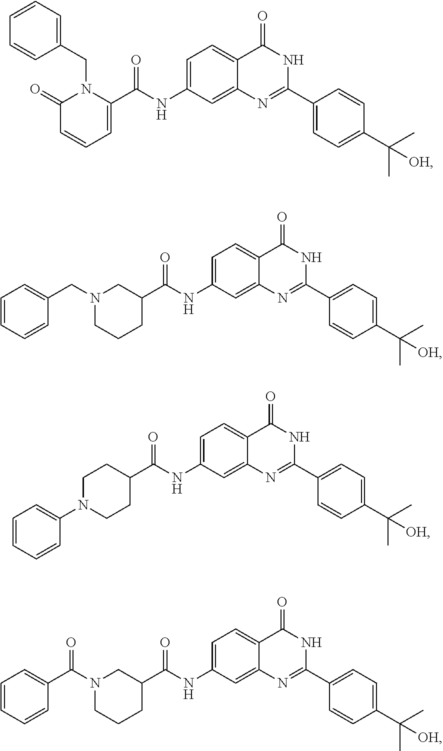 [SMILES: CC(C)(O)c1ccc(-c2nc3cc(NC(=O)c4cccc(=O)n4Cc4ccccc4)ccc3c(=O)[nH]2)cc1 ; CC(C)(O)c1ccc(-c2nc3cc(NC(=O)C4CCCN(Cc5ccccc5)C4)ccc3c(=O)[nH]2)cc1 ; CC(C)(O)c1ccc(-c2nc3cc(NC(=O)C4CCN(c5ccccc5)CC4)ccc3c(=O)[nH]2)cc1 ; CC(C)(O)c1ccc(-c2nc3cc(NC(=O)C4CCCN(C(=O)c5ccccc5)C4)ccc3c(=O)[nH]2)cc1]

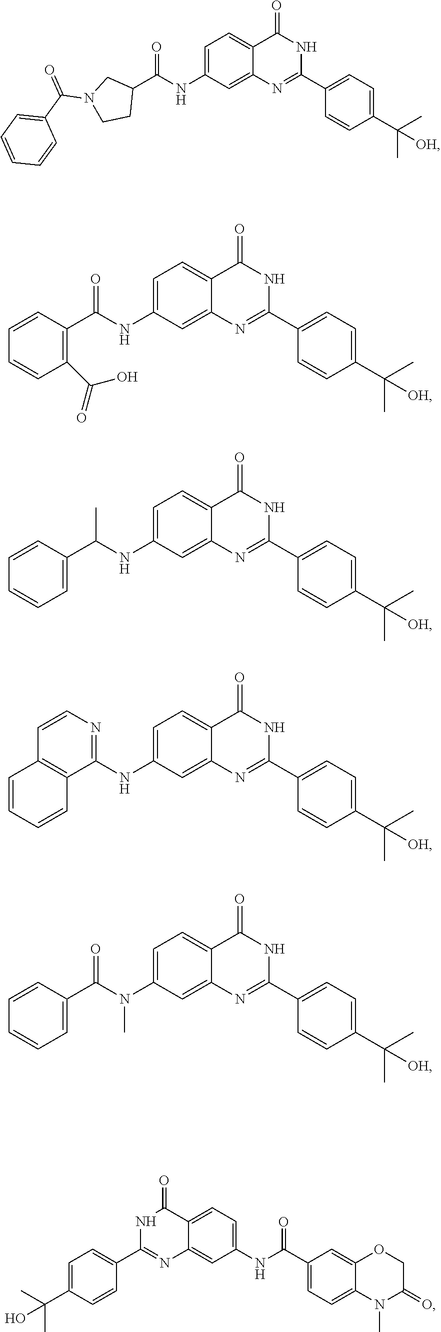 [SMILES: CC(C)(O)c1ccc(-c2nc3cc(NC(=O)C4CCN(C(=O)c5ccccc5)C4)ccc3c(=O)[nH]2)cc1 ; CC(C)(O)c1ccc(-c2nc3cc(NC(=O)c4ccccc4C(=O)O)ccc3c(=O)[nH]2)cc1 ; CC(Nc1ccc2c(=O)[nH]c(-c3ccc(C(C)(C)O)cc3)nc2c1)c1ccccc1 ; CC(C)(O)c1ccc(-c2nc3cc(Nc4nccc5ccccc45)ccc3c(=O)[nH]2)cc1 ; CN(C(=O)c1ccccc1)c1ccc2c(=O)[nH]c(-c3ccc(C(C)(C)O)cc3)nc2c1 ; CN1C(=O)COc2cc(C(=O)Nc3ccc4c(=O)[nH]c(-c5ccc(C(C)(C)O)cc5)nc4c3)ccc21]

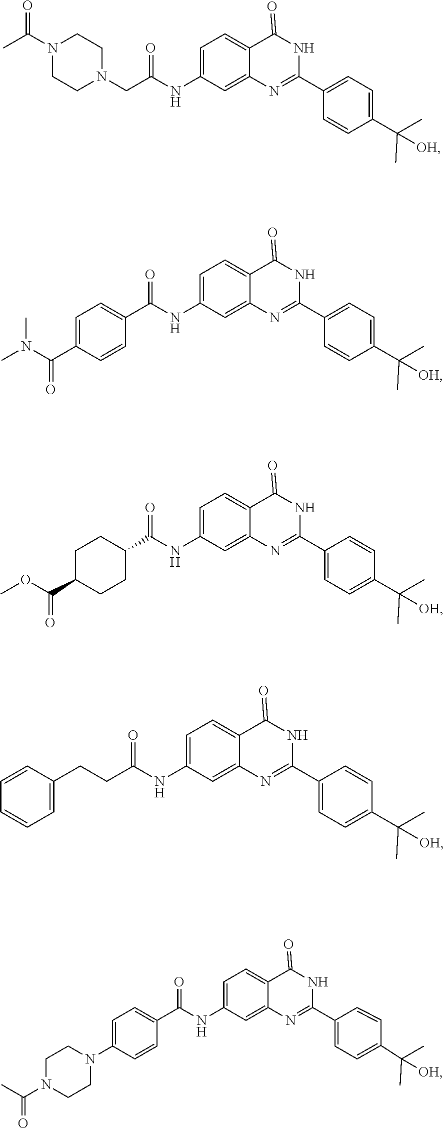 [SMILES: CC(=O)N1CCN(CC(=O)Nc2ccc3c(=O)[nH]c(-c4ccc(C(C)(C)O)cc4)nc3c2)CC1 ; CN(C)C(=O)c1ccc(C(=O)Nc2ccc3c(=O)[nH]c(-c4ccc(C(C)(C)O)cc4)nc3c2)cc1 ; COC(=O)[C@H]1CC[C@H](C(=O)Nc2ccc3c(=O)[nH]c(-c4ccc(C(C)(C)O)cc4)nc3c2)CC1 ; CC(C)(O)c1ccc(-c2nc3cc(NC(=O)CCc4ccccc4)ccc3c(=O)[nH]2)cc1 ; CC(=O)N1CCN(c2ccc(C(=O)Nc3ccc4c(=O)[nH]c(-c5ccc(C(C)(C)O)cc5)nc4c3)cc2)CC1]

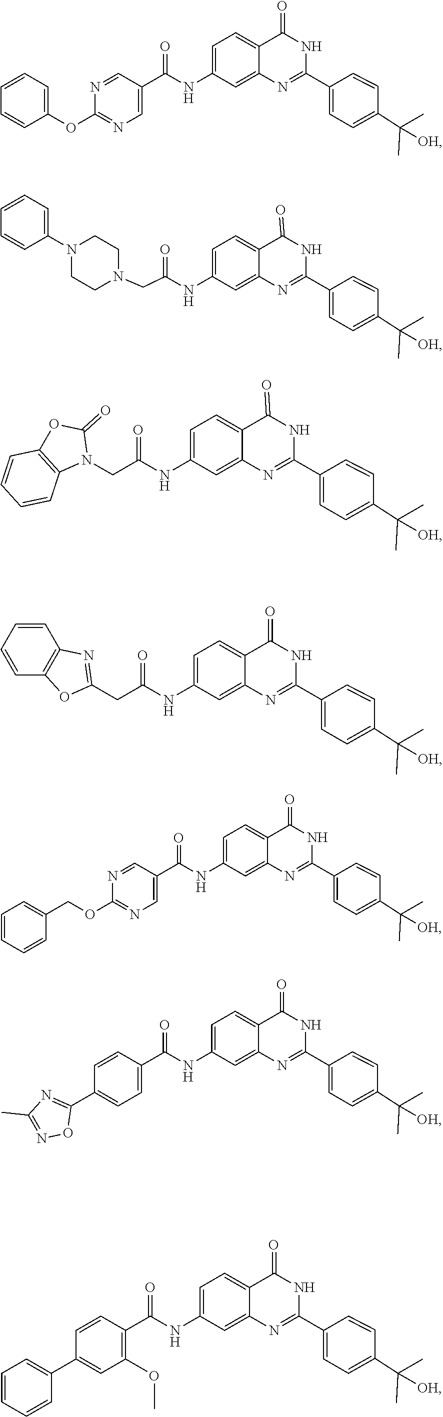

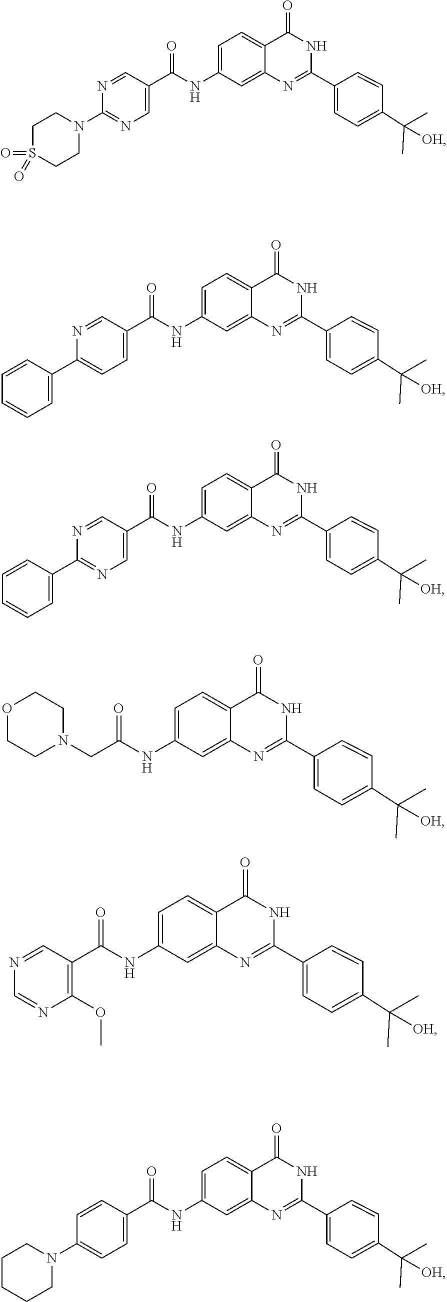 [SMILES: CC(C)(O)c1ccc(-c2nc3cc(NC(=O)c4cnc(N5CCS(=O)(=O)CC5)nc4)ccc3c(=O)[nH]2)cc1 ; CC(C)(O)c1ccc(-c2nc3cc(NC(=O)c4ccc(-c5ccccc5)nc4)ccc3c(=O)[nH]2)cc1 ; CC(C)(O)c1ccc(-c2nc3cc(NC(=O)c4cnc(-c5ccccc5)nc4)ccc3c(=O)[nH]2)cc1 ; CC(C)(O)c1ccc(-c2nc3cc(NC(=O)CN4CCOCC4)ccc3c(=O)[nH]2)cc1 ; COc1ncncc1C(=O)Nc1ccc2c(=O)[nH]c(-c3ccc(C(C)(C)O)cc3)nc2c1 ; CC(C)(O)c1ccc(-c2nc3cc(NC(=O)c4ccc(N5CCCCC5)cc4)ccc3c(=O)[nH]2)cc1]

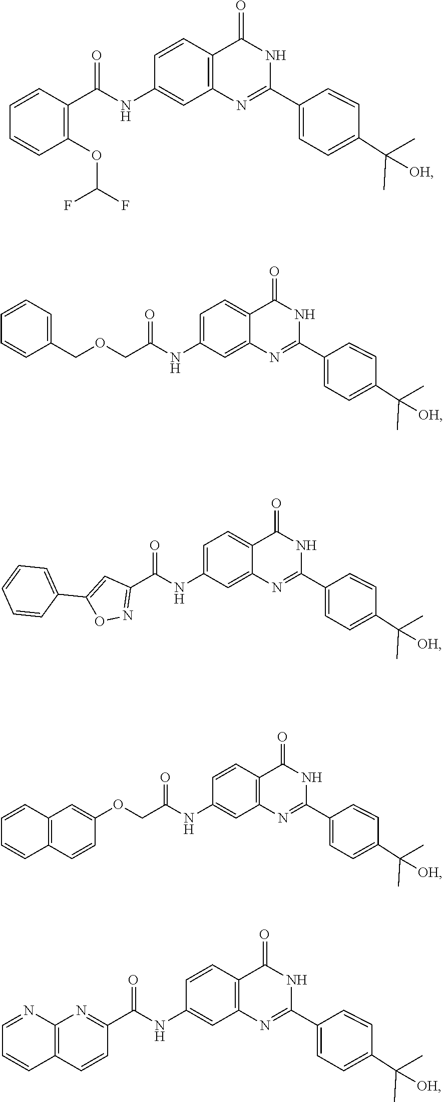 [SMILES: CC(C)(O)c1ccc(-c2nc3cc(NC(=O)c4ccccc4OC(F)F)ccc3c(=O)[nH]2)cc1 ; CC(C)(O)c1ccc(-c2nc3cc(NC(=O)COCc4ccccc4)ccc3c(=O)[nH]2)cc1 ; CC(C)(O)c1ccc(-c2nc3cc(NC(=O)c4cc(-c5ccccc5)on4)ccc3c(=O)[nH]2)cc1 ; CC(C)(O)c1ccc(-c2nc3cc(NC(=O)COc4ccc5ccccc5c4)ccc3c(=O)[nH]2)cc1 ; CC(C)(O)c1ccc(-c2nc3cc(NC(=O)c4ccc5cccnc5n4)ccc3c(=O)[nH]2)cc1]

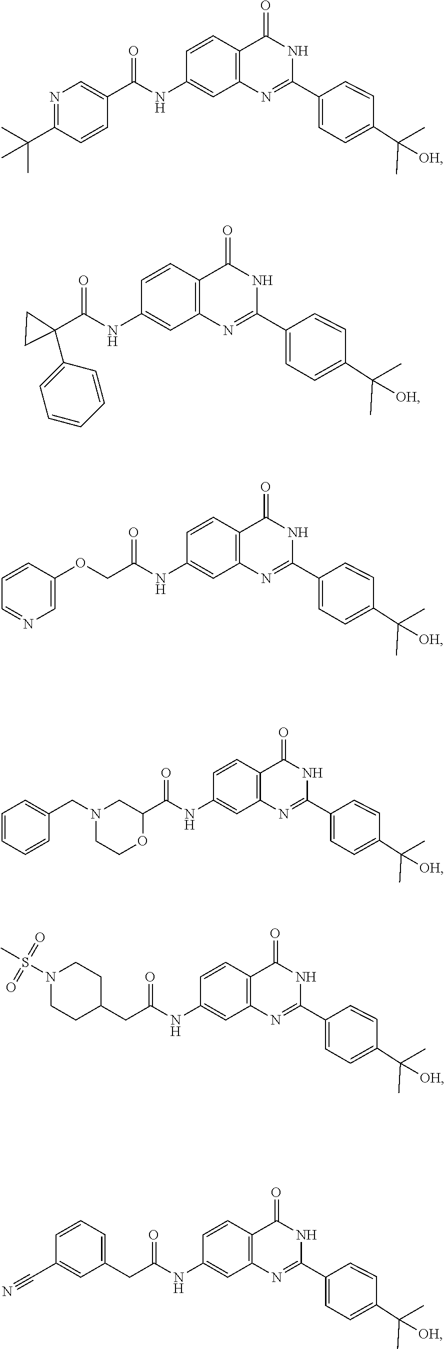 [SMILES: CC(C)(C)c1ccc(C(=O)Nc2ccc3c(=O)[nH]c(-c4ccc(C(C)(C)O)cc4)nc3c2)cn1 ; CC(C)(O)c1ccc(-c2nc3cc(NC(=O)C4(c5ccccc5)CC4)ccc3c(=O)[nH]2)cc1 ; CC(C)(O)c1ccc(-c2nc3cc(NC(=O)COc4cccnc4)ccc3c(=O)[nH]2)cc1 ; CC(C)(O)c1ccc(-c2nc3cc(NC(=O)C4CN(Cc5ccccc5)CCO4)ccc3c(=O)[nH]2)cc1 ; CC(C)(O)c1ccc(-c2nc3cc(NC(=O)CC4CCN(S(C)(=O)=O)CC4)ccc3c(=O)[nH]2)cc1 ; CC(C)(O)c1ccc(-c2nc3cc(NC(=O)Cc4cccc(C#N)c4)ccc3c(=O)[nH]2)cc1]

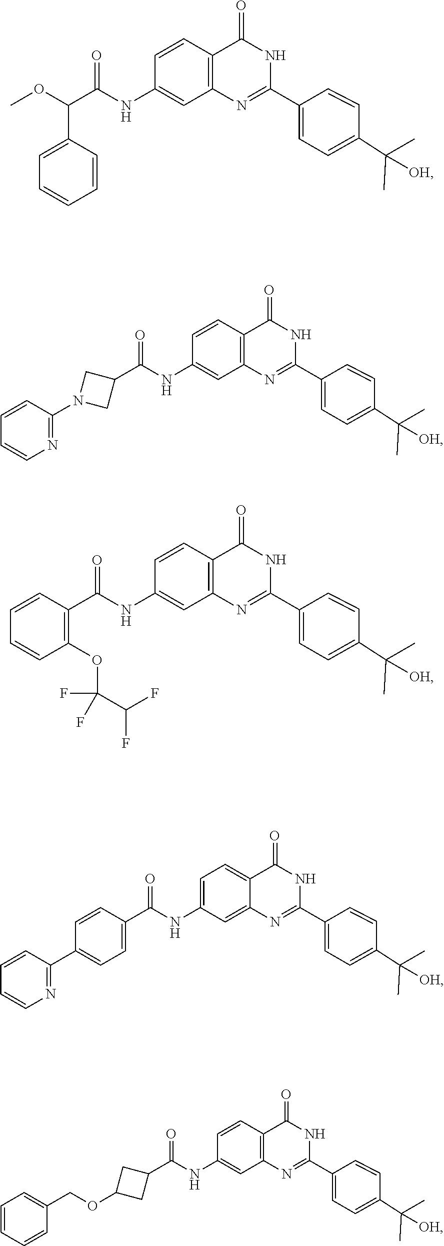 [SMILES: COC(C(=O)Nc1ccc2c(=O)[nH]c(-c3ccc(C(C)(C)O)cc3)nc2c1)c1ccccc1 ; CC(C)(O)c1ccc(-c2nc3cc(NC(=O)C4CN(c5ccccn5)C4)ccc3c(=O)[nH]2)cc1 ; CC(C)(O)c1ccc(-c2nc3cc(NC(=O)c4ccccc4OC(F)(F)C(F)F)ccc3c(=O)[nH]2)cc1 ; CC(C)(O)c1ccc(-c2nc3cc(NC(=O)c4ccc(-c5ccccn5)cc4)ccc3c(=O)[nH]2)cc1 ; CC(C)(O)c1ccc(-c2nc3cc(NC(=O)C4CC(OCc5ccccc5)C4)ccc3c(=O)[nH]2)cc1]

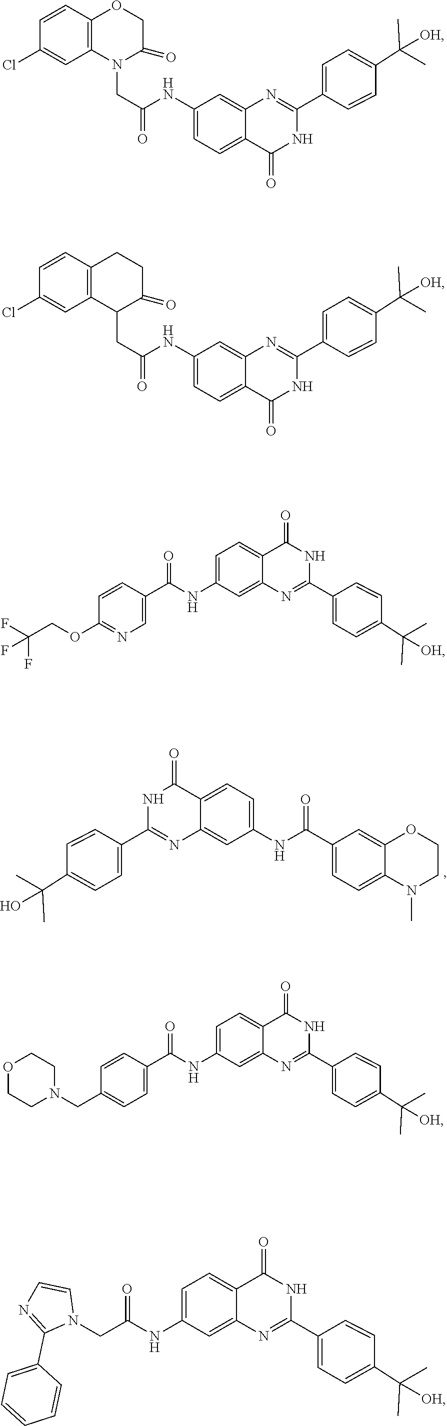 [SMILES: CC(C)(O)c1ccc(-c2nc3cc(NC(=O)CN4C(=O)COc5ccc(Cl)cc54)ccc3c(=O)[nH]2)cc1 ; CC(C)(O)c1ccc(-c2nc3cc(NC(=O)CC4C(=O)CCc5ccc(Cl)cc54)ccc3c(=O)[nH]2)cc1 ; CC(C)(O)c1ccc(-c2nc3cc(NC(=O)c4ccc(OCC(F)(F)F)nc4)ccc3c(=O)[nH]2)cc1 ; CN1CCOc2cc(C(=O)Nc3ccc4c(=O)[nH]c(-c5ccc(C(C)(C)O)cc5)nc4c3)ccc21 ; CC(C)(O)c1ccc(-c2nc3cc(NC(=O)c4ccc(CN5CCOCC5)cc4)ccc3c(=O)[nH]2)cc1 ; CC(C)(O)c1ccc(-c2nc3cc(NC(=O)Cn4ccnc4-c4ccccc4)ccc3c(=O)[nH]2)cc1]

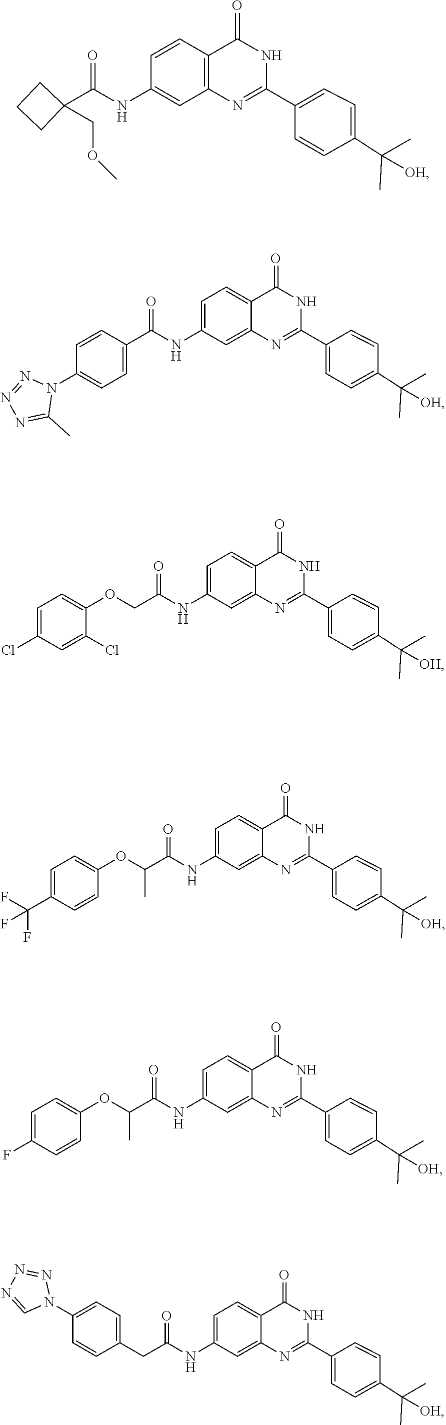 [SMILES: COCC1(C(=O)Nc2ccc3c(=O)[nH]c(-c4ccc(C(C)(C)O)cc4)nc3c2)CCC1 ; Cc1nnnn1-c1ccc(C(=O)Nc2ccc3c(=O)[nH]c(-c4ccc(C(C)(C)O)cc4)nc3c2)cc1 ; CC(C)(O)c1ccc(-c2nc3cc(NC(=O)COc4ccc(Cl)cc4Cl)ccc3c(=O)[nH]2)cc1 ; CC(Oc1ccc(C(F)(F)F)cc1)C(=O)Nc1ccc2c(=O)[nH]c(-c3ccc(C(C)(C)O)cc3)nc2c1 ; CC(Oc1ccc(F)cc1)C(=O)Nc1ccc2c(=O)[nH]c(-c3ccc(C(C)(C)O)cc3)nc2c1 ; CC(C)(O)c1ccc(-c2nc3cc(NC(=O)Cc4ccc(-n5cnnn5)cc4)ccc3c(=O)[nH]2)cc1]

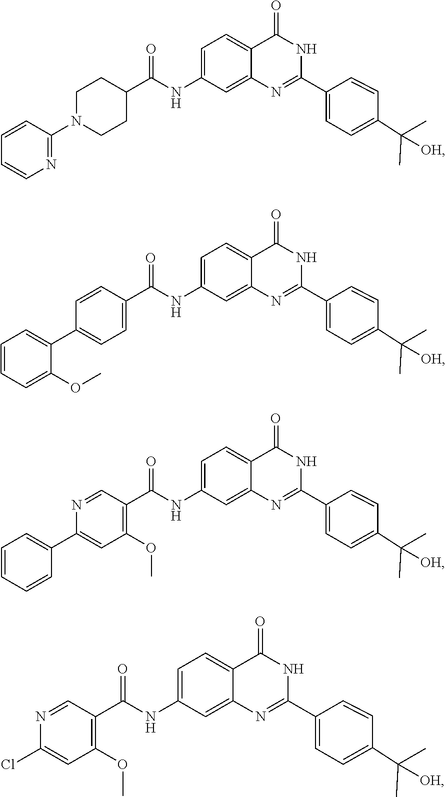 [SMILES: CC(C)(O)c1ccc(-c2nc3cc(NC(=O)C4CCN(c5ccccn5)CC4)ccc3c(=O)[nH]2)cc1 ; COc1ccccc1-c1ccc(C(=O)Nc2ccc3c(=O)[nH]c(-c4ccc(C(C)(C)O)cc4)nc3c2)cc1 ; COc1cc(-c2ccccc2)ncc1C(=O)Nc1ccc2c(=O)[nH]c(-c3ccc(C(C)(C)O)cc3)nc2c1 ; COc1cc(Cl)ncc1C(=O)Nc1ccc2c(=O)[nH]c(-c3ccc(C(C)(C)O)cc3)nc2c1]

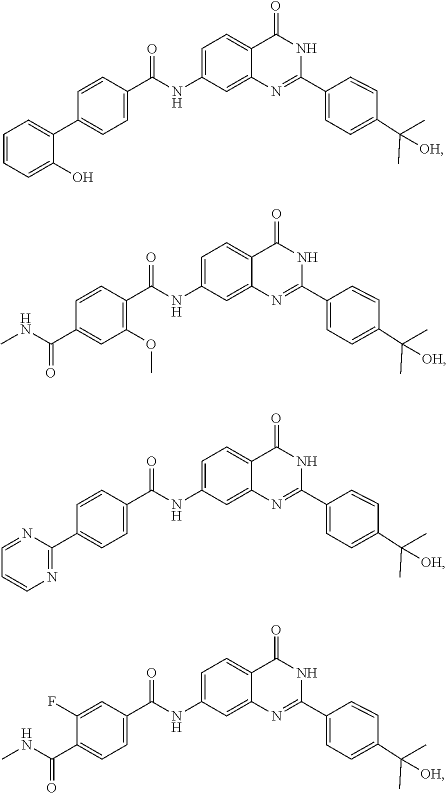 [SMILES: CC(C)(O)c1ccc(-c2nc3cc(NC(=O)c4ccc(-c5ccccc5O)cc4)ccc3c(=O)[nH]2)cc1 ; CNC(=O)c1ccc(C(=O)Nc2ccc3c(=O)[nH]c(-c4ccc(C(C)(C)O)cc4)nc3c2)c(OC)c1 ; CC(C)(O)c1ccc(-c2nc3cc(NC(=O)c4ccc(-c5ncccn5)cc4)ccc3c(=O)[nH]2)cc1 ; CNC(=O)c1ccc(C(=O)Nc2ccc3c(=O)[nH]c(-c4ccc(C(C)(C)O)cc4)nc3c2)cc1F]

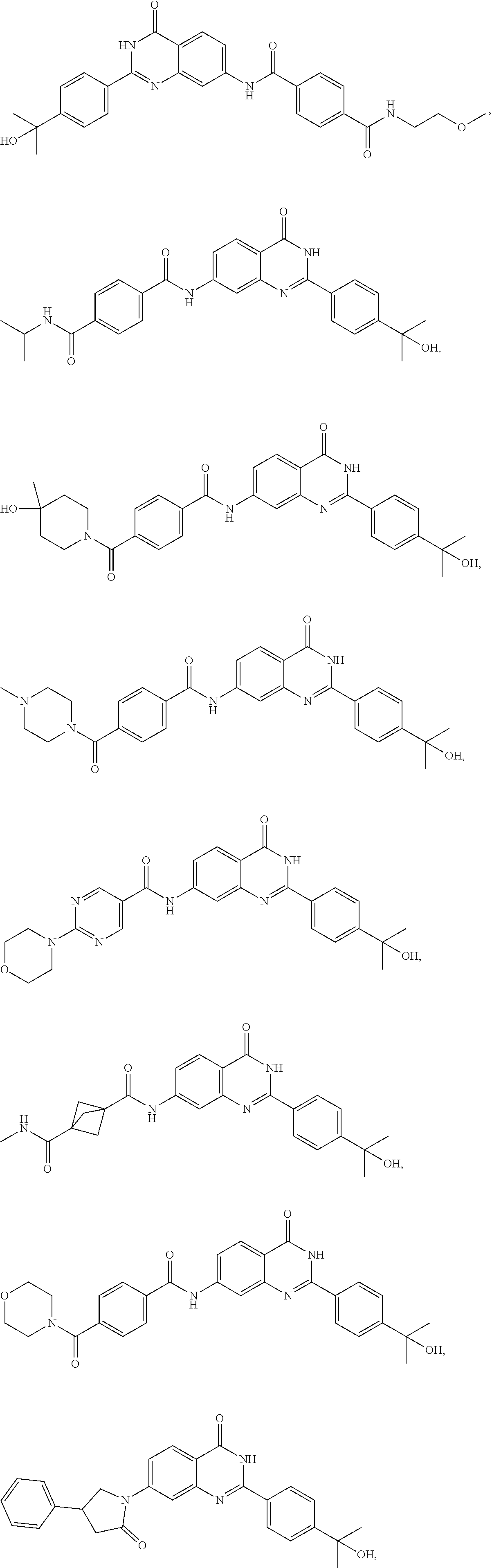

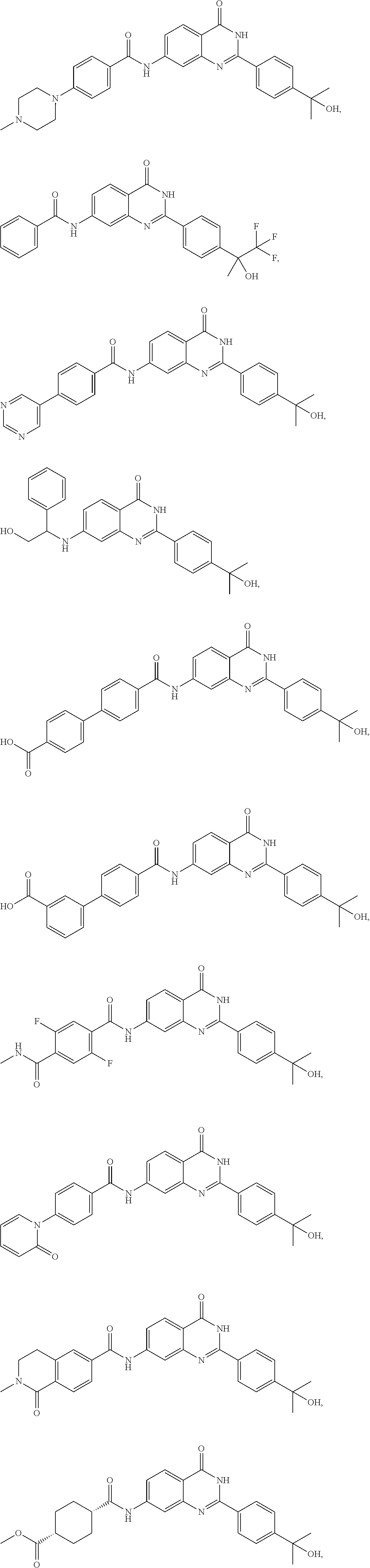

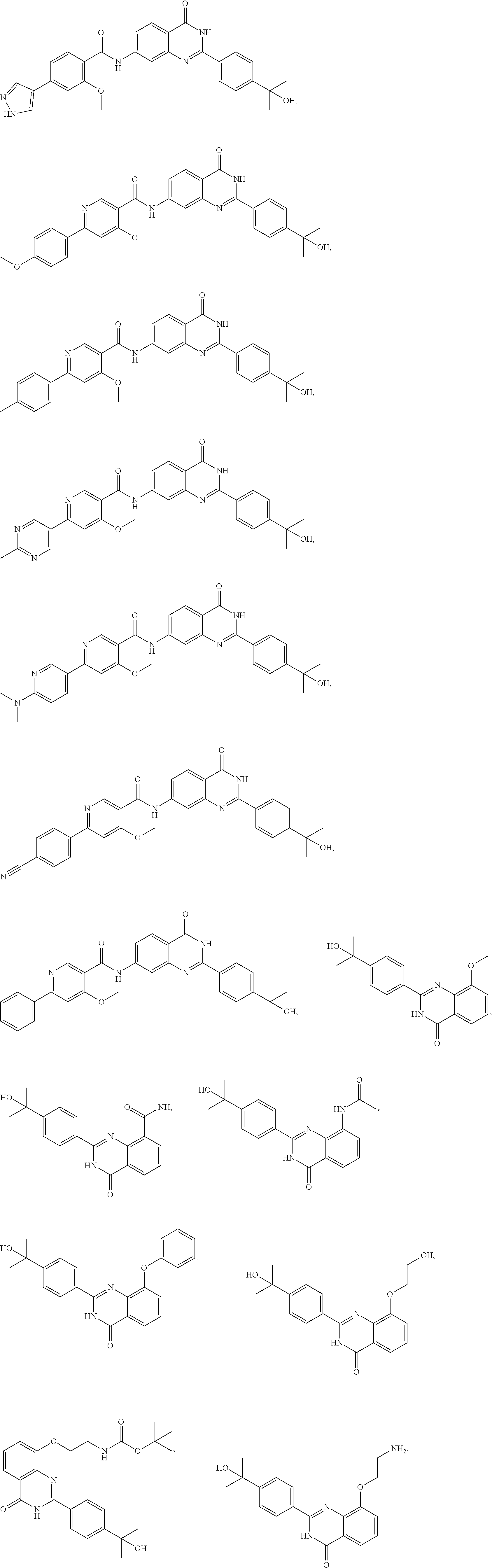

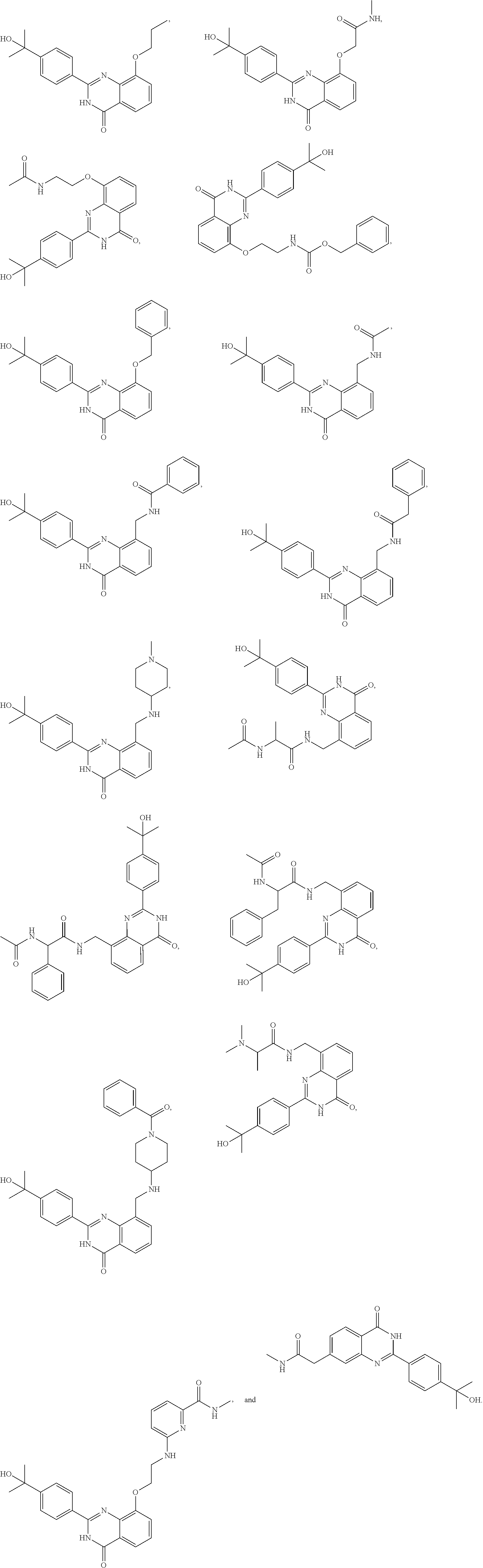

10. A pharmaceutical composition comprising a therapeutically effective amount of the compound according to claim 1 and a pharmaceutically acceptable carrier.

11. A method of treating cancer in a subject, said method comprising:
administering to the subject in need thereof the compound according to claim 1.

12. The method of claim 11, wherein the cancer is selected from the group consisting of small cell lung carcinoma, non-small cell lung carcinoma, esophageal cancer, melanoma, liver cancer, lung cancer, sarcoma, cholangiocarcinoma, mesothelioma, gastric cancer, hepatocellular cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

13. A method of inhibiting Tankyrase 2 (TNKS2) activity, said method comprising:
contacting a TNKS2 with the compound according to claim 1 under conditions effective to inhibit TNKS2 activity.

14.-15. (canceled)

16. A method of treating a condition where it is desired to inhibit TNKS2 activity, said method comprising:
administering to the subject in need thereof the compound according to claim 1.

17. A method of treating a proliferation-related disorder in a subject, said method comprising:
administering to the subject in need thereof the compound according to claim 1.

18. A method of treating abnormal cell growth in a subject, said method comprising:
administering to the subject in need thereof the compound according to claim 1.

19. The method of claim 18, wherein the abnormal cell growth is a benign growth or a malignant growth.

20. The method of claim 18, wherein the abnormal cell growth is a psoriasis, benign prostatic hypertrophy, or restenosis.

21. The method of claim 18, wherein the abnormal cell growth is carcinoma, sarcoma, lymphoma, or leukemia.

22. The method of claim 18, wherein the abnormal cell growth is a cancer.

23.-27. (canceled)


SEQUENCE LISTING (60 sequences)

SEQ ID NO: 1 - DNA, 20 bp, synthetic construct
  source 1..20: mol_type = other DNA
  misc_feature 1..20: note = hRPL27_F1 qPCR Primer
catgggcaagaagaagatcg

SEQ ID NO: 2 - DNA, 20 bp, synthetic construct
  source 1..20: mol_type = other DNA
  misc_feature 1..20: note = hRPL27_R1 qPCR Primer
tccaaggggatatccacaga

SEQ ID NO: 3 - DNA, 20 bp, synthetic construct
  source 1..20: mol_type = other DNA
  misc_feature 1..20: note = Krt20_F1 qPCR Primer
cccagaagaacctgcaagag

SEQ ID NO: 4 - DNA, 20 bp, synthetic construct
  source 1..20: mol_type = other DNA
  misc_feature 1..20: note = Krt20_R1 qPCR Primer
cctccgtgttcactgtgact

SEQ ID NO: 5 - DNA, 97 bp, synthetic construct
  source 1..97: mol_type = other DNA
  misc_feature 1..97: note = shRenilla.713 Cloning Primer
tgctgttgacagtgagcgcaggaattataatgcttatctatagtgaagccacagatgtatagataagcattataattcctatgcctactgcctcgga

SEQ ID NO: 6 - DNA, 97 bp, synthetic construct
  source 1..97: mol_type = other DNA
  misc_feature 1..97: note = shTNKS.2658 Cloning Primer
tgctgttgacagtgagcgcagcggctttattgataaaatatagtgaagccacagatgtatattttatcaataaagccgctatgcctactgcctcgga

SEQ ID NO: 7 - DNA, 97 bp, synthetic construct
  source 1..97: mol_type = other DNA
  misc_feature 1..97: note = shTNKS2.3287 Cloning Primer
tgctgttgacagtgagcgccagaaggtttgtaacaagaaatagtgaagccacagatgtatttcttgttacaaaccttctgatgcctactgcctcgga

SEQ ID NO: 8 - DNA, 97 bp, synthetic construct
  source 1..97: mol_type = other DNA
  misc_feature 1..97: note = Tnks_1385 Cloning Primer
tgctgttgacagtgagcgaaagtctgttctctgttacttatagtgaagccacagatgtataagtaacagagaacagacttctgcctactgcctcgga

SEQ ID NO: 9 - DNA, 97 bp, synthetic construct
  source 1..97: mol_type = other DNA
  misc_feature 1..97: note = Tnks_3341 Cloning Primer
tgctgttgacagtgagcgcccagaagataaagaatatcaatagtgaagccacagatgtattgatattctttatcttctggatgcctactgcctcgga

SEQ ID NO: 10 - DNA, 97 bp, synthetic construct
  source 1..97: mol_type = other DNA
  misc_feature 1..97: note = Tnks2_1328 Cloning Primer
tgctgttgacagtgagcgcgctgatgttacaagaatcaaatagtgaagccacagatgtatttgattcttgtaacatcagcttgcctactgcctcgga

SEQ ID NO: 11 - DNA, 97 bp, synthetic construct
  source 1..97: mol_type = other DNA
  misc_feature 1..97: note = Tnks2_3004 Cloning Primer
tgctgttgacagtgagcgcaccttagatgtcctagttgaatagtgaagccacagatgtattcaactaggacatctaaggtatgcctactgcctcgga

SEQ ID NO: 12 - DNA, 22 bp, synthetic construct
  source 1..22: mol_type = other DNA
  misc_feature 1..22: note = Tnks_5-2_F Cloning Primer
caccgggggagtccgaagatgg

SEQ ID NO: 13 - DNA, 22 bp, synthetic construct
  source 1..22: mol_type = other DNA
  misc_feature 1..22: note = Tnks_5-2_R Cloning Primer
aaacccatcttcggactccccc

SEQ ID NO: 14 - DNA, 24 bp, synthetic construct
  source 1..24: mol_type = other DNA
  misc_feature 1..24: note = Apc.1405_A Cloning Primer
caccgttcagagtgagccatgtag

SEQ ID NO: 15 - DNA, 24 bp, synthetic construct
  source 1..24: mol_type = other DNA
  misc_feature 1..24: note = Apc.1405_B Cloning Primer
aaacctacatggctcactctgaac

SEQ ID NO: 16 - DNA, 24 bp, synthetic construct
  source 1..24: mol_type = other DNA
  misc_feature 1..24: note = Trp53.Q97_A Cloning Primer
caccgcttctcaaaaaacttacca

SEQ ID NO: 17 - DNA, 24 bp, synthetic construct
  source 1..24: mol_type = other DNA
  misc_feature 1..24: note = Trp53.Q97_B Cloning Primer
aaactggtaagttttttgagaagc

SEQ ID NO: 18 - DNA, 19 bp, synthetic construct
  source 1..19: mol_type = other DNA
  misc_feature 1..19: note = Tnks 5' Cloning Primer
gtacgggggagtgggaaga

SEQ ID NO: 19 - DNA, 20 bp, synthetic construct
  source 1..20: mol_type = other DNA
  misc_feature 1..20: note = Tnks 3' Cloning Primer
gctagagtgacagtgcagag

SEQ ID NO: 20 - DNA, 145 bp, synthetic construct
  source 1..145: mol_type = other DNA
  misc_feature 1..145: note = SFFV_3xFlag_TNKS2 Cloning Primer
aaagagctcacaacccctcactcggcgcgccagtcctccgacaggtggatccaccatggattataaggatcacgatggcgattataaggaccacgacatcgactacaaggacgacgacgacaagtcgggtcgccgctgcgccggc

SEQ ID NO: 21 - DNA, 25 bp, synthetic construct
  source 1..25: mol_type = other DNA
  misc_feature 1..25: note = GA_Flag_TNKS2_F Cloning Primer
cgacaagtcgggtcgccgctgcgcc

SEQ ID NO: 22 - DNA, 51 bp, synthetic construct
  source 1..51: mol_type = other DNA
  misc_feature 1..51: note = GA_Flag_TNKS2_R Cloning Primer
agtaaacaagataattgctcgaattttatccatcgaccataccttcaggcc

SEQ ID NO: 23 - DNA, 21 bp, synthetic construct
  source 1..21: mol_type = other DNA
  misc_feature 1..21: note = TNKS2_F Cloning Primer
agtacagttcgagagcacaga

SEQ ID NO: 24 - DNA, 20 bp, synthetic construct
  source 1..20: mol_type = other DNA
  misc_feature 1..20: note = TNKS2_int_Rev Cloning Primer
tgctaggccatttacactgg

SEQ ID NO: 25 - DNA, 43 bp, synthetic construct
  source 1..43: mol_type = other DNA
  misc_feature 1..43: note = TNKS2_L1136Y_For Cloning Primer
ccagtgtaaatggcctagcatatgctgaatatgttatttacag

SEQ ID NO: 26 - DNA, 21 bp, synthetic construct
  source 1..21: mol_type = other DNA
  misc_feature 1..21: note = TNKS2_Rev Cloning Primer
aagaacggagccggttggcgc

SEQ ID NO: 27 - DNA, 37 bp, synthetic construct
  source 1..37: mol_type = other DNA
  misc_feature 1..37: note = miRE-XhoI-Fw_for oligo Cloning Primer
tgaactcgagaaggtatattgctgttgacagtgagcg

SEQ ID NO: 28 - DNA, 38 bp, synthetic construct
  source 1..38: mol_type = other DNA
  misc_feature 1..38: note = miRE-EcoRI-Rev_for oligo Cloning Primer
tctcgaattctagccccttgaagtccgaggcagtaggc

SEQ ID NO: 29 - DNA, 54 bp, synthetic construct
  source 1..54: mol_type = other DNA
  misc_feature 1..54: note = TOP_FF_Fwd Cloning Primer
cgatcacgagactagcctcgatcgagctagctgccagaacatttctctatcgat

SEQ ID NO: 30 - DNA, 60 bp, synthetic construct
  source 1..60: mol_type = other DNA
  misc_feature 1..60: note = TOP_FF_Rev Cloning Primer
ttagtaaacaagataattgctcccgccccgtctccaattattacacggcgatctttccgc

SEQ ID NO: 31 - DNA, 50 bp, synthetic construct
  source 1..50: mol_type = other DNA
  misc_feature 1..50: note = Ren-P2A-iRFP_F Cloning Primer
tggtataaattaaatcacttttttcaattgctcgagtggctccggtgccc

SEQ ID NO: 32 - DNA, 48 bp, synthetic construct
  source 1..48: mol_type = other DNA
  misc_feature 1..48: note = Ren-P2A-iRFP_R Cloning Primer
tgtaatccagaggttgattgttccaggctagctcactcttccatcacg

SEQ ID NO: 33 - DNA, 47 bp, synthetic construct
  source 1..47: mol_type = other DNA
  misc_feature 1..47: note = GA_SFFV_Fwd Cloning Primer
gatcacgagactagcctcgatcgagctagctgcagtaacgccatttt

SEQ ID NO: 34 - DNA, 47 bp, synthetic construct
  source 1..47: mol_type = other DNA
  misc_feature 1..47: note = GA_SFFV_Rev Cloning Primer
ccggtagatccaccggccgactcagtctgtcggaggactggcgcgcc

SEQ ID NO: 35 - DNA, 51 bp, synthetic construct
  source 1..51: mol_type = other DNA
  misc_feature 1..51: note = GA_BadKozak_GFP_Fwd Cloning Primer
gtcggccggtggatctaccggtggataacaaatggtgagcaagggcgagga

SEQ ID NO: 36 - DNA, 45 bp, synthetic construct
  source 1..45: mol_type = other DNA
  misc_feature 1..45: note = GA_GFP_Rev Cloning Primer
ccaccgatcccctccggacttgtacagctcgtccatgccgagagt

SEQ ID NO: 37 - DNA, 45 bp, synthetic construct
  source 1..45: mol_type = other DNA
  misc_feature 1..45: note = U6-TNKS-F Cloning Primer
tcgatcacgagactagcctcgatcggagggcctatttcccatgat

SEQ ID NO: 38 - DNA, 45 bp, synthetic construct
  source 1..45: mol_type = other DNA
  misc_feature 1..45: note = U6-TNKS-R Cloning Primer
caaaatggcgttactgcagctagctatactgccatttgtctcaag

SEQ ID NO: 39 - DNA, 45 bp, synthetic construct
  source 1..45: mol_type = other DNA
  misc_feature 1..45: note = NLS-GFP-F Cloning Primer
ccggtggatctaccggtggatccactcggctggcggttccacagg

SEQ ID NO: 40 - DNA, 45 bp, synthetic construct
  source 1..45: mol_type = other DNA
  misc_feature 1..45: note = NLS-GFP-R Cloning Primer
ctacccggtagaattacgcgtcgaacccaaccctataaaactagg

SEQ ID NO: 41 - DNA, 21 bp, synthetic construct
  source 1..21: mol_type = other DNA
  misc_feature 1..21: note = Rosa_A Mouse Genotyping Primer
aaagtcgctctgagttgttat

SEQ ID NO: 42 - DNA, 21 bp, synthetic construct
  source 1..21: mol_type = other DNA
  misc_feature 1..21: note = Rosa_B Mouse Genotyping Primer
gcgaagagtttgtcctcaacc

SEQ ID NO: 43 - DNA, 21 bp, synthetic construct
  source 1..21: mol_type = other DNA
  misc_feature 1..21: note = Rosa_C5 Mouse Genotyping Primer
cctccaattttacacctgttc

SEQ ID NO: 44 - DNA, 27 bp, synthetic construct
  source 1..27: mol_type = other DNA
  misc_feature 1..27: note = Col1a1_F Mouse Genotyping Primer
aatcatcccaggtgcacagcattgcgg

SEQ ID NO: 45 - DNA, 26 bp, synthetic construct
  source 1..26: mol_type = other DNA
  misc_feature 1..26: note = Col1a1_R Mouse Genotyping Primer
ctttgagggctcatgaacctcccagg

SEQ ID NO: 46 - DNA, 25 bp, synthetic construct
  source 1..25: mol_type = other DNA
  misc_feature 1..25: note = SAdpA_R Mouse Genotyping Primer
atcaaggaaaccctggactactgcg

SEQ ID NO: 47 - DNA, 26 bp, synthetic construct
  source 1..26: mol_type = other DNA
  misc_feature 1..26: note = shRen_GT_F Mouse Genotyping Primer
aagccacagatgtatagataagcatt

SEQ ID NO: 48 - DNA, 20 bp, synthetic construct
  source 1..20: mol_type = other DNA
  misc_feature 1..20: note = RBG_R1 (Rev43) Mouse Genotyping Primer
gaaagaacaatcaagggtcc

SEQ ID NO: 49 - DNA, 24 bp, synthetic construct
  source 1..24: mol_type = other DNA
  misc_feature 1..24: note = GT_T2.3004 Mouse Genotyping Primer
agccacagatgtattcaactagga

SEQ ID NO: 50 - DNA, 24 bp, synthetic construct
  source 1..24: mol_type = other DNA
  misc_feature 1..24: note = GT_T.1385 Mouse Genotyping Primer
agccacagatgtataagtaacaga

SEQ ID NO: 51 - DNA, 26 bp, synthetic construct
  source 1..26: mol_type = other DNA
  misc_feature 1..26: note = GT_T.3341 Mouse Genotyping Primer
aagccacagatgtattgatattcttt

SEQ ID NO: 52 - DNA, 20 bp, synthetic construct
  source 1..20: mol_type = other DNA
  misc_feature 1..20: note = RBG_R2 (Rev45) Mouse Genotyping Primer
caccctgaaaactttgcccc

SEQ ID NO: 53 - DNA, 20 bp, synthetic construct
  source 1..20: mol_type = other DNA
  misc_feature 1..20: note = Tnks_3_F Mouse Genotyping Primer
cggcagccatttcctcaaag

SEQ ID NO: 54 - DNA, 20 bp, synthetic construct
  source 1..20: mol_type = other DNA
  misc_feature 1..20: note = Tnks-5-2_R Mouse Genotyping Primer
ctccactactaagccacgcc

SEQ ID NO: 55 - DNA, 20 bp, synthetic construct
  source 1..20: mol_type = other DNA
  misc_feature 1..20: note = Tnks_5F2_geno Mouse Genotyping Primer
tggcggagccgaagcccctg

SEQ ID NO: 56 - DNA, 23 bp, synthetic construct
  source 1..23: mol_type = other DNA
  misc_feature 1..23: note = LSL-Kras (Y116-common) Mouse Genotyping Primer
tccgaattcagtgactacagatg

SEQ ID NO: 57 - DNA, 20 bp, synthetic construct
  source 1..20: mol_type = other DNA
  misc_feature 1..20: note = LSL-Kras (Y118-WT) Mouse Genotyping Primer
atgtctttccccagcacagt

SEQ ID NO: 58 - DNA, 20 bp, synthetic construct
  source 1..20: mol_type = other DNA
  misc_feature 1..20: note = LSL-Kras (Y117-LSL) Mouse Genotyping Primer
ctagccaccatggcttgagt

SEQ ID NO: 59 - DNA, 22 bp, synthetic construct
  source 1..22: mol_type = other DNA
  misc_feature 1..22: note = 7a1_miseq_F1 Mouse Genotyping Primer
tgttgagttttcttcaggagcc

SEQ ID NO: 60 - DNA, 20 bp, synthetic construct
  source 1..20: mol_type = other DNA
  misc_feature 1..20: note = 7a1_miseq_R2 Mouse Genotyping Primer
tggtctgcccaggactatct